(19) United States
(12) Patent Application Publication
(10) Pub. No.: US 2026/0263638 A1
(43) Pub. Date: Sep. 10, 2026

(54) COMPOSITIONS AND METHODS TO IMPROVE RNA PROPERTIES USING BASE, PHOSPHODIESTER LINKAGE, SUGAR BACKBONE, AND CAP MODIFICATIONS

(71) Applicants: The Broad Institute, Inc., Cambridge, MA (US); Massachusetts Institute of Technology, Cambridge, MA (US)

(72) Inventors: Xiao Wang, Cambridge, MA (US); Hongyu Chen, Cambridge, MA (US); Dangliang Liu, Cambridge, MA (US)

(73) Assignees: The Broad Institute, Inc., Cambridge, MA (US); Massachusetts Institute of Technology, Cambridge, MA (US)

(21) Appl. No.: 19/545,537

(22) Filed: Feb. 20, 2026

Related U.S. Application Data
(63) Continuation of application No. PCT/US2024/043419, filed on Aug. 22, 2024.
(60) Provisional application No. 63/578,300, filed on Aug. 23, 2023.
(60) Provisional application No. 63/610,892, filed on Dec. 15, 2023.
(60) Provisional application No. 63/640,571, filed on Apr. 30, 2024.
(60) Provisional application No. 63/652,449, filed on May 28, 2024.

(51) Int. Cl. A61K 48/00; C12P 19/34

(52) U.S. Cl. CPC A61K 48/005; A61K 48/0091; C12P 19/34

(57) ABSTRACT

Disclosed herein are capped RNA transcripts comprising one or more modified nucleotides at position +3 or higher with reference to a 5′ terminus of the RNA molecule, and methods of making the same. Also provided are compositions comprising one or more of the capped RNA transcripts provided herein, and methods of using said compositions for therapeutic applications.

### CROSS-REFERENCE TO RELATED APPLICATIONS

This application is a Continuation of International Application No. PCT/US2024/043419, filed on Aug. 22, 2024, and claims the benefit under 35 U.S.C. § 119(e) of U.S. Provisional Patent Application No. 63/578,300, filed Aug. 23, 2023, U.S. Provisional Patent Application No. 63/610,892, filed Dec. 15, 2023, U.S. Provisional Patent Application No. 63/640,571, filed Apr. 30, 2024, and U.S. Provisional Patent Application No. 63/652,449, filed May 28, 2024, the entire contents of each of which is incorporated herein by reference in their entireties.

### REFERENCE TO AN ELECTRONIC SEQUENCE LISTING

The instant application contains a Sequence Listing which has been submitted electronically in XML file format and is hereby incorporated by reference in its entirety. Said XML copy, created on May 22, 2026, is named 114203-1208_SL.xml and is 462,625 bytes in size

### BACKGROUND

The following discussion is merely provided to aid the reader in understanding the disclosure and is not admitted to describe or constitute prior art thereto.

Messenger RNA (mRNA) technology is an emerging alternative to conventional small molecule, DNA, and protein therapeutics and conventional vaccine approaches because it is potent, programmable, and capable of rapid production of mRNA with desired sequences. mRNA therapy is a rapidly developing field and has been used for the expression of therapeutic proteins, ranging from vascular regeneration factors (e.g., vascular endothelial growth factor A (VEGF-A), erythropoietin (EPO), GATA Binding Protein 4 (GATA4), Myocyte Enhancer Factor 2C (MEF2C), T-Box Transcription Factor 5 (TBX5), Myocardin (MYOCD)), to vaccines for COVID-19, influenza, and Zika virus. Despite recent clinical successes, mRNA therapy still faces challenges of instability, toxicity, short-term efficacy, and potential immunological responses. Increasing the stability and translation efficiency of mRNAs to enhance their efficiency in vivo remains an important problem that must be solved to increase the feasibility of mRNA therapeutics for clinical applications.

### SUMMARY

Naturally occurring chemical modifications to the 5′ cap of mRNA have well-characterized biochemical functions and can be incorporated co-transcriptionally into synthetic mRNA using modified cap analogues. However, such a strategy is limited to modification in the first two bases of the mRNA that can be tolerated by RNA polymerase during in vitro transcription (IVT) or capping enzymes. Further diversifying and expanding chemical modifications on the cap and in the 5′ untranslated region (5′ UTR) beyond the first two bases (i.e., at or beyond position +3) is highly desirable but unexplored due to a lack of methods capable of generating such modifications.

Thus, provided herein are mRNAs with extensively modified 5′ cap regions to improve the stability and translation efficiency of the mRNAs in cells and in vitro translation systems, thereby enhancing protein production, as well as methods of making and using such modified mRNAs.

Accordingly, the present disclosure provides, in some aspects, provides a method of producing a composition of capped RNA transcripts, comprising: (a) synthesizing a 5′-phosphorylated RNA oligonucleotide in a solvent system comprising a nonpolar counterion; (b) capping the 5′ phosphorylated RNA oligonucleotide, thereby producing a capped RNA oligonucleotide; (c) purifying the capped RNA oligonucleotide, thereby producing a composition comprising a purified capped RNA oligonucleotide, wherein at least 95% of the total RNA oligonucleotides in the composition are capped RNA oligonucleotides; and (d) ligating the purified capped RNA oligonucleotide to an RNA precursor, thereby producing a capped RNA transcript.

In some embodiments, at least 98% of the total RNA oligonucleotides in the composition produced in step (c) of the method above are capped RNA oligonucleotides. In some embodiments, at least 99% of the total RNA oligonucleotides in the composition produced in step (c) of the method above are capped RNA oligonucleotides. In some embodiments, at least 99.5% of the total RNA oligonucleotides in the composition produced in step (c) of the method above are capped RNA oligonucleotides.

In some embodiments, the capping is performed chemically. In some embodiments, the chemical capping is performed through an anhydrous reaction between the 5′-phosphorylated RNA oligonucleotide and a capping nucleotide conjugated to imidazole in the presence of 1-methylimidazole.

In some embodiments, the RNA precursor comprises a 5′ untranslated region (5′ UTR) comprising an unstructured region. In some embodiments, the unstructured region comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more consecutive adenosine, cytosine, guanine, or uridine nucleotides. In some embodiments, the unstructured region comprises no more than 100, no more than 90, no more than 80, no more than 70, no more than 60, no more than 50, no more than 40, no more than 30, no more than 20, or no more than 10 consecutive adenosine, cytosine, guanine, or uridine nucleotides. In some embodiments, the unstructured region comprises between 5 and 60, between 10 and 55, between 15 and 50, between 20 and 45, between 25 and 40, or between 30 and 35 consecutive adenosine, cytosine, guanine, or uridine nucleotides.

In some embodiments, the purifying is done by high-performance liquid chromatography (HPLC).

In some embodiments, at least 95% of the capped RNA transcripts produced in step (d) of the method above comprise a 5′ cap. In some embodiments, at least 98% of the capped RNA transcripts produced in step (d) of the method above comprise a cap. In some embodiments, at least 99% of the capped RNA transcripts produced in step (d) of the method above comprise a cap. In some embodiments, at least 99.5% of the capped RNA transcripts produced in step (d) of the method above comprise a cap.

In some embodiments, the method further comprises circularizing the capped RNA transcript. In some embodiments, the circularizing is performed by intron back-splicing.

In some embodiments, the method further comprises: (i) introducing one or more azide handles onto the 5′-phosphorylated RNA oligonucleotide, and (ii) conjugating one or more 5′ cap regions to the 5′-phosphorylated RNA oligonucleotide via click chemistry with the one or more azide handles. In some embodiments, the introducing is performed by tRNA guanine transglycosylase (TGT). In some embodiments, the introducing is performed during the in vitro synthesizing of step (a) of the method above with an azide-containing phosphoramidate.

In some embodiments, the 5′-phosphorylated RNA oligonucleotide is between 3 and 160, between 5 and 150, between 7 and 140, between 9 and 130, between 11 and 120, between 13 and 110, between 15 and 100, between 17 and 90, between 19 and 80, between 21 and 70, between 23 and 60, between 25 and 50, between 27 and 40, or between 30 and 35 nucleotides in length. In some embodiments, the 5′-phosphorylated RNA oligonucleotide is between 25 and 35 nucleotides in length.

In some embodiments, the nonpolar counterion is ammonium (NH4).

In some embodiments, the synthesizing of step (a) of the method above is performed on a solid-phase support.

In some embodiments, the 5′-phosphorylated RNA oligonucleotide comprises one or more modified nucleotides.

In some embodiments, the RNA precursor comprises one or more modified nucleotides.

In some embodiments, the one or more modified nucleotides comprises a modified sugar. In some embodiments, the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose. In some embodiments, the modified sugar is selected from the following:

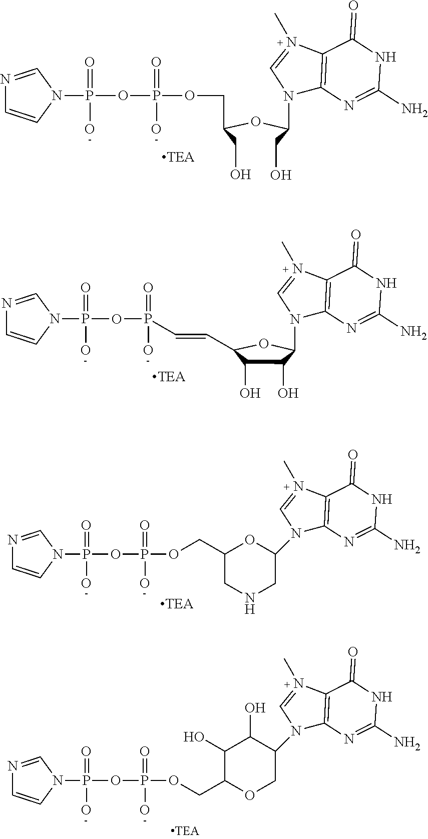

In some embodiments, the 5′-phosporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified sugars. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified sugars. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

In some embodiments, the one or more modified nucleotides comprises a modified phosphate. In some embodiments, the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, the 5′-phosphorylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified phosphates. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified phosphates. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

In some embodiments, the one or more modified nucleotides comprise a modified nucleobase. In some embodiments, the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, the 5′-phoshporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified nucleobases. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified nucleobases. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

In some embodiments, the one or more modified nucleotides comprise one or more modified sugars, one or more modified phosphates, one or more modified nucleobases, or any combination thereof.

In some embodiments, the capped RNA oligonucleotide comprises a 5′ cap selected from the group consisting of 7-methyguanosine (m7G), N7,3′-O-dimethyl-guanosine-5′-triphosphate-5′-guanosine (m7G-3′m-ppp-G), N7,2′-O-dimethyl-guanosine-5′-triphosphate-5′-guanosine (m7Gm-ppp-G), 7-benzylguanosine (Bn7G), chlorobenzylguanosine (ClBn7G), m7G bearing an LNA sugar (m7G-LNA), chlorobenzyl-O-ethoxyguanosine (ClBnOEt7G), 7-(4-chlorophenoxyethyl)-guanosine, 7-ethyl guanosine (e7G), 7-propyl guanosine (p7G), 7-isopropyl guanosine (ip7G), 7-butyl guanosine (b7G), 7-isobutyl guanosine (ib7G), 7-cyclopentyl guanosine (cp7G), 7-(carboxymethyl) guanosine (cm7G), 7-(2-phenylethyl) guanosine [7-(2-PhEt)G], 7-(1-phenylethyl) guanosine [7-(1-PhEt)G], m⁷Gppp_{BH3}G (D1 and D2 stereoisomers), m⁷Gpp_{BH3}G (D1 and D2 stereoisomers), m⁷Gp_{BH3}G (D1 and D2 stereoisomers), m⁷Gpp_{BH3}pm⁷G, m₂^{7,2′-OGppp}_{BH3}G (D1 and D2 stereoisomers), m₂7^{,2′-O}Gpp_{BH3}pG (D1 and D2 diastereomers), m₂7^{,2′-O}GppspG (D1 and D2 diastereomers), N-Arylmethyl analogs, glyceryl, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2′-inverted nucleotide moiety, 3′-2 ‘-inverted abasic moiety, 1,4-butanediol phosphate, 3’-phosphoramidate, hexylphosphate, aminohexyl phosphate, 3′-phosphate, 3′-phosphorothioate, phosphorodithioate, cap1, cap2, cap3, cap4, ARCA, modified ARCA, inosine, N1-methylguanosine, LNA-guanosine, 2-azido-guanosine, and a bridging or non-bridging methylphosphonate moiety.

In some embodiments, the ligating is performed using a T4 RNA Ligase.

In some embodiments, the HPLC comprises 10-200 mM hydrophobic hexyl ammonium ions.

In some embodiments, the RNA precursor comprises a 3′ exonuclease-resistant modification. In some embodiments, the 3′ exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

In some embodiments, the RNA precursor further comprises a poly-A tail. In some embodiments, the poly-A tail comprises between 25 and 500 nucleotides (SEQ ID NO: 168). In some embodiments, the poly-A tail comprises between 50 and 100, between 100 and 150, between 150 and 200, between 200 and 300, between 300 and 400, or between 400 or 500 nucleotides. In some embodiments, the poly-A tail comprises 10 or more adenosine nucleotides (SEQ ID NO: 169). In some embodiments, 25-100%, 30-100%, 40-100%, 50-100%, 60-100%, 70-100%, 80-100%, 90-100%, 95-100%, 96-100%, 97-100%, 98-100%, or 99-100% of nucleotides of the poly-A tail are adenosine nucleotides. In some embodiments, 100% of the nucleotides of the poly-A tail are adenosine nucleotides. In some embodiments, the poly-A tail comprises one or more modified nucleotides.

In some embodiments, the RNA precursor further comprises an open reading frame (ORF). In some embodiments, the ORF is operably connected to a promoter. In some embodiments, the ORF encodes at least one protein. In some embodiments, the at least one protein is a therapeutic protein. In some embodiments, the at least one protein is an antigen.

In some embodiments, the RNA precursor further comprises a therapeutic nucleic acid. In some embodiments, the therapeutic nucleic acid is chosen from the group consisting of an antisense oligonucleotide (ASO), an aptamer, an RNA decoy, an siRNA, a shRNA, a miRNA, or a gRNA.

In some embodiments, the 5′-phosphorylated RNA oligonucleotide comprises a promoter sequence.

In some embodiments, provided herein are capped RNA transcripts produced by any one of the methods above.

In some embodiments, provided herein are delivery agents comprising the capped RNA transcript produced by any one of the methods above, wherein the delivery agent comprises a lipid, a peptide, a protein, an antibody, a carbohydrate, a nanoparticle, or a microparticle. In some embodiments, the nanoparticle or microparticle is a lipid nanoparticle or microparticle, a polymer nanoparticle or a polymer microparticle, a protein nanoparticle or a protein microparticle, or a solid nanoparticle or a solid microparticle.

In some embodiments, provided herein are cells comprising the capped RNA transcript produced by any one of the methods above. In some embodiments, the cell is a mammalian cell.

In some embodiments, provided herein is a composition comprising the capped RNA transcript produced by any one of the methods above, the delivery agent described above, or the cell described above. In some embodiments, the composition described above further comprises an additional agent. In some embodiments, the additional agent is an agent which has a therapeutic effect when administered to a subject. In some embodiments, the additional agent is chosen from the list consisting of a nucleotide, a nucleic acid, an amino acid, a peptide, a protein, a small molecule, an aptamer, a lipid, or a carbohydrate. In some embodiments, the additional agent is a shRNA, a siRNA, or an antisense oligonucleotide (ASO). In some embodiments, the additional agent is an antigen or adjuvant. In some embodiments, the composition is a pharmaceutical composition, wherein the pharmaceutical composition comprises a pharmaceutically acceptable excipient.

In some embodiments, provided herein are methods of preventing or treating a disease in a subject, comprising introducing an effective amount of the capped RNA transcript produced by any one of the methods above, the delivery agent described above, the cell described above, or the composition described above to the subject. In some embodiments, the subject is a human.

In some embodiments, the capped RNA transcript produced by any one of the methods above, the delivery agent described above, the cell described above, or the composition described above is used in preventing or treating a disease in a subject.

In some embodiments, provided herein are kits comprising the capped RNA oligonucleotide and/or the RNA precursor described above. In some embodiments, the kit further comprises an RNA ligase.

In some embodiments, provided herein are kits comprising the composition described above, a device for administering the composition to a subject, and instructions for administering the composition to the subject.

In some aspects, the present disclosure provides a method of screening for altered translation efficiency conferred by one or more nucleotide modifications in an RNA transcript, comprising: (a) synthesizing a 5′-phosphorylated RNA oligonucleotide comprising one or more modified nucleotides in a solvent system comprising a nonpolar counterion; (b) chemically capping the 5′-phosphorylated RNA oligonucleotide, thereby producing a capped RNA oligonucleotide; (c) purifying the capped RNA oligonucleotide; (d) ligating the capped RNA oligonucleotide to an RNA precursor comprising an open reading frame (ORF) encoding a protein, thereby producing a capped RNA transcript; (e) expressing the capped RNA transcript in a cell; and (f) measuring expression of the protein in the cell.

In some embodiments, the synthesizing of step (a) of the method above is performed on a solid-phase support.

In some embodiments, the purifying of step (c) of the method above is done by high-performance liquid chromatography (HPLC). In some embodiments, the HPLC comprises 10-200 mM hydrophobic hexyl ammonium ions.

In some embodiments, the RNA precursor further comprises a 5′ untranslated region (5′ UTR) comprising an unstructured region. In some embodiments, the unstructured region comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more consecutive adenosine, cytosine, guanine, or uridine nucleotides. In some embodiments, the unstructured region comprises no more than 100, no more than 90, no more than 80, no more than 70, no more than 60, no more than 50, no more than 40, no more than 30, no more than 20, or no more than 10 consecutive adenosine, cytosine, guanine, or uridine nucleotides. In some embodiments, the unstructured region comprises between 5 and 60, between 10 and 55, between 15 and 50, between 20 and 45, between 25 and 40, or between 30 and 35 consecutive adenosine, cytosine, guanine, or uridine nucleotides.

In some embodiments, the RNA precursor further comprises a 3′ exonuclease-resistant modification. In some embodiments, the 3′ exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

In some embodiments, the RNA precursor comprises one or more modified nucleotides.

In some embodiments, the one or more modified nucleotides comprises a modified sugar. In some embodiments, the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose. In some embodiments, the modified sugar is selected from the following:

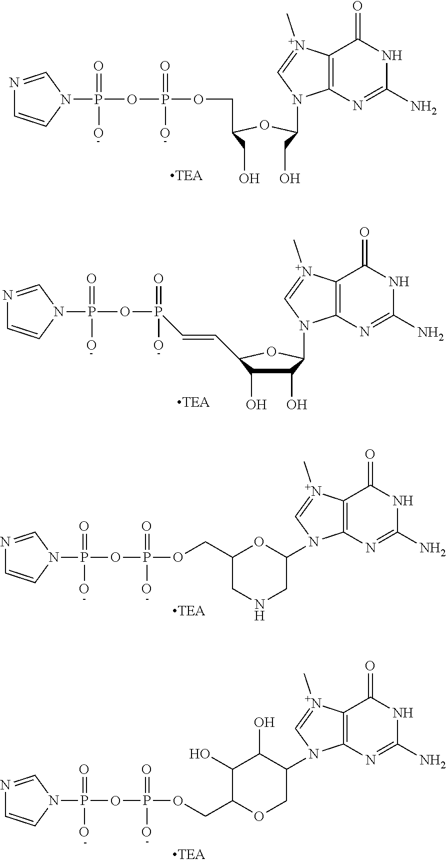

In some embodiments, the 5′-phosporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified sugars. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified sugars. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

In some embodiments, the one or more modified nucleotides comprises a modified phosphate. In some embodiments, the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, the 5′-phosphorylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified phosphates. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified phosphates. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

In some embodiments, the one or more modified nucleotides comprise a modified nucleobase. In some embodiments, the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, the 5′-phoshporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified nucleobases. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified nucleobases. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

In some embodiments, the one or more modified nucleotides comprise one or more modified sugars, one or more modified phosphates, one or more modified nucleobases, or any combination thereof.

In some embodiments, the chemical capping is performed through an anhydrous reaction between the 5′-phosphorylated RNA oligonucleotide and a capping oligonucleotide conjugated to imidazole in the presence of 1-methylimidazole.

In some embodiments, the RNA precursor further comprises a poly-A tail. In some embodiments, the poly-A tail comprises between 25 and 500 nucleotides (SEQ ID NO: 168). In some embodiments, the poly-A tail comprises between 50 and 100, between 100 and 150, between 150 and 200, between 200 and 300, between 300 and 400, or between 400 and 500 nucleotides. In some embodiments, the poly-A tail comprises 10 or more adenosine nucleotides (SEQ ID NO: 169). In some embodiments, 25-100%, 30-100%, 40-100%, 50-100%, 60-100%, 70-100%, 80-100%, 90-100%, 95-100%, 96-100%, 97-100%, 98-100%, or 99-100% of nucleotides of the poly-A tail are adenosine nucleotides.

In some embodiments, the cell is a mammalian cell.

In some embodiments, the measuring is done by Western blotting, flow cytometry, mass spectrometry, enzyme-linked immunosorbent assay (ELISA), microscopy, bicinchoninic acid (BCA) assay, Coomassie protein assay, fluorometry, and/or spectrofluorometry.

In some embodiments, the protein is a reporter protein or a therapeutic protein. In some embodiments, the reporter protein is chosen from the group consisting of β-galactosidase, chloramphenicol acetyltransferase, green fluorescent protein (e.g., GFP or EGFP), red fluorescent protein (e.g., RFP, ERFP, or dsRed), yellow fluorescent protein (e.g., YFP or EYFP), cyan fluorescent protein (e.g., CFP or ECFP), mOrange, tdTomato, mCherry, and luciferase.

In some aspects, provided herein is a method of screening for altered RNA stability conferred by one or more nucleotide modifications in an RNA transcript, comprising: (a) synthesizing a 5′-phosphorylated RNA oligonucleotide comprising one or more modified nucleotides in a solvent system comprising a nonpolar counterion; (b) chemically capping the 5′-phosphorylated RNA oligonucleotide, thereby producing a capped RNA oligonucleotide; (c) purifying the capped RNA oligonucleotide; (d) ligating the capped RNA oligonucleotide to an RNA precursor comprising an open reading frame (ORF) encoding a protein, thereby producing a capped RNA transcript; (e) expressing the purified capped RNA transcript in a cell; and (f) measuring levels of the capped RNA transcript in the cell.

In some embodiments, the synthesizing of step (a) of the method above is performed on a solid-phase support.

In some embodiments, the purifying of step (c) of the method above is done by high-performance liquid chromatography (HPLC). In some embodiments, the HPLC comprises 10-200 mM hydrophobic hexyl ammonium ions.

In some embodiments, the RNA precursor further comprises a 5′ untranslated region (5′ UTR) comprising an unstructured region. In some embodiments, the unstructured region comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more consecutive adenosine, cytosine, guanine, or uridine nucleotides. In some embodiments, the unstructured region comprises no more than 100, no more than 90, no more than 80, no more than 70, no more than 60, no more than 50, no more than 40, no more than 30, no more than 20, or no more than 10 consecutive adenosine, cytosine, guanine, or uridine nucleotides. In some embodiments, the unstructured region comprises between 5 and 60, between 10 and 55, between 15 and 50, between 20 and 45, between 25 and 40, or between 30 and 35 consecutive adenosine, cytosine, guanine, or uridine nucleotides.

In some embodiments, the RNA precursor further comprises a 3′ exonuclease-resistant modification. In some embodiments, the 3′ exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

In some embodiments, the RNA precursor comprises one or more modified nucleotides.

In some embodiments, the one or more modified nucleotides comprises a modified sugar. In some embodiments, the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose. In some embodiments, the modified sugar is selected from the following:

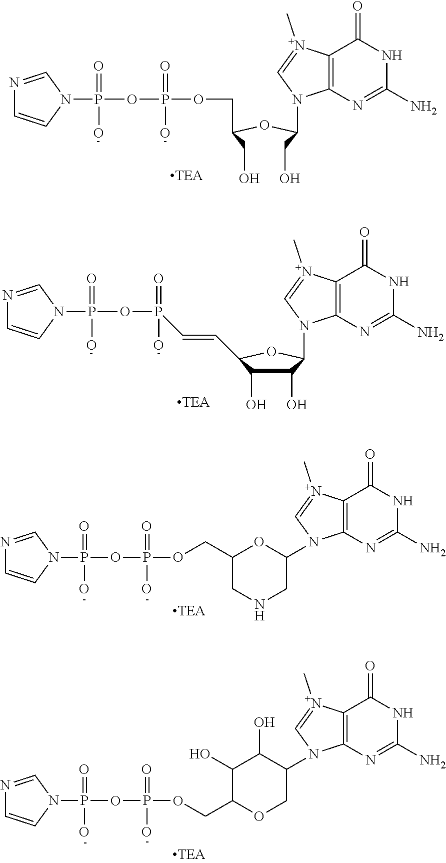

In some embodiments, the 5′-phosporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified sugars. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified sugars. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

In some embodiments, the one or more modified nucleotides comprises a modified phosphate. In some embodiments, the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, the 5′-phosphorylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified phosphates. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified phosphates. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

In some embodiments, the one or more modified nucleotides comprise a modified nucleobase. In some embodiments, the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, the 5′-phoshporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified nucleobases. In some embodiments, the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified nucleobases. In some embodiments, the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases. In some embodiments, the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

In some embodiments, the one or more modified nucleotides comprise one or more modified sugars, one or more modified phosphates, one or more modified nucleobases, or any combination thereof.

In some embodiments, the chemical capping is performed through an anhydrous reaction between the 5′-phosphorylated RNA oligonucleotide and a capping oligonucleotide conjugated to imidazole in the presence of 1-methylimidazole.

In some embodiments, the RNA precursor further comprises a poly-A tail. In some embodiments, the poly-A tail comprises between 25 and 500 nucleotides (SEQ ID NO: 168). In some embodiments, the poly-A tail comprises between 50 and 100, between 100 and 150, between 150 and 200, between 200 and 300, between 300 and 400, or between 400 and 500 nucleotides. In some embodiments, the poly-A tail comprises 10 or more adenosine nucleotides (SEQ ID NO: 169). In some embodiments, 25-100%, 30-100%, 40-100%, 50-100%, 60-100%, 70-100%, 80-100%, 90-100%, 95-100%, 96-100%, 97-100%, 98-100%, or 99-100% of nucleotides of the poly-A tail are adenosine nucleotides.

In some embodiments, the cell is a mammalian cell.

In some embodiments, the measuring is done by Western blotting, PC R, real-time PCR, next-generation sequencing (NGS), flow cytometry, mass spectrometry, enzyme-linked immunosorbent assay (ELISA), microscopy, bicinchoninic acid (BCA) assay, Coomassie protein assay, fluorometry, spectrofluorometry, RNA-seq, single-cell RNA-seq, and/or nanoscale secondary ion mass spectrometry (NanoSIMS).

In some embodiments, the protein is a reporter protein or a therapeutic protein. In some embodiments, the reporter protein is chosen from the group consisting of β-galactosidase, chloramphenicol acetyltransferase, green fluorescent protein (e.g., GFP or EGFP), red fluorescent protein (e.g., RFP, ERFP, or dsRed), yellow fluorescent protein (e.g., YFP or EYFP), cyan fluorescent protein (e.g., CFP or ECFP), mOrange, tdTomato, mCherry, and luciferase.

In some aspects, provided herein are RNA molecules comprising (i) one or more modified nucleotides at position +3 or higher with reference to a 5′ terminus of the RNA molecule, and (ii) at least one 5′ cap. In some embodiments, the RNA molecule comprises two or more 5′ caps.

In some embodiments, the two or more 5′ caps are conjugated to a 5′ UTR of the RNA molecule. In some embodiments, the two or more 5′ caps are conjugated to the RNA molecule via click chemistry. In some embodiments, the two or more 5′ caps are conjugated to the RNA molecule via a photochemical crosslinking reaction.

In some embodiments, the one or more modified nucleotides comprises a modified sugar. In some embodiments, the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose. In some embodiments, the modified sugar is selected from the following:

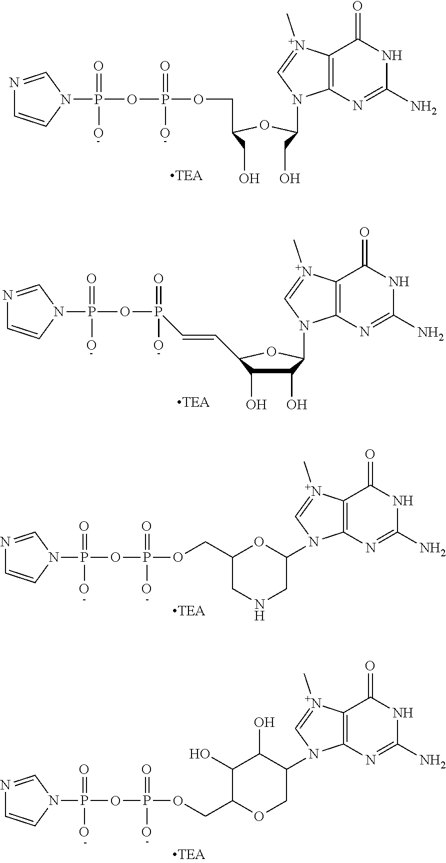

In some embodiments, the RNA molecule comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars. In some embodiments, the RNA molecule comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

In some embodiments, the one or more modified nucleotides comprises a modified phosphate. In some embodiments, the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, the RNA molecule comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates. In some embodiments, the RNA molecule comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

In some embodiments, the one or more modified nucleotides comprises a modified nucleobase. In some embodiments, the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, the RNA molecule comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases. In some embodiments, the RNA molecule comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

In some embodiments, the one or more modified nucleotides comprise one or more modified sugars, one or more modified phosphates, one or more modified nucleobases, or any combination thereof.

In some embodiments, the 5′ cap is selected from the group consisting of 7-methyguanosine (m7G), N7,3′-O-dimethyl-guanosine-5′-triphosphate-5′-guanosine (m7G-3′m-ppp-G), N7,2′-O-dimethyl-guanosine-5′-triphosphate-5′-guanosine (m7Gm-ppp-G), 7-benzylguanosine (Bn7G), chlorobenzylguanosine (ClBn7G), m7G bearing an LNA sugar (m7G-LNA), chlorobenzyl-O-ethoxyguanosine (ClBnOEt7G), 7-(4-chlorophenoxyethyl)-guanosine, 7-ethyl guanosine (e7G), 7-propyl guanosine (p7G), 7-isopropyl guanosine (ip7G), 7-butyl guanosine (b7G), 7-isobutyl guanosine (ib7G), 7-cyclopentyl guanosine (cp7G), 7-(carboxymethyl) guanosine (cm7G), 7-(2-phenylethyl) guanosine [7-(2-PhEt)G], 7-(1-phenylethyl) guanosine [7-(1-PhEt)G], m⁷Gppp_{BH3}G (D1 and D2 stereoisomers), m⁷Gpp_{BH3}G (D1 and D2 stereoisomers), m⁷Gp_{BH3}G (D1 and D2 stereoisomers), m⁷Gpp_{BH3}pm⁷G, m₂^{7,2′-O}Gppp_{BH3}G (D1 and D2 stereoisomers), m₂^{7,2′-O}Gpp_{BH3}PG (D1 and D2 diastereomers), m₂^{7,2′-O}GppspG (D1 and D2 diastereomers), N-Arylmethyl analogs, glyceryl, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2′-inverted nucleotide moiety, 3′-2 ‘-inverted abasic moiety, 1,4-butanediol phosphate, 3’-phosphoramidate, hexylphosphate, aminohexyl phosphate, 3′-phosphate, 3′-phosphorothioate, phosphorodithioate, cap1, cap2, cap3, cap4, ARCA, modified ARCA, inosine, N1-methylguanosine, LNA-guanosine, 2-azido-guanosine, and a bridging or non-bridging methylphosphonate moiety.

In some embodiments, the RNA molecule comprises at least one poly-A tail. In some embodiments, the at least one poly-A tail comprises between 25 and 500 nucleotides (SEQ ID NO: 168). In some embodiments, the at least one poly-A tail comprises between 50 and 100, between 100 and 150, between 150 and 200, between 200 and 300, between 300 and 400, or between 400 and 500 nucleotides. In some embodiments, the at least one poly-A tail comprises 10 or more adenosine nucleotides (SEQ ID NO: 169). In some embodiments, 25-100%, 30-100%, 40-100%, 50-100%, 60-100%, 70-100%, 80-100%, 90-100%, 95-100%, 96-100%, 97-100%, 98-100%, or 99-100% of nucleotides of the at least one poly-A tail are adenosine nucleotides.

In some embodiments, the 5′ cap is added to the RNA molecule through a chemical capping method. In some embodiments, the chemical capping method is an anhydrous reaction between a 5′-phosphorylated RNA molecule and a capping nucleotide conjugated to imidazole in the presence of 1-methylimidazole.

In some embodiments, the RNA molecule further comprises a 5′ untranslated region (5′ UTR). In some embodiments, the 5′ UTR comprises a promoter.

In some embodiments, the RNA molecule further comprises a 3′ untranslated region (3′ UTR). In some embodiments, the 3′ UTR comprises at least one exonuclease-resistant modification. In some embodiments, the exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

In some embodiments, the RNA molecule comprises two or more 5′ caps.

In some embodiments, the RNA molecule comprises two or more poly-A tails.

In some embodiments, the RNA molecule further comprises an open reading frame (ORF). In some embodiments, the ORF encodes a protein. In some embodiments, the protein is a therapeutic protein. In some embodiments, the protein is an antigen.

In some embodiments, the RNA molecule further comprises a sequence encoding a therapeutic nucleic acid. In some embodiments, the therapeutic nucleic acid is an antisense oligonucleotide (ASO), an aptamer, an RNA decoy, an siRNA, a shRNA, a miRNA, or a gRNA.

In some embodiments, the RNA molecule further comprises a 3′ cap.

In some embodiments, the RNA molecule is a circular RNA molecule.

In some embodiments, provided herein are vectors comprising the RNA molecule described above.

In some embodiments, provided herein are delivery agents comprising the RNA molecule described above, wherein the delivery agent comprises a lipid, a peptide, a protein, an antibody, a carbohydrate, a nanoparticle, or a microparticle. In some embodiments, the nanoparticle or microparticle is a lipid nanoparticle or a lipid microparticle, a polymer nanoparticle or a polymer microparticle, a protein nanoparticle or a protein microparticle, or a solid nanoparticle or a solid microparticle.

In some embodiments, provided herein are cells comprising the RNA molecule described above or the vector described above. In some embodiments, the cell is a mammalian cell.

In some embodiments, provided herein are compositions comprising the RNA molecule described above, the vector described above, the delivery agent described above, or the cell described above. In some embodiments, the composition further comprises an additional agent. In some embodiments, the additional agent is an agent which has a therapeutic effect when administered to a subject. In some embodiments, the additional agent is a nucleotide, a nucleic acid, an amino acid, a peptide, a protein, a small molecule, an aptamer, a lipid, or a carbohydrate. In some embodiments, the nucleotide is a shRNA, a siRNA, a miRNA, or an antisense oligonucleotide (ASO). In some embodiments, the additional agent is an antigen or adjuvant. In some embodiments, the composition is a pharmaceutical composition comprising a pharmaceutically acceptable excipient.

In some embodiments, provided herein are methods of preventing or treating a disease in a subject, comprising introducing an effective amount of the RNA molecule described above, the vector described above, the delivery agent described above, or the composition described above to the subject. In some embodiments, the subject is a human.

In some embodiments, the RNA molecule described above, the vector described above, or the composition described above is used in preventing or treating a disease in a subject. In some embodiments, the subject is a human.

In some embodiments, provided herein are kits comprising the composition described above, a device for administering the composition to a subject, and/or instructions for administering the composition to the subject.

The foregoing general description and following detailed description are examples and are intended to provide further explanation of the disclosure as claimed. Other objects, advantages, and novel features will be readily apparent to those skilled in the art from the following brief description of the drawings and detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

FIGS. 1A-1H show conceptualization of capped circular mRNA (QRNA). FIG. 1A shows schematics of regular circular RNA structure and IRES mediated translation initiation.

FIG. 1B shows schematics of regular linear RNA structure and m7G-cap dependent translation initiation. FIG. 1C illustrates design of a capped-circular mRNA (QRNA): a circular RNA bearing a click reaction handle in its 5′ untranslated region (UTR) is chemically conjugated to the 3′-end of a linear oligo, which is chemically capped. QRNA hijacks the cap-dependent translation initiation mechanism to increase ribosome loading on the circular transcript. FIG. 1D and FIG. 1E show generic structures of type 1&2 QRNAs. FIG. 1F illustrates the synthetic scheme of a Type 3 Flag-encoding QRNA, wherein Oligo 3.1 is a sequence-designed RNA containing twister ribozymes (red), tRNA-like hairpin (green), 5′UTR (blue), and 3×Flag-peptide coding sequences (orange); Oligo 3.2 is a circularized RNA; Oligo 3.3 is a circular RNA bearing a 5-methyl-tetrazine click chemistry handle; Oligo 3.4 is a capped oligo with a 3′-TCO click chemistry handle; and Oligo 3.5: is the desired 3×Flag encoding Type 2 QRNA product with click chemistry linkage shown in pink. FIG. 1G illustrates the structure of 5-methyl-tetrazine containing preQ1 cofactor analogue. FIG. 1H shows the structure of the 3′-TCO click chemistry handle. Figure discloses SEQ ID NO: 179.

FIGS. 2A-2H show a general synthesis workflow of Type 1 QRNA. FIG. 2A shows representative chemical structures of various components in the capped circular mRNA: cap, alkyne handles, phosphate group, and azide handles, each is color coded and shown in the following figures. FIG. 2B illustrates a chemically synthesized oligo bearing a 3′-end click chemistry handle is chemically capped and high-performance liquid chromatography (HPLC) purified. FIG. 2C shows alternatively a chemically synthesized oligo bearing a 3′-end click chemistry handle can be enzymatically capped. FIG. 2D illustrates a chemically/enzymatically synthesized mRNA transcript bearing a click chemistry handle in its 5′-UTR and a 5′-phosphate can be circularized using T4 RNA ligase; complementary sequences in both 5′ and 3′ UTRs facilitate the circularization. CircRNA bearing the click chemistry handle is chemically conjugated to the capped oligo. FIG. 2E shows a chemically/enzymatically synthesized mRNA transcript bearing a click chemistry handle in its 5′-UTR and a 5′-phosphate can be circularized using T4 RNA ligase II using a DNA splint probe complementary to the 5′/3′-ends. CircRNA bearing the click chemistry handle is chemically conjugated to the capped oligo. FIG. 2F shows mRNA constructs bearing a RNA stem-loop in its 5′-UTR can be synthesized by IVT and circularized using methods in 2C/2D, or circularized using ribozyme mediated backsplicing. The stem-loop can be modified using RNA-modifying enzymes such as tRNA transferases to introduce a click reaction handle on the circular RNA. CircRNA bearing the click chemistry handle is chemically conjugated to the capped oligo. FIG. 2G demonstrates in vitro transcribed mRNA having 3′/5′-hydroxyl groups can be enzymatically ligated to a chemically synthesized oligo bearing a click chemistry handle and phosphates on both 5′ and 3′ ends. Ligation of 5′-phosphate and 3′-OH is achieved using T4 RNA ligase II and ligation of 5′-OH and 3′-phosphate is achieved using RNA ligase RtcB. Both ligation is facilitated by DNA splint. CircRNA bearing the click chemistry handle is chemically conjugated to the capped oligo. FIG. 2H demonstrates in vitro transcribed mRNA 5′ triphosphate/3′ hydroxyl groups can be enzymatically ligated to a chemically synthesized oligo bearing a click chemistry handle and phosphates on both 5′ and 3′ ends. Ligation of 5′-phosphate and 3′-OH is achieved using T4 RNA ligase II. 5′-triphosphate is hydrolyzed using calf intestinal alkaline phosphatase (CIAP) to a 5′-hydroxyl. Ligation of 5′-OH and 3′-phosphate is achieved using RNA ligase RtcB. Both ligation is facilitated by DNA splint. CircRNA bearing the click chemistry handle is chemically conjugated to the capped oligo. FIG. 2I shows alternatively a chemically synthesized oligo bearing a click chemistry handle can be used as primers annealing to the DNA template and in vitro transcribed using an RNA polymerase engineered from DNA polymerase. The IVT-synthesized mRNA can be then used for QRNA synthesis as outlined in FIG. 2D/FIG. 2E.

FIGS. 3A-3B demonstrates a general synthesis workflow of Type 2 QRNA. FIG. 3A shows that a capped RNA having a stem-loop in its 5′-UTR is synthesized by IVT and subsequently ligated to chemically synthesized oligos having 5′-phosphate and 3′-click chemistry handles using T4 RNA ligase. The product is intramolecularly circularized to yield type 2 QRNA. FIG. 3B shows alternatively a chemically synthesized oligo bearing a click chemistry handle is capped and used as primers annealing to the DNA template and in vitro transcribed using an RNA polymerase engineered from DNA polymerase. The IVT-synthesized mRNA can be then used for type 2 QRNA synthesis.

FIG. 4 shows synthesis of type 1 HiBit QRNA. HiBit-encoding RNA was codon optimized such that it only contains one single C in the 5′-UTR and an azide handle was incorporated by full replacement of CTP with azide CTP during IVT.

FIGS. 5A-5B show a summary of oligonucleotide chemical conjugation methods. Screening was performed using 15-nt dA model substrates at micromolar concentrations. Modification handles were incorporated through solid phase synthesis, followed by amine-NHS labeling and HPLC purification if necessary. FIG. 5C is gel electrophoresis of crude thiol-ene/yne oligonucleotide conjugation of 15-nt model substrates containing only one conjugation handle. FIG. 5D is gel electrophoresis of crude CuAAC and IEDDA 30-nt oligonucleotides bearing three EU/TCO handles reacting with 30-nt N₃/Tz modified oligo.

FIG. 6 is an illustration of QRNA synthesis workflow by combining enzymatic labeling and click chemistry. Figure discloses SEQ ID NO: 180.

FIG. 7 depicts the synthesis and purification of ~100% 5′ capped modified oligonucleotides. Panel A shows the development of a chemical capping and HPLC workflow allowing effective separation of capped/uncapped oligonucleotides. The bottom plot demonstrates that oligonucleotides synthesized with ammonium counterions were effectively capped at a higher scale compared to other methods (e.g., triethylammonium/sodium). Panel B shows a HPLC chromatogram demonstrating the concentration-dependent scalability of oligo capping using the methods provided herein. Panel C shows a PAGE gel demonstrating the ability to differentiate between capped and uncapped oligonucleotides (15% acrylamide gel). Panel D shows a HPLC chromatogram demonstrating the ability to use this purification method with a variety of different nucleotide modifications.

FIG. 8 shows multidimensional chemical modifications of the mRNA cap and 5′ UTR possible using the methods described herein. Panel A depicts accessing the 5′ mRNA chemical modification landscape using an integrated chemo-enzymatic approach. Panels B-E show bar plots of time-course dual luciferase assay screening of chemical modifications on first base identity (Panel B), phosphodiester linkage (Panel C), sugar backbone (Panel D), and 5′cap (Panel E and Panel F). Protein expression was measured by Firefly luciferase luminescence normalized to Renilla luciferase luminescence (transfection control) then to the wild-type cap (m⁷G-rG) construct (dotted line) at 8-, 24-, and 48-hours post-transfection. Mean±sem. P values were calculated by ordinary one-way ANOVA (alpha=0.05), with multiple comparisons to m⁷G-rG (panel B)/m⁷G-rA (panels C-E) at 24 hours. *P<0.0332, **P<0.0021, ***P<0.0002, ****P<0.0001, n.s. P>0.05.

FIG. 9 shows the biochemical evaluation of chemically and topologically augmented capped oligonucleotides. Oligonucleotides comprising various chemical modifications were conjugated to an AF647 fluorophore and EMSA was used to quantify their binding to eIF4E (Panel A). Modifications tested included m7G-LNA (Panel B), m7G-6×2OMe (Panel C), m7GLNA-LNA (Panel D), m7GLNA-6×2OMe (Panel E), m7G-OU (1×m7G) (Panel F), and m7G-OU (2×m7G) Panel G). Panel H shows a competitive EMSA of equimolar mono-capped and dual-capped oligonucleotides with eIF4E (4~20% acrylamide). Panel I shows denaturing PAGE analysis of chemically modified capped oligonucleotides by hDcp2 at various time points (15% acrylamide). Panel J depicts the immune toxicity in HeLa cells induced by chemically and topologically modified mRNAs as assessed by qPCR detection of key factors involved in RIG-1 signaling pathways (Mx1 and ISG15), normalized to GAPDH levels. Panel K shows a graph illustrating the expression of mono-capped RLuc when transfected in wild-type and eIF3D-knockdown HeLa cells. Knockdown was achieved by siRNA transfection. n=3, biological replicates. P values were calculated by two-sided unpaired t-test. Panel L shows a graph illustrating the expression of dual-capped FLuc when transfected in wild-type and eIF3D-knockdown HeLa cells. Knockdown was achieved by siRNA transfection. n=3, biological replicates. P values were calculated by two-sided unpaired t-test. Panel M shows a graph illustrating the expression of mono-capped RLuc and dual-capped FLuc when co-transfected in wild-type and eIF3D-knockdown HeLa cells. Knockdown was achieved by siRNA transfection. n=3, biological replicates. P values were calculated by two-sided unpaired t-test. Panel N shows a graph illustrating the in vitro translation of mono-capped FLuc with or without 4EGI-1 in rabbit reticulocyte lysates (RRL). n=3, biological replicates. P values were calculated by two-sided unpaired t-test. Panel O shows a graph illustrating the in vitro translation of dual-capped NLuc with or without 4EGI-1 in rabbit reticulocyte lysates (RRL). n=3, biological replicates. P values were calculated by two-sided unpaired t-test. Panel P shows a graph illustrating the in vitro translation of mono-capped FLuc and dual-capped NLuc with or without 4EGI-1 in rabbit reticulocyte lysates (RRL). n=3, biological replicates. P values were calculated by two-sided unpaired t-test.

FIG. 10 shows chemo-topological engineering of multi-capped branched mRNA. Panel A depicts a schematic of the cap-dependent translation initiation model via eIF4E/eIF4G recognition. Panel B depicts the conceptualization of a branched mRNA as used herein, covalently bearing two caps for enhanced translation initiation. Panel C shows HPLC purification of a branched oligonucleotide containing two caps. Panel D shows a denaturing PAGE characterization of a dual-capped oligonucleotide (15% acrylamide). Panel E shows a comparison of mRNA with (0) no cap, (1) branched-cap, or (2) regular cap measured by bioluminometry 8 hours post-transfection. Panel F shows bioluminescence decay of a branch cap versus a regular cap from 8- to 24-hours post-transfection. Panels G-I show the topological optimization of dual-capped branched mRNA in terms of OU handle position, branching point, and branching length. Panel J shows phosphorothioate (PS) modification on the branched oligonucleotide minimally contributed to stability. Panel K shows multi-dimensional optimization dramatically enhanced mRNA translation capacity. P values were calculated by unpaired t test. ***P<0.001; ****P<0.0001; n.s. P>0.05. Figure discloses SEQ ID NO: 179.

FIG. 11 shows the topological augmentation of minimal HiBiT peptide-encoding mRNAs. Panel A depicts a schematic of dual-capped HiBiT mRNA. Panel B shows denaturing PAGE characterization of branched and linear HiBiT mRNAs (15% acrylamide). M, marker. Panel C shows the relative HiBiT luminescence in HeLa cells normalized to chemically capped linear HiBiT mRNA. P values were calculated by unpaired t test. ***P<0.001; ****P<0.0001; n.s. P>0.05. Panel D depicts a schematic of the synthesis of HiBiT circRNA with single azide handle and HiBiT QRNA. Panel E shows denaturing PAGE characterization of topologically augmented HiBiT mRNAs (15% acrylamide). Panel F shows relative HiBiT luminescence in HeLa cells normalized to HiBiT circRNA. P values were calculated by unpaired t test. ***P<0.001; ****P<0.0001; n.s. P>0.05.

FIG. 12 depicts the effects of secondary structures on 5′-ligation efficiency. Panel A shows a schematic of a 5′-RNase H assay for quantification of ligation efficiency. Panel B shows the effect of different reaction conditions on the ligation efficiency of human alpha-globin 5′ UTR (15% TBU—electrophoresis gel containing tris base (T), boric acid (B), EDTA, acrylamide (15%), bisacrylamide, TEMED, APS, and 7M urea, Invitrogen™, Novex™ TBE-Urea Gels, 15% Catalog number: EC6885BOX.). Panel C demonstrates the structural prediction of mRNA secondary structure before (left) and after (right) the insertion of an unstructured spacer near the 5′ terminus. Prediction generated using RNAFold. Panel D shows the quantification of ligation efficiency at different concentrations of 5′ capped UTR oligonucleotides to 5P-mRNA after the insertion of the unstructured spacer region (15% TBU). Panel E shows the quantification of RNA self-ligation at different concentrations of 5′ capped UTR oligonucleotides to 5P-mRNA after the insertion of the unstructured spacer region. 2% agarose gel; M=marker. Figure discloses SEQ ID NOS 181-189 and 169, respectively, in order of appearance.

FIG. 13 shows the workflow developed for LEGO, a method for highly efficient production of 5′ capped modified mRNAs. Panel A shows a schematic demonstrating the synthesis of mRNA with defined 3′ and 5′ characteristics. mRNA comprising an unstructured region in its 5′ end is transcribed via in vitro transcription (IVT), then is ligated on its 3′ end to a chemically synthesized oligonucleotide comprising nuclease-resistant modifications. The triphosphorylated mRNA is then treated with RppH to generate singly phosphorylated 5P-mRNA. Synthetic 5′ capped UTR oligonucleotides are then ligated to the 5P-mRNA to generate a modified mRNA molecule with the desired 5′ cap and UTR modifications. Panel B shows a representative HPLC chromatogram of modified mRNA isolation and oligonucleotide recovery. Panel C shows the quantification of 5′ ligation efficiency of control (left) with non-ligated mRNA transcript or modified (right) mRNA. Panel D shows the quantification of self-ligation of control (left) with non-ligated mRNA transcript and modified (right) mRNA.15% acrylamide, 2% agarose. M=marker. Panel E shows the effects of tail stabilization on Fluc mRNA translation.

FIG. 14 depicts mechanistic insights on enhanced translation of dual-capped mRNA. Panel A shows a schematic of a SILAC proteomics pulldown-MS analysis. Magnetic beads coated with either modified or unmodified synthetic oligos that are 5′-capped and 3′-biotinylated were incubated in HEK293T cell cytosolic extracts. Pulled down proteins were equally mixed, separated by electrophoresis, trypsin digested, and analyzed by quantitative mass spectrometry, where enrichment across conditions were calculated by the ratio of heavy/light peptides. Experiments were repeated by pulling down the heavy proteins with modified oligos, the light proteins with unmodified oligos (forward), and pulling down the light proteins with modified oligos, the heavy proteins with unmodified oligos (reverse). The unmodified oligo contains the wide-type cap 0 structure and the modified oligo contains the LNAm⁷G cap, first base LNA backbone, and 2′-O-methylation for the next 5 bases. Panel B shows an enrichment analysis of proteins pulled down by modified/unmodified oligos. Translation regulators, ribosomal proteins, and other proteins that were enriched/decreased by >4 folds are highlighted. Panel C shows an ingenuity pathway analysis (IPA) of the eukaryotic translation initiation pathway using protein enrichment data from the SILAC experiment (modified/unmodified). Panel D depicts the effects of chemical and topological modifications on oligonucleotide affinity towards eIF4E by EMSA. Panel E shows the effects of chemical modifications on capped-oligonucleotide resistance against hDcp2. Panel F shows schematics of evaluating the translation efficiency of the engineered Firefly luciferase reporter mRNAs using paired STARmap and RIBOmap for in situ profiling the total mRNA and the ribosome-bound mRNA, respectively. Unmodified Renilla luciferase mRNA was only profiled by STARmap in all replicates as internal transfection control. Panel G shows representative STARmap/RIBOmap images taken under the same confocal imaging settings. DAPI (blue), nuclei; Firefly luciferase amplicons (green); Renilla luciferase amplicons (red). Panel H shows single-cell quantification of total Fluc mRNA normalized to Rluc mRNA control (numbers of Fluc STARmap amplicons per cell/numbers of Rluc RIBOmap amplicons per cell). n=1100/712, biological replicates. Mean±sem. P values were calculated by unpaired two-sided t-test. Panel I shows single-cell quantification of ribosome-bound Fluc mRNA normalized to Rluc mRNA control (numbers of Fluc RIBOmap amplicons per cell/numbers of Rluc RIBOmap amplicons per cell). n=1054/1083, biological replicates. Mean±sem. P values were calculated by unpaired two-sided t-test. Panel J shows relative translation efficiency (RTE) measured by (Fluc RIBOmap/Rluc STARmap)/(Fluc STARmap/Rluc STARmap) in each field of view. n=12, biological replicates. Mean #sem. P values were calculated by unpaired two-sided t-test. Panel K shows that dual-capped mRNA exhibited better translatability at reduced eIF concentrations in vitro. Panel L shows that 4EGI-1 inhibited translation of dual-capped mRNA in vitro. P values were calculated by unpaired t-test. ***P<0.001; ****P<0.0001; n.s. P>0.05.

FIG. 15 shows eIF3D knockdown significantly decreased translation of both regular and multi-capped mRNA. Panel A shows a Western blot of eIF3D in HeLa cells treated with increasing concentrations of siRNAs targeting eIF3d. M, marker. Panels B-Cshow the quantification of expression of mono-capped Rluc and dual-capped Fluc co-transfected in wild-type and eIF3D-knockdown HeLa cells. Panel D shows the quantification of expression of dual-capped Fluc normalized to co-transfected mono-capped Rluc in wild-type and eIF3D-knockdown HeLa cells.

FIG. 16 depicts the synthesis of capped-circular mRNA via branched topology. Panel A depicts a schematic showing IRES-based circRNA translation initiation through the eIF4G-4F complex. Panel B depicts a schematic showing branched-cap-induced translation lacking protection of mRNA from exonuclease degradation. Panel C depicts a schematic conceptualizing the capped-circular mRNA (QRNA) to make possible the utilization of eIF4E-dependent pathways of translation initiation in circRNA. Panel D demonstrates a QRNA synthesis workflow described herein, which combines site-specific enzymatic labeling and click chemistry technology. The QRNA construct contains hemoglobin-derived UTRs and template-encoded poly-A sequences. Panel E shows double RNase H characterization of QRNA (15% acrylamide). Panel F shows that QRNA effectively induced translation of circRNA with or without the inclusion of an IRES. m7G-capped linear mRNA with wild-type uridine served as a positive control. Panel G demonstrates that uridine reduced mRNA translation globally. m7G-capped linear mRNA (with and without m¹ψ) and QRNA (with uridine) encoding Nluc were co-transfected with Fluc mRNA (m7G-capped, m¹ψ-modified). Expression of co-transfection control Fluc mRNA was also reduced when mRNA containing uridine was transfected, regardless of topology. P values were calculated by unpaired t-test. **P<0.01; ***P<0.001; ****P<0.0001; n.s. P>0.05. Figure discloses SEQ ID NO: 180.

FIG. 17 shows the characterization of circRNAs. Panel A shows agarose gel electrophoresis characterization of Nluc circRNA. The same gel was split for labeling purposes. M, marker. 2% agarose. Panel B shows the expression and purification of TGT-His. M, marker. 4~12% Bis-Tris gel. Panels C-D show NMR characterization of preQ1-peg1-azide.

FIG. 18 shows the RNase H characterization of Nluc (Panel A) AND hEPO (Panel B) mRNA used for in vivo mouse experiments (15% acrylamide). Panel C shows the flow cytometry gating strategy used to count reticulocytes from peripheral mouse plasma.

FIG. 19 demonstrates that multi-capped mRNA exhibited enhanced and prolonged translation in vivo. Panel A depicts a schematic of the modified mRNA used encoding Nluc, which contained two LNAm7G caps, an LNA-2OMe-modified 5′ UTR, and a PS-2MOE-modified poly-A tail. The mRNA was encapsulated in a lipid nanoparticle (LNP) and administered via retro-orbital (R.O.) injection. Expression was assayed at the indicated time points. Panel B shows the quantification of luminescence per mouse. Optimized mRNA was compared with 100% m7G-rG capped linear mRNA with full m1ψ replacement and 100-A poly-A tails (termed “control”). Both mRNAs had the same sequences with different modification patterns. Panel C shows representative in vivo luminescence images as 6-, 72-, 144-, and 240-hours post-injection. Panel D depicts a schematic outlining an experiment wherein control and optimized mRNA encoding hEPO was administered via R.O. injection to mice. Plasma hEPO expression was assayed in one cohort using ELISA, and reticulocytes were counted in another cohort at 144 hours (day 6 post-injection). Poly(C)-LNP was used as negative control. Panel E shows the quantification of hEPO levels at the indicated time points. Panel F shows the quantification of reticulocyte percentage among red blood cells 144 hours after RNA-LNP administration. Panel G depicts representative reticulocyte counting for each condition as measured by flow cytometry on day 6 post-injection. Panels H-I show the quantification of plasma levels of TNA-α and ALT on day 6 post-injection quantified by ELISA. P values were calculated by unpaired t-test. **P<0.01; ***P<0.001; ****P<0.0001; n.s. P>0.05. Panel J shows experimental results following an experiment in which m1Ψ-modified NLuc circRNA and QRNA (with LNAm7G cap) were formulated in lipid nanoparticles (LNP) and administered via retro-orbital (R.O.) injection. Expression was assayed at indicated time points. n=4, biological replicates. Mean±sem. P values were calculated by unpaired two-sided t-test.

FIG. 20 shows a tandem ligation workflow that enables chemical and topological modifications of circRNA. Panel A shows a schematic of synthetic linear mRNA prepared from DNA template and IVT, allowing incorporation of either unmodified NTP or modified NTP such as m1Ψ. Panel B is a schematic showing that the chemically synthesized oligo described in Panel A could be further replaced by a branched cap oligo conjugated through click chemistry. Panel C is a schematic showing that the branched capped oligo in Panel B could be further replaced by a multi-branched oligo with both branched cap and branched poly(A) tails, where both the branched capped oligo and the branched poly(A) oligo are chemically synthesized and modified/unmodified. Panel D is a schematic showing that the branched capped oligo in Panel B could be further replaced by a modified branched oligo, which consists of a capped oligo with an internal click chemistry handle and 3′phosphate conjugated to a poly(A) oligo with 5′phosphate and 3′click chemistry handle.

FIG. 21 shows experimental data supporting the production and characterization of chemically modified QRNA. Panel A shows that tandem ligation enabled synthesis of m1ψ modified QRNA and site-specific circRNA modifications. Synthetic linear mRNA encoding NanoLuciferase (NLuc) was prepared from DNA template by IVT with 100% m1ψ substitution. The triphosphorylated mRNA after IVT was then treated with Calf Intestinal Alkaline Phosphatase (CIAP) to yield 5′/3′-hydroxyl mRNA. A chemically synthesized oligonucleotide with a branched m7G cap and 5′/3′-phosphates was then ligated to the 5′/3′-hydroxyl mRNA by T4 RNA ligase, which ligates the 5′-phosphate on the oligo and 3′-hydroxyl of the mRNA. The ligation product was then further treated with RtcB ligase, which ligates the 5′-hydroxyl and 3′-phosphate ends to enable intramolecular circularization. Panel B shows an HPLC purification and denature gel electrophoresis characterization of QRNA posted tandem ligation circularization. Panel C shows a Double RNase H characterization of QRNA followed by PAGE gel assay to confirm branched-circular topology. Using one primer only produced no fragmentation but only linearization, confirming successful circularization. Using double primers produced fragments flanking the branched region, where branched topology was confirmed by gel shift. Panel D shows QRNA/circRNA with site-specific modifications or branched capping enhanced the translation. NLuc bioluminometry was measured 8 hrs post transfection and normalized Firefly luciferase as transfection control. Mean±s.e.m., n=3. P values were calculated by ordinary one-way ANOVA (alpha=0.05), with multiple comparisons to circ-NLuc with only 5-octadiynyl deoxyuridine (OU) handle (construct 45). ****P<0.0001, n.s. P>0.05. Panel E shows induction of NLuc circRNA translation by covalent or noncovalent internal caps. m1ψ-modified circRNA were prepared by LEGO and cotransfected with varying amounts of a capped oligo complementary to the ligated sequence at the circRNA 5′-UTR. QRNA with m7G (construct 48) was compared. n=3, biological replicates. Mean±sem. P values were calculated by ordinary one-way ANOVA with multiple comparisons against construct 61 without any cotransfected capped oligos (Dunnett's multiple comparison test). Panel F shows that an internal branched cap effectively induces translation of both the upstream open reading frame (ORF) and the downstream open reading frame (ORF). A linear mRNA was designed to contain a 5′ full length FLuc ORF and a 3′ 3×HiBIT ORF with an out of frame start codon from the FLuc ORF (construct 62). The dual-ORF mRNA was circularized (construct 63) and contained a branched cap (construct 64) where the LNAm7G cap was located upstream of the FLuc ORF and downstream of the 3×HiBIT ORF. n=3, biological replicates. Mean±sem. P values were calculated by unpaired two-sided t-test.

FIG. 22 shows schematics and experimental results supporting models of translation initiation by an internal branched cap. Panel A shows a schematic illustrating “threading mode”: eIF4E loads the branched cap from the entrance site of 40S and dissociates from the complex, but the downstream 40S scanning is blocked by the branched topology. Panel B shows a schematic illustrating “slot-in mode”: eIF4E-cap complex approaches the 40S from the exit side, and laterally slots the mRNA stem into the 40S mRNA-binding cleft, where the branched topology is tolerated. Panel C shows a schematic and experimental data illustrating the investigation of branched-cap induced ribosome slot-in position using alternative start codons. A firefly luciferase construct was designed to have an in-frame start codon (yellow) followed by an out-of-frame start codon (red) where distance between the branching point depicts the choice of translation start site selection by the branched cap. Synthetically, the Fluc reporter mRNA was capped on the “stem” by a functional m7G cap or a non-functional adenosine cap (A cap) to block 5′ degradation. The branched m7G cap is introduced Jul. 17, 2027 nt upstream from the in-frame FLuc start codon while the total length of the 5′ UTR is 30 nt. n=3, biological replicates. Mean±sem. P values were calculated by unpaired two-sided t-test. Panel D shows a schematic and experimental data illustrating the investigation of translation induction by a cis-acting branched cap on the 3′ UTR and a ploy (A) sequence at the 5′ UTR that were brought together by UTR-encoded homology. n=3, biological replicates. Mean±sem. P values were calculated by unpaired two-sided t-test. Panel E shows a schematic illustrating “cap-proximal” mechanisms of translation initiation through the lateral slot-in mode, which permits cap and 5′ UTR sequences connected to mRNA via either natural phosphodiester backbone or unnatural covalent linkages (proximal cis-acting caps). Furthermore, translation initiation could be driven by non-covalent cap structures in proximity of the 5′ end of mRNA via intramolecular (a distal cis-acting “3′ UTR-cap”) or intermolecular (a trans-acting, non-covalent 5′-cap) base-pairing. Figure discloses SEQ ID NOS 190-191 and 190, respectively, in order of appearance.

FIG. 23 shows the results of an expanded mocRNA modification screen in cultured HeLa cells. Panel A shows a series of time-delineated graphs showing Firefly luminescence for each condition and timepoint normalized to the mock ligation average for each timepoint. mocRNAs were prepared by ligation of modified oligos to Firefly luciferase mRNA. Untreated mRNA, mock ligation, or mocRNAs were co-transfected with unmodified Renilla luciferase mRNA as an internal control. Statistical significance was calculated for each sample with respect to the mock ligation sample at each timepoint. Statistical test used: Brown forsythe and Welch one-way ANOVA test, with Dunnett's T3 multiple comparisons test. *P*<0.05, **<0.01, **<0.001, ****<0.0001. Mean±S.D. Panel B shows a series of time-delineated graphs showing a Renilla luciferase internal control signal for each condition normalized to the corresponding mock ligation average for each timepoint. The statistical analysis was the same as described for Panel A.

FIG. 24 shows results of experiments assessing in vivo delivery of mocRNAs to mice. Panel A shows IVIS luminescence measurements taken at 6 hours or 24 hours post-injection with luciferase-encoding mRNA and 2MOE+PS_ddC mocRNA. 6 or 24 hours after injection with mRNA, D-luciferin reagent was injected intraperitoneally into each mouse (3 replicates for untreated vs. mocRNA, and allowed to incubate for 10 minutes prior to imaging. Panel B shows graphs depicting the total flux quantification over the imaging period. Statistical test: unpaired t-test, *P<0.05. Mean±S.D.

FIG. 25 shows results of experiments assessing the efficacy of various routes of administration and RNA formulation in mice. Panel A is a graph showing a quantification of IVIS luminescence taken at 6, 24, 48, and 72 hours post injection via intramuscular administration. Panel B is a graph showing a quantification of IVIS luminescence taken at 6, 24, 48, and 72 hours post injection via subcutaneous administration. Panel C is a graph showing a quantification of IVIS luminescence taken at 6, 24, 48, and 72 hours post injection via intradermal administration. Panel D is a graph showing a quantification of IVIS luminescence taken at 6, 24, 48, and 72 hours post injection via intratumoral administration. Under all experimental conditions, D-luciferin reagent was injected intraperitoneally into each mouse (3 mice for each route of injection) and allowed to incubate for 10 minutes prior to imaging. Statistical significance was calculated for each sample with respect to the PBS negative control for each corresponding timepoint. Statistical test: 2-way ANOVA, Sidak's multiple comparisons, *P<0.01, **<0.0043, ***<0.0001. Mean±S.D.

FIG. 26 shows a schematic and experimental results following 3′-capping of multi-capped RNA. Panel A is a schematic illustrating a conceptualization of a 5′/3′ multi-capped mRNA with nucleic acid direction inversion by a click chemistry linkage (green pentagon); the dashed lines indicate the ligation junction by T4 RNA ligase. Panel B is a schematic illustrating a conceptualization of a 5′/3′ multi-capped mRNA with nucleic acid direction inversion by a 3′-3′ inverted phosphodiester linkage. Panel C is a schematic illustrating exemplary synthesis methods of 5′-capped oligo with 3′-3′ inverted phosphodiester linkage. The oligo (/5PC/rArArArArA/invdT//invdT//5Phos/) was synthesized on solid phase using standard phosphoramidite chemistry on a Mermade6 DNA/RNA synthesizer. The oligo was then chemically capped with m7GDP-imidazole using our previous procedures, and purified by HPLC. After concentrating and desalting, the capped oligo was resuspended in water, and photo deprotected by UV irradiation at 366 nm for 15 min at room temperature. Panel D is a schematic illustrating the workflow for adding the 3′ cap to mRNA by ligation. Panel E is a plot showing characterization of/5PC/rArArArArA/invdT//invdT//5Phos/by MALDI-ToF on a Bruker Autoflex LRF Speed mass spectrometer. Some photo-cleaved products were observed due to the instrument laser, while the actual product is intact as indicated by HPLC. Panel F is a plot showing reverse phase HPLC purification and characterization of the oligo after capping and photo deprotection (stationary phase=PLRP-S, solvent phase=acetonitrile+100 mM hexylamine acetic acid in water, pH 7). The final product was at >98% purity. Panel G is a graph showing translation activity evaluated in Hela cells following lipofectamine transfection. An mRNA synthesized by in vitro transcription and capped by a non-functional adenosine cap was ligated to the oligo described in FIG. 26H. n=3, biological replicates. Mean±sem.

FIG. 27 shows schematics illustrating a photo-chemical crosslinking method to produce RNA/DNA hybrid molecules. Panel A is a schematic illustrating the photo-chemical crosslink reaction of RNA/DNA using cnvK as an example. The (capped) synthetic oligonucleotide can have a sequence that is partially or fully complementary to that of the other (capped, modified) synthetic oligo. The cnvK base is within the complementary region, and it is able to crosslink to a purine or other bases with an activated alkene (such as the electrophilic alkene in U, m5C, or C; or the thiophene alkene in TPT3, indicated in red) upon UV-A (~366 nm) irradiation for 1 to 60 seconds to undergo a [2+2] cycloaddition that covalently link the two oligos together. Panel B is a schematic depicting exemplary illustrations of topologically augmented RNAs including multi-capped RNA, 3′/5′ capped RNA, multi-tailed RNA, and capped circular RNAs, where the branched linkages were introduced by photo-crosslinking. Panel C is a schematic illustrating an exemplary workflow for synthesizing the multi-capped mRNA using photo-crosslinking and ligation. Synthetic oligos were chemically capped, and the oligos with/without the cnvK crosslinker were hybridized and crosslinked by UV-A. The branched, multi-capped oligo is then ligated using T4 RNA ligase to the 5′-phosphorylated mRNA (generated by in vitro transcription, followed by RppH treatment). Panel D is a schematic illustrating an exemplary workflow for synthesizing the capped circular mRNA using photo-crosslinking and ligation. mRNA is generated by in vitro transcription and digested with CIAP to yield a 3′/5′ hydroxyl mRNA. Synthetic oligonucleotide with phosphate groups on both 5′ and 3′ ends is hybridized with another capped synthetic oligo with cnvK to yield a branched, capped oligo with two phosphate groups. The branched capped oligo is then ligated to the 3′/5′ hydroxyl mRNA by T4 RNA ligase (ligating the 5′ phosphate and 3′ hydroxyl), and then circularized by RtcB (ligating the 3′ phosphate and 5′ hydroxyl).

FIG. 28 shows the structure of thiomorpholino oligo (TMO) ribose modifications, and experimental data describing the effect of thiomorpholino oligo (TMO) ribose modification on mRNA translation efficiency. The effect of TMO modification within the 5′ UTR (first nucleotide) on mRNA translation efficiency was evaluated by quantifying the luminescence signal of Fluc reporter mRNA at indicated time point. The result from TMO-modified mRNA was normalized to the unmodified control. n=3, biological replicates. Mean±sem.

FIG. 29 shows a schematic illustrating a model of ribosome landing position, and experimental data regarding the same. Panel A is a model illustrating “back” slot-ins for internal branched caps. When the branching site is >25 nt away from the 5′-terminus, the 40S slot-in may occur either upstream (“back” slot-in, indicated in blue) or downstream (normal slot-in, indicated in red) of the branching site. Panel B shows designs of six two-AUG HiBiT reporter mRNA constructs, and an evaluation of translation activity of the same in the rabbit reticulocyte lysate (RRL) system. All constructs contained 5′-exonuclease resistant modifications, a fixed branching site at the +4 base, a fixed out-of-frame start codon 46 nt downstream the branching site, a 30-A polyA tail, and an in-frame start codon encoding the HiBiT tag 26/29/32/35/38 nt downstream the branching site. 200 ng of the mRNA was translated in the rabbit reticulocyte lysate (RRL) for 1 hrs before assayed for HiBiT activity. n=3, independent experiments. Mean±sem. P values were calculated by unpaired two-sided t-test with Welch's correction. Figure discloses SEQ ID NOS 192-198, respectively, in order of appearance.

### DETAILED DESCRIPTION

Provided herein are modified mRNAs comprising modified 5′ cap regions comprising one or more modified nucleotides in order to improve stability and/or translation efficiency of the modified mRNA in cells and thereby enhance the production of encoded gene products, such as proteins. Also provided are methods of making the modified mRNAs described herein by adding to the 5′ end of an mRNA a 5′ cap region comprising one or more modified nucleotides at position +3 or higher. Also provided are methods of screening for altered mRNA stability and/or translation efficiency conferred by one or more modified nucleotides in a 5′ cap. Furthermore, provided herein are methods of producing a composition of RNA transcripts wherein at least 95% of the RNA transcripts in the composition comprise a 5′ cap.

In some embodiments, an RNA transcript comprising a modified 5′ cap region comprises a 5′ cap region comprising one or more modified nucleobases, phosphodiester linkages, sugar backbones, and/or 5′ caps.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. All references, patents and patent applications disclosed herein are incorporated by reference with respect to the subject matter for which each is cited, which in some cases may encompass the entirety of the document.

It should also be understood that, unless clearly indicated to the contrary, in any methods claimed herein that include more than one step or act, the order of the steps or acts of the method is not necessarily limited to the order in which the steps or acts of the method are recited.

### Definitions

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

In the claims, as well as in the specification, recitation of the phrase “between X and Y”, wherein X and Y are two separate values, it should be appreciated that these ranges include the use of these end values. For example, if a claim recites a range of between 1 and 10, this includes the values of 1, 10, and any value in between (e.g., 2, 3, 4, 5, 6, 7, 8, 9, 1.1, 1.2, 1.3, 1.4, 1.5, 1.6, 1.7, 1.8, 1.9, 1.01, 1.02, 1.03, 1.04, 1.05, 1.06, 1.07, 1.08, 1.09, etc.).

A “messenger RNA” (“mRNA”) as used herein refers to a nucleic acid comprising an open reading frame (ORF) encoding a gene product, such as a protein. An mRNA may comprise a poly-A region that is 3′ to the ORF. An mRNA may also comprise a 5′ untranslated region (5′ UTR) that is 5′ to (upstream of) the ORF, and a 3′ untranslated region (3′ UTR) that is 3′ to (downstream of) the ORF. A mRNA may also comprise a 5′ cap at the 5′ end of the mRNA.

An “open reading frame” (“ORF”), such as an ORF encoding a protein, as used herein refers to a nucleic acid sequence comprising a coding sequence that leads to the production of the protein when the ORF is translated. The nucleic acid sequence may be an RNA sequence, in which case translation of the RNA sequence produces a polypeptide with the amino acid sequence of the protein. The nucleic acid sequence may be a DNA sequence, in which case the protein is produced when an RNA polymerase uses the DNA sequence to transcribe an RNA molecule comprising an RNA sequence that is complementary to the DNA sequence, and translation of the RNA sequence produces a polypeptide with the amino acid sequence of the protein. An ORF typically begins with a START codon, such as AUG in the RNA sequence (ATG in the DNA sequence), and ends with a STOP codon, such as UAG, UAA, or UGA in the RNA sequence (TAG, TAA, or TGA in the DNA sequence), with the number of bases between the G of the start codon and the T or U of the STOP codon being a multiple of 3 (e.g., 3, 6, 9, 12, etc.).

With reference to numbering of the nucleotide positions within a nucleic acid molecule, a position of +1 refers to the first nucleotide of the nucleic acid molecule (e.g., of the RNA molecule), +2 is the second nucleotide, +3 is the third nucleotide, and so on.

In some embodiments of the modified mRNAs provided herein, the mRNA comprises a 5′ untranslated region (5′ UTR) and a 3′ untranslated region (3′ UTR). 5′ and 3′ UTRs are sequences within an mRNA that do not encode amino acids of the protein encoded by the mRNA, and are thus not part of the open reading frame. The 5′ UTR is 5′ to (upstream of) the open reading frame. The 3′ UTR is 3′ to (downstream of) the open reading frame. In some embodiments, the 3′ UTR comprises one or more nucleotides that are 3′ to the open reading frame and 5′ to (upstream of) the poly-A region of the mRNA.

In some embodiments of the modified mRNAs provided herein, the mRNA comprises, in 5′-to-3′ order: 1) a 5′ cap, optionally modified; 2) a modified 5′ UTR; 3) an open reading frame (ORF); 4) a 3′ UTR; and 5) a poly-A region. In some embodiments, the first nucleotide of the 5′ UTR is 3′ to (downstream of) the 5′ cap, and the last nucleotide of the 5′ UTR is 5′ to (upstream of) the first nucleotide of the ORF. In some embodiments, the first nucleotide of the ORF is 3′ to (downstream of) the last nucleotide of the 5′ UTR, and the last nucleotide of the ORF is 5′ to (upstream of) the first nucleotide of the 3′ UTR. In some embodiments, the ORF is between the last nucleotide of the 5′ UTR and the first nucleotide of the 3′ UTR. In some embodiments, the first nucleotide of the 3′ UTR is 3′ to (downstream of) the last nucleotide of the ORF, and the last nucleotide of the 3′ UTR is 5′ to (upstream of) the first nucleotide of the poly-A region. In some embodiments, the 5′ UTR is between the 5′ cap and the first nucleotide of the ORF. In some embodiments, the 3′ UTR is between the ORF and the poly-A region. In some embodiments, the 5′ cap is 5′ to (upstream of) the first nucleotide of the 5′ UTR. In some embodiments, the first nucleotide of the poly-A region is 3′ to (downstream of) the last nucleotide of the 3′ UTR.

In some embodiments, the RNA is a linear RNA. A linear RNA is an RNA with a 5′ terminal nucleotide and a 3′ terminal nucleotide. The 5′ terminal nucleotide of a linear RNA is covalently bonded to only one adjacent nucleotide of the RNA, with the adjacent nucleotide occurring 3′ to the 5′ terminal nucleotide in the nucleic acid sequence of the RNA. The 3′ terminal nucleotide of a linear RNA is covalently bonded to only one adjacent nucleotide of the RNA, with the adjacent nucleotide occurring 5′ to the 3′ terminal nucleotide in the nucleic acid sequence of the RNA. In a nucleic acid sequence comprising every nucleotide of a linear RNA in 5′-to-3′ order, the 5′ terminal nucleotide is the first nucleotide in the sequence, and the 3′ terminal nucleotide is the last nucleotide in the sequence.

In some embodiments, the mRNA is a circular mRNA. A circular mRNA is an mRNA with no 5′ terminal nucleotide or 3′ terminal nucleotide. Every nucleotide in a circular mRNA is covalently bonded to both 1) a 5′ adjacent nucleotide; and 2) a 3′ adjacent nucleotide. In a circular mRNA with a nucleic acid sequence comprising every nucleotide of the circular mRNA in 5′-to-3′ order, the last nucleotide of the nucleic acid sequence is covalently bonded to the first nucleotide of the nucleic acid sequence. In some embodiments of circular mRNAs with a 5′ cap region, a 5′ UTR, a 3′ UTR, and a poly-A region, the poly-A region is 3′ to (downstream from) the 3′ UTR and 5′ to (upstream of) the 5′ cap region.

An RNA molecule that can be translated is referred to as a messenger RNA, or mRNA. A DNA or RNA sequence encodes a gene through codons. A codon refers to a group of three nucleotides within a nucleic acid, such as DNA or RNA, sequence. An anticodon refers to a group of three nucleotides within a nucleic acid, such as a transfer RNA (tRNA), that are complementary to a codon, such that the codon of a first nucleic acid associates with the anticodon of a second nucleic acid through hydrogen bonding between the bases of the codon and anticodon. For example, the codon 5′-AUG-3′ on an mRNA has the corresponding anticodon 3′-UAC-5′ on a tRNA. During translation, a tRNA with an anticodon complementary to the codon to be translated associates with the codon on the mRNA, generally to deliver an amino acid that corresponds to the codon to be translated, or to facilitate termination of translation and release of a translated polypeptide from a ribosome.

Translation is the process in which the RNA coding sequence is used to direct the production of a polypeptide. The first step in translation is initiation, in which a ribosome associates with an mRNA, and a first transfer RNA (tRNA) carrying a first amino acid associates with the first codon, or START codon. The next phase of translation, elongation, involves three steps. First, a second tRNA with an anticodon that is complementary to codon following the START codon, or second codon, and carrying a second amino acid, associates with the mRNA. Second, the carbon atom of terminal, non-side chain carboxylic acid moiety of the first amino acid reacts with the nitrogen of the terminal, non-side chain amino moiety of the second amino acid carried, forming a peptide bond between the two amino acids, with the second amino acid being bound to the second tRNA, and the first amino acid bound to the second amino acid, but not the first tRNA. Third, the first tRNA dissociates from the mRNA, and the ribosome advances along the mRNA, such that the position at which the first tRNA associated with the ribosome is now occupied by the second tRNA, and the position previously occupied by the second tRNA is now free for an additional tRNA carrying an additional amino acid to associate with the mRNA. These three steps of 1) association of a tRNA carrying amino acid, 2) formation of a peptide bond, which adds an additional amino acid to a growing polypeptide, and 3) advancement of the ribosome along the mRNA, continue until the ribosome reaches a STOP codon, which results in termination of translation. Generally, tRNAs that associate with STOP codons do not carry an amino acid, so the association of a tRNA that does not carry an amino acid during the elongation step results in cleavage of the bond between the polypeptide and the tRNA carrying the final amino acid in the polypeptide, such that the polypeptide is released from the ribosome. Alternatively, ribosomes may dissociate from the mRNA and release the polypeptide if no tRNA associates with the STOP codon.

A “nucleic acid,” or “polynucleotide,” as used herein, refers to an organic molecule comprising two or more covalently bonded nucleotides. A “nucleotide,” as used herein, refers to an organic molecule comprising a 1) a nucleoside comprising a sugar covalently bonded to a nitrogenous base (nucleobase); and 2) a phosphate group that is covalently bonded to the sugar of the nucleoside. Nucleotides in a polynucleotide are typically joined by a phosphodiester bond, in which the 3′ carbon of the sugar of a first nucleotide is linked to the 5′ carbon of the sugar of a second nucleic acid by a bridging phosphate group. Typically, the bridging phosphate comprises two non-bridging oxygen atoms, which are bonded only to a phosphorus atom of the phosphate, and two bridging oxygen atoms, each of which connects the phosphorus atom to either the 3′ carbon of the first nucleotide or the 5′ carbon of the second nucleotide. In a nucleic acid sequence describing the order of nucleotides in a nucleic acid, a first nucleotide is said to be 5′ to (upstream of) a second nucleotide if the 3′ carbon of first nucleotide is connected to the 5′ carbon of the second nucleotide. Similarly, a second nucleotide is said to be 3′ to (downstream of) a first nucleotide if the 5′ carbon of the second nucleotide is connected to the 3′ carbon of the first nucleotide. Nucleic acid sequences are typically read in 5′->3′ order, starting with the 5′ nucleotide and ending with the 3′ nucleotide.

A “modified nucleotide,” as used herein, refers to a nucleotide with a structure that is not the canonical structure of an adenosine nucleotide, cytidine nucleotide, guanine nucleotide, or uracil nucleotide. A canonical structure of a molecule refers to a structure that is generally known in the art to be the structure referred to by the name of the molecule. A canonical structure of an adenosine nucleotide, which comprises an adenine base, ribose sugar, and one or more phosphate groups, is shown below, in the form of adenosine monophosphate:

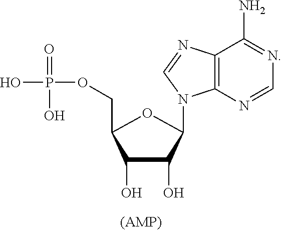

The canonical structure of AMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The canonical structure of a cytosine nucleotide which comprises a cytosine base, ribose sugar, and one or more phosphate groups, is shown below, in the form of cytidine monophosphate:

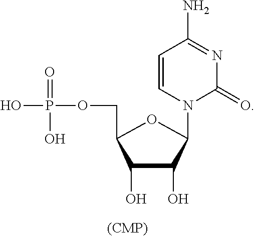

The canonical structure of CMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The canonical structure of a guanine nucleotide which comprises a guanine base, ribose sugar, and one or more phosphate groups, is shown below, in the form of guanosine monophosphate:

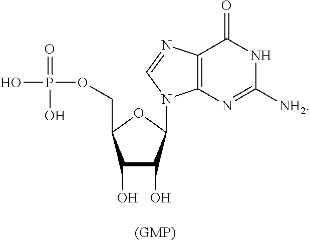

The canonical structure of GMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The canonical structure of a uracil nucleotide which comprises a uracil base, ribose sugar, and one or more phosphate groups, is shown below, in the form of uridine monophosphate:

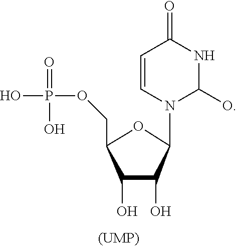

The canonical structure of UMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The structure of a modified nucleotide may differ from the structure of a canonical nucleotide due to one or more modifications in the sugar, nitrogenous base, or phosphate of the nucleotide. In some embodiments, the modified nucleotide comprises a modified nucleoside that is not the canonical structure of an adenine nucleoside, cytosine nucleoside, guanine nucleoside, or uracil nucleoside.

An example of a canonical structure of adenosine, an adenine nucleoside, is reproduced below:

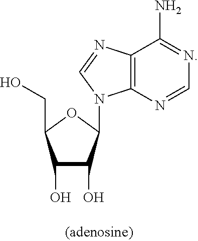

The canonical structure of adenosine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

An example of a canonical structure of cytidine, a cytosine nucleoside, is reproduced below:

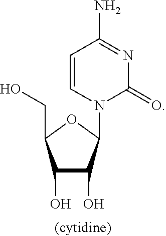

The canonical structure of cytidine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

An example of a canonical structure of guanosine, a guanine nucleoside, is reproduced below:

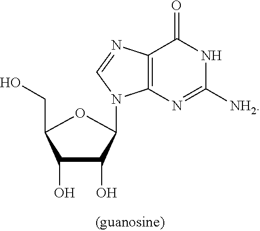

The canonical structure of guanosine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

An example of a canonical structure of uridine, a uracil nucleoside, is reproduced below:

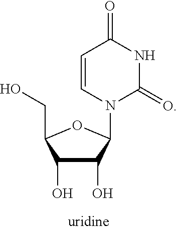

The canonical structure of uridine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

A “ligase,” as used herein, refers to an enzyme that is capable of forming a covalent bond between two nucleotides, and the process of “ligation” refers to the formation of the covalent bond between the two nucleotides.

A “poly-A tail,” as used herein, refers to a nucleic acid sequence comprising adenosine nucleotides that is attached to the 3′ end of a nucleic acid, such as an RNA. A poly-A tail or poly-A region may consist of nucleotides that are 25-100%, 30-100%, 40-100%, 50-100%, 60-100%, 70-100%, 80-100%, 90-100%, 95-100%, 96-100%, 97-100%, 98-100%, or 99-100% adenosine nucleotides. As used herein, the terms “poly-A tail” and “poly-A region” are used interchangeably. The adenosine nucleotides comprised by a poly-A tail may be canonical adenosine nucleotides or modified (non-canonical) adenosine nucleotides.

A “5′ cap,” as used herein, refers to one or more nucleotides that are covalently attached to the 5′ end of a nucleic acid, such as an RNA molecule. A “5′ cap region,” as used herein, refers to a nucleic acid comprising a 5′ nucleotide cap and one or more modified nucleotides. A 5′ cap may comprise a 5′ capping nucleotide that is attached to the 5′ end of a mRNA by a 5′ to 5′ triphosphate internucleotide linkage. In some embodiments, a nucleotide attached to a mRNA by a 5′ to 5′ triphosphate internucleotide linkage is referred to as a “native” 5′ capping nucleotide. In some embodiments, a native 5′ capping nucleotide is a 7-methylguanosine (m7G) nucleotide. In some embodiments, a 5′ cap is a modified 5′ cap, comprising one or more modified nucleotides, such as the 5′ capping nucleotide, or one or more modified internucleotide modifications, such as modifications to the 5′ to 5′ triphosphate internucleotide linkage. In some embodiments, a 5′ cap comprises one or more nucleotides with a sugar modification, such as 2′-O-methylation.

An example of a canonical structure of 7-methylguanosine (m7G) attached to a ribonucleic acid sequence (e.g., a mRNA) by a 5′ to 5′ triphosphate internucleotide linkage is reproduced below:

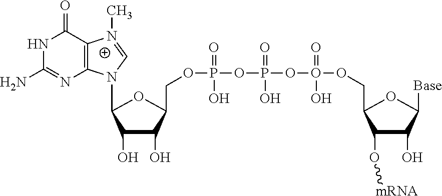

A “counterion” or “anionic counterion” is a negatively charged group associated with a positively charged group in order to maintain electronic neutrality. In some embodiments, an anionic counterion is monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

Modified mRNAs

In some aspects, the present disclosure provides modified mRNAs comprising a 5′ cap region, wherein the 5′ cap region comprises a 5′ nucleotide cap and one or more modified nucleotides. In some embodiments, a modified mRNA is a modified linear mRNA. In some embodiments, a modified mRNA is a modified circular mRNA.

The “5′ cap region”, as used herein, refers to a region of an mRNA that is 5′ to (upstream of) the ORF. In some embodiments, the 5′ cap region comprises a 5′ untranslated region (5′ UTR). In some embodiments, the 5′ cap region comprises a 5′ cap. In eukaryotic cells, mRNAs possess a cap structure in which an N7-methylguanine (m7G) moiety is linked to the first transcribed nucleotide by a 5′-5′-triphosphate bridge. The 5′ cap plays multiple roles in pre-mRNA splicing, mRNA export, RNA stability through blocking degradation by the 5′-3′ exoribonuclease (ExoN), escaping recognition of the cellular innate immune system, and the production of proteins encoded by mRNAs. The presence of a 5′ cap in an mRNA facilitates the initiation of translation (see, e.g., Gallie. Genes & Dev. 1991. 5:2108-2116, and Munroe et al. Mol Cell Biol. 1990. 10 (7): 3441-3455). The 5′ cap is added by a 5′ capping enzyme, such as mRNA guanylyltransferase. Translation initiation is a rate-limiting step of mRNA translation and heavily depends on the 5′ N7-methylguanosine (m7G) cap and its interaction with eukaryotic translation initiation factors (eIFs), including the cap-binding eIF4E protein. Chemical modification on or near the 5′ cap influence binding of eIFs and decapping enzymes, which subsequently impact downstream mRNA translation and stability. For example, the presence of 2′ O-methyl(2′OMe) groups on the first and second transcribed nucleotides (known as Cap-0/1/2, referring to zero, one, or two 2′OMe groups) reduces mRNA immunogenicity and increases protein expression. Additionally, N6-methyladenosine (m6A) on the first base controls mRNA stability through increased resistance to decapping by Dcp2. Furthermore, the 5′ cap stabilizes the mRNA by protecting the ORF from the activity of exonucleases, such as polynucleotide phosphorylase (PNPase), which can remove 3′ and 5′ nucleotides from an mRNA. As an exonuclease removes nucleotides, the mRNA becomes progressively shorter, and once all the nucleotides downstream of the open reading frame are removed, the nucleotides removed by the exonuclease will be nucleotides of the ORF. Removal of nucleotides from the ORF prevents translation of the encoded protein. Additionally, the association of an exonuclease with the mRNA near the ORF can inhibit translation by sterically hindering ribosomes and tRNAs from associating with the mRNA. The composition of a 5′ cap typically comprises a 5′ m7G attached to the mRNA by a 5′ to 5′ triphosphate internucleotide linkage.

In some embodiments of the modified mRNAs provided herein, the modified mRNA comprises one or more modified nucleotides in the 5′ cap region of the mRNA. In some embodiments, the 5′ cap region includes one or more nucleotides that are not canonical adenosine, cytidine, guanosine, or uridine nucleotides. In some embodiments, the 5′ cap region comprises between 1 and 3, between 3 and 5, between 5 and 7, or between 7 and 10 5′ caps. In some embodiments, the 5′ cap region comprises between 10-500 nucleotides. In some embodiments, the 5′ cap region comprises between 10 and 15, between 15 and 20, between 20 and 25, between 25 and 50, between 50 and 100, between 100 and 150, between 150 and 200, between 200 and 300, between 300 and 400, or between 400 and 500 nucleotides.

### Chemical Synthesis of Cap Regions

Existing methods for preparing capped linear mRNA do not accommodate modifications that are not tolerated by RNA polymerase or capping enzymes, nor modifications that extend beyond the first two bases, creating a screening bias due to differing cap incorporation efficiencies. To overcome these challenges, the capping process was decoupled from mRNA synthesis as described herein.

In some embodiments of the methods provided herein, the method comprises first synthesizing a 5′-phosphorylated RNA oligonucleotide with a specific sequence and/or desired modifications. In the methods described herein, the synthesized 5′-phosphorylated RNA oligonucleotide defines the 5′ UTR when ligated to an RNA transcript. Thus, as used herein, the terms “5′-phosphorylated RNA oligonucleotide,” “5′-phosphorylated oligonucleotide,” and “5′-phosphorylated UTR” are used interchangeably. In some embodiments, the 5′-phosphorylated RNA oligonucleotide comprises one or more modified nucleotides which may affect RNA translation and/or stability.

In some embodiments, the 5′-phosphorylated oligonucleotide comprises a modified phosphate, resulting in a modified internucleotide linkage. Modified phosphates used in the present disclosure may be, but are not limited to, phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, more than one modified phosphate is used. In some embodiments, the 5′-phosphorylated oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified phosphates. In some embodiments, the 5′-phosphorylated oligonucleotide comprises between 1 and 3, between 3 and 5, between 5 and 10, between 10 and 15, between 15 and 30, between 30 and 50, between 50 and 100, or between 100 and 200 modified phosphates. In some embodiments, the modified phosphates of the 5′-phosphorylated oligonucleotide comprise about 3%, about 5%, about 10%, about 15%, about 20%, about 30%, about 40%, about 50%, about 60%, about 70%, about 80%, about 90%, or 100% of the total phosphates in the 5′-phosphorylated oligonucleotide.

In some embodiments, the 5′-phosphorylated oligonucleotide comprises a modified sugar. Modified sugars used in the present disclosure may be, but are not limited to, 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose. L-adenosine (LA) refers to the enantiomer of D-adenosine. In some embodiments, the 5′-phosphorylated oligonucleotide comprises a modified sugar backbone. In some embodiments, the modified sugar backbone is selected from the following:

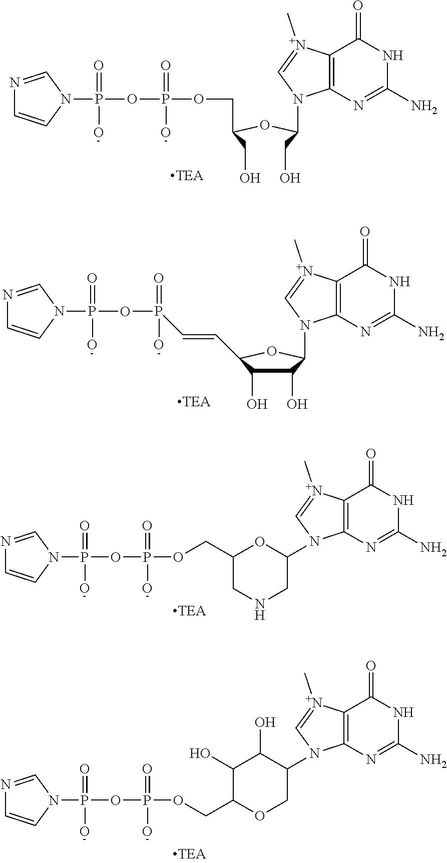

A locked nucleic acid is a nucleotide having a modified ribose moiety in which the ribose moiety comprises an extra bridge connecting the 2′ and 4′ carbons. This structure effectively “locks” the ribose in the 3′-endo structural conformation. In some embodiments, the 5′-phosphorylated oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified sugars. In some embodiments, the 5′-phosphorylated oligonucleotide comprises between 1 and 3, between 3 and 5, between 5 and 10, between 10 and 15, between 15 and 30, between 30 and 50, between 50 and 100, or between 100 and 200 modified sugars. In some embodiments, the modified sugars of the 5′-phosphorylated oligonucleotide comprise about 3%, about 5%, about 10%, about 15%, about 20%, about 30%, about 40%, about 50%, about 60%, about 70%, about 80%, about 90%, or 100% of the total sugars in the 5′-phosphorylated oligonucleotide.

In some embodiments, the 5′-phosphorylated oligonucleotide comprises a modified nucleobase. Modified nucleobases used in the present disclosure may be, but are not limited to, inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, the 5′-phosphorylated oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified nucleobases. In some embodiments, the 5′-phosphorylated oligonucleotide comprises between 1 and 3, between 3 and 5, between 5 and 10, between 10 and 15, between 15 and 30, between 30 and 50, between 50 and 100, or between 100 and 200 modified nucleobases. In some embodiments, the modified nucleobases of the 5′-phosphorylated oligonucleotide comprise about 3%, about 5%, about 10%, about 15%, about 20%, about 30%, about 40%, about 50%, about 60%, about 70%, about 80%, about 90%, or 100% of the total nucleobases in the 5′-phosphorylated oligonucleotide.

In some embodiments, the 5′-phosphorylated oligonucleotide is synthesized on a solid-phase support. In some embodiments, the solid support is controlled-pore glass (CPG) or polystyrene (PS). In some embodiments, the 5′-phosphorylated oligonucleotide is synthesized via phosphoramidite oligonucleotide synthesis. In some embodiments, the 5′-phosphorylated oligonucleotide is synthesized in a solvent system comprising a nonpolar counterion. In some embodiments, the nonpolar counterion used in oligonucleotide synthesis is ammonium. In some embodiments, the nonpolar counterion used in oligonucleotide synthesis is ammonium.

In some embodiments, a 5′ cap is added to the 5′-phosphorylated oligonucleotide to produce a 5′-capped oligonucleotide (i.e., a 5′-capped UTR). A 5′ cap can be added to an RNA oligonucleotide via enzymatic or chemical reactions. In some embodiments, the cap is added to the 5′-phosphorylated oligonucleotide through chemical capping methods. Chemical capping may be performed by any method known in the art. Preferably, the chemical capping reaction is performed through an anhydrous reaction between the 5′-phosphorylated RNA oligonucleotide and a capping nucleotide conjugated to imidazole in the presence of 1-methylimidazole (see Abe et al., “Complete Chemical Synthesis of Minimal Messenger RNA by Efficient Chemical Capping Reaction” ACS Chem. Biol. 2022, 17:1308-1314). In this method, the cap of interest is first conjugated to imidazole. A chemical reaction is then performed between the imidazole-conjugated capping oligonucleotide and a 5′-phoshporylated oligonucleotide under anhydrous conditions and in the presence of 1-methylimidazole. In some embodiments, the capping reaction is performed in dimethyl sulfoxide (DMSO). The desired product of this reaction is an oligonucleotide capped on its 5′ end with the cap of interest.

In some embodiments, the 5′ cap used in the present disclosure may be, but is not limited to, 7-methyguanosine (m7G), N7,3′-O-dimethyl-guanosine-5′-triphosphate-5′-guanosine (m7G-3′m-ppp-G), N7,2′-O-dimethyl-guanosine-5′-triphosphate-5′-guanosine (m7Gm-ppp-G), 7-benzylguanosine (Bn7G), chlorobenzylguanosine (ClBn7G), m7G bearing an LNA sugar (m7G-LNA), chlorobenzyl-O-ethoxyguanosine (ClBnOEt7G), 7-(4-chlorophenoxyethyl)-guanosine, 7-ethyl guanosine (e7G), 7-propyl guanosine (p7G), 7-isopropyl guanosine (ip7G), 7-butyl guanosine (b7G), 7-isobutyl guanosine (ib7G), 7-cyclopentyl guanosine (cp7G), 7-(carboxymethyl) guanosine (cm7G), 7-(2-phenylethyl) guanosine [7-(2-PhEt)G], 7-(1-phenylethyl) guanosine [7-(1-PhEt)G], m⁷Gppp_{BH3}G (D1 and D2 stereoisomers), m⁷Gpp_{BH3}G (D1 and D2 stereoisomers), m⁷Gp_{BH3}G (D1 and D2 stereoisomers), m⁷Gpp_{BH3}pm⁷G, m₂^{7,2′-O}GpppBH3G (D1 and D2 stereoisomers), m₂^{7,2′-O}Gpp_{BH3}PG (D1 and D2 diastereomers), m₂^{7,2′-O}GppspG (D1 and D2 diastereomers), N-Arylmethyl analogs, glyceryl, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2′-inverted nucleotide moiety, 3′-2 ‘-inverted abasic moiety, 1,4-butanediol phosphate, 3’-phosphoramidate, hexylphosphate, aminohexyl phosphate, 3′-phosphate, 3′-phosphorothioate, phosphorodithioate, cap1, cap2, cap3, cap4, ARCA, modified ARCA, inosine, N1-methylguanosine, LNA-guanosine, 2-azido-guanosine, and a bridging or non-bridging methylphosphonate moiety.

In some embodiments, the 5′-phosphorylated oligonucleotide comprises a 5′ cap comprising a modified sugar backbone. In some embodiments, the 5′ cap comprising a modified sugar backbone is a modified m7G cap. In some embodiments, the 5′ cap comprising a modified sugar backbone comprises a modified sugar backbone selected from the following:

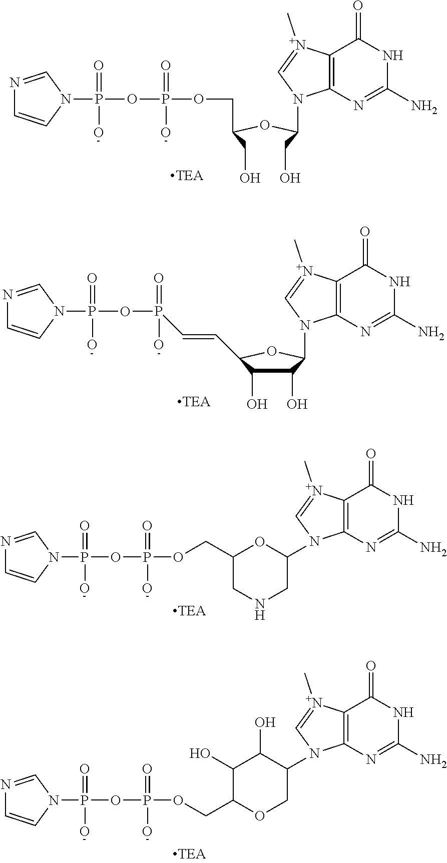

Thus, in some embodiments, a 5′ cap region provided herein comprises a 5′-capped oligonucleotide (i.e., a 5′-capped UTR) synthesized as described above. In some embodiments, the 5′ cap region comprises a modified 5′ cap, one or more modified phosphates, one or more modified sugars, and/or one or more modified nucleobases. The 5′ cap region may comprise any combination of modifications. In some embodiments, the 5′ cap region is between 5 and 50, between 10 and 45, between 15 and 40, between 20 and 35, between 25 and 30, or more than 30 nucleotides in length. In some embodiments, the 5′ cap region comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified nucleotides. In some embodiments, the 5′ cap region comprises between 1 and 3, between 3 and 5, between 5 and 10, between 10 and 15, between 15 and 30, between 30 and 50, between 50 and 100, or between 100 and 200 modified nucleotides. In some embodiments, the modified nucleotides of the 5′ cap region comprise about 3%, about 5%, about 10%, about 15%, about 20%, about 30%, about 40%, about 50%, about 60%, about 70%, about 80%, about 90%, or 100% of the total nucleotides in the 5′ cap region.

In some embodiments of the methods provided herein, the method comprises first synthesizing a 3′-phosphorylated RNA oligonucleotide with a specific sequence and/or desired modifications. In the methods described herein, the synthesized 3′-phosphorylated RNA oligonucleotide defines the 3′ UTR when ligated to an RNA transcript. Thus, as used herein, the terms “3′-phosphorylated RNA oligonucleotide,” “3′-phosphorylated oligonucleotide,” and “3′-phosphorylated UTR” are used interchangeably. In some embodiments, the 3′-phosphorylated RNA oligonucleotide comprises one or more modified nucleotides which may affect RNA translation and/or stability.

In some embodiments, the 3′-phosphorylated oligonucleotide comprises a modified phosphate, resulting in a modified internucleotide linkage. Modified phosphates used in the present disclosure may be, but are not limited to, phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, more than one modified phosphate is used. In some embodiments, the 3′-phosphorylated oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified phosphates. In some embodiments, the 3′-phosphorylated oligonucleotide comprises between 1 and 3, between 3 and 5, between 5 and 10, between 10 and 15, between 15 and 30, between 30 and 50, between 50 and 100, or between 100 and 200 modified phosphates. In some embodiments, the modified phosphates of the 3′-phosphorylated oligonucleotide comprise about 3%, about 5%, about 10%, about 15%, about 20%, about 30%, about 40%, about 50%, about 60%, about 70%, about 80%, about 90%, or 100% of the total phosphates in the 3′-phosphorylated oligonucleotide.

In some embodiments, the 3′-phosphorylated oligonucleotide comprises a modified sugar. Modified sugars used in the present disclosure may be, but are not limited to, 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose. L-adenosine (LA) refers to the enantiomer of D-adenosine. In some embodiments, the 3′-phosphorylated oligonucleotide comprises a modified sugar backbone. In some embodiments, the modified sugar backbone is selected from the following:

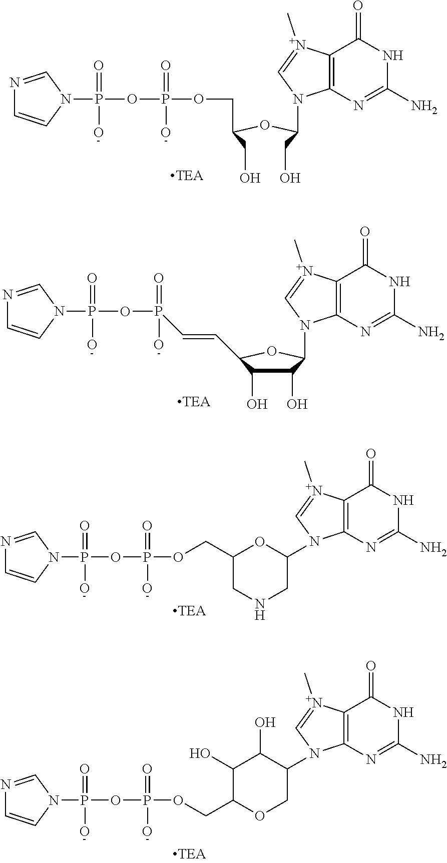

A locked nucleic acid is a nucleotide having a modified ribose moiety in which the ribose moiety comprises an extra bridge connecting the 2′ and 4′ carbons. This structure effectively “locks” the ribose in the 3′-endo structural conformation. In some embodiments, the 3′-phosphorylated oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified sugars. In some embodiments, the 3′-phosphorylated oligonucleotide comprises between 1 and 3, between 3 and 5, between 5 and 10, between 10 and 15, between 15 and 30, between 30 and 50, between 50 and 100, or between 100 and 200 modified sugars. In some embodiments, the modified sugars of the 3′-phosphorylated oligonucleotide comprise about 3%, about 5%, about 10%, about 15%, about 20%, about 30%, about 40%, about 50%, about 60%, about 70%, about 80%, about 90%, or 100% of the total sugars in the 3′-phosphorylated oligonucleotide.

In some embodiments, the 3′-phosphorylated oligonucleotide comprises a modified nucleobase. Modified nucleobases used in the present disclosure may be, but are not limited to, inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, the 3′-phosphorylated oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified nucleobases. In some embodiments, the 3′-phosphorylated oligonucleotide comprises between 1 and 3, between 3 and 5, between 5 and 10, between 10 and 15, between 15 and 30, between 30 and 50, between 50 and 100, or between 100 and 200 modified nucleobases. In some embodiments, the modified nucleobases of the 3′-phosphorylated oligonucleotide comprise about 3%, about 5%, about 10%, about 15%, about 20%, about 30%, about 40%, about 50%, about 60%, about 70%, about 80%, about 90%, or 100% of the total nucleobases in the 3′-phosphorylated oligonucleotide.

In some embodiments, the 3′-phosphorylated oligonucleotide is synthesized on a solid-phase support. In some embodiments, the solid support is controlled-pore glass (CPG) or polystyrene (PS). In some embodiments, the 3′-phosphorylated oligonucleotide is synthesized via phosphoramidite oligonucleotide synthesis. In some embodiments, the 3′-phosphorylated oligonucleotide is synthesized in a solvent system comprising a nonpolar counterion. In some embodiments, the nonpolar counterion used in oligonucleotide synthesis is ammonium. In some embodiments, the nonpolar counterion used in oligonucleotide synthesis is ammonium.

In some embodiments, a 3′ cap is added to the 3′-phosphorylated oligonucleotide to produce a 3′-capped oligonucleotide (i.e., a 3′-capped UTR). A 3′ cap can be added to an RNA oligonucleotide via enzymatic or chemical reactions. In some embodiments, the cap is added to the 3′-phosphorylated oligonucleotide through chemical capping methods. Chemical capping may be performed by any method known in the art. Preferably, the chemical capping reaction is performed through an anhydrous reaction between the 3′-phosphorylated RNA oligonucleotide and a capping nucleotide conjugated to imidazole in the presence of 1-methylimidazole (see Abe et al., “Complete Chemical Synthesis of Minimal Messenger RNA by Efficient Chemical Capping Reaction” ACS Chem. Biol. 2022, 17:1308-1314). In this method, the cap of interest is first conjugated to imidazole. A chemical reaction is then performed between the imidazole-conjugated capping oligonucleotide and a 3′-phoshporylated oligonucleotide under anhydrous conditions and in the presence of 1-methylimidazole. In some embodiments, the capping reaction is performed in dimethyl sulfoxide (DMSO). The desired product of this reaction is an oligonucleotide capped on its 3′ end with the cap of interest.

In some embodiments, the 3′ cap used in the present disclosure may be, but is not limited to, 7-methyguanosine (m7G), N7,3′-O-dimethyl-guanosine-5′-triphosphate-5′-guanosine (m7G-3′m-ppp-G), N7,2′-O-dimethyl-guanosine-5′-triphosphate-5′-guanosine (m7Gm-ppp-G), 7-benzylguanosine (Bn7G), chlorobenzylguanosine (ClBn7G), m7G bearing an LNA sugar (m7G-LNA), chlorobenzyl-O-ethoxyguanosine (ClBnOEt7G), 7-(4-chlorophenoxyethyl)-guanosine, 7-ethyl guanosine (e7G), 7-propyl guanosine (p7G), 7-isopropyl guanosine (ip7G), 7-butyl guanosine (b7G), 7-isobutyl guanosine (ib7G), 7-cyclopentyl guanosine (cp7G), 7-(carboxymethyl) guanosine (cm7G), 7-(2-phenylethyl) guanosine [7-(2-PhEt)G], 7-(1-phenylethyl) guanosine [7-(1-PhEt)G], m⁷Gppp_{BH3}G (D1 and D2 stereoisomers), m⁷Gpp_{BH3}G (D1 and D2 stereoisomers), m⁷Gp_{BH3}G (D1 and D2 stereoisomers), m⁷Gpp_{BH3}pm⁷G, m₂^{7,2′-O}GpppBH3G (D1 and D2 stereoisomers), m₂^{7,2′-O}Gpp_{BH3}PG (D1 and D2 diastereomers), m₂^{7,2′-O}GppspG (D1 and D2 diastereomers), N-Arylmethyl analogs, glyceryl, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2′-inverted nucleotide moiety, 3′-2 ‘-inverted abasic moiety, 1,4-butanediol phosphate, 3’-phosphoramidate, hexylphosphate, aminohexyl phosphate, 3′-phosphate, 3′-phosphorothioate, phosphorodithioate, cap1, cap2, cap3, cap4, ARCA, modified ARCA, inosine, N1-methylguanosine, LNA-guanosine, 2-azido-guanosine, and a bridging or non-bridging methylphosphonate moiety.

In some embodiments, the 3′-phosphorylated oligonucleotide comprises a 3′ cap comprising a modified sugar backbone. In some embodiments, the 3′ cap comprising a modified sugar backbone is a modified m7G cap. In some embodiments, the 3′ cap comprising a modified sugar backbone comprises a modified sugar backbone selected from the following:

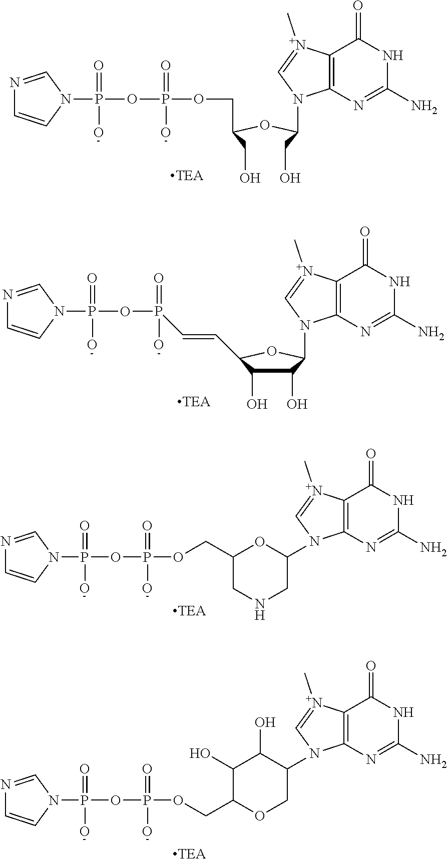

Thus, in some embodiments, a 3′ cap region provided herein comprises a 3′-capped oligonucleotide (i.e., a 3′-capped UTR) synthesized as described above. In some embodiments, the 3′ cap region comprises a modified 3′ cap, one or more modified phosphates, one or more modified sugars, and/or one or more modified nucleobases. The 3′ cap region may comprise any combination of modifications. In some embodiments, the 3′ cap region is between 5 and 50, between 10 and 45, between 15 and 40, between 20 and 35, between 25 and 30, or more than 30 nucleotides in length. In some embodiments, the 3′ cap region comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more modified nucleotides. In some embodiments, the 3′ cap region comprises between 1 and 3, between 3 and 5, between 5 and 10, between 10 and 15, between 15 and 30, between 30 and 50, between 50 and 100, or between 100 and 200 modified nucleotides. In some embodiments, the modified nucleotides of the 3′ cap region comprise about 3%, about 5%, about 10%, about 15%, about 20%, about 30%, about 40%, about 50%, about 60%, about 70%, about 80%, about 90%, or 100% of the total nucleotides in the 3′ cap region.

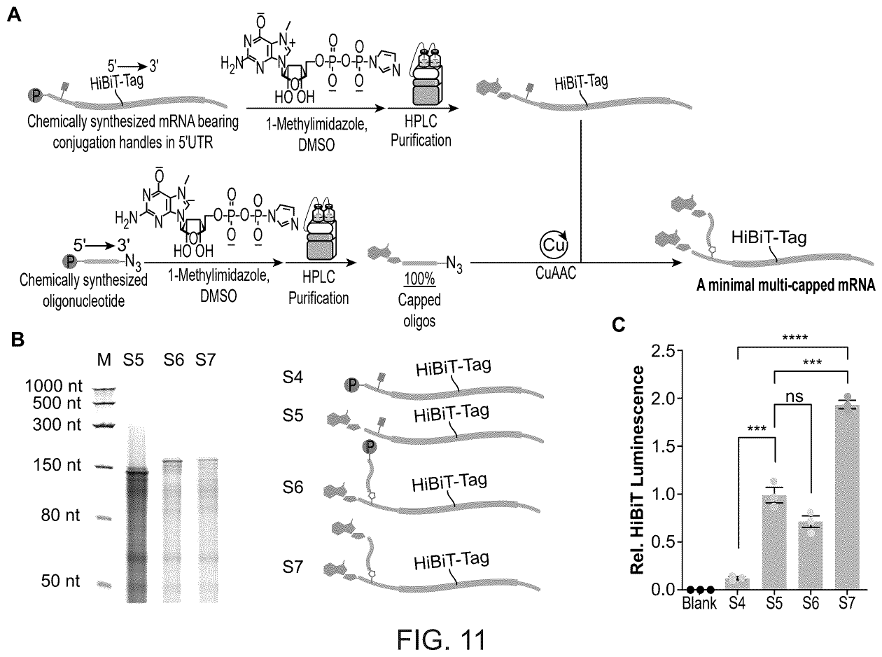

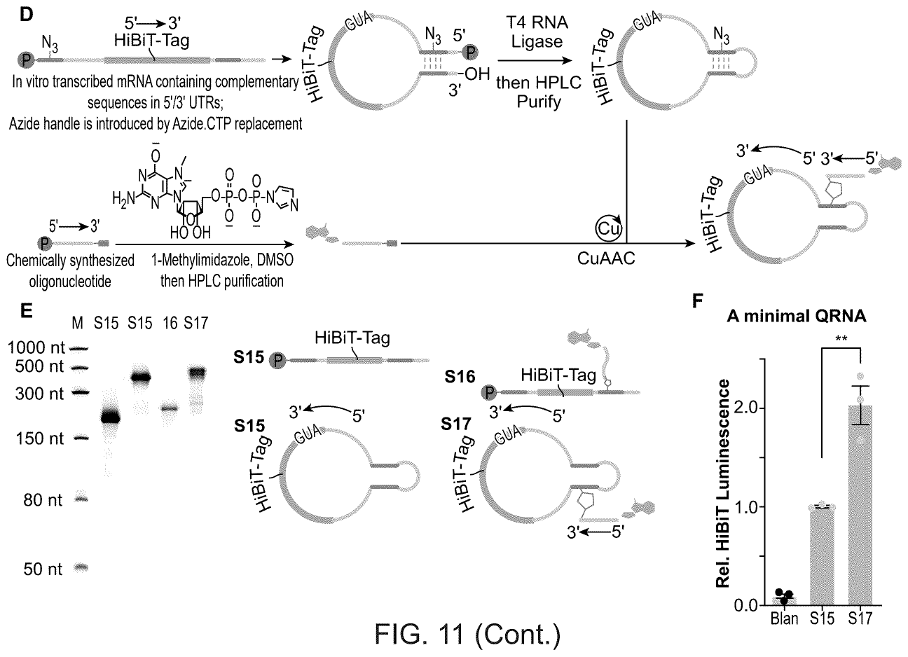

In some embodiments, a capped RNA transcript as provided herein comprises more than one 5′ cap or 5′ UTR region. In some embodiments, a capped RNA transcript as provided herein comprises more than one 3′ cap or 3′ UTR region. In some embodiments, a capped RNA transcript as provided herein comprises more than one poly-A tail. Methods of producing multi-capped RNA strands have been described in U.S. Patent Application No. 63/300,602, the contents of which are incorporated herein in their entirety. These methods comprise incorporating azide handles into an RNA molecule such that it is compatible with an alkyne-containing nucleotide to undergo a click chemistry reaction. The present application builds upon these these techniques. In some embodiments, an azide handle is introduced into the RNA molecule through tRNA guanine transglycosylase (TGT) in combination with a pre-queuosine 1 (preQ1) substrate (Ehret et al. “Site-specific covalent conjugation of modified mRNA by tRNA guanine transglycosylase.” Mol. Pharm. 15, 737-742 (2018)). In some embodiments, an azide handle is incorporated into an RNA molecule (e.g., 5′-phosphorylated RNA oligonucleotide or a capped RNA transcript) through during transcription, providing an azide-linked nucleotide as substrate for incorporation into a growing RNA strand (e.g., 5-Azido-PEG4-CTP). In some embodiments, more than one azide handle is introduced into an RNA molecule. In some embodiments, more than one azide handle is introduced into an RNA molecule using more than one introduction technique (e.g., both TGT and IVT). In some embodiments, a synthetic oligonucleotide can contain a photoactive alkene capable of undergoing a photochemical crosslinking reaction. Examples of photoactive alkenes include the cyanovinyl moiety in cnvK and cnvD (see cambio.co.uk/library/images/html_images/Glen_structures/GR30-2.pdf), and the alkene in furan or pyrone in coumarin or psoralen (See pubs.rsc.org/en/content/articlepdf/2022/ra/d1ra05951c FIGS. 11, 12, 13, and 14). A crosslinking acceptor can be, without limitation, a derivative of U, C (such as m5C), or TPT3. In some embodiments of a photocrosslinking reaction between two oligonucleotides, at least about at least about 1 to 10 bases 5′ (upstream) of the photocrosslinker should be complementary to the other oligonucleotide. In some embodiments of a photocrosslinking reaction between two oligonucleotides, at least about at least about 1 to 10 bases 3′ (downstream) of the photocrosslinker should be complementary to the other oligonucleotide.

Producing RNA Transcripts with a Modified 5′ Cap Region

In some aspects, the methods provided herein produce a capped RNA transcript with a modified 5′ cap region. In some embodiments, the methods comprise attaching a 5′ cap region as described herein to an RNA precursor, thereby producing a capped RNA transcript comprising a modified 5′ cap and UTR.

In some aspects, the present disclosure provides methods of producing modified RNAs comprising ligating an RNA (e.g., an RNA precursor) to a 5′ cap region comprising a 5′ cap and a 5′ UTR in the presence of a ligase, whereby the ligase forms a covalent bond between the 3′ nucleotide of the 5′ cap region and the 5′ nucleotide of the RNA (e.g., the RNA precursor) to produce capped RNA transcript. (e.g., a modified capped RNA transcript). In some embodiments, a 5′ cap region is produced as described herein. When a ligase forms a covalent bond between two linear nucleic acids, a new nucleic acid is produced, with the produced nucleic acid comprising the nucleic acid sequences of both nucleic acids. Ligation of the 3′ terminal nucleotide of a first nucleic acid to the 5′ terminal nucleotide of a second nucleic acid produces a third nucleic acid, with the third nucleic acid comprising the sequence of the first nucleic acid and the second nucleic acid, and the second nucleic acid being 3′ to (downstream of) the first nucleic acid sequence. Ligation by an RNA ligase occurs in several steps. First, an amino (—NH₂) group of an amino acid (e.g., a lysine) of the ligase bonds to a phosphate group of adenosine triphosphate (ATP), such that an adenosine monophosphate (AMP) group is bound to the RNA ligase. Second, a 5′ terminal phosphate of the second nucleic acid displaces the phosphate of the RNA ligase-bound AMP. Finally, an oxygen of the 3′ terminal hydroxyl group of the first nucleic acid binds to the phosphorus atom of the 5′ terminal phosphate of the second nucleic acid. This final step forms a phosphodiester bond between terminal nucleotides of the nucleic acids, thereby forming a single nucleic acid with a continuous sugar-phosphate backbone. In some embodiments, the ligase is T4 RNA Ligase I, T4 RNA Ligase II, or RtcB. In some embodiments, the ligation is performed using a split ribozyme (see, e.g., Gambill et al., “A split ribozyme that links detection of a native RNA to orthogonal protein outputs.” Nat Commun 14, 543 (2023)).

In some embodiments, the RNA precursor comprises an open reading frame (ORF). In some embodiments, the ORF encodes a therapeutic protein. As used herein, a “therapeutic protein” refers to a protein that prevents, reduces, or alleviates one or more signs or symptoms of a disease or disorder when expressed in a subject, such as a human subject that has, for example, an essential enzyme, clotting factor, transcription factor, growth factor, cytokine, chemokine, antibody (or antibody fragment thereof), protein hormone, signaling protein, structural protein, or cell surface receptor encoded by a gene that is mutated in a subject. A mutation in a gene encoding such a protein may cause diminished levels of the protein to be expressed in one or more cells of the subject. For example, IPEX syndrome in humans is caused by a mutation in the FOXP3 gene, which hinders development of FOXP3+ regulatory T cells and results in increased susceptibility to autoimmune and inflammatory disorders. Expression of an essential enzyme, clotting factor, transcription factor, growth factor, cytokine, chemokine, antibody (or antibody fragment thereof), protein hormone, signaling protein, structural protein, or cell surface receptor from an RNA may therefore compensate for a mutation in the gene encoding such a protein in a subject. In some embodiments, the therapeutic protein is a protein that is expressed in one or more cells of a subject a level that is less than (e.g., significantly less than) that of a reference value, such as the level of expression of the protein that is typical in cells of one or more healthy subjects (i.e., subjects who do not have and are not at risk for developing the disease or disorder). Non-limiting examples of therapeutic proteins include base editors (e.g., adenine base editors or RNA base editors), CRISPR-associated proteins (Cas1, Cas2, Cas3, Cas4, Cas5, Cas6, Cas7, Cas8, Cas9, Cas10, Cas12 [Cpf1], or Cas13 [C2c2] endonuclease), RNase proteins (e.g., RNase III), hormones (e.g., insulin, renin, parathyroid hormone, thyroid hormone), thrombin, fibrinogen, metabolic enzymes, erythropoietin (EPO), growth hormone (e.g., GSH), interferons, antibodies (e.g., monoclonal antibodies), colony-stimulating factors (CSFs, e.g., granulocyte colony-stimulating factor [G-CSF]), tissue plasminogen activator (tPA), Factor VIII, Factor IX, enzymes (e.g., for conditions such as Gaucher's disease or Fabry disease), interleukins, bone morphogenic proteins (BMPs), relaxin, alpha-1 antitrypsin, filgrastim, oxytocin, somatostatin, calcitonin, glucagon, liraglutide, vasopressin, epigenetic modulating proteins, and growth factors.

In some embodiments, the ORF encodes an antigen. As used herein, “antigen” refers to a molecule (e.g., a protein) that, when expressed in a subject, elicits the generation of antibodies in the subject that bind to the antigen. In some embodiments, the antigen is a protein derived from a pathogen, such as a pathogenic virus, bacterium, protozoan, or fungus. In some embodiments, the antigen is a protein derived from a virus (viral antigen) or a fragment thereof. In some embodiments, the antigen is a protein derived from a bacterium (bacterial antigen) or a fragment thereof. In some embodiments, the antigen is a protein derived from a protozoan (protozoal antigen) or a fragment thereof. In some embodiments, the antigen is a protein derived from a fungus (fungal antigen) or a fragment thereof. A fragment of a full-length protein refers to a protein with an amino acid sequence that is present in, but shorter than, the amino acid sequence of the full-length protein. Thus, in some embodiments, the RNA transcripts produced by the methods provided herein may be used for prophylactic purposes, such as for vaccination of a subject.

In another aspect, the methods disclosed herein provide an RNA precursor and/or a capped RNA transcript comprising one or more noncoding genes. In some embodiments, the noncoding heterologous genes are therapeutic nucleic acids. As used herein, a therapeutic nucleic acid is a nucleic acid or related compound that alters gene expression to prevent or treat diseases or disorders. In some embodiments, the therapeutic nucleic acid is an antisense oligonucleotide (ASO), N-acetylgalactosamine (GalNAc) ligand-modified short interfering RNA (siRNA) conjugate, DNA aptamer, RNA aptamer, ribozyme, RNA decoy, siRNA, shRNA, miRNA, gRNA, or CRISPRi molecule.

In some embodiments, a composition provided herein (e.g., a pharmaceutical composition) further comprises one or more additional agents. In some embodiments, the additional agent is a nucleotide, a nucleic acid, an amino acid, a peptide, a protein, a small molecule, an aptamer, a lipid, or a carbohydrate. In some embodiments, the additional agent is an agent which has a therapeutic effect when administered to a subject. In some embodiments, the additional agent is an agent that is capable of modulating expression of a gene and/or protein is a subject, such as a short hairpin RNA (shRNA), a small interfering RNA (siRNA), or an antisense oligonucleotide (ASO). In some embodiments, the additional agent is a small molecular inhibitor. In some embodiments, the additional agent is an agent that is capable of eliciting or enhancing an immune response in a subject. In some embodiments, the additional agent is an antigen (e.g., a viral antigen, a bacterial antigen). In some embodiments, the additional agent is an adjuvant, which is defined as an agent that is sufficient for enhancing an immune response in a subject when administered at an effective amount, but does not elicit an immune response in a subject when administered alone. In some embodiments, the additional agent is an enzyme, such as an enzyme that is capable of catalyzing one or more chemical reactions in a subject or in cells of a subject.

In some embodiments of the methods provided herein, an RNA precursor for which 5′ capping is desired must first be chemically prepared for capping. In vitro transcription (IVT) of RNA results in uncapped 5′-triphosphorylated RNA. The 5′-triphosphate of the in vitro transcribed RNA is incompatible with enzymatic ligation on its 5′ end, as enzymatic ligation requires 5′-monophosphorylated RNA. For example, T4 RNA Ligase 1 catalyzes the ligation of a 5′-phosphoryl-terminated nucleic acid donor (e.g., the RNA precursor described herein) to a 3′ hydroxyl-terminated nucleic acid acceptor (e.g., the 5′ cap region described herein) through the formation of a 3′□ 5′ phosphodiester bond with hydrolysis of adenosine triphosphate (ATP) to adenosine monophosphate (AMP) and pyrophosphate (PPi). Thus, in some embodiments, an RNA precursor is prepared for capping by removing pyrophosphate from the 5′ end of the triphosphorylated RNA, leaving a 5′-monophosphorylated RNA to be used in a ligation reaction. In some embodiments of the methods provided herein, RNA 5′ Pyrophosphohydrolase (RppH) is used to produce 5′-monophosphorylated RNA from 5′-triphosphorylated RNA.

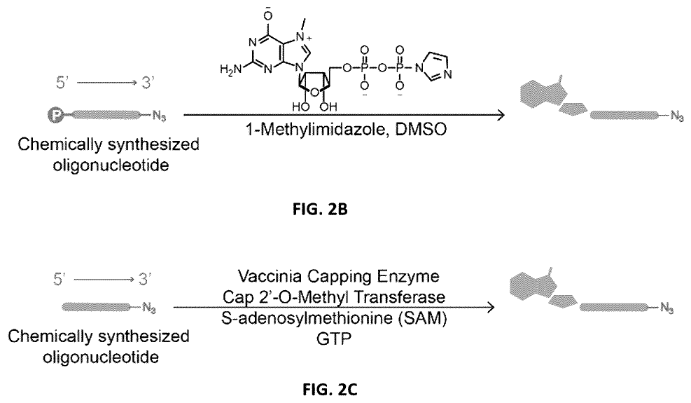

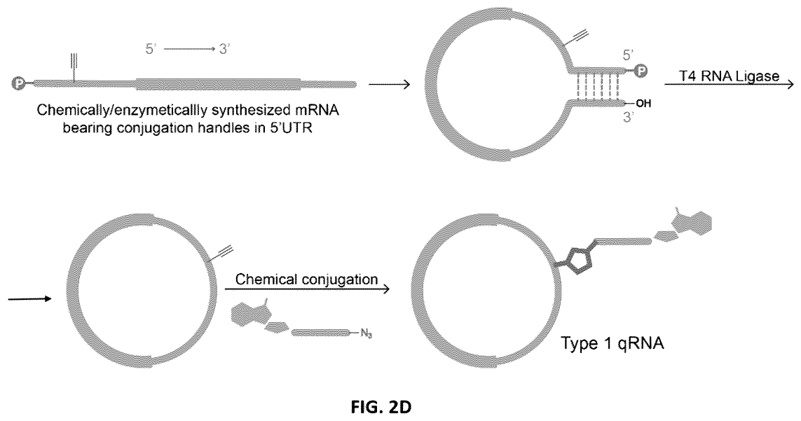

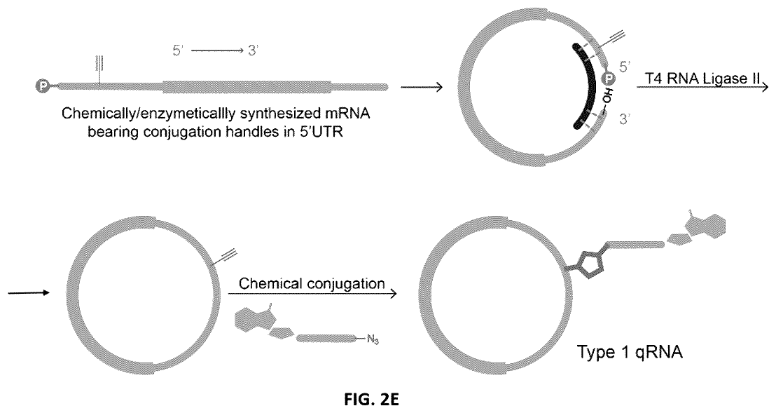

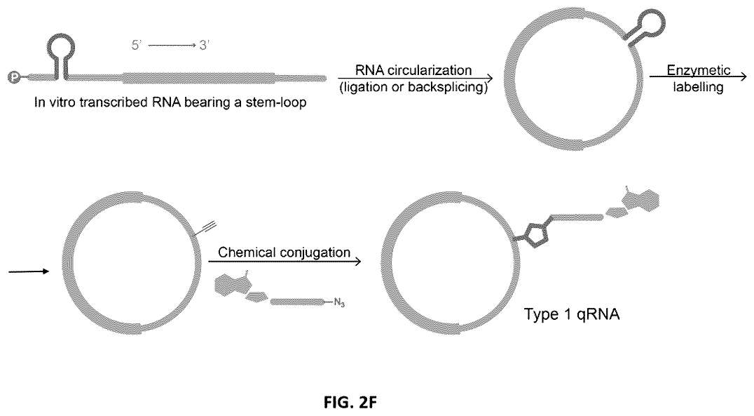

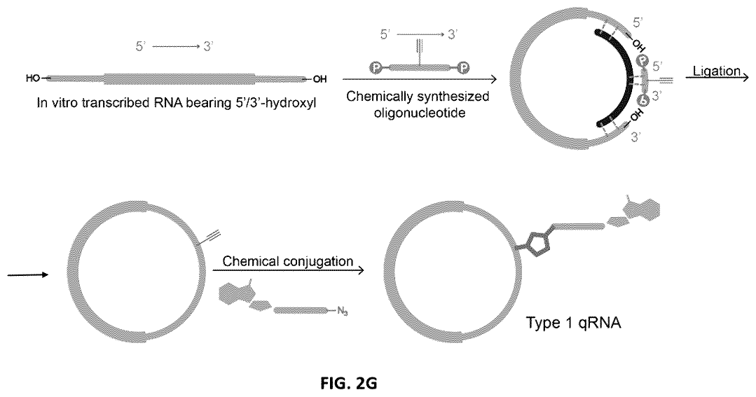

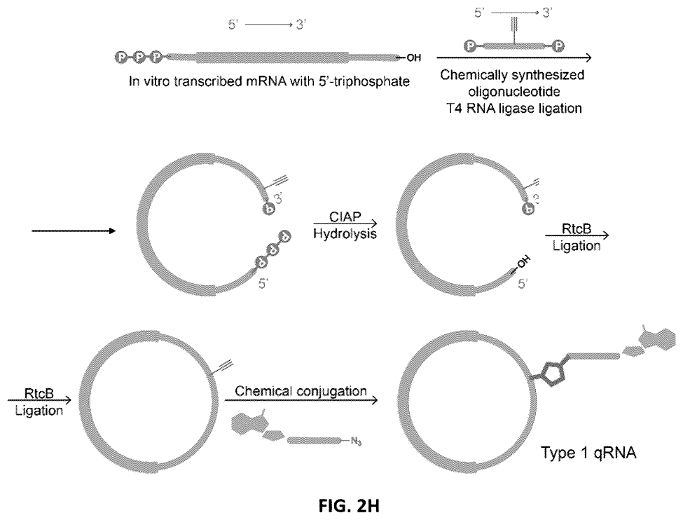

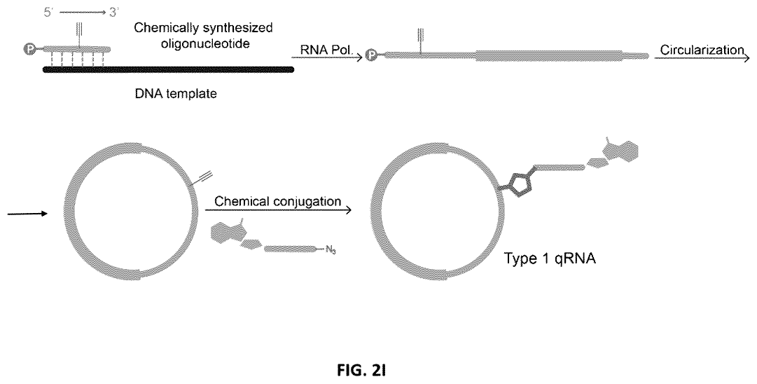

Ligation of the 5′ cap region to the RNA precursor using conventional enzymatic ligation methods demonstrated the requirement of a high ratio (>200) of 5′ cap region oligo: RNA precursor (oligo: mRNA) to achieve complete labeling of the RNA precursor (see FIG. 2B). The requirement for such high levels of 5′ cap region oligos is inconsistent with scalability of such methods. Thus, to address this issue, in some embodiments a short unstructured spacer region was introduced into the 5′ end of the RNA precursor such that the 5′ end was exposed, allowing enzymes (e.g., RNA ligase) better access to the 5′ end.

In some embodiments, the spacer region comprises a plurality of identical consecutive nucleotides. In some embodiments, the spacer region comprises between 5 and 10, between 10 and 20, between 15 and 30, between 20 and 50, between 50 and 100, or between 100 and 200 consecutive adenosine, cytosine, guanine, or thymine nucleotides. In some embodiments, the spacer region comprises about 15 consecutive adenosine nucleotides. In some embodiments, the spacer region comprises at least one non-canonical nucleotide (e.g., inosine).

In some embodiments, the RNA precursor to which the 5′ cap region is ligated comprises one or more exonuclease-resistant nucleotide modifications in its 3′ end. Modified nucleotides containing one or more structural modifications to the nucleobase, sugar, or phosphate linkage of the RNA can interfere with 3′ and 5′ exonuclease activity, rendering the RNA more stable. Nucleotide modifications conferring exonuclease resistance are known in the art. In some embodiments, the RNA precursor comprises one or more modified phosphates, sugars, and/or nucleobases to confer exonuclease resistance. In some embodiments, the RNA precursor comprises one or more 2′-O-Methyl(2′OMe) modifications. In some embodiments, the RNA precursor comprises one or more 2′-fluoro bases. In some embodiments, the RNA precursor comprises one or more phosphorothioate (PS) or thiophosphate (SP) linkages. In some embodiments, the 3′ end of the RNA precursor comprises a phosphate group. In some embodiments, the RNA precursor comprises a C3 spacer incorporated internally or at its 3′ end. A C3 spacer modification adds a 3-carbon spacer to the 3′ terminus of an oligonucleotide. In some embodiments, the RNA precursor comprises a 2′-O-methoxy-ethyl base (2′-MOE), a G-quadruplex, or a 2′-3′-dideoxy nucleotide (ddN). In some embodiments, the RNA precursor comprises one or a combination of any of the modifications known in the art to confer exonuclease resistance (see, e.g., Clave et al., “Modified internucleoside linkages for nuclease-resistant oligonucleotides.” RSC Chem. Biol. (2021) 2:94-150)

Capped-Circular mRNA (QRNA)

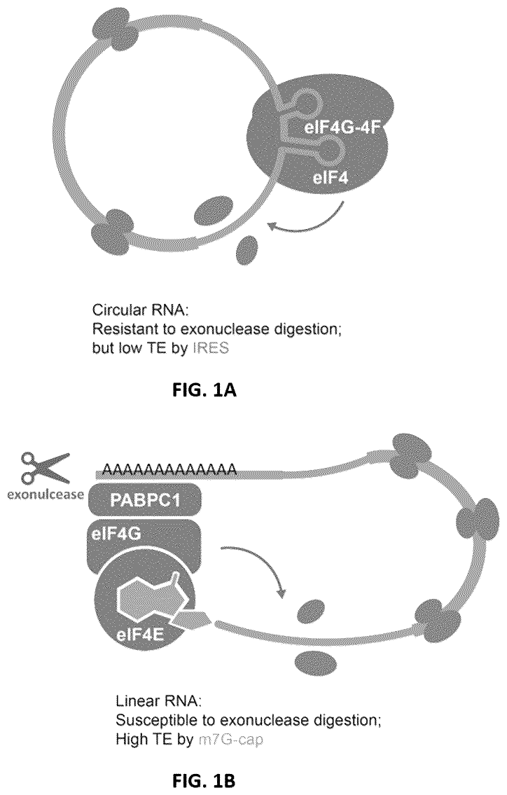

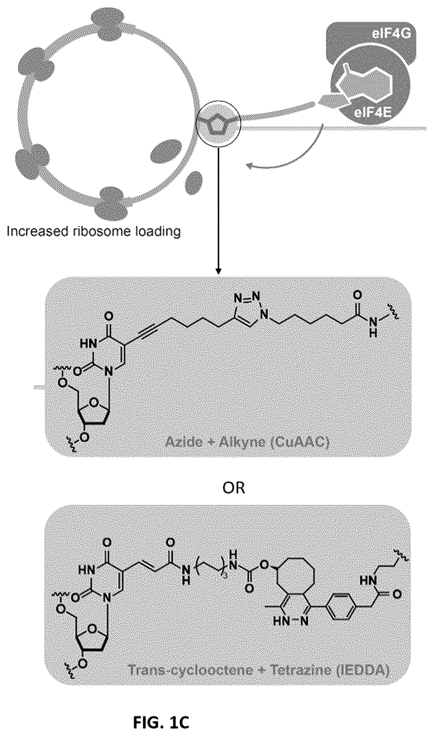

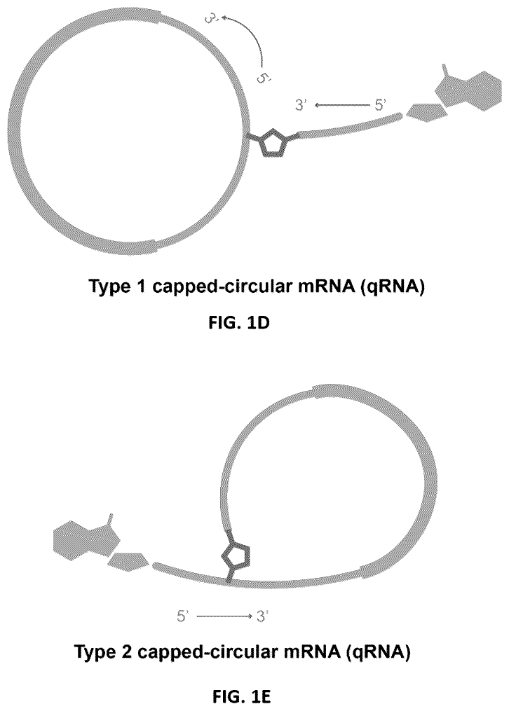

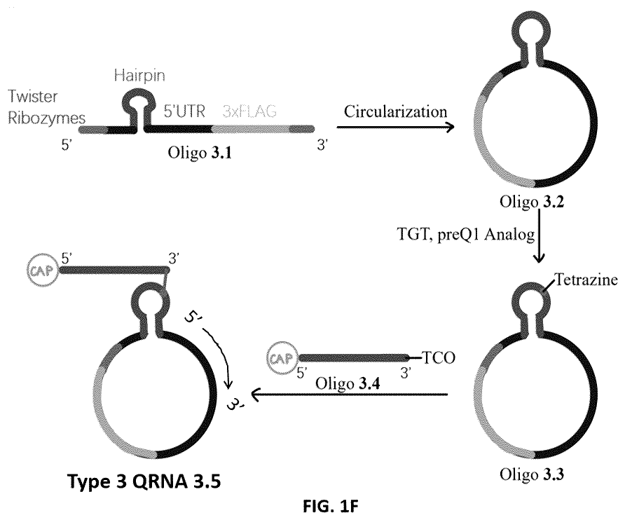

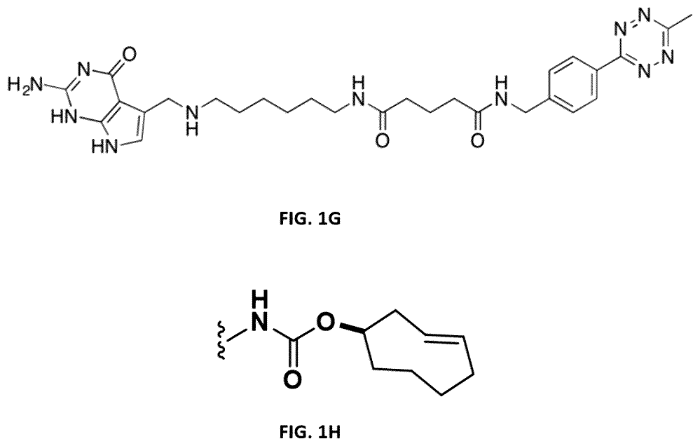

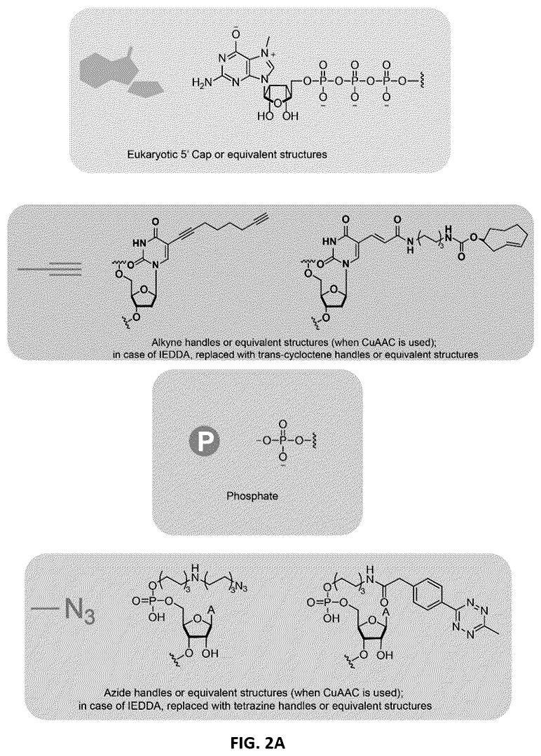

Despite advances in circular RNA (circRNA) engineering, current constructs rely on IRES (Internal Ribosome Entry Site) or TEE (Translation Enhancing Element)-mediated translation, which are embodiments that enable cap-independent translation (FIG. 1A). Linear mRNAs are capable of undergoing cap-dependent translation through interaction with eIF4E and other eukaryotic translation initiation factors (FIG. 1B), which is the predominant form of translation in cells (Sonenberg and Hinnebusch, 2009, Cell 136:731-745) and is generally more efficient than cap-independent translation (Koch et al., 2020, Nat. Struct. Mol. Biol. 27:1095-1104).

As described herein, a “capped-circular mRNA” is a circular mRNA characterized by one or more covalent linkages to one or more cap structures (or a derivative thereof). The circular mRNA can contain all the canonical elements of a linear mRNA: (1) Cap, (2) 5′ UTR (untranslated region), (3) protein-coding regions (CDS), (4) 3′ UTR, and (5) poly(A) tail. By circularizing these features into a capped-circular RNA, it is intended to enhance half-life (increased nuclease resistance) of a canonical circular mRNA, while retaining the benefits of efficient cap-dependent translation, such as in linear mRNA.

The RNA embodiments and methods disclosed herein take advantage of the exonuclease-resistant feature of circRNA while utilizing the strong m7G-cap dependent translation initiation machinery. Such features can be achieved via chemical conjugation of a capped oligonucleotide with a circRNA through click chemistries such as copper catalyzed azide-alkyne cycloaddition (CuAAC) or tetrazine-trans cyclooctene inverse electron demand Diels-Alder reaction (IEDDA) (FIG. 1C). As shown in FIG. 1D and FIG. 1E, the disclosure contemplates two generic structures of capped circular messenger RNAs (QRNAs): Type 1 QRNA and Type 2 QRNA. In Type 1 QRNA, a circular poly-phosphodiester backbone is present while capping is achieved via chemical ligation of a short, capped oligonucleotide to an internal handle on the circular mRNA through click chemistry. The 5′ cap may comprise of a 7-methylguanylate that enables efficient translation of an mRNA or alternative common mRNA cap structures, as shown, for example, in Mccaffreyanton, 2019, Genetic Engineering & Biotechnology News. 39. In Type 2 QRNA, a continuous mRNA poly-phosphodiester backbone is present; circularization is achieved via chemical conjugation between the 3′-end and 5′-UTR of the mRNA through click chemistry.

Various components of the circRNA are depicted in FIG. 2A. The 5′ capping and 3′ poly(A) tailing steps are useful in producing active synthetic mRNA; these modifications prevent mRNA degradation and facilitate translation initiation in eukaryotic cells. As used herein, “capping” means modification at the 5′ end of an mRNA by an addition of a “cap” molecule such as a 7-methylguanosine (m7G) cap. Other cap structures and modifications of the cap as described below can be used to optimize the translation efficiency.

Enzymes capable of catalyzing the reaction of linking a cap molecule to the mRNA include, but are not limited to, Vaccinia capping system including 2′-O-Methyl Transferase (FIG. 2C), tRNA guanine transglycosylase (TGT), Faustovirus capping enzyme, and T4-RNA ligase. Capping can also occur during the synthesis of mRNA called co-transcriptional capping.

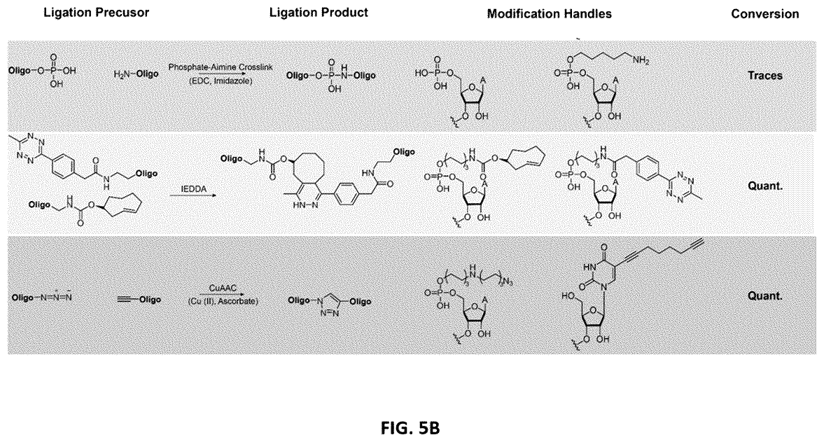

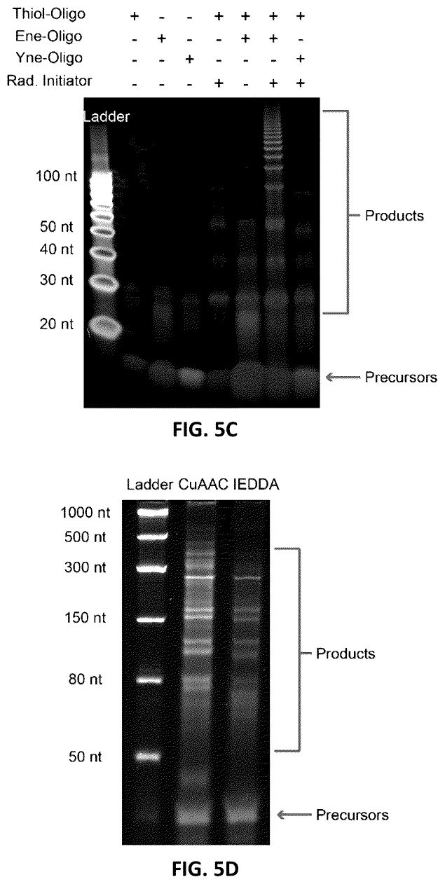

As used herein, the term “molecular handle” or “handle” refers to a chemical group that is attached to a nucleotide on mRNA and can form a covalent bond to another molecule that is separate from the mRNA to link this other molecule to the mRNA. The covalent bond can be formed via various appropriate functional crosslinking reactions. In some embodiments described herein, the crosslinking reaction is click chemistry. As used herein, the term “click handle” refers to a molecule on mRNA that can covalently bind to another molecule via click chemistry reaction. Examples of a handle include, but are not limited to, alkyne or azide (when CuAAC is used in click chemistry), or trans-cyclotene or tetrazine (when IEDDA is used in click chemistry), or hydrozone or oxime, or any equivalent structures thereof. Other crosslinking chemistries including thio-ene and tiol-yne reactions (Escorihuela et al., 2014, Bioconjug. Chem. 25:618-627), a phosphate-amine based reaction (El-Sagheer and Brown, 2017, Chem. Commun. 53:10700-10702; Kalinowski et al., 2016, Chembiochem. 17:1150-1155) (shown in FIG. 5A and FIG. 5B respectively), thiol-yne, amino-yne, and hydroxyl-yne reactions (Worch et al., 2021, Chem Rev. 121 (12): 6744-6776), and other bioconjugation reactions (Gassensmith, chem.libretexts.org/Bookshelves/Organic_Chemistry/Supplemental_Modules_(Organic_Chemistry)/Reactions/Introduction_to_Bioconjugation, accessed Jun. 23, 2023) have also been contemplated. In some embodiments described herein, the crosslinking reaction is a photochemical crosslinking reaction. In some embodiments, one of the synthetic oligos can contain a photoactive alkene such as the cyanovinyl moiety in cnvK and cnvD (see cambio.co.uk/library/images/html_images/Glen_structures/GR30-2.pdf), or the alkene in furan or pyrone in coumarin or psoralen (see pubs.rsc.org/en/content/articlepdf/2022/ra/d1ra05951c FIGS. 11, 12, 13, and 14).As used herein, the term “hairpin” or “hairpin oligonucleotide” refers to a single-stranded oligonucleotide that has a sequence of complementary base pairs at both ends capable of forming a “stem-and-loop” structure.

As used herein and understood in the art, the term “click chemistry” is intended to encompass chemical methods for linking chemical components together, including but not limited to nucleotides into polynucleotides and amino acids into peptides and polypeptides, that are “simple to perform, have high yields, require no or minimal purification, and are versatile in joining diverse structures without the prerequisite of protection steps” (see, for example, Hein et al., 2006, Pharm. Res. 10:2216-2230). In current chemical synthetic practice four primary reactions are employed: 1) cycloadditions (including for example monovalent copper-catalyzed Huisgen 1,3-dipolar cycloadditions of azides and alkynes, the most widely used); 2) nucleophilic ring openings (including ring systems comprising strained heterocyclic electrophiles); 3) non-Aldol carbonyl chemistry (including for example hydrazone/oxime ether formation); and 4) carbon multiple bond additions (including for example certain Michael additions and formation of various three-membered rings by inter alia epoxidations). Click chemistry has been found to be particularly useful for polymeric substances such as proteins and nucleic acids as illustrated herein.

As used herein, the term “equivalent structure” means any molecule that are sufficiently structurally similar and perform the same function in a chemical reaction.

As used herein, the terms “derivatized” or “functionalized” means modification of a nucleotide that leads to some functional consequences in its chemical properties or reactivity or both. Both terms shall be understood to be equivalent to the extent that particular embodiments of the capped, circular RNA molecules have by benefit of derivatization thereof a function, particularly with regard to crosslink-dependent circularization embodiments provided herein. In some embodiments, a derivatized nucleotide is a nucleotide that is modified to comprise a chemical group/handle can participate in a cross-linking reaction.

As used herein, the term “QRNA” is intended as a generic term meaning capped circular messenger RNAs. Particularly encompassed by this term are the various species of circularized RNA molecules and in particular circularized mRNA molecules disclosed herein, but these examples are not intended to be limiting.

In some embodiments, the synthesis pathway of Type 1 and Type 3 QRNA enables multiple oligonucleotides containing 5′ cap binding to the circular RNA. For example, circular RNA can include multiple derivatized nucleotides that can covalently bind to multiple oligonucleotides containing 5′ cap. Alternatively, a single circular RNA backbone can encode multiple TGT sites to enable binding of multiple oligonucleotides containing 5′ cap onto the circular RNA simultaneously.

In some embodiments, the capped, circular RNA molecule comprises an mRNA region encoding one or a plurality of peptides or polypeptides.

As provided herein, the cap used in the capped, circularized RNA molecules of the disclosure can include 7-methylguanine (m7G) but in addition cap analogues as set forth, inter alia, in U.S. patent application No. 2020/0055891 to Walczak et al.; Holstein et al., 2016, Agnew Chem. Int. Ed. Engl. 55:10899-10903; Walczak et al., 2017, Chem. Sci. 8:260-267; Muttach et al., 2017, J. Org. Chem. 13:2819-2832) can be incorporated into the circular RNA molecule precursors to create the capped, circularized RNA molecules provided herein.

### Cap Modifications for QRNA

Several variations of the cap structure have been contemplated here to optimize translation efficiency of QRNA. These variations include: including multiple cap structures (cap 0, 1, and 2; Shanmugasundaram et al., 2022, Chem Rec. 22 (8): e202200005); including N⁶, 2′-O-dimethyladenosine (m⁶Am) as a terminal modification adjacent to the mRNA cap (Sun et al., 2021, Nat Commun. 12 (1): 4778); using cap structures with modified triphosphate bridges (Sun et al., 2021, Nat Commun. 12 (1): 4778; Wojtczak et al., 2018, J Am Chem Soc. 140 (18): 5987-5999); incorporating Locked Nucleic Acid (LNA)-modified cap analogs (Kore et al., 2009, J Am Chem Soc. 131 (18): 6364-5); introducing cap analogs with alternative functionalities such as light reactivity and click groups (Klocker et al., 2022, Nat Chem. 14 (8): 905-913; Nowakowska et al., 2014, Org. biomol. Chem. 12:4841-4847); hydrophobic cap analogs (WO 2017066782 A1); and others (Wojcik et al., 2021, Pharmaceutics 13 (11): 1941; Grudzien et al., RNA 10 (9): 1479-1487; Grzela et al., 2023, RNA 29 (2): 200-216).

In some embodiments, the methyl group in 7-methylguanosine (m7G) cap structure can be modified to produce 7-benzylguanosine (Bn⁷G), 7-chlorobenzylguanosine (ClBn⁷G), and chlorobenzyl-O-ethoxyguanosine (ClBnOEt⁷G). Introduction of one or more Locked Nucleic Acid (LNA), 2′-methoxy (2OMe), and 2-methoxyethoxy (2MOE) into m7G structure significantly increase mRNA translation. In some embodiments, the cap structures include, but are not limited to, m⁷G-LNA, LNAm⁷G-LNA, LNAm⁷G-LNAx6, LNAm⁷G-2OMex6. In some embodiments, the cap structure is m⁷G diphosphate imidazolide (m⁷GDP-Im).

### Nucleotide Modifications

In some embodiments, as disclosed and recognized herein it is beneficial to alter the type of nucleotide/nucleotide identity, specifically incorporation of adenosine (A), guanosine (G), 6-methyladenosine (m⁶A), or the non-canonical inosine (I) in the mRNA, preferably, at the +1 position, increases translation efficiency. In some embodiments, substitution of some or all uridine residues to N1-methylpseudouridine (m¹ψ) in the mRNA also boosts the translation. The nucleotides are numbered according to their position immediately downstream of the cap structure. For example, the cap structure found at the 5′ end of eukaryotic mRNAs consists of a 7-methylguanosine (m⁷G) moiety linked to the first nucleotide (+1 position) of the transcript via a 5′-5′ triphosphate bridge.

Other modified nucleotides include, but are not limited to, pseudouridine, 5-methylcytidine, 2-thiouridine, 5-methoxyuridine, 4-acetylcytidine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A) have also been contemplated at +1 and other positions.

In some embodiments, the modified phosphate backbone can be phosphorothioate (PS), thiophosphate, 5′-O-methylphosphonate, 3′-O-methylphosphonate, 5-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, or guanidinopropyl phosphoramidate.

In some embodiments, introduction of locked nucleic acid (LNA), 2′-methoxyribose (2-OMe), and 2-methoxyethoxy (2-MOE) into the ribose sugar backbone increases mRNA translation. Addition of multiple 2-OMe and 2-MOE modified bases increases translation further. LNA specifically increased expression at the +1 position.

In some embodiments, the modified sugar can be 2-thioribose, 2,3-dideoxyribose, 2-amino-2-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2,3-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, or 2′-O,4′-C-thio-linked ribose.

In these backbone modifications, stereoisomer structures are also considered since they have been shown to impact the RNA's nuclease-resistance properties (Iwamoto et al., 2017, Nat. Biotech. 35:845-851; Jahns et al., 2022, Nucleic Acids Res. 50 (3): 1221-1240).

Modification of nucleotides on traditional circRNA is limited because not all of them are compatible with the internal ribosome entry site (IRES). QRNA translation does not require an IRES; thus, is tolerable to more modified nucleotides in a wide range of percentage. These modifications could be spiked into the circular backbone in varying percentages (m6A is typically spiked in at 5%). And the “stem” oligo containing the cap, or the 5′/3′ UTR and tails could likely tolerate a higher percentage of modifications. Alternatively, these modifications can be present in different percentages along different regions of the circular RNA backbone (e.g. in the 5′ UTR, or 3′ UTR, or CDS, or close to the cap structure, or combinations thereof).

Furthermore, the “stem” oligo of a Type 1 QRNA (the oligonucleotide containing the cap) is chemically synthesized and could potentially tolerate more complex structures that are difficult to enzymatically incorporate, such as locked nucleic acids (LNAs), 2′ O-methyl nucleotides, peptide nucleic acids (PNAs), morpholinos, and various internal chemical linkers as provided herein.

### Peptides and Polypeptides Encoded by RNA

Polypeptides encoded by the capped RNA molecules (linear or circularized) provided by the disclosure include any therapeutically useful polypeptide for treatment or intervention of any disease process associated with or dependent on polymorphic or mutant polypeptide species, heritable or acquired as a result of environmental insult or injury. Linear or QRNA can encode multiple polypeptides, for example, self-amplifying mRNA cassettes, or multiple therapeutic peptides or polypeptides. In some embodiments, the capped RNA molecule (circular or linear) comprises an mRNA region encoding one or a plurality of peptides or polypeptides. A plurality of polypeptides include multiple copies of the same polypeptide or multiple copies of different polypeptides.

An IRES, or self-cleaving peptide such as T2A sequence, can exist between the multiple polypeptide coding sequences on the RNA (linear or QRNA). Alternatively, an RNA oligonucleotide containing a cap residue site is located before each polypeptide coding sequence, which ultimately will result in a RNA with multiple cap residue-containing RNA oligonucleotides and ensure that all coding sequences are translated efficiently.

Peptides encoded by capped RNA molecules (linear or circular) of the disclosure can include but are not limited to therapeutic peptides or antigenic peptides, particularly antigenic peptides suitable for presentation by antigen-presenting cells to humoral (B cells) or cellular (T cells) immune system cells. In certain embodiments these antigenic peptides are adapted to and effective for use as vaccines. In other embodiments the antigenic peptides are adapted to or effective in suppressing immune responses, for example in autoimmune diseases or transplant patients. In additional embodiments the antigenic peptides are adapted to and effective for eliciting specific antitumor immune responses in tumor cells or in attracting cytotoxic native (natural killer cells) or engineered (e.g., CAR-T) cells. Therapeutic peptides encoded by capped, circularized RNA molecules of the disclosure can include but are not limited to human parathyroid hormone, filgrastim, oxytocin, somatostatin, calcitonin, glucagon, insulin, liraglutide, vasopressin, and the like (see, Fosgerau & Hoffman, 2015, Drug Discovery Today 20:122-128; al Musaimi et al., 2021, Pharmaceuticals (Basil) 14:145; Wang et al., 2022, Signal Transduct, and Targeted Therap. 7: 1-27).

In some embodiments, peptides encoded by the capped RNA molecules (linear or circular) of the disclosure can include, but are not limited, to Cas9 or derivatives (Rothgangl et al., 2021, Nat. Biotechnol. 39:949-957) and adenine base editors or other base editors (Gaudelli et al., 2017, Nature 551:464-471), or RNA base editors for delivery of genome or epigenome editing therapies.

In some embodiments, peptides encoded by the capped RNA molecules (linear or circular) of the disclosure can be selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, or targeting moieties.

### Synthesis of QRNA

Type 2: The disclosure also provides methods for producing a type 2 capped, circularized RNA molecules of this aspect of the disclosure, the methods comprising: synthesizing an RNA oligonucleotide comprising a 5′ end containing a cap structure, an mRNA encoding a peptide or polypeptide, a derivatized nucleotide located between the cap structure and the mRNA region encoding the polypeptide, and a 3′ end containing moiety; and reacting the derivatized nucleotide with the 3′ end moiety to form the covalently linked capped circular RNA molecule.

Type 1: The disclosure also provides methods for producing a type 1 capped, circularized RNA molecules of this aspect of the disclosure, the methods comprising the steps of: producing a circularized RNA molecule comprising an mRNA region encoding a peptide or polypeptide, and a derivatized nucleotide outside the mRNA region; synthesizing an RNA oligonucleotide comprising a 5′ end containing cap structure and a 3′ end containing a moiety reactive with the derivatized nucleotide; and reacting the derivatized nucleotide with the 3′ end moiety of the RNA oligonucleotide form a covalently link between the RNA oligonucleotide and the circular RNA. In certain embodiments, the derivatized nucleotide comprises a moiety that can react with the 3′ end moiety by bioconjugation chemistry, wherein in the bioconjugation chemistry is click chemistry. In addition, the circularized RNA is produced by ribozyme-mediated splicing, enzymatic ligation, or click chemistry-mediated circularization.

Type 3: The disclosure also provides methods for producing a type 3 capped, circularized RNA molecules of this aspect of the disclosure, the methods comprising the steps of: producing a circularized RNA molecule comprising an mRNA region encoding a peptide or polypeptide, and a derivatized nucleotide outside the mRNA region; synthesizing an RNA oligonucleotide comprising a 5′ end containing cap structure and a 3′ end containing a moiety reactive with the derivatized nucleotide; and reacting the derivatized nucleotide with the 3′ end moiety of the RNA oligonucleotide form a covalently link between the RNA oligonucleotide and the circular RNA, wherein the synthesis of the circular RNA oligonucleotide, further comprises the steps of: synthesizing an RNA oligonucleotide comprising the mRNA region encoding a peptide or polypeptide, a hairpin structure containing an enzyme-recognition site, and a twister ribozyme sequence on both 5′ and 3′ ends; reacting the RNA oligonucleotide with the enzyme to produce the derivatized nucleotide within the hairpin structure; circularizing the RNA oligonucleotide using the twister ribozyme sequence.

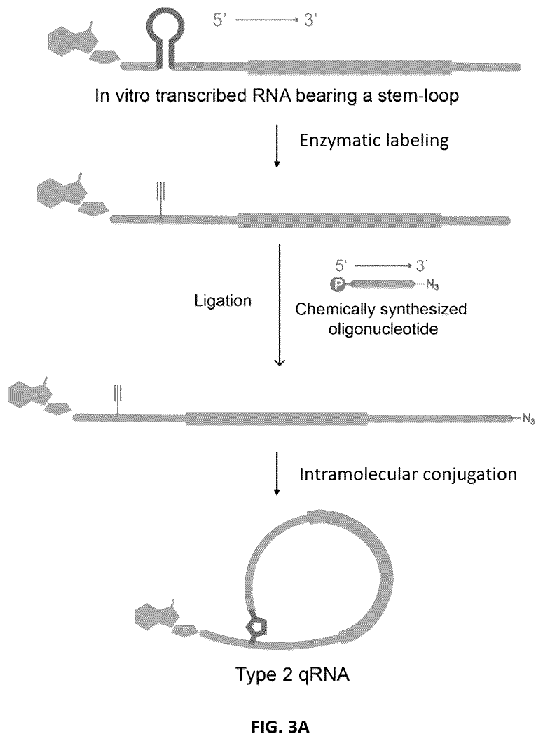

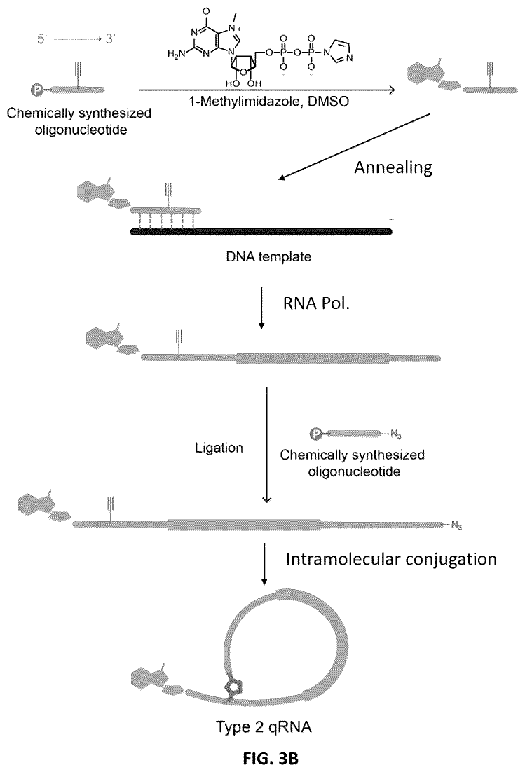

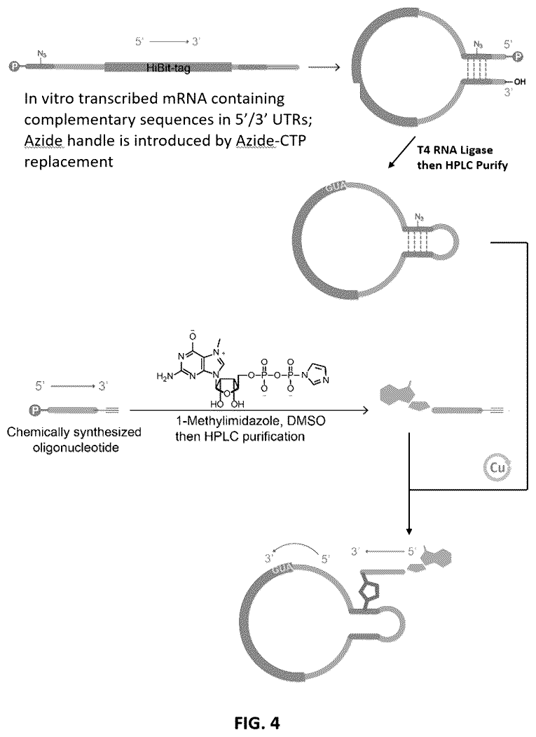

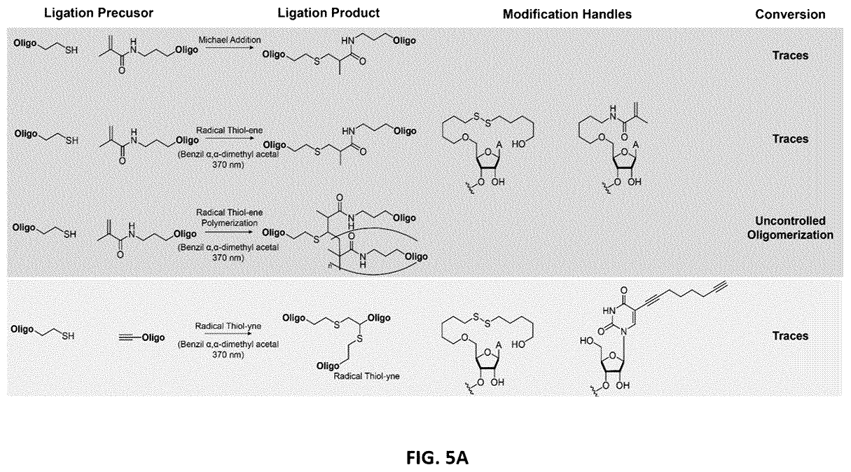

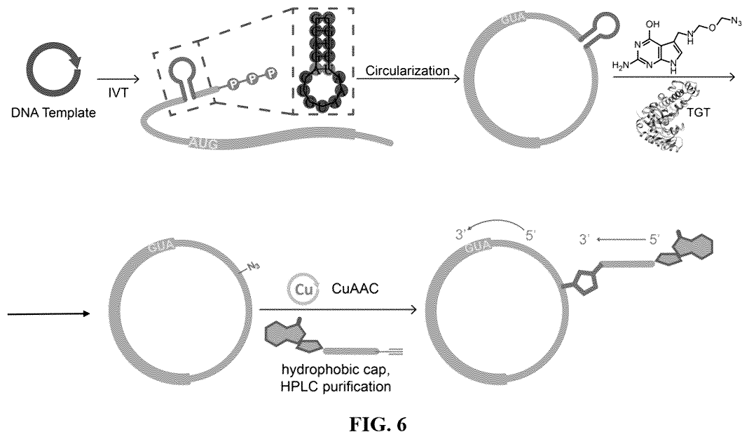

The derivatized nucleotide in these 3 types of QRNA can be generated using different strategies as demonstrated in FIG. 2D-2I, FIG. 4, and FIG. 6 (type 1), FIG. 3A and FIG. 3B (type 2), and FIG. 1F (type 3). Namely, the derivatized nucleotide can be specifically targeted by having a hairpin structure containing a specific enzyme-recognition site. The enzyme described in some examples in tRNA guanine transferases (TGT). In other examples, the derivatized nucleotide is generated by replacement of a single cytidine with azide-cytidine.

Circularization of RNA molecule in type 1 and type 3 can be achieved by ligation with T4 ligase, RtcB ligase, or ribozyme-mediated splicing. In these embodiments, the 5′ end and 3′ end of the linear oligonucleotide comprise the appropriate moiety to participate in the enzymatic reaction to form the circular RNA. Alternatively, the click chemistry moiety has also been contemplated for the circularization. In some embodiments, additional splint probe containing complementary sequences to the 5′ and 3′ ends of the linear oligonucleotide can be used as demonstrated in the FIG. 2E or FIG. 2G to bring the two ends in proximity and facilitate circularization.

### Purification of Capped RNA

In some embodiments, the nucleic acids described herein are purified by any method known in the art to remove undesired components from IVT or associated reactions (including unincorporated rNTPs, protein enzymes, salts, metal ions, etc.). Techniques for the isolation of RNA molecules are well known in the art. Well-known procedures include phenol/chloroform extraction and or precipitation with alcohol (ethanol, isopropanol) in the presence of monovalent cations or lithium chloride. Additional non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v.10, 889-893), silica-based affinity chromatography and polyacrylamide gel electrophoresis (Bowman, et al. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek). Techniques to remove contaminants, such as dsDNA, have been developed and are known in the art including but not limited to scalable HPLC purification (see, e.g., Kariko, et al., 2011, Nucl Acid Res, v. 39 e142; Weissman, et al., 2012, Synthetic Messenger RNA and Cell Metabolism Modulation v.969 (Rabinovich, P.H. Ed)). In a preferred embodiment, the capped RNA described herein is purified through HPLC, as HPLC-purified RNA has been reported to be translated at much greater levels compared to other purification methods, particularly in primary cells and in vivo.

In some aspects, the capped RNA oligonucleotides provided herein (e.g., comprising one or more modified oligonucleotides) are purified by high-performance liquid chromatography (HPLC). In some embodiments, the capped RNA oligonucleotide is purified by reverse-phase HPLC (RP-HPLC). The addition of a counterion to the mobile phase of a HPLC setup can improve separation of the desired product from unwanted products. In a preferred embodiment, HPLC gradients used to isolate the capped RNA oligonucleotides comprise hydrophobic hexylammonium ions. In some embodiments, the gradient is chosen from ethyl ammonium, diethyl ammonium, triethyl ammonium, propyl ammonium, dipropyl ammonium, hexyl ammonium, dihexyl ammonium, octyl ammonium, dioctyl ammonium, etc. The number and lengths of carbon chains may be altered based on the lengths of the oligonucleotide to be captured and the desired feature for separation. In some embodiments, the concentration of hydrophobic ions (e.g., hexylammonium ions) used for HPLC purification of RNA oligonucleotides is between 10 mM to 200 mM. In some embodiments, the concentration of hydrophobic ions is between 10 mM and 20 mM, between 20 mM and 30 mM, between 30 mM and 40 mM, between 40 mM and 50 mM, between 50 mM and 60 mM, between 60 mM and 70 mM, between 70 mM and 80 mM, between 80 mM and 90 mM, between 90 mM and 100 mM, between 100 mM and 110 mM, between 110 mM and 120 mM, between 120 mM and 130 mM, between 130 mM and 140 mM, between 140 mM and 150 mM, between 150 mM and 160 mM, between 160 mM and 170 mM, between 170 mM and 180 mM, between 180 mM and 190 mM, or between 190 mM and 200 mM. In some embodiments, the concentration of hydrophobic ions is greater than 200 mM.

### Compositions Comprising Capped RNA Transcripts

In some aspects, the present disclosure provides a delivery reagent comprising any of the capped RNA transcripts provided herein. In some embodiments, any of the capped RNA transcripts provided herein are conjugated to a delivery agent. Any of the capped RNA transcripts provided herein may be conjugated to a delivery agent that includes, for example, to a lipid, a peptide, a protein, an antibody, or a carbohydrate. Lipids used in the conjugation and delivery of modified mRNAs are generally known in the art, and include, for example, cholesterol. Peptides, proteins, antibodies, and carbohydrates used in the conjugation and delivery of modified mRNAs are generally known in the art and include, for example, any peptide, protein, antibody, or carbohydrate known to bind specifically to a moiety (e.g., a protein) on the surface of a target cell type. Methods for conjugating a lipid, peptide, protein, antibody, or carbohydrate to a capped RNA transcript include, for example, methods of conjugating a lipid, peptide, protein, antibody, or carbohydrate to a capped RNA transcript at a 5′ or 3′ terminus, and are generally known in the art.

In some embodiments, any of the capped RNA transcripts provided herein are conjugated to or encapsulated by a delivery agent that includes, for example, a nanoparticle, a microparticle, or an exosome. A nanoparticle refers to a particle having a diameter between approximately 10 nm and 1000 nm. A microparticle is defines as a particle having a diameter greater than 1000 nm (1 μm), such as a particle having a diameter between approximately 1 μm and 100 μm. In some embodiments, a nanoparticle or microparticle is approximately spherical. In some embodiments, a nanoparticle or microparticle is hollow, comprising an internal core. In some embodiments, a nanoparticle or microparticle is a lipid nanoparticle or lipid microparticle, respectively. A lipid nanoparticle or lipid microparticle refers to a composition comprising one or more lipids that form an aggregate of lipids, or an enclosed structure with an interior surface and an exterior surface. In some embodiments, a lipid nanoparticle or lipid microparticle comprises a lipid bilayer that encloses an aqueous core. Lipids used in the formulation of lipid nanoparticles and lipid microparticles for delivering RNAs are generally known in the art, and include, but are not limited to, ionizable amino lipids, non-cationic lipids, sterols, and polyethylene glycol-modified lipids. See, e.g., Buschmann et al. Vaccines. 2021. 9 (1): 65. In some embodiments, the capped RNA transcript is surrounded by the lipids of the lipid nanoparticle or the lipid microparticle and are present in the interior of the lipid nanoparticle or lipid microparticle. In some embodiments, the capped RNA transcript is dispersed throughout the lipids of the lipid nanoparticle or lipid microparticle. In some embodiments, the lipid nanoparticle or lipid microparticle comprises an ionizable amino lipid, a non-cationic lipid, a sterol, and/or a polyethylene glycol (PEG)-modified lipid. Lipid nanoparticles and lipid microparticles comprising modified mRNAs may be prepared by any means generally known in the art, such as, for example, detergent dialysis, emulsion, centrifugation, evaporation, thin film hydration, or ethanol dilution. See, e.g., Barba et al. Pharmaceutics. 2019. 11 (8): 360. An exosome refers to a type of lipid nanoparticle produced by eukaryotic cells as a result of the inward budding of vesicles within multivesicular bodies and are generally between 30 nm and 150 nm in diameter. Exosomes comprise a heterogenous mixture of endogenous lipids, such as phospholipids, membrane-anchored proteins, and carbohydrates present in eukaryotic cells, and enclose an aqueous core. Exosomes may have beneficial features that are difficult to achieve with synthetically produced lipid nanoparticles, such as, for example, the ability to pass through the blood brain barrier and deliver capped RNA transcripts to tissues within the brain. Exosomes comprising capped RNA transcripts may be produced by any means generally known in the art, such as, for example, by sonicating or electroporating isolated exosomes in the presence of a capped RNA transcript, or mixing exosomes with a lipid-conjugated capped RNA transcript, such as, for example, a capped RNA transcript that has been conjugated to cholesterol. See, e.g., Roberts et al. Nat Rev Drug Discov. 2020. 19 (10): 673-694.

In some embodiments, a nanoparticle or microparticle is a polymeric nanoparticle or polymeric microparticle, respectively. A polymeric nanoparticle or polymeric microparticle refers to a nanoparticle or microparticle composition, respectively, comprising one or more polymers that form an aggregate of polymers, or an enclosed structure with an interior surface and an exterior surface. In some embodiments, a polymeric nanoparticle or polymeric microparticle comprises a polymeric layer that encloses an aqueous core. Polymers used in the formulation of polymeric nanoparticles and polymeric microparticles for delivering RNA are generally known in the art, and include cationic polymers such as, but are not limited to, polyethylenimine (PEI), poly-amido-amine (PAA), poly-beta amino-esters (PBAEs), polylysine (PLL), spermine, chitosan, polyurethane, and derivatives thereof (e.g., PEI stearic acid (PSA) copolymer). See, e.g., Liu et al. Front Bioeng Biotechnol. 2021. 9:718753. In some embodiments, the capped RNA transcript is surrounded by the polymers of the polymeric nanoparticle or the polymeric microparticle and are present in the interior of the polymeric nanoparticle or polymeric microparticle. In some embodiments, the capped RNA transcript is dispersed throughout the polymers of the polymeric nanoparticle or polymeric microparticle.

In some embodiments, a nanoparticle or microparticle is a protein nanoparticle or protein microparticle, respectively. A protein nanoparticle or protein microparticle refers to a nanoparticle or microparticle composition, respectively, comprising one or more proteins that form an aggregate of proteins, or an enclosed structure with an interior surface and an exterior surface. In some embodiments, a protein nanoparticle or protein microparticle comprises a protein layer that encloses an aqueous core. Proteins used in the formulation of protein nanoparticles and protein microparticles for delivering RNA are generally known in the art, and include but are not limited to, viral coat proteins and ferritin. See, e.g., Wang et al. Nat Nanotechnol. 2020. 15 (5): 406-416. In some embodiments, the capped RNA transcript is surrounded by the proteins of the protein nanoparticle or the protein microparticle and are present in the interior of the protein nanoparticle or protein microparticle. In some embodiments, the capped RNA transcript is external to the proteins of the protein nanoparticle or the protein microparticle and are attached to the exterior surface of the protein nanoparticle or protein microparticle. In some embodiments, the capped RNA transcript is conjugated to proteins of the protein nanoparticle or protein microparticle through a covalent linkage, such as, for example, that formed by a click chemistry reaction, or by fusing the capped RNA transcript and protein each to a protein or peptide of a protein/peptide pair known to react to form a covalent linkage.

In some embodiments, a nanoparticle or microparticle is a solid nanoparticle or solid microparticle. A solid nanoparticle or solid microparticle refers to a nanoparticle or microparticle composition, respectively, comprising one or more materials that form a solid structure, which has an external surface and may or may not comprise an internal surface. A solid nanoparticle or solid microparticle may comprise any suitable material that is generally known in the art, such as, for example, gold, silver, or silicon dioxide (silica). In some embodiments, a capped RNA transcript is conjugated to the external surface of a solid nanoparticle or solid microparticle. Solid nanoparticles and solid microparticles comprising capped RNA transcripts may be produced by any means generally known in the art, such as, for example, by linking the capped RNA transcripts to the surface of the solid nanoparticle or solid microparticle through thiol linkages (e.g., modifying the DNA to comprise cyclic disulfide-anchoring groups), or by modifying the external surface of the solid nanoparticle or solid microparticle with one or more cationic materials (e.g., PEI) within which capped RNA transcripts are present. See, e.g., Roberts et al. Nat Rev Drug Discov. 2020. 19 (10): 673-694, Lee et al. Nano Lett. 2007, 7 (7): 2112-2115, and Paris and Vallet-Regi. Pharmaceutics. 2020, 12 (6): 526.

In some aspects, the present disclosure provides cells comprising any of the capped RNA transcripts provided herein. In some embodiments, the cell is a human cell comprising any one of the capped RNA transcripts provided herein. A “cell” is the basic structural and functional unit of all known independently living organisms. It is the smallest unit of life that is classified as a living thing. Some organisms, such as most bacteria, are unicellular (consist of a single cell). Other organisms, such as plants, fungi, and animals, including cattle, horses, chickens, turkeys, sheep, swine, dogs, cats, and humans, are multicellular. In some embodiments, the half-life of the capped RNA transcript in the cell is 15-900 minutes. In some embodiments, the half-life of the capped RNA transcript in the cell is 30-600 minutes. In some embodiments, the half-life of the capped RNA transcript in the cell is 60-300 minutes. In some embodiments, the half-life of the capped RNA transcript is at least 15, at least 20, at least 25, at least 30, at least 35, at least 40, at least 45, at least 50, at least 55, at least 60 minutes. In some embodiments, the half-life of the capped RNA transcript in the cell is at least 30, at least 60, at least 90, at least 120, at least 150, at least 180, at least 210, at least 240, at least 270, at least 300, at least 330, at least 360, at least 390, at least 420, at least 450, at least 480, at least 510, at least 540, at least 570, at least 600, at least 630, at least 660, at least 690, at least 720, at least 750, at least 780, at least 810, at least 840, or at least 870 minutes. In some aspects, the present disclosure provides compositions comprising any of the modified mRNAs, delivery agents, or cells provided herein. In some embodiments, the composition further comprises one or more additional agents, such as a nucleotide, a nucleic acid, an amino acid, a peptide, a protein, a small molecule, an aptamer, a lipid, or a carbohydrate. In some embodiments, the additional agent has a therapeutic effect when administered to a subject. In some embodiments, the additional agent is an agent for use in modulating the expression and/or activity of one or more gene products (e.g., proteins) in a subject. In some embodiments, the additional agent is a nucleic acid for use in decreasing the expression and/or activity of one or more gene products (e.g., proteins), such as a short hairpin RNA (shRNA), small interfering RNA (siRNA), or an antisense oligonucleotide (ASO). In some embodiments, the additional agent is an inhibitor for decreasing the activity of one or more gene products (e.g., proteins). In some embodiments, the agent is a small molecular inhibitor. In some embodiments, the additional agent is an agent for enhancing an immune response in a subject. In some embodiments, the additional agent is an antigen, such as a nucleic acid antigen, a protein antigen, or a phospholipid antigen. In some embodiments, the additional agent is an adjuvant, such as, for example, aluminum hydroxide or potassium aluminum sulfate (alum), monophosphoryl lipid A (MPL), an oil-in-water emulsion (e.g., a squalene emulsion), a cytosine phosphoguanine (CpG) oligodeoxynucleotide, or another adjuvant that is known in the art. See, e.g., Di Pasquale, A et al. Vaccines. 2015. 3 (2): 320-343. In some embodiments, the composition is a pharmaceutical composition comprising any one of the capped RNA transcripts, delivery agents, or cells provided herein, and a pharmaceutically acceptable excipient. Pharmaceutically acceptable excipients, carriers, buffers, stabilisers, isotonicising agents, preservatives or antioxidants, or other materials well known to those skilled in the art. Such materials should be non-toxic and should not interfere with the efficacy of the active ingredient. The precise nature of the carrier or other material may depend on the route of administration, e.g., parenteral, intramuscular, intradermal, sublingual, buccal, ocular, intranasal, subcutaneous, intrathecal, intratumoral, oral, vaginal, or rectal.

In some aspects, the present disclosure provides a method of administering to a subject any of the capped RNA transcripts, delivery agents, cells, compositions, or pharmaceutical compositions provided herein. In some embodiments, the subject is a human. In some embodiments, the administration is parenteral, intramuscular, intradermal, sublingual, buccal, ocular, intranasal, subcutaneous, intrathecal, intratumoral, oral, vaginal, or rectal. In some embodiments, the composition is to be stored below 50° C., below 40° C., below 30° C., below 20° C., below 10° C., below 0° C., below −10° C., below −20° C., below −30° C., below −40° C., below −50° C., below-60° C., below-70° C., or below-80° C., such that the nucleic acids are relatively stable over time. In some embodiments, the capped RNA transcript is introduced into a cell in a subject by in vivo electroporation. In vivo electroporation is the process of introducing nucleic acids or other molecules into a cell of a subject using a pulse of electricity, which promote passage of the nucleic acids or other molecules through the cell membrane and/or cell wall. See, e.g., Somiari et al. Molecular Therapy., 2000. 2 (3): 178-187. The capped RNA transcript to be delivered is administered to the subject, such as by injection, and a pulse of electricity is applied to the injection site, whereby the electricity promotes entry of the nucleic acid into cells at the site of administration. In some embodiments, the capped RNA transcript is delivered to and taken up by cells of the subject (e.g., cells local to the site of administration or throughout the subject) via a delivery agent that is associated with (e.g., conjugated to) the capped RNA transcript. In some embodiments, the capped RNA transcript is administered with other elements, such as buffers and/or excipients, that increase the efficiency of electroporation.

In some aspects, the present disclosure provides a kit comprising any of the capped RNA oligonucleotides, RNA precursors, or capped RNA transcripts provided herein. The capped RNA oligonucleotide and RNA precursor can be combined in the presence of an RNA ligase to produce a capped RNA transcript, such as one of the capped RNA transcripts provided herein. In some embodiments, the kit comprises a ligase. In some embodiments, the kit comprises an RNA ligase. In some embodiments, the kit comprises a T4 RNA ligase. In some embodiments, a kit comprises a T4 RNA ligase 1. In some embodiments, a kit comprises a T4 RNA ligase 2. In some embodiments, the kit comprises an RtcB RNA ligase. In some embodiments, the kit further comprises a buffer for carrying out the ligation. In some embodiments, the kit further comprises a nucleotide triphosphate, such as ATP, to provide energy required by the ligase. In some embodiments, the kit is to be stored below 50° C., below 40° C., below 30° C., below 20° C., below 10° C., below 0° C., below −10° C., below −20° C., below −30° C., below −40° C., below −50° C., below −60° C., below −70° C., or below −80° C., such that the nucleic acids are relatively stable over time.

In some aspects, the present disclosure provides a kit comprising any of the pharmaceutical compositions provided herein and a delivery device. A delivery device refers to machine or apparatus suitable for administering a composition to a subject, such as a syringe or needle. In some embodiments, the kit is to be stored below 50° C., below 40° C., below 30° C., below 20° C., below 10° C., below 0° C., below −10° C., below −20° C., below −30° C., below −40° C., below −50° C., below-60° C., below-70° C., or below −80° C., such that the nucleic acids of the pharmaceutical composition are relatively stable over time. In some embodiments, the kit further comprises instructions for administering any of the pharmaceutical compositions provided herein to a subject.

### Pharmaceutical Compositions for Delivery and Methods Therefore

This disclosure provides pharmaceutical compositions comprising capped RNA molecules of the disclosure, particularly linear and circularized mRNA molecules. In certain embodiments pharmaceutical compositions of the disclosure further comprise pharmaceutically acceptable excipients and in certain other embodiments comprise one or more additional therapeutics agents.

In some embodiments, the compositions are suitable to be administered to a human subject in need thereof. In the context of the present disclosure, “active ingredient” refers generally to the capped RNA molecules described herein, particularly linear and circularized mRNA molecules as well as any additional therapeutic agents provided therewith.

It is generally understood by a person of ordinary skill in the art that the compositions described herein are also suitable for administration to any non-human subjects as well. A person of ordinary skill in the veterinary arts will understand that pharmaceutical compositions described herein can be suitable for administration to mammals including but not limited to primates, cattle, pigs, horses, sheep, goats, cats, dogs, mice, rats, whales, and other mammals. A person of ordinary skill in the veterinary arts also will understand that pharmaceutical compositions described herein can be suitable for administration to birds including by not limited to chickens, ducks, geese, turkey, and other domesticated birds, as well as wild birds particularly endangered species of such birds. Additionally, a person of ordinary skill in the veterinary arts will understand that pharmaceutical compositions described herein can be suitable for administration to a wide variety of fish including commercial or wild salmon, tuna, cod, sardine, zebra fish, shark, or the like.

Pharmacological compositions described herein can be prepared by any method known or developed in the art of pharmacology, immunology, virology, or in biotechnology in general.

In some embodiments, the formulations of a pharmacological composition described herein can comprise a unit dose of at least one RNA, in addition to at least one other pharmaceutically acceptable excipient. Such excipients can include but are not limited to, solvents, dispersions, buffers, diluents, surfactants, emulsifiers, isotonic agents, preservatives, thickeners, lubricating agents, oils, or the like.

In some embodiments, the pharmacological composition can comprise a delivery mechanism further comprising a lipid nanoparticle. The size of the lipid nanoparticle can be altered to counteract immunogenic response from the subject, or to allow for increased potency and pharmacological activity.

In other embodiments, the pharmacological composition can comprise a delivery mechanism further comprising a lipidoid as previously described in the art. See Akinc et al., 2008, Nat Biotechnol. 26:561-596; Frank-Kamenetsky et al., Proc Natl Acad Sci USA. 2008 105:11915-11920; Akinc et al., 2009, Mol Ther. 17:872-879; Love et al., 2010. Proc Natl Acad Sci USA 107:1864-1869; Leuschner et al., 2011, Nat Biotechnol. 29:1005-1010, all of which is incorporated herein in their entirety. Lipidoids refers broadly to lipid nanoparticles, liposomes, lipid emulsions, lipid micelles and the like. Lipidoids containing the pharmacological composition comprising the derivatized RNA can be administered parenterally by means including but not limited to, intravenous injection, intramuscular injection, subcutaneous injection, via dialysate, intrathecal injection, or intracranial injection.

A person of ordinary skill in the art would also recognize that other nucleotide delivery mechanisms exist such as the use of viral like, or viral derived particles. See Rohovie et al., 2016, Bioengineering & Translational Med. 2 (1): 43-57. Virus like particles can include coat proteins or viral capsids of a virus. Such particles can be PEGylated or further annealed to compounds that avoid phagocytotic clearance. Additionally, the surface of the virus like particle can be further functionalized to provide cellular specific targeting, facilitate extravasation, facilitate radio labeling, improve permeability across cellular boundaries, or to transcytose the blood-brain barrier. The virus like particles can be derived for animal viruses, bacteriophages, or plant viruses. Examples of suitable virus for derivation of a virus like particle delivery mechanism include but are not limited to cowpea chlorotic mottle virus, cowpea mosaic virus, hepatitis B virus (core), enterobacteria phage MS2, Salmonella typhimurium P22, enterobacteria phage Qβ amongst other suitable viruses. Derivatized RNA payloads can be loaded into the virus like particles by electrostatic adsorption or any other suitable method known to a person of ordinary skill in the art.

Various exemplary embodiments of compositions and methods according to this disclosure are now described in the following non-limiting Examples. The Examples are offered for illustrative purposes only and are not intended to limit the scope of the invention in any way. Indeed, various modifications of the invention in addition to those shown and described herein will become apparent to those skilled in the art from the foregoing description and the following examples and fall within the scope of the appended claims.

### EXAMPLES OF EMBODIMENTS

The following examples are given to illustrate the present disclosure. It should be understood, however, that the disclosure is not to be limited to the specific conditions or details described in these examples.

Embodiment 1: A method of producing a composition of capped RNA transcripts, comprising:
(a) synthesizing a 5′-phosphorylated RNA oligonucleotide in a solvent system comprising a nonpolar counterion;
(b) capping the 5′ phosphorylated RNA oligonucleotide, thereby producing a capped RNA oligonucleotide;
(c) purifying the capped RNA oligonucleotide, thereby producing a composition comprising a purified capped RNA oligonucleotide, wherein at least 95% of the total RNA oligonucleotides in the composition are capped RNA oligonucleotides; and
(d) ligating the purified capped RNA oligonucleotide to an RNA precursor, thereby producing a capped RNA transcript.

Embodiment 2: The method of claim 1, wherein at least 98% of the total RNA oligonucleotides in the composition produced in step (c) are capped RNA oligonucleotides.

Embodiment 3: The method of claim 2, wherein at least 99% of the total RNA oligonucleotides in the composition produced in step (c) are capped RNA oligonucleotides.

Embodiment 4: The method of claim 3, wherein at least 99.5% of the total RNA oligonucleotides in the composition produced in step (c) are capped RNA oligonucleotides.

Embodiment 5: The method of any one of claims 1-4, wherein the capping is performed chemically.

Embodiment 6: The method of claim 5, wherein the chemical capping is performed through an anhydrous reaction between the 5′-phosphorylated RNA oligonucleotide and a capping nucleotide conjugated to imidazole in the presence of 1-methylimidazole.

Embodiment 7: The method of any one of claims 1-6, wherein the RNA precursor comprises a 5′ untranslated region (5′ UTR) comprising an unstructured region.

Embodiment 8: The method of claim 7, wherein the unstructured region comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more consecutive adenosine, cytosine, guanine, or uridine nucleotides.

Embodiment 9: The method of claim 7, wherein the unstructured region comprises no more than 100, no more than 90, no more than 80, no more than 70, no more than 60, no more than 50, no more than 40, no more than 30, no more than 20, or no more than 10 consecutive adenosine, cytosine, guanine, or uridine nucleotides.

Embodiment 10: The method of claim 7, wherein the unstructured region comprises between 5 and 60, between 10 and 55, between 15 and 50, between 20 and 45, between 25 and 40, or between 30 and 35 consecutive adenosine, cytosine, guanine, or uridine nucleotides.

Embodiment 11: The method of any one of claims 1-10, wherein the purifying is done by high-performance liquid chromatography (HPLC).

Embodiment 12: The method of any one of claims 1-11, wherein at least 95% of the capped RNA transcripts produced in step (d) comprise a 5′ cap.

Embodiment 13: The method of claim 12, wherein at least 98% of the capped RNA transcripts produced in step (d) comprise a cap.

Embodiment 14: The method of claim 13, wherein at least 99% of the capped RNA transcripts produced in step (d) comprise a cap.

Embodiment 15: The method of claim 14, where at least 99.5% of the capped RNA transcripts produced in step (d) comprise a cap.

Embodiment 16: The method of any one of claims 1-15, further comprising circularizing the capped RNA transcript.

Embodiment 17: The method of claim 16, wherein the circularizing is performed by intron back-splicing.

Embodiment 18: The method of any one of claims 1-17 further comprising: (i) introducing one or more azide handles onto the 5′-phosphorylated RNA oligonucleotide, and (ii) conjugating one or more 5′ cap regions to the 5′-phosphorylated RNA oligonucleotide via click chemistry with the one or more azide handles.

Embodiment 19: The method of claim 18, wherein the introducing is performed by tRNA guanine transglycosylase (TGT).

Embodiment 20: The method of claim 18 or 19, wherein the introducing is performed during the in vitro synthesizing of step (a) with an azide-containing phosphoramidate.

Embodiment 21: The method of any one of claims 1-20, wherein the 5′-phosphorylated RNA oligonucleotide is between 3 and 160, between 5 and 150, between 7 and 140, between 9 and 130, between 11 and 120, between 13 and 110, between 15 and 100, between 17 and 90, between 19 and 80, between 21 and 70, between 23 and 60, between 25 and 50, between 27 and 40, or between 30 and 35 nucleotides in length.

Embodiment 22: The method of claim 21, wherein the 5′-phosphorylated RNA oligonucleotide is between 25 and 35 nucleotides in length.

Embodiment 23: The method of any one of claims 1-22, wherein the nonpolar counterion is ammonium (NH₄).

Embodiment 24: The method of any one of claims 1-23, wherein the synthesizing of step (a) is performed on a solid-phase support.

Embodiment 25: The method of any one of claims 1-24, wherein the 5′-phosphorylated RNA oligonucleotide comprises one or more modified nucleotides.

Embodiment 26: The method of any one of claims 1-25, wherein the RNA precursor comprises one or more modified nucleotides.

Embodiment 27: The method of claim 25 or 26, wherein the one or more modified nucleotides comprises a modified sugar.

Embodiment 28: The method of claim 27, wherein the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-0,4′-C-methylene-linked, 2′-O,4′-C-amino-linked ribose, 2′-O,4′-C-thio-linked ribose, and thiomorpholino oligo (TMO)-linked ribose.

Embodiment 29: The method of claim 27 or 28, wherein the modified sugar is selected from the following:

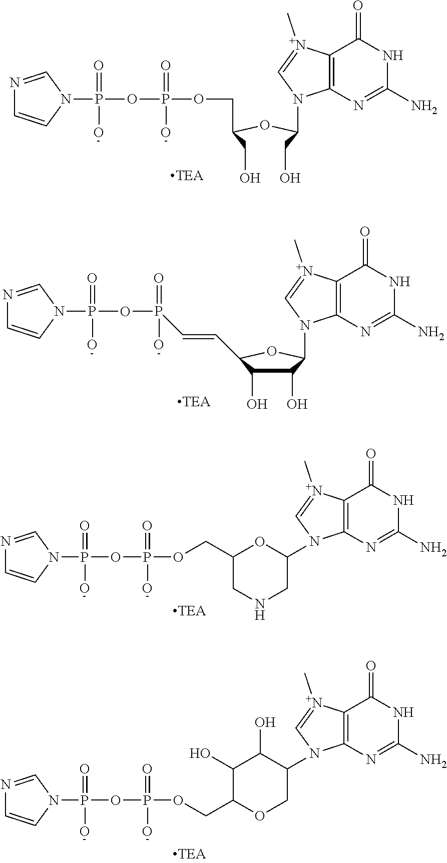

Embodiment 30: The method of any one of claims 27-29, wherein the 5′-phosporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified sugars.

Embodiment 31: The method of any one of claims 27-29, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified sugars.

Embodiment 32: The method of any one of claims 27-29, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars.

Embodiment 33: The method of any one of claims 27-29, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

Embodiment 34: The method of any one of claims 25-33, wherein the one or more modified nucleotides comprises a modified phosphate.

Embodiment 35: The method of claim 34, wherein the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

Embodiment 36: The method of claim 34 or 35, wherein the 5′-phosphorylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified phosphates.

Embodiment 37: The method of claim 34 or 35, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified phosphates.

Embodiment 38: The method of claim 34 or 35, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates.

Embodiment 39: The method of claim 34 or 35, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

Embodiment 40: The method of any one of claims 25-39, wherein the one or more modified nucleotides comprise a modified nucleobase.

Embodiment 41: The method of claim 40, wherein the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

Embodiment 42: The method of claim 40 or 41, wherein the 5′-phoshporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified nucleobases.

Embodiment 43: The method of claim 40 or 41, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified nucleobases.

Embodiment 44: The method of claim 40 or 41, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases.

Embodiment 45: The method of claim 40 or 41, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

Embodiment 46: The method of any one of claims 25-45, wherein the one or more modified nucleotides comprise one or more modified sugars, one or more modified phosphates, one or more modified nucleobases, or any combination thereof.

Embodiment 47: The method of any one of claims 1-46, wherein the capped RNA oligonucleotide comprises a 5′ cap selected from the group consisting of 7-methyguanosine (m7G), N7,3′-O-dimethyl-guanosine-5′-triphosphate-5′-guanosine (m7G-3′m-ppp-G), N7,2′-O-dimethyl-guanosine-5′-triphosphate-5′-guanosine (m7Gm-ppp-G), 7-benzylguanosine (Bn7G), chlorobenzylguanosine (ClBn7G), m7G bearing an LNA sugar (m7G-LNA), chlorobenzyl-O-ethoxyguanosine (ClBnOEt7G), 7-(4-chlorophenoxyethyl)-guanosine, 7-ethyl guanosine (e7G), 7-propyl guanosine (p7G), 7-isopropyl guanosine (ip7G), 7-butyl guanosine (b7G), 7-isobutyl guanosine (ib7G), 7-cyclopentyl guanosine (cp7G), 7-(carboxymethyl) guanosine (cm7G), 7-(2-phenylethyl) guanosine [7-(2-PhEt)G], 7-(1-phenylethyl) guanosine [7-(1-PhEt)G], m⁷Gppp_{BH3}G (D1 and D2 stereoisomers), m⁷Gpp_{BH3}G (D1 and D2 stereoisomers), m⁷Gp_{BH3}G (D1 and D2 stereoisomers), m⁷Gpp_{BH3}pm⁷G, m₂^{7,2′-O}GpppBH3G (D1 and D2 stereoisomers), m₂^{7,2′-O}Gpp_{BH3}PG (D1 and D2 diastereomers), m₂^{7,2′-OGppspG (D}1 and D2 diastereomers), N-Arylmethyl analogs, glyceryl, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2′-inverted nucleotide moiety, 3′-2 ‘-inverted abasic moiety, 1,4-butanediol phosphate, 3’-phosphoramidate, hexylphosphate, aminohexyl phosphate, 3′-phosphate, 3′-phosphorothioate, phosphorodithioate, cap1, cap2, cap3, cap4, ARCA, modified ARCA, inosine, N1-methylguanosine, LNA-guanosine, 2-azido-guanosine, and a bridging or non-bridging methylphosphonate moiety.

Embodiment 48: The method of any one of claims 1-47, wherein the ligating is performed using a T4 RNA Ligase.

Embodiment 49: The method of claim 11, wherein the HPLC comprises 10-200 mM hydrophobic hexyl ammonium ions.

Embodiment 50: The method of any one of claims 1-49, wherein the RNA precursor comprises a 3′ exonuclease-resistant modification.

Embodiment 51: The method of claim 50, wherein the 3′ exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

Embodiment 52: The method of any one of claims 1-51, wherein the RNA precursor further comprises a poly-A tail.

Embodiment 53: The method of claim 52, wherein the poly-A tail comprises between 25 and 500 nucleotides (SEQ ID NO: 168).

Embodiment 54: The method of claim 53, wherein the poly-A tail comprises between 50 and 100, between 100 and 150, between 150 and 200, between 200 and 300, between 300 and 400, or between 400 or 500 nucleotides.

Embodiment 55: The method of any one of claims 52-54, wherein the poly-A tail comprises 10 or more adenosine nucleotides (SEQ ID NO: 169).

Embodiment 56: The method of any one of claims 52-54, wherein 25-100%, 30-100%, 40-100%, 50-100%, 60-100%, 70-100%, 80-100%, 90-100%, 95-100%, 96-100%, 97-100%, 98-100%, or 99-100% of nucleotides of the poly-A tail are adenosine nucleotides.

Embodiment 57: The method of any one of claims 52-55, wherein 100% of the nucleotides of the poly-A tail are adenosine nucleotides.

Embodiment 58: The method of any one of claims 52-57, wherein the poly-A tail comprises one or more modified nucleotides.

Embodiment 59: The method of any one of claims 1-58, wherein the RNA precursor further comprises an open reading frame (ORF).

Embodiment 60: The method of claim 59, wherein the ORF is operably connected to a promoter.

Embodiment 61: The method of claim 59 or 60, wherein the ORF encodes at least one protein.

Embodiment 62: The method of claim 61, wherein the at least one protein is a therapeutic protein.

Embodiment 63: The method of claim 61 or 62, wherein the at least one protein is an antigen.

Embodiment 64: The method of any one of claims 1-63, wherein the RNA precursor further comprises a therapeutic nucleic acid.

Embodiment 65: The method of claim 64, wherein the therapeutic nucleic acid is chosen from the group consisting of an antisense oligonucleotide (ASO), an aptamer, an RNA decoy, an siRNA, a shRNA, a miRNA, or a gRNA.

Embodiment 66: The method of any one of claims 1-65, wherein the 5′-phosphorylated RNA oligonucleotide comprises a promoter sequence.

Embodiment 67: A capped RNA transcript produced by any one of claims 1-66.

Embodiment 68: A delivery agent comprising the capped RNA transcript of claim 67, wherein the delivery agent comprises a lipid, a peptide, a protein, an antibody, a carbohydrate, a nanoparticle, or a microparticle.

Embodiment 69: The delivery agent of claim 68, wherein the nanoparticle or microparticle is a lipid nanoparticle or microparticle, a polymer nanoparticle or a polymer microparticle, a protein nanoparticle or a protein microparticle, or a solid nanoparticle or a solid microparticle.

Embodiment 70: A cell comprising the capped RNA transcript of claim 67.

Embodiment 71: The cell of claim 70, wherein the cell is a mammalian cell.

Embodiment 721: A composition comprising the capped RNA transcript of claim 66, the delivery agent of claim 67 or 68, or the cell of claim 70 or 71.

Embodiment 73: The composition of claim 72 further comprising an additional agent.

Embodiment 74: The composition of claim 73, wherein the additional agent is an agent which has a therapeutic effect when administered to a subject.

Embodiment 75: The composition of claim 72 or 73, wherein the additional agent is chosen from the list consisting of a nucleotide, a nucleic acid, an amino acid, a peptide, a protein, a small molecule, an aptamer, a lipid, or a carbohydrate.

Embodiment 76: The composition of claim 75, wherein the additional agent is a shRNA, a siRNA, or an antisense oligonucleotide (ASO).

Embodiment 77: The composition of any one of claims 73-75, wherein the additional agent is an antigen or adjuvant.

Embodiment 78: The composition of any one of claims 73-77, wherein the composition is a pharmaceutical composition, wherein the pharmaceutical composition comprises a pharmaceutically acceptable excipient.

Embodiment 79: A method of preventing or treating a disease in a subject, comprising introducing an effective amount of the capped RNA transcript of claim 67, the delivery agent of claim 68 or 69, the cell of claim 70 or 71, or the composition of any one of claims 71-77 to the subject.

Embodiment 80: The method of claim 79, wherein the subject is a human.

Embodiment 81: The capped RNA transcript of claim 67, the delivery agent of claim 68 or 69, the cell of claim 70 or 71, or the composition of any one of claims 72-78 for use in preventing or treating a disease in a subject.

Embodiment 82: A kit comprising the capped RNA oligonucleotide and/or the RNA precursor of any one of claims 1-66.

Embodiment 83: The kit of claim 82 further comprising an RNA ligase.

Embodiment 84: A kit comprising the composition of claim 78, a device for administering the composition to a subject, and instructions for administering the composition to the subject.

Embodiment 85: A method of screening for altered translation efficiency conferred by one or more nucleotide modifications in an RNA transcript, comprising:
(a) synthesizing a 5′-phosphorylated RNA oligonucleotide comprising one or more modified nucleotides in a solvent system comprising a nonpolar counterion;
(b) chemically capping the 5′-phosphorylated RNA oligonucleotide, thereby producing a capped RNA oligonucleotide;
(c) purifying the capped RNA oligonucleotide;
(d) ligating the capped RNA oligonucleotide to an RNA precursor comprising an open reading frame (ORF) encoding a protein, thereby producing a capped RNA transcript;
(e) expressing the capped RNA transcript in a cell; and
(f) measuring expression of the protein in the cell.

Embodiment 86: The method of claim 85, wherein the synthesizing of step (a) is performed on a solid-phase support.

Embodiment 87: The method of claim 85 or 86, wherein the purifying of step (c) is done by high-performance liquid chromatography (HPLC).

Embodiment 88: The method of claim 87, wherein the HPLC comprises 10-200 mM hydrophobic hexyl ammonium ions.

Embodiment 89: The method of any one of claims 85-88, wherein the RNA precursor further comprises a 5′ untranslated region (5′ UTR) comprising an unstructured region.

Embodiment 90: The method of claim 89, wherein the unstructured region comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more consecutive adenosine, cytosine, guanine, or uridine nucleotides.

Embodiment 91: The method of claim 89, wherein the unstructured region comprises no more than 100, no more than 90, no more than 80, no more than 70, no more than 60, no more than 50, no more than 40, no more than 30, no more than 20, or no more than 10 consecutive adenosine, cytosine, guanine, or uridine nucleotides.

Embodiment 92: The method of claim 89, wherein the unstructured region comprises between 5 and 60, between 10 and 55, between 15 and 50, between 20 and 45, between 25 and 40, or between 30 and 35 consecutive adenosine, cytosine, guanine, or uridine nucleotides.

Embodiment 93: The method of any one of claims 85-92, wherein the RNA precursor further comprises a 3′ exonuclease-resistant modification.

Embodiment 94: The method of claim 93, wherein the 3′ exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

Embodiment 95: The method of any one of claims 85-94, wherein the RNA precursor comprises one or more modified nucleotides.

Embodiment 96: The method of any one of claims 85-95, wherein the one or more modified nucleotides comprises a modified sugar.

Embodiment 97: The method of claim 96, wherein the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-0,4′-C-methylene-linked, 2′-O,4′-C-amino-linked ribose, 2′-O,4′-C-thio-linked ribose, and thiomorpholino oligo (TMO)-linked ribose.

Embodiment 98: The method of claim 96 or 97, wherein the modified sugar is selected from the following:

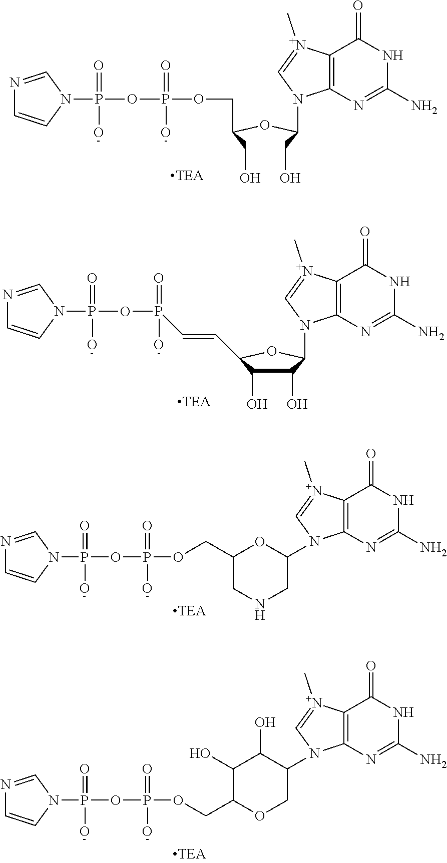

Embodiment 99: The method of any one of claims 96-98, wherein the 5′-phosporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified sugars.

Embodiment 100: The method of any one of claims 96-98, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified sugars.

Embodiment 101: The method of any one of claims 96-100, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars.

Embodiment 102: The method of any one of claims 96-100, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

Embodiment 103: The method of any one of claims 85-102, wherein the one or more modified nucleotides comprises a modified phosphate.

Embodiment 104: The method of claim 103, wherein the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

Embodiment 105: The method of claim 103 or 104, wherein the 5′-phosphorylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified phosphates.

Embodiment 106: The method of claim 103 or 104, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified phosphates.

Embodiment 107: The method of any one of claims 103-106, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates.

Embodiment 108: The method of any one of claims 103-106, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

Embodiment 109: The method of any one of claims 85-108, wherein the one or more modified nucleotides comprise a modified nucleobase.

Embodiment 110: The method of claim 109, wherein the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

Embodiment 111: The method of claim 109 or 110, wherein the 5′-phoshporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified nucleobases.

Embodiment 112: The method of claim 109 or 110, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified nucleobases.

Embodiment 113: The method of any one of claims 88-112, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases.

Embodiment 114: The method of any one of claims 88-113, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

Embodiment 115: The method of any one of claims 85-114, wherein the one or more modified nucleotides comprise one or more modified sugars, one or more modified phosphates, one or more modified nucleobases, or any combination thereof.

Embodiment 116: The method of any one of claims 85-115, wherein the chemical capping is performed through an anhydrous reaction between the 5′-phosphorylated RNA oligonucleotide and a capping oligonucleotide conjugated to imidazole in the presence of 1-methylimidazole.

Embodiment 117: The method of any one of claims 85-116, wherein the RNA precursor further comprises a poly-A tail.

Embodiment 118: The method of claim 117, wherein the poly-A tail comprises between 25 and 500 nucleotides (SEQ ID NO: 168).

Embodiment 119: The method of claim 118, wherein the poly-A tail comprises between 50 and 100, between 100 and 150, between 150 and 200, between 200 and 300, between 300 and 400, or between 400 and 500 nucleotides.

Embodiment 120: The method of any one of claims 117-119, wherein the poly-A tail comprises 10 or more adenosine nucleotides (SEQ ID NO: 169).

Embodiment 121: The method of any one of claims 117-119, wherein 25-100%, 30-100%, 40-100%, 50-100%, 60-100%, 70-100%, 80-100%, 90-100%, 95-100%, 96-100%, 97-100%, 98-100%, or 99-100% of nucleotides of the poly-A tail are adenosine nucleotides.

Embodiment 122: The method of any one of claims 85-121, wherein the cell is a mammalian cell.

Embodiment 123: The method of any one of claims 85-122, wherein the measuring is done by Western blotting, flow cytometry, mass spectrometry, enzyme-linked immunosorbent assay (ELISA), microscopy, bicinchoninic acid (BCA) assay, Coomassie protein assay, fluorometry, and/or spectrofluorometry.

Embodiment 124: The method of any one of claims 85-123, wherein the protein is a reporter protein or a therapeutic protein.

Embodiment 125: The method of claim 124, wherein the reporter protein is chosen from the group consisting of β-galactosidase, chloramphenicol acetyltransferase, green fluorescent protein (e.g., GFP or EGFP), red fluorescent protein (e.g., RFP, ERFP, or dsRed), yellow fluorescent protein (e.g., YFP or EYFP), cyan fluorescent protein (e.g., CFP or ECFP), mOrange, tdTomato, mCherry, and luciferase.

Embodiment 126: A method of screening for altered RNA stability conferred by one or more nucleotide modifications in an RNA transcript, comprising:
(a) synthesizing a 5′-phosphorylated RNA oligonucleotide comprising one or more modified nucleotides in a solvent system comprising a nonpolar counterion;
(b) chemically capping the 5′-phosphorylated RNA oligonucleotide, thereby producing a capped RNA oligonucleotide;
(c) purifying the capped RNA oligonucleotide;
(d) ligating the capped RNA oligonucleotide to an RNA precursor comprising an open reading frame (ORF) encoding a protein, thereby producing a capped RNA transcript;
(e) expressing the purified capped RNA transcript in a cell; and
(f) measuring levels of the capped RNA transcript in the cell.

Embodiment 127: The method of claim 126, wherein the synthesizing of step (a) is performed on a solid-phase support.

Embodiment 128: The method of claim 126 or 127, wherein the purifying of step (c) is done by high-performance liquid chromatography (HPLC).

Embodiment 129: The method of claim 128, wherein the HPLC comprises 10-200 mM hydrophobic hexyl ammonium ions.

Embodiment 130: The method of any one of claims 126-130, wherein the RNA precursor further comprises a 5′ untranslated region (5′ UTR) comprising an unstructured region.

Embodiment 131: The method of claim 130, wherein the unstructured region comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more consecutive adenosine, cytosine, guanine, or uridine nucleotides.

Embodiment 132: The method of claim 130, wherein the unstructured region comprises no more than 100, no more than 90, no more than 80, no more than 70, no more than 60, no more than 50, no more than 40, no more than 30, no more than 20, or no more than 10 consecutive adenosine, cytosine, guanine, or uridine nucleotides.

Embodiment 133: The method of claim 130, wherein the unstructured region comprises between 5 and 60, between 10 and 55, between 15 and 50, between 20 and 45, between 25 and 40, or between 30 and 35 consecutive adenosine, cytosine, guanine, or uridine nucleotides.

Embodiment 134: The method of any one of claims 126-133, wherein the RNA precursor further comprises a 3′ exonuclease-resistant modification.

Embodiment 135: The method of claim 134, wherein the 3′ exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

Embodiment 136: The method of any one of claims 126-135, wherein the RNA precursor comprises one or more modified nucleotides.

Embodiment 137: The method of any one of claims 126-136, wherein the one or more modified nucleotides comprises a modified sugar.

Embodiment 138: The method of claim 137, wherein the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose.

Embodiment 139: The method of claim 137 or 138, wherein the modified sugar is selected from the following:

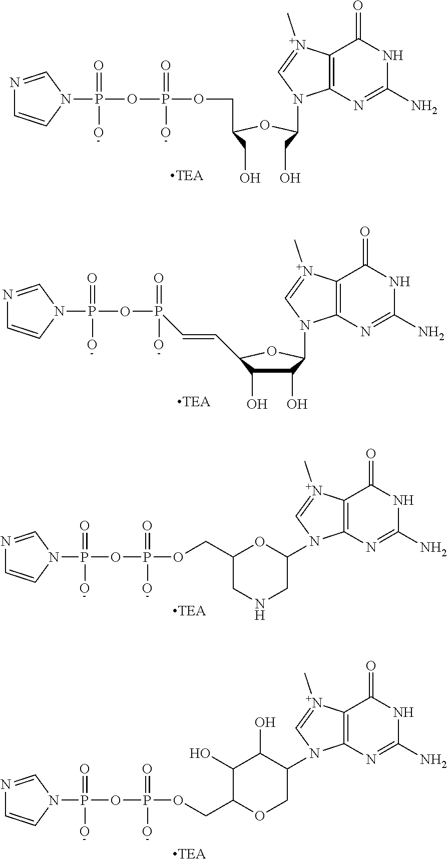

Embodiment 140: The method of any one of claims 137-139, wherein the 5′-phosporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified sugars.

Embodiment 141: The method of any one of claims 137-139, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified sugars.

Embodiment 142: The method of any one of claims 137-141, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars.

Embodiment 143: The method of any one of claims 137-141, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

Embodiment 144: The method of any one of claims 126-143, wherein the one or more modified nucleotides comprises a modified phosphate.

Embodiment 145: The method of claim 144, wherein the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

Embodiment 146: The method of claim 144 or 145, wherein the 5′-phosphorylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified phosphates.

Embodiment 147: The method of claim 144 or 145, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified phosphates.

Embodiment 148: The method of any one of claims 144-145, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates.

Embodiment 149: The method of any one of claims 144-145, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

Embodiment 150: The method of any one of claims 126-149, wherein the one or more modified nucleotides comprise a modified nucleobase.

Embodiment 151: The method of claim 150, wherein the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

Embodiment 152: The method of claim 150 or 151, wherein the 5′-phoshporylated RNA oligonucleotide comprises between 1 and 3, between 3 and 5, between 5 and 10, between 10 and 15, between 15 and 30, between 30 and 50, between 50 and 75, between 75 and 100, between 100 and 125, between 125 and 150, or between 135 and 160 modified nucleobases.

Embodiment 153: The method of claim 150 or 152, wherein the 5′-phosphorylated RNA oligonucleotide comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 120, at least 140, at least 160, or more modified nucleobases.

Embodiment 154: The method of any one of claims 150-153, wherein the RNA precursor comprises between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases.

Embodiment 155: The method of any one of claims 150-154, wherein the RNA precursor comprises at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

Embodiment 156: The method of any one of claims 126-155, wherein the one or more modified nucleotides comprise one or more modified sugars, one or more modified phosphates, one or more modified nucleobases, or any combination thereof.

Embodiment 157: The method of any one of claims 126-155, wherein the chemical capping is performed through an anhydrous reaction between the 5′-phosphorylated RNA oligonucleotide and a capping oligonucleotide conjugated to imidazole in the presence of 1-methylimidazole.

Embodiment 158: The method of any one of claims 126-157, wherein the RNA precursor further comprises a poly-A tail.

Embodiment 159: The method of claim 158, wherein the poly-A tail comprises between 25 and 500 nucleotides (SEQ ID NO: 168).

Embodiment 160: The method of claim 159, wherein the poly-A tail comprises between 50 and 100, between 100 and 150, between 150 and 200, between 200 and 300, between 300 and 400, or between 400 and 500 nucleotides.

Embodiment 161: The method of any one of claims 158-160, wherein the poly-A tail comprises 10 or more adenosine nucleotides (SEQ ID NO: 169).

Embodiment 162: The method of any one of claims 158-160, wherein 25-100%, 30-100%, 40-100%, 50-100%, 60-100%, 70-100%, 80-100%, 90-100%, 95-100%, 96-100%, 97-100%, 98-100%, or 99-100% of nucleotides of the poly-A tail are adenosine nucleotides.

Embodiment 163: The method of any one of claims 126-162, wherein the cell is a mammalian cell.

Embodiment 164: The method of any one of claims 126-163, wherein the measuring is done by Western blotting, PC R, real-time PCR, next-generation sequencing (NGS), flow cytometry, mass spectrometry, enzyme-linked immunosorbent assay (ELISA), microscopy, bicinchoninic acid (BCA) assay, Coomassie protein assay, fluorometry, spectrofluorometry, RNA-seq, single-cell RNA-seq, and/or nanoscale secondary ion mass spectrometry (NanoSIMS).

Embodiment 165: The method of any one of claims 126-164, wherein the protein is a reporter protein or a therapeutic protein.

Embodiment 166: The method of claim 165, wherein the reporter protein is chosen from the group consisting of β-galactosidase, chloramphenicol acetyltransferase, green fluorescent protein (e.g., GFP or EGFP), red fluorescent protein (e.g., RFP, ERFP, or dsRed), yellow fluorescent protein (e.g., YFP or EYFP), cyan fluorescent protein (e.g., CFP or ECFP), mOrange, tdTomato, mCherry, and luciferase.

Embodiment 167: An RNA molecule comprising (i) one or more modified nucleotides at position +3 or higher with reference to a 5′ terminus of the RNA molecule, and (ii) at least one 5′ cap.

Embodiment 168: The RNA molecule of claim 167 comprising two or more 5′ caps.

Embodiment 169: The RNA molecule of claim 168, wherein the two or more 5′ caps are conjugated to a 5′ UTR of the RNA molecule.

Embodiment 170: The RNA molecule of claim 168 or 169, wherein the two or more 5′ caps are conjugated to the RNA molecule via click chemistry.

Embodiment 171: The RNA molecule of any one of claims 168-170, wherein the two or more 5′ caps are conjugated to the RNA molecule via a photochemical crosslinking reaction.

Embodiment 172: The RNA molecule of any one of claims 167-171, wherein the one or more modified nucleotides comprises a modified sugar.

Embodiment 173: The RNA molecule of claim 172, wherein the modified sugar is selected from the group consisting of 2′-deoxy fluoro (2FA), L-adenosine (LA), 2′-deoxyadenosine (dA), locked nucleic acid (LNA), 2′-methoxy (2OMe), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, 2′-O,4′-C-thio-linked ribose, and thiomorpholino oligo (TMO)-linked ribose.

Embodiment 174: The RNA molecule of claim 172 or 173, wherein the modified sugar is selected from the following:

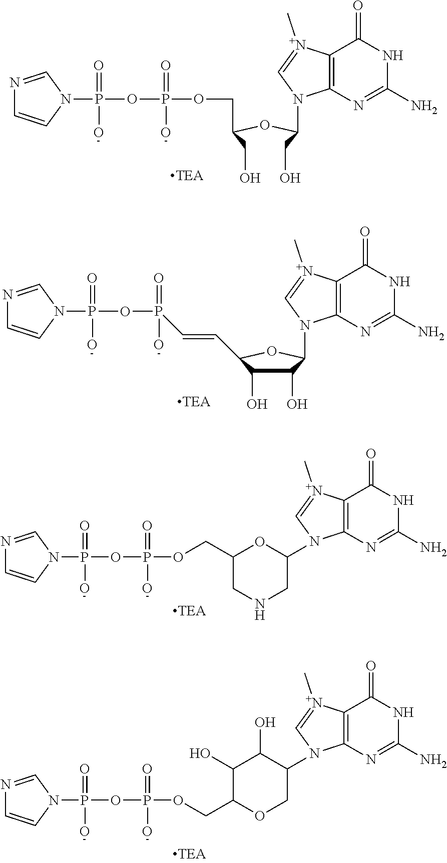

Embodiment 175: The RNA molecule of any one of claims 172-174, comprising between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified sugars.

Embodiment 176: The RNA molecule of any one of claims 172-174, comprising at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified sugars.

Embodiment 177: The RNA molecule of any one of claims 167-176, wherein the one or more modified nucleotides comprises a modified phosphate.

Embodiment 178: The RNA molecule of claim 177, wherein the modified phosphate is selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

Embodiment 179: The RNA molecule of claim 177 or 178 comprising between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified phosphates.

Embodiment 180: The RNA molecule of claim 177 or 178 comprising at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified phosphates.

Embodiment 181: The RNA molecule of any one of claims 167-180, wherein the one or more modified nucleotides comprises a modified nucleobase.

Embodiment 182: The RNA molecule of claim 181, wherein the modified nucleobase is selected from the group consisting of inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (16A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

Embodiment 183: The RNA molecule of claim 181 or 182 comprising between 1 and 3, between 3 and 5, between 5 and 10, between 10 and 15, between 15 and 30, between 30 and 50, between 50 and 100, between 100 and 200, between 200 and 300, between 400 and 500, between 600 and 700, between 800 and 900, or between 900 and 1000 modified nucleobases.

Embodiment 184: The RNA molecule of claim 181 or 182 comprising at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 750, at least 1000, or more modified nucleobases.

Embodiment 185: The RNA molecule of any one of claims 167-184, wherein the one or more modified nucleotides comprise one or more modified sugars, one or more modified phosphates, one or more modified nucleobases, or any combination thereof.

Embodiment 186: The RNA molecule of any one of claims 167-185, wherein the 5′ cap is selected from the group consisting of 7-methyguanosine (m7G), N7,3′-O-dimethyl-guanosine-5′-triphosphate-5′-guanosine (m7G-3′m-ppp-G), N7,2′-O-dimethyl-guanosine-5′-triphosphate-5′-guanosine (m7Gm-ppp-G), 7-benzylguanosine (Bn7G), chlorobenzylguanosine (ClBn7G), m7G bearing an LNA sugar (m7G-LNA), chlorobenzyl-O-ethoxyguanosine (ClBnOEt7G), 7-(4-chlorophenoxyethyl)-guanosine, 7-ethyl guanosine (e7G), 7-propyl guanosine (p7G), 7-isopropyl guanosine (ip7G), 7-butyl guanosine (b7G), 7-isobutyl guanosine (ib7G), 7-cyclopentyl guanosine (cp7G), 7-(carboxymethyl) guanosine (cm7G), 7-(2-phenylethyl) guanosine [7-(2-PhEt)G], 7-(1-phenylethyl) guanosine [7-(1-PhEt)G], m⁷Gppp_{BH3}G (D1 and D2 stereoisomers), m⁷Gpp_{BH3}G (D1 and D2 stereoisomers), m⁷Gp_{BH3}G (D1 and D2 stereoisomers), m⁷Gpp_{BH3}pm⁷G, m₂^{7,2′-O}GpppBH3G (D1 and D2 stereoisomers), m₂^{7,2′-O}Gpp_{BH3}pG (D1 and D2 diastereomers), m2^{7,2′-OGppspG (D}1 and D2 diastereomers), N-Arylmethyl analogs, glyceryl, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2′-inverted nucleotide moiety, 3′-2 ‘-inverted abasic moiety, 1,4-butanediol phosphate, 3’-phosphoramidate, hexylphosphate, aminohexyl phosphate, 3′-phosphate, 3′-phosphorothioate, phosphorodithioate, cap1, cap2, cap3, cap4, ARCA, modified ARCA, inosine, N1-methylguanosine, LNA-guanosine, 2-azido-guanosine, and a bridging or non-bridging methylphosphonate moiety.

Embodiment 187: The RNA molecule of any one of claims 167-186 further comprising at least one poly-A tail.

Embodiment 188: The RNA molecule of claim 187, wherein the at least one poly-A tail comprises between 25 and 500 nucleotides (SEQ ID NO: 168).

Embodiment 189: The RNA molecule of claim 188, wherein the at least one poly-A tail comprises between 50 and 100, between 100 and 150, between 150 and 200, between 200 and 300, between 300 and 400, or between 400 and 500 nucleotides.

Embodiment 190: The RNA molecule of any one of claims 188-189, wherein the at least one poly-A tail comprises 10 or more adenosine nucleotides (SEQ ID NO: 169).

Embodiment 191: The RNA molecule of any one of claims 188-189, wherein 25-100%, 30-100%, 40-100%, 50-100%, 60-100%, 70-100%, 80-100%, 90-100%, 95-100%, 96-100%, 97-100%, 98-100%, or 99-100% of nucleotides of the at least one poly-A tail are adenosine nucleotides.

Embodiment 192: The RNA molecule of any one of claims 167-191, wherein the 5′ cap is added to the RNA molecule through a chemical capping method.

Embodiment 193: The RNA molecule of claim 192, wherein the chemical capping method is an anhydrous reaction between a 5′-phosphorylated RNA molecule and a capping nucleotide conjugated to imidazole in the presence of 1-methylimidazole.

Embodiment 194: The RNA molecule of any one of claims 167-193 further comprising a 5′ untranslated region (5′ UTR).

Embodiment 195: The RNA molecule of claim 194, wherein the 5′ UTR comprises a promoter.

Embodiment 196: The RNA molecule of any one of claims 167-195 further comprising a 3′ untranslated region (3′ UTR).

Embodiment 197: The RNA molecule of claim 196, wherein the 3′ UTR comprises at least one exonuclease-resistant modification.

Embodiment 198: The RNA molecule of claim 197, wherein the exonuclease-resistant modification is selected from the group consisting of phosphorothioate (PS) linkage, 2′-O-methyl (2OMe), 2′ Fluoro, inverted deoxythymidine (dT), inverted dideoxythymidine (ddT), 3′ phosphorylation, C3 spacer, 2′-O-methoxy-ethyl(2′-MOE), G-quadruplex, and 2′-3′-dideoxy nucleotide (ddN).

Embodiment 199: The RNA molecule of any one of claims 167-198 comprising two or more 5′ caps.

Embodiment 200: The RNA molecule of any one of claims 167-199 comprising two or more poly-A tails.

Embodiment 201: The RNA molecule of any one of claims 167-200 further comprising an open reading frame (ORF).

Embodiment 202: The RNA molecule of claim 201, wherein the ORF encodes a protein.

Embodiment 203: The RNA molecule of claim 202, wherein the protein is a therapeutic protein.

Embodiment 204: The RNA molecule of claim 203, wherein the protein is an antigen.

Embodiment 205: The RNA molecule of any one of claims 199-204 further comprising a sequence encoding a therapeutic nucleic acid.

Embodiment 206: The RNA molecule of claim 205, wherein the therapeutic nucleic acid is an antisense oligonucleotide (ASO), an aptamer, an RNA decoy, an siRNA, a shRNA, a miRNA, or a gRNA.

Embodiment 207: The RNA molecule of any one of claims 167-206, further comprising a 3′ cap.

Embodiment 208: The RNA molecule of any one of claims 167-207, wherein the RNA molecule is a circular RNA molecule.

Embodiment 209: A vector comprising the RNA molecule of any one of claims 167-208.

Embodiment 210: A delivery agent comprising the RNA molecule of any one of claims 167-209, wherein the delivery agent comprises a lipid, a peptide, a protein, an antibody, a carbohydrate, a nanoparticle, or a microparticle.

Embodiment 211: The delivery agent of claim 210, wherein the nanoparticle or microparticle is a lipid nanoparticle or a lipid microparticle, a polymer nanoparticle or a polymer microparticle, a protein nanoparticle or a protein microparticle, or a solid nanoparticle or a solid microparticle.

Embodiment 212: A cell comprising the RNA molecule of any one of claims 167-208 or the vector of claim 203.

Embodiment 213: The cell of claim 212, wherein the cell is a mammalian cell.

Embodiment 214: A composition comprising the RNA molecule of any one of claims 167-208, the vector of claim 209, the delivery agent of claim 210 or 211, or the cell of claim 212 or 213.

Embodiment 215: The composition of claim 214 further comprising an additional agent.

Embodiment 216: The composition of claim 215, wherein the additional agent is an agent which has a therapeutic effect when administered to a subject.

Embodiment 217: The composition of claim 215 or 216, wherein the additional agent is a nucleotide, a nucleic acid, an amino acid, a peptide, a protein, a small molecule, an aptamer, a lipid, or a carbohydrate.

Embodiment 218: The composition of claim 217, wherein the nucleotide is a shRNA, a siRNA, a miRNA, or an antisense oligonucleotide (ASO).

Embodiment 219: The composition of any one of claims 214-218, wherein the additional agent is an antigen or adjuvant.

Embodiment 220: The composition of any one of claims 214-219, wherein the composition is a pharmaceutical composition comprising a pharmaceutically acceptable excipient.

Embodiment 221: A method of preventing or treating a disease in a subject, comprising introducing an effective amount of the RNA molecule of any one of claims 167-208, the vector of claim 209, the delivery agent of claim 210 or 211, or the composition of any one of claims 214-220 to the subject.

Embodiment 222: The method of 221, wherein the subject is a human.

Embodiment 223: The RNA molecule of any one of claims 167-208, the vector of claim 209, the delivery agent of claim 210 or 211, or the composition of any one of claims 214-220 for use in preventing or treating a disease in a subject.

Embodiment 224: The RNA molecule of claim 223, wherein the subject is a human.

Embodiment 225: A kit comprising the composition of claim 220, a device for administering the composition to a subject, and/or instructions for administering the composition to the subject.

### EXAMPLES

### Example 1: Systematic Interrogation of 5′ Modifications by Divergent Synthesis

Existing methods for preparing capped linear mRNA involve co-transcriptional incorporation of cap analogues during in vitro transcription (IVT) or enzymatic capping and methylation post-IVT (Ramanathan et al. “mRNA capping: biological functions and application.” Nucleic Acids Res. 44, 7511-7526 (2016)). Traditional dinucleotide cap analogue m⁷G(5′)ppp(5′) G often results in an unintended “reversed” m⁷G cap, a problem that was later addressed by the development of the anti-reverse cap analogue (ARCA) (Grudzien-Nogalska et al. “Synthesis of Anti-Reverse Cap Analogs (ARCAs) and their Applications in mRNA Translation and Stability.” Methods in Enzymology (Academic Press, 2007), 43, 203-227; Stepinski et al. “Synthesis and properties of mRNAs containing the novel ‘anti-reverse’ cap analogs 7-methyl (3′-O-methyl)GpppG and 7-methyl (3′-deoxy)GpppG.” RNA 7, 1486-1495 (2001)). Recent developments of trinucleotide/tetranucleotide cap analogues facilitate direct incorporation of Cap-1/Cap-2 structures (with 2′OMe at the first and second base of 5′ UTR) and have been used to screen most cap modifications. However, these methods do not accommodate modifications that are not tolerated by RNA polymerase or capping enzymes, nor extend beyond the first two bases, creating a screening bias due to differing cap incorporation efficiencies.

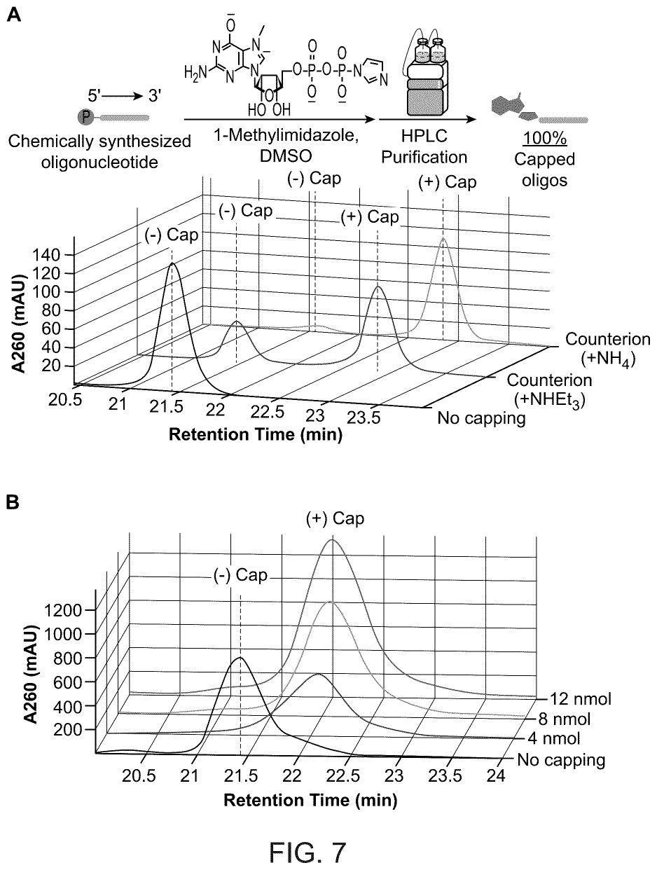

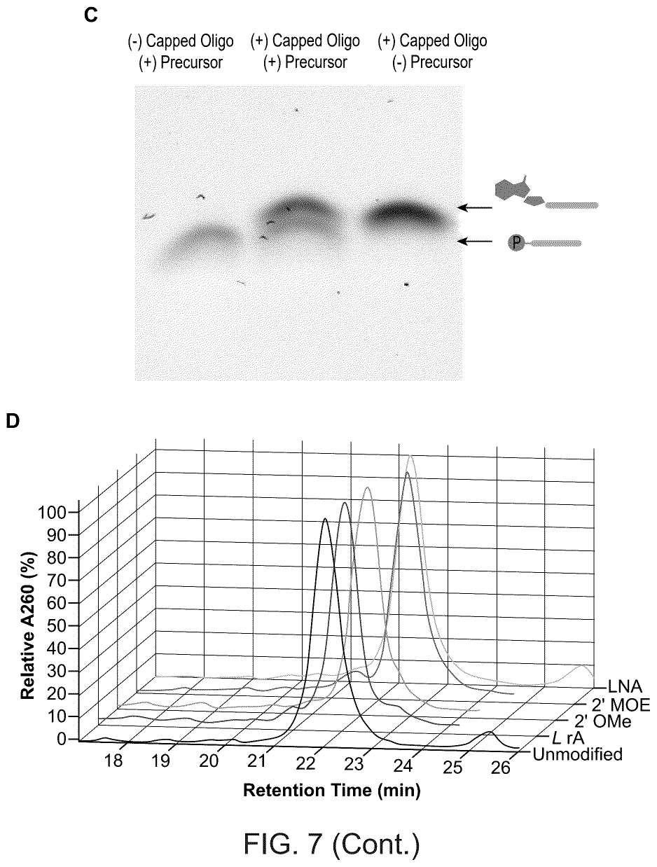

To overcome these challenges, the capping process was decoupled from mRNA synthesis. 5′-phosphorylated oligonucleotides with diverse chemical modifications were prepared using solid-phase synthesis, and subsequently capped via a chemical reaction using m7G diphosphate imidazolide (m7GDP-Im) or derivatives (Abe et al. “Complete Chemical Synthesis of Minimal Messenger RNA by Efficient Chemical Capping Reaction.” ACS Chem. Biol. 17, 1308-1314 (2022)). It was discovered that switching the oligonucleotide counterion to ammonium allowed robust capping without any divalent ion additives. Furthermore, by fine-tuning the reversed-phase high-performance liquid chromatography (RP-HPLC) gradients with relatively hydrophobic hexylammonium ions, fully capped products were isolated at scale (FIGS. 7A-7D). These capped oligonucleotides were subsequently ligated to 5′-monophosphorylated mRNAs containing N¹-methypseudouridine (m¹ψ′) produced from IVT followed by RNA 5′ pyrophosphohydrolase (RppH) treatment.

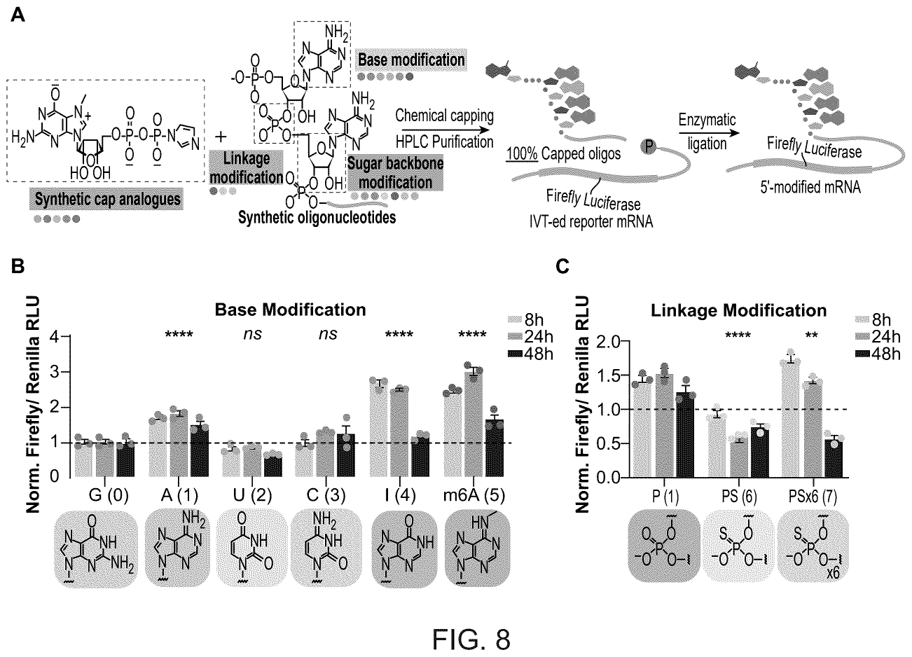

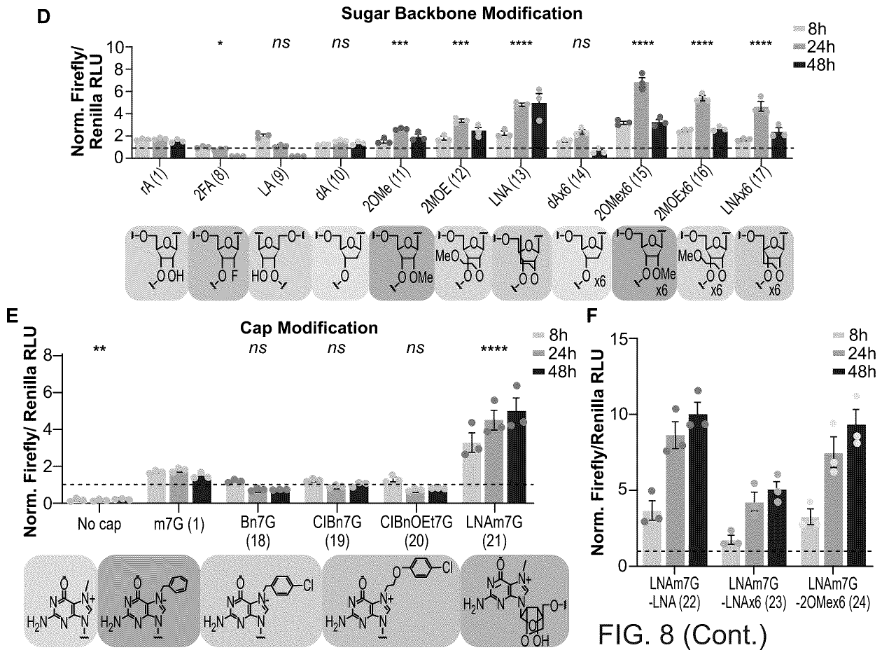

The modularity of this workflow enabled divergent construction and evaluation of mRNAs featuring four different types of cap and 5′ UTR modifications (FIG. 8A): (1) base identities within the 5′ UTR, including as locations beyond the third base (+3 position), (2) phosphodiester linkages within the 5′ UTR, (3) sugar backbone modifications within the 5′ UTR, and (4) cap modifications. The effects of all of these different types of modifications on protein translation were assayed using Firefly luciferase reporter at 8-, 24-, and 48-hours post-transfection.

Protein yield varied in the order of A>G~C~U with the corresponding nucleotides at the +1 position. Changing A to m6A, or introducing non-canonical bases such as Inosine (I) enhanced protein production (FIG. 8B). For subsequent screenings, m7G-rG (the “wild-type” base identities in ARCA-capped synthetic mRNAs) was used as a benchmark. Surprisingly, introducing phosphorothioate (PS) linkages at various positions within the 5′ UTR region did not yield beneficial results (FIG. 8C). When modifying the sugar of the backbone, results showed that locked nucleic acid (LNA), 2′-methoxy (2′OMe), and 2′-methoxyethoxy (2′MOE) modifications significantly elevated mRNA translation: single-base substitutions at the +1 position led to a 4.8-fold increase, while increasing the number of 2′OMe- and 2′MOE-modified bases to 6 (from positions +1 to +6) resulted in 6.9-fold and 5.4-fold increases, respectively. Substitution with 2′-deoxyfluoro (2′FA) hindered translation, whereas chirality-inverted L-adenosine (LA) or 2′-deoxyadenosine (dA) caused negligible changes (FIG. 8D).

To evaluate cap modifications, various m7GDP-Im analogues were synthesized. Surprisingly, aromatic cap modifications, such as substitution of the m7G methyl group with benzyl (Bn7G), chlorobenzyl (ClBn7G), or a high-affinity eIF4E inhibitor, chlorobenzyl-O-ethoxy (ClBnOEt7G), did not outperform m7G. The LNA cap analogue (m7G-LNA) successfully enhanced translation by 4.5-fold (FIG. 8E).

Finally, optimal modifications from all four categories were combined to demonstrate the modularity of these effects. Simultaneous introduction of m7G-LNA and LNA at the +1 position or 2′OMe from +1 to +6 further enhanced translation to 8.6- and 7.5-fold, respectively (FIG. 8F).

### Plasmid Cloning, Characterization, and Purification (Linear+Circular)

mRNA expression vectors were cloned as previously described (Aditham et al. “Chemically Modified mocRNAs for Highly Efficient Protein Expression in Mammalian Cells.” ACS Chem. Biol. 17, 3352-3366 (2022)), with minor updates. The coding sequences (CDS) for the proteins of interest were inserted into an optimized backbone containing (from 5′ to 3′) a T7 promoter sequence, a 5′ human alpha globin UTR, a CDS of interest, a 3′ human alpha globin UTR, a 100×A template-encoded poly(A) tail, and an Esp3I linearization site. The CDS-containing plasmid/gene blocks were PCR amplified, gel-purified, and assembled into the optimized backbone using NEBuilder HiFi DNA Assembly Master Mix [NEB, E2621S], transformed into NEB Stable cells, and sequence-verified with whole plasmid and Sanger sequencing.

The Firefly luciferase construct was obtained from pmirGLO Dual-Luciferase miRNA Target Expression Vector (Promega, E1330). Renilla luciferase constructs were obtained from pmirGLO without cloning into the optimized vector. The NanoLuc constructs were obtained by gene synthesis from Genewiz. The hEPO construct was obtained by gene synthesis from Genewiz according to the previously reported sequence (Chen et al. “Engineering circular RNA for enhanced protein production.” Nat. Biotechnol. (2022), doi: 10.1038/s41587-022-01393-0).

Linear mRNA Synthesis and Characterization

DNA plasmids were obtained aforementioned and linearized by Esp3I (NEB, R0734S). Linearized plasmids are purified with the DNA Clean & Concentrator-25 kit [Zymo Research, D4033] and characterized with agarose gel electrophoresis. mRNA constructs were synthesized by IVT using HiScribe T7 High Yield RNA Synthesis Kit [NEB, E2040S] per manufacturer's protocol except 100% replacement of UTP with N1-methyl pseudouridine-5′-triphosphate [Trilink, N-1081-1] and addition of 1:50 SUPERase-In RNase inhibitor [ThermoFisher Scientific, AM2694]. Following IVT reaction, DNA templates were digested by TURBO DNase and purified using Monarch RNA cleanup kit [NEB, T2040L]. mRNA concentrations were quantified using the Qubit RNA HS Assay [ThermoFisher Scientific, Q32852] or the Qubit RNA BR Assay [ThermoFisher Scientific, Q10210]. Unless otherwise specified, mRNA products are suspended in 1:50 (v/v) RNase inhibitor-containing RNase-free water (subsequently referred to as RNase-free water) and stored at −80° C.

### Synthesis of Capping Reagents for Cap Analogs

m7G-Im, Bn7G-Im, ClBn7G-Im, ClBnOEt7G-Im and LNAm7G-Im capping reagents were synthesized following previously reported protocol (Abe et al. “Complete Chemical Synthesis of Minimal Messenger RNA by Efficient Chemical Capping Reaction.” ACS Chem. Biol. 17, 1308-1314 (2022)). m7GDP, Bn7GDP, ClBn7GDP, CIBnOEt7GDP were synthesized by treating the GDP sodium salt with dimethyl sulfoxide or corresponding alkylation reagents, followed by HPLC purification (Chen et al. “Structure-guided design, synthesis, and evaluation of guanine-derived inhibitors of the eIF4E mRNA-cap interaction.” J. Med. Chem. 55, 3837-3851 (2012)). LNAm7GDP was synthesized by introducing the phosphate to the 5′ hydroxyl group of LNA guanosine (Kore et al. “Locked nucleic acid (LNA)-modified dinucleotide mRNA cap analogue: synthesis, enzymatic incorporation, and utilization.” J. Am. Chem. Soc. 131, 6364-6365 (2009)). Then, GDP-Imidazole derivatives were prepared according to the general protocol (Abe et al., 2022) and purified through acetone precipitation to give the capping reagent that can be used directly in the capping reaction.

### Capped Oligonucleotide Synthesis

12 nmol of solid phase synthesized oligonucleotide (with ammonium as counterion) was dissolved in a solution of 40 mM m7GDP-Im (or corresponding cap analogue) in 42 μL of anhydrous DMSO, and 8 μL of 1-methyl-imidazole was added. The reaction was mixed well and heated at 55° C. for 3 hrs. The reaction was then quenched by addition of 50 μL of water and directly subjected to HPLC purification using method 1. Fractions containing the capped products were pooled, lyophilized, and resuspended in RNase-free water and stored at −80° C. until being used. Concentrations of capped oligos were quantified using Qubit microRNA assay kit [Invitrogen, Q32880] and nanodrop.

### General Conditions for RNA RP-HPLC Purification

All purification was conducted on an Agilent 1260 Infinity II HPLC. Acetonitrile (solvent A) [Sigma Aldrich, 34851], 100 mM hexylamine/acetic acid in water (pH 7.0, with 20% acetonitrile w/v) (solvent B), 50 mM diethylamine/acetic acid+50 mM ammonium acetate in water (pH 7.0) (solvent C) were used as the mobile phases and PLRP-S column as the stationary phase.

Method 1: 100 Å pore size was used, 0% A+100% B (0~5 mins, hold); 10% A+90% B (5~10 mins, linear increase); 25% A+75% B (10~55 mins, linear increase).

Method 2: 300 Å pore size was used, 0% A+100% B (0~5 mins, hold); 15% A+85% B (5~10 mins, linear increase); 50% A+50% B (10~45 mins, linear increase).

Method 3: 4000 Å pore size was used, 0% A+100% B (0 mins); 20% A+80% B (0~2 mins, linear increase); 70% A+30% B (2~30 mins, linear increase).

Method 4: 4000 Å pore size was used, 0% A+100% C (0 mins); 25% A+75% B (0~25 mins, linear increase).

Enzymatic Ligation of Modified Oligonucleotides to mRNAs

5′-triphosphorylated mRNA was first treated with RppH [NEB, M0356S] per manufacturer's protocol to generate 5P-mRNA and purified with the Monarch RNA cleanup kit. Synthetic oligo and 5P-mRNA were mixed at a molar ratio of 25:1, and diluted in 2× 50% PEG-8000, 10× T4 RNA ligase buffer, 10× T4 RNA ligase [Promega, M1051], and RNase-free water. The reaction was incubated at 37° C. for 30 mins and inactivated by the addition of 50×500 mM EDTA (pH 8.0). Products were purified first by the Monarch RNA cleanup kit and then by RNase-free HPLC (method 3). Purified fractions were pulled and desalted using the Monarch RNA cleanup kit and ligation efficiency was characterized using RNase H assay as described previously (Aditham et al., 2022). In case of incomplete ligation, a second round of reaction was performed.

Modification Screening with Time-Course Dual Luciferase Assay

HeLa cells [ATCC, CCL-2] were maintained in DMEM culture media [ThermoFisher Scientific, 119951] containing 10% FBS and 1% penicillin-streptomycin [ThermoFisher Scientific, 15070063] in a 37° C. incubator with 5% CO₂ and passaged at a ratio of 1:10 every 3 days. On the day before mRNA transfection, HeLa cells were seeded at 90% confluence in individual wells on 24-well plates. The following day, 50 ng of Renilla luciferase (internal control) mRNA and 50 ng of modified Firefly luciferase mRNA were transfected using Lipofectamine MessengerMAX Transfection Reagent [ThermoFisher Scientific, LMRNA003] per manufacturer's protocols. Additional controls that contain only Renilla luciferase mRNA or lipofection reagent only were included. Three individual transfections were conducted for each condition. 6 hours after transfection, the transfection media was removed, and cells were trypsinized and reseeded to three white clear-bottom 96-well plates [Corning, 3610] in phenol-red-free media. At 8-, 24-, and 48-hours post-transfection, cell culture media was removed and cells were rinsed with DPBS. Cells were lysed and luciferase activity was measured using the Promega Dual Glo Luciferase Assay System [Promega, E2920]. Firefly luminescence/Renilla luminescence for each well was used as mRNA activity readout. In cases where Nluc and Fluc were used, the protocol was conducted similarly using the Nano-Glo Dual-Luciferase Reporter Assay System [Promega, N1610].

### Example 2: Mechanistic Characterization of 5′ Modifications on mRNA Translation

The 5′ chemical modifications described in Example 1 may impact protein production via two potential mechanisms: altered translation efficiency, which is regulated by eIF binding, and/or translation duration, which is determined by resistance to decapping enzymes. Accordingly, the effects of the modifications on eIF4E affinity was investigated. eIF4E is a direct cap-binder essential for canonical translation initiation and resistance against human decapping enzyme 2 (hDcp2), the primary decapping enzyme responsible for the first irreversible step in 5′-to-3′ mRNA decay.

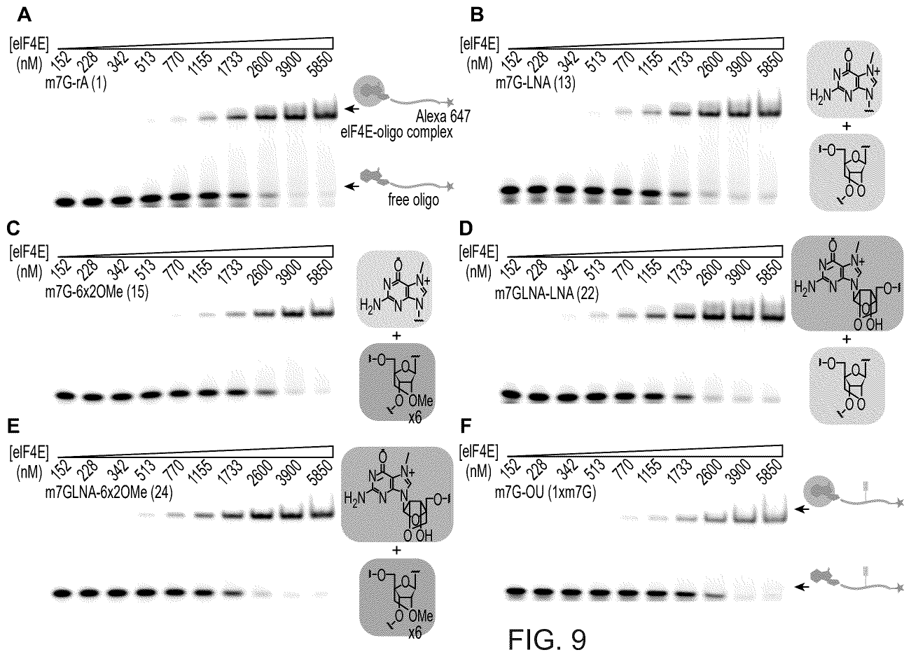

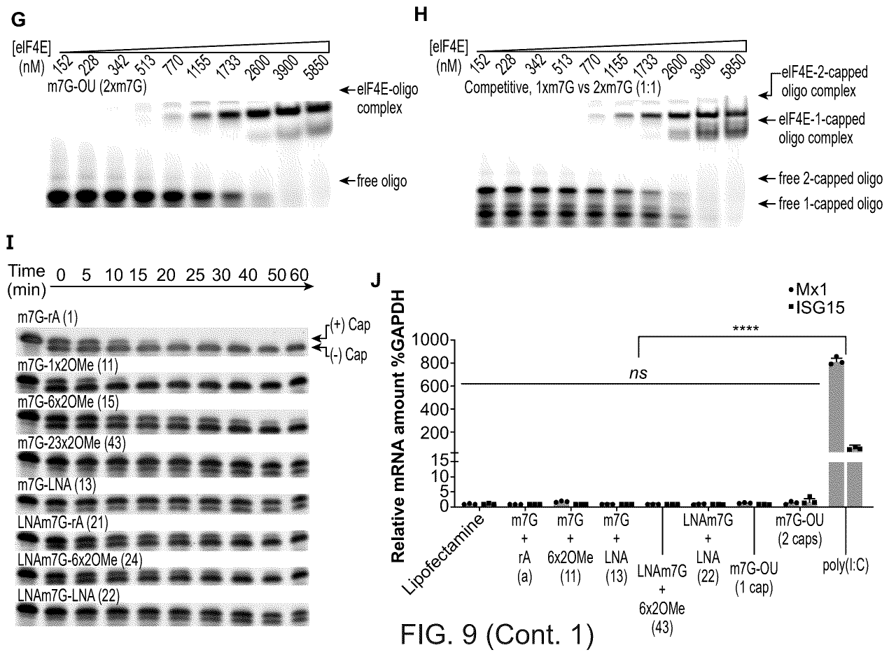

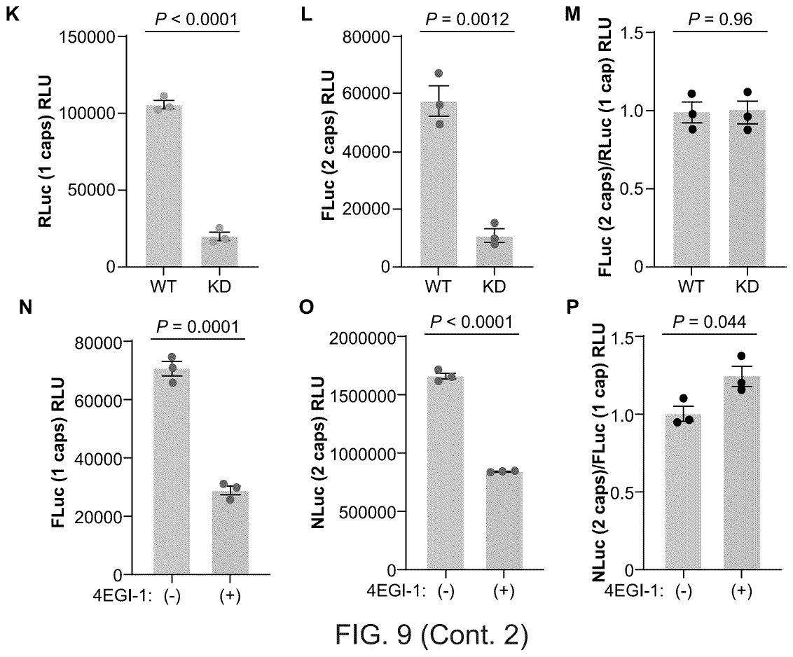

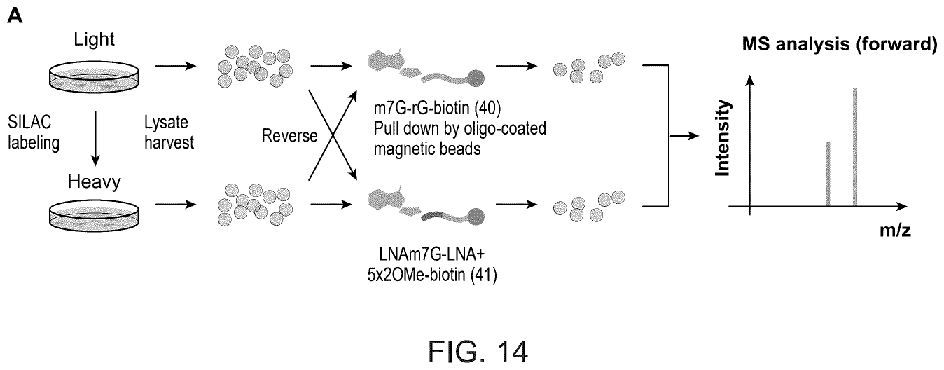

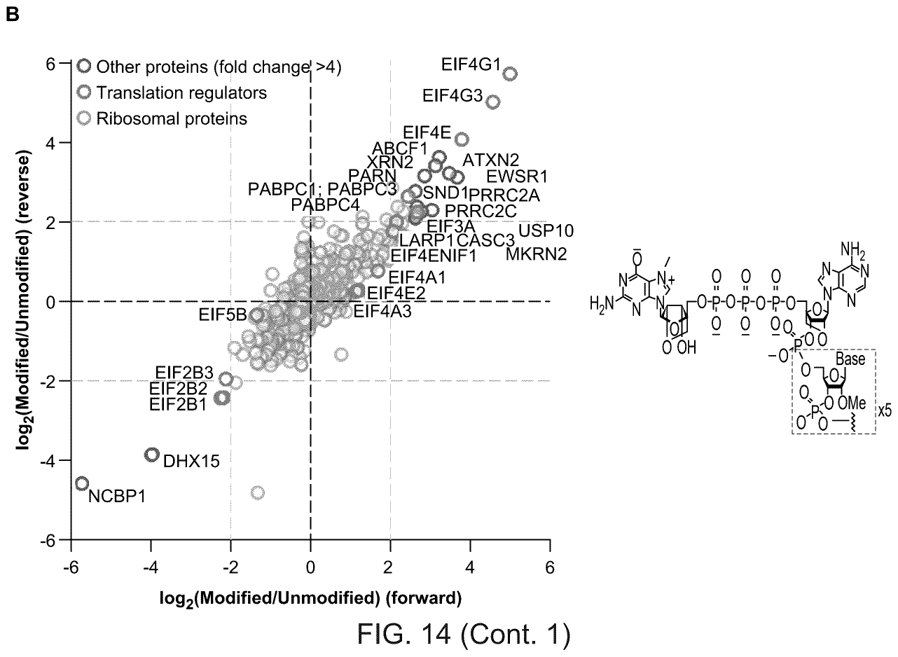

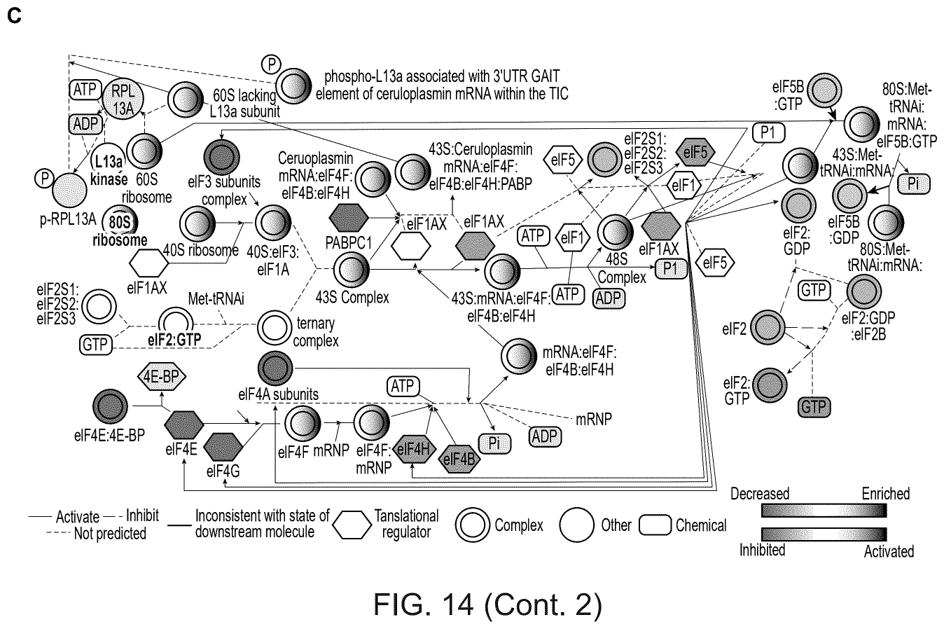

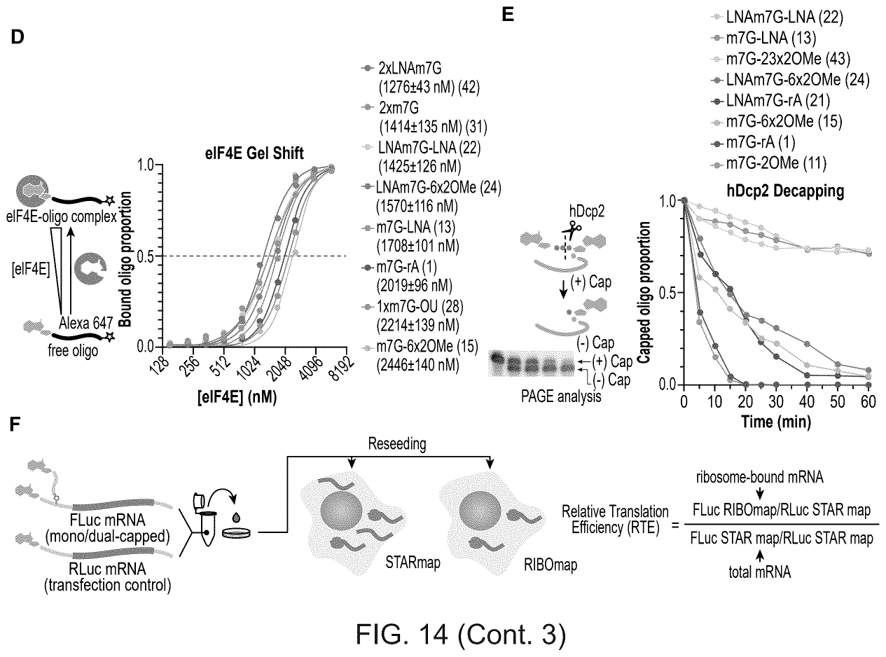

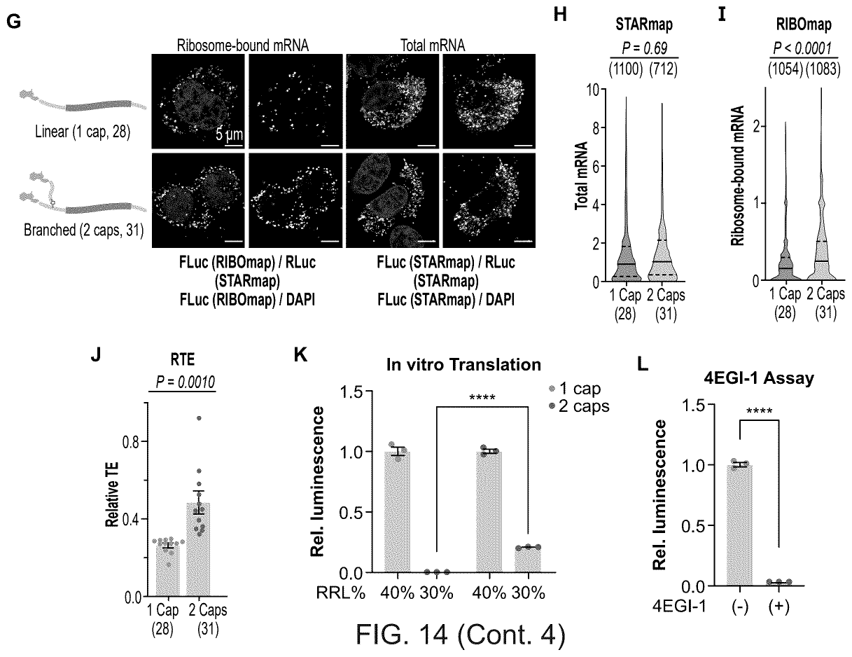

eIF4E affinity to fluorophore-labeled capped RNA oligonucleotides was quantified through electrophoretic mobility shift assay (EMSA) (FIGS. 14D and 9A-9E). The incorporation of LNA on both the m7G cap and the +1 base significantly increased eIF4E binding affinity to the RNA, lowering the dissociation constant (K_{d}) from 2019-96 nM to 1425±126 nM (FIGS. 9B and 9D). Interestingly, the 6×2′OMe 5′ UTR modification slightly reduced eIF4E affinity to 2446±140 nM (FIG. 9C) despite significantly enhancing protein expression in cell culture., and its K_{d} was rescued by the addition of the m7G-LNA cap (1570±116 nM) (FIG. 9E).

Next, the influence of modifications on decapping activity was examined. To this end, 23-nucleotide (23-nt) oligonucleotides with various modification patterns were subjected to decapping reactions and analyzed at different time intervals (FIGS. 14E and 9I). LNA modifications on both the cap and +1 nucleotide dramatically increased resistance against hDcp2 capping, with 71%~73% of capped transcripts remaining after 60 minutes, compared to complete decapping of the unmodified transcript within just 15 minutes. Conversely, the introduction of 2′OMe at the +1 position increased susceptibility towards decapping, but additional 2′OMe incorporation within the first 6 bases (positions +1 to +6) increased decapping resistance, which may be relevant to a site-dependent effect due to the potential roles of cap-1/cap-2 in marking “aged” mRNA for turnover (Despic and Jaffrey, “mRNA ageing shapes the Cap2 methylome in mammalian mRNA.” Nature. 614, 358-366 (2023)).

Collectively, both increased affinity for eIF4E and resistance against hDcp2 decapping contributed to the superior performance of LNA-modified oligonucleotides in cell-based expression tests. Lastly, the potential immunogenicity of 5′-modified mRNAs were assessed, revealing that both LNA and 2′OMe modifications led to marginal changes in immunogenicity when introduced to m¹Ψ-modified mRNA, suggesting their potentials for therapeutic applications (FIG. 9J).

eIF4E Electrophoretic Mobility Shift Assay (EMSA)

Capped oligonucleotides were labeled on the 3′ end using AF647 labeled cytidine-5′-phosphate-3′-(6-aminohexyl) phosphate [Jena Bioscience, NU-1706-AF647] and T4 RNA ligase ligated AF647-labeled cytidine onto the capped oligos using manufacturer's protocols. Oligos were then incubated in the binding buffer (1 mM DTT, 50 mM KCl, 10 mM Tris-HCl (pH 7.6), Novex Hi-Density TBE Sample Buffer [Invitrogen, LC6678]) with varying eIF4E-His [Cayman Chemical Inc, 25150] concentrations prepared by sequential dilutions from the stock solution. The oligos and PABP were incubated at 4° C. for 30 mins and characterized by EMSA with Novex TBE Gels, 4-20% [Invitrogen, EC6225BOX] at 4° C. AF647 labeled oligos were imaged with BioRad ChemiDoc MP Imaging System [12003154] and band intensities were quantified using ImageJ. For Kd calculation, the proportions of bound were plotted against eIF4E concentration using GraphPad Prism (Specific binding with Hill slope).

hDcp2 Decapping Assay

500 ng of capped oligos were incubated in 75 μL decapping reaction buffer (10 mM Tris-HCl (pH 7.5), 100 mM NaCl, 2 mM MgCl2, 1 mM DTT, 2 mM MnCl2, 1:25 SUPERaseIn (v/v)) with 300 ng of hDcp2 [Enzymax LLC, 86] at 37° C. At indicated time points, 5 μL of reaction mixture was aliquoted out into 10 μL of denaturing gel-loading buffer [NEB, B0363S, with urea added to a final concentration of 5 M] and cooled to −20° C. until all time points were collected. The fractions were analyzed by PAGE using Novex 15% TBE-Urea gel [Invitrogen, EC6885BOX] by running at 100 V for 2 hours and stained by 1×SYBR Gold [ThermoFisher Scientific, S11494], and visualized using BioRad ChemiDoc MP Imaging System [12003154]. Band intensities were quantified using ImageJ.

RNA Extraction and cDNA Preparation

For the evaluation of innate immune response induced by the 5′ modified mRNA constructs, the cell culture media was removed post transfection for 24 hours. Then, 300 μL of Trizol reagent was added to each well, followed by extracting the total mRNA from cell lysate with RNA Miniprep Kit [Zymo Research: R2051] according to the manufacturer's protocol. The optional DNase digestion was performed, also according to the manufacturer's protocol. The isolated RNA was then quantified using Nanodrop. Reverse transcription of total RNA was performed using the LunaScript RT SuperMix Kit [New England Biolab, Inc., E3010L] according to the manufacturer's protocol with 1 μg of total RNA.

### RT-qPCR

qPCR was performed using the Luna Universal qPCR Master Mix [New England Biolabs, Inc., M3003] according to the manufacturer's protocol. Briefly, the reactions were set up with 250 nM primers for GAPDH, Mx1 and ISG15 and 80 nM for IFNB 1 in 20 μL. The amplifications were conducted with the following protocol: 95° C. for 5 min, 40 cycles of 95° C. for 15 s, 60° C. for 30 s. The specificity of primer pairs was tested with melting curves at the end of the 40th amplification cycle. All the gene expressions were calculated and normalized to GAPDH.

### Example 3: Synthesis and Chemo-Topological Optimization of Multi-Capped mRNA

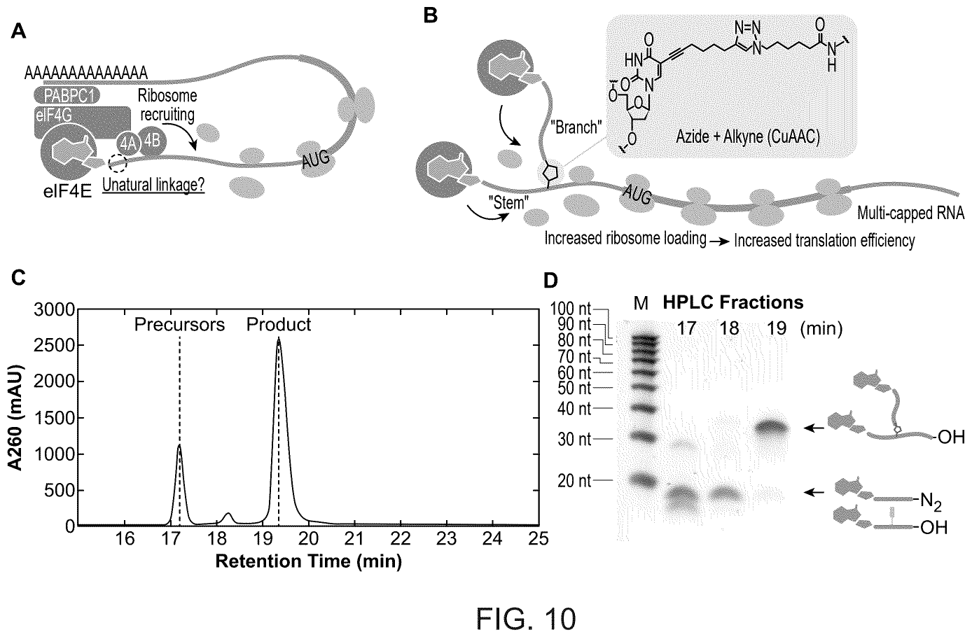

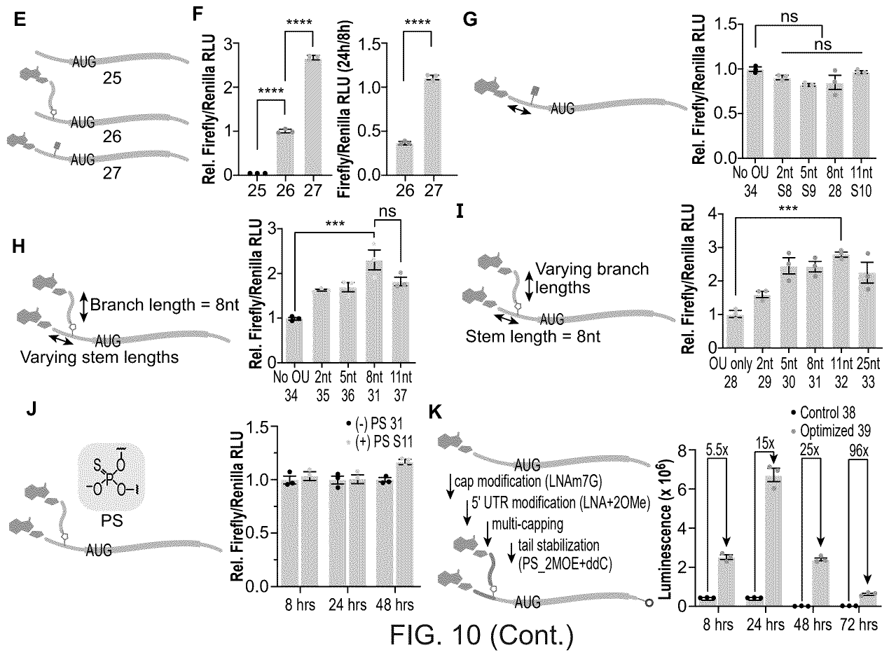

To investigate beyond traditional chemical modifications encoded directly in oligonucleotides, the ligation-based approach for introducing 5′ UTR regions described herein was combined with click chemistry moieties to enable topological engineering of the mRNAs. It was hypothesized that introducing multiple caps at the 5′ end of an mRNA could enhance translation efficiency (TE) by increasing valency for eIF binding events. Existing knowledge of cap-dependent translation initiation suggests that eIF4E binding to m7G triggers downstream recruitment of other eIFs through protein-protein interactions (FIG. 10A). Given that the m7G cap acts as an anchor to guide the translation initiation complex (TIC) formation in a proximity-based manner (Aitken and Lorsch, “A mechanistic overview of translation initiation in eukaryotes.” Nat. Struct. Mol. Biol. 19, 568-576 (2012)), it was reasoned that a continuous phosphodiester backbone between the cap and the rest of the mRNA may not be necessary for translation initiation, and that adding extra internal caps to the 5′ UTR region of a single mRNA transcript may enhance the RNA-TIC interaction valency, thus boosting TE (FIG. 10B).

As proof-of-concept, a completely chemically synthesized minimal mRNA was produced containing a 5-octadiynyl dU (OU) handle and coding an 11-amino acid HiBiT peptide that could be sensitively quantified using luminometry. This short mRNA was chemically capped, HPLC-purified, and attached to a fully capped oligonucleotide with a 3′-azide moiety (FIGS. 11A and 11B). The branching in the 5′ UTR region minimally affected translation, and incorporating an additional internal cap increased luminescence by approximately 2-fold (FIG. 11C).

Next, dual-capped oligonucleotides were synthesized using a similar workflow, conjugating chemically capped azide/alkyne-labeled oligos via copper catalyzed azide-alkyne cycloaddition (CuAAC) followed by HPLC purification (FIGS. 10C and 10D). These branched-capped oligonucleotides were ligated to the full-length Firefly luciferase (Fluc) mRNA. Using this pipeline, the topological parameters of the branched mRNA were tested. First, it was found that an internal branched cap could effectively induce mRNA translation, even without a 5′ terminal cap, albeit less effectively than a cap in the natural topology (FIGS. 10E and 10F). It is possible that this is due to the inability of the branched cap to stabilize the mRNA from 5′ to 3′ decay, as indicated by the rapid drop in luminescence between 8- and 24-hours (FIG. 10F).

Next, a two-dimensional screening of branching positions (distance between 5′ terminus and the branching point) and the lengths of the capped branching oligonucleotides was conducted. Introduction of a single OU handle at various positions within the 5′ UTR region minimally affected translation (FIG. 10G). Adding the extra capped branch after the second base (at the +3 position or higher) was beneficial (FIG. 10H). Capped branch oligos with lengths of 5~15 nucleotides yielded optimal translation (FIG. 101). Similar to the screening of 5′ UTR modifications shown in Example 1, a PS linkage introduced into the branched oligo did not further increase protein expression, indicating that it is decapping, not 5′ UTR exonuclease degradation, that abolished protein translation (FIG. 10J).

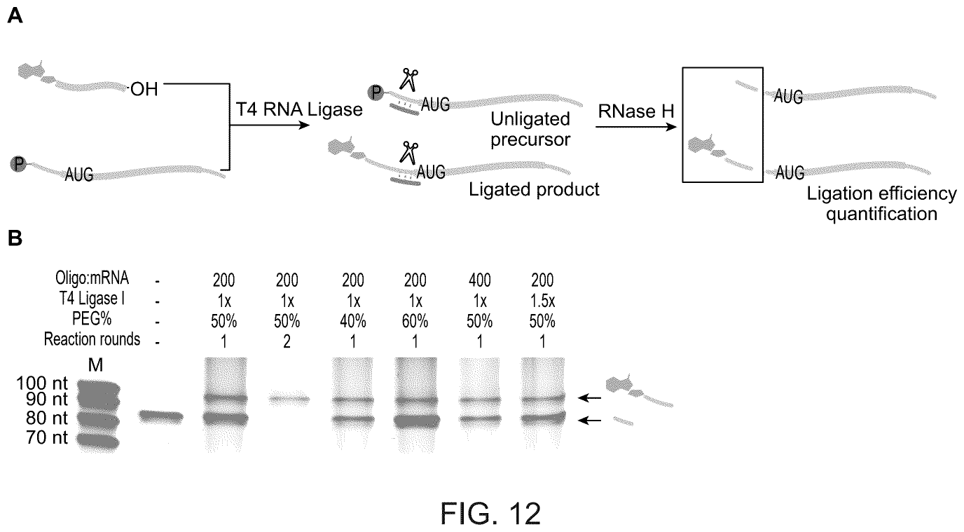

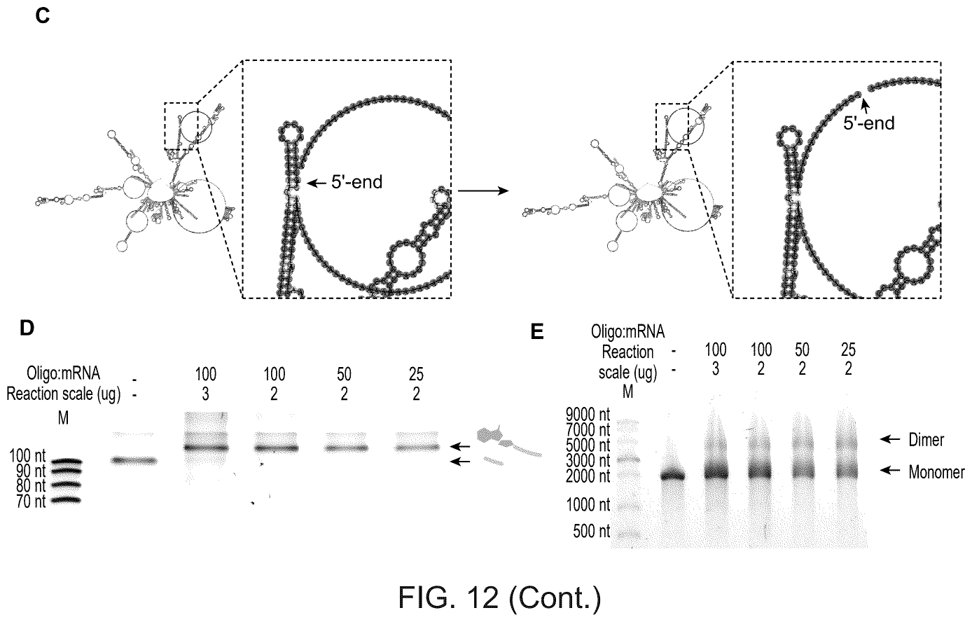

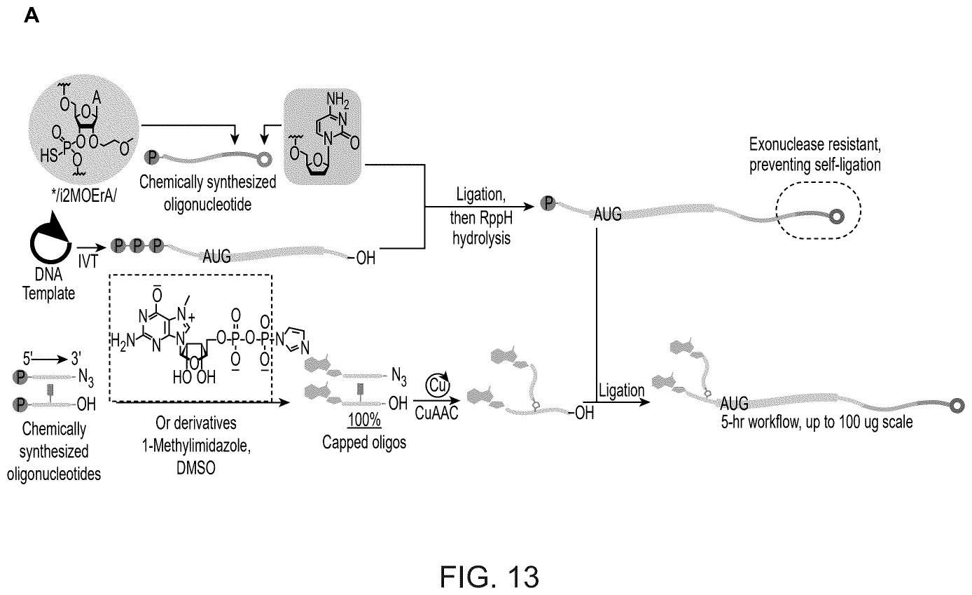

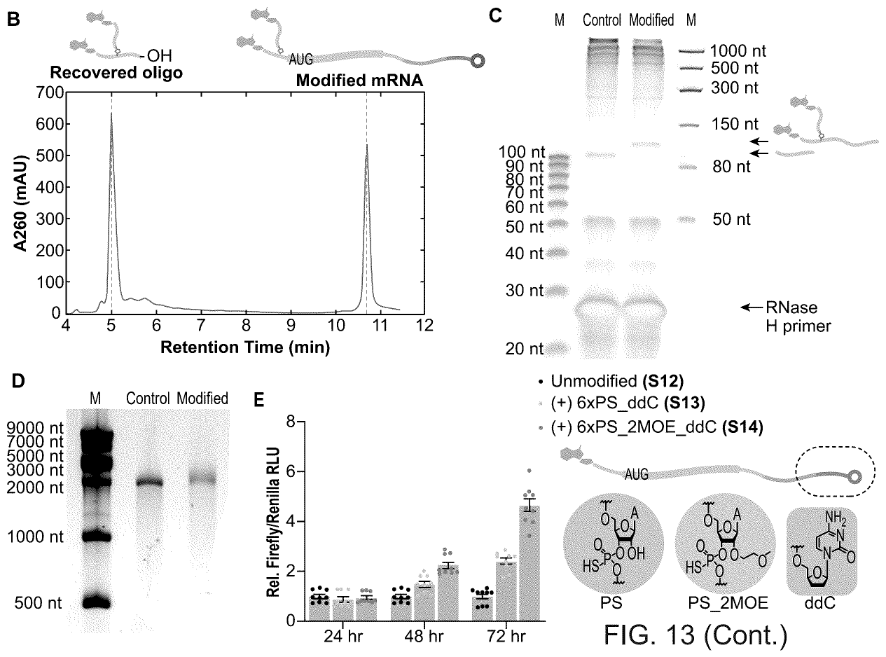

Following the testing of mRNA 5′ chemical and topological modifications, there remained two significant challenges for applying the technology described herein to its full potential: synthesis scalability and compatibility with 3′ modification methods. Regarding scalability, the hemoglobin alpha 5′ UTR used in the screening in Example 1 required two rounds of mRNA ligation at high oligo: mRNA (5′-phosphorylated RNA oligonucleotide: RNA precursor) ratios (>200) to achieve complete labeling (FIGS. 12A and 12B). To test whether this was due to a strong secondary structure of the hemoglobin alpha 5′UTR, a short unstructured space was introduced into the 5′ end of the mRNA (FIG. 12C). This adjustment effectively promoted complete ligation at low oligo: mRNA ratios, but also resulted in ~15 mRNA self-ligation, which had been previously thwarted by the structured 5′ UTRs (FIGS. 12D and 12E). To address these issues, an extra round of ligation prior to RppH hydrolysis was tested to introduce a nuclease-resistant PS linkage and a terminal 2,3-dideoxyC (ddC) at the 3′ end of the mRNA (RNA precursor), which slowed mRNA deadenylation and prevented mRNA self-ligation. Testing of different modifications to confer 3′ end stabilization showed that a combination of PS-2′MOE was the most beneficial (FIG. 13E). Subsequently, the triphosphate was hydrolyzed, and the modified capped oligonucleotide was ligated to the 5′ end of the mRNA precursor to generate 3′/5′-modified mRNA. This workflow was named Ligation Enabled messenGerRNA-Oligonucleotide assembly (LEGO), which allows for scalable synthesis of mRNA extensively modified on both its 5′ and 3′ ends in 5 hours with a >50% recovery rate (FIG. 13). Using LEGO, a modular set of chemical modifications and topological structures in the cap, 5′ UTR region, and 3′ poly-A tail regions were integrated into one single mRNA transcript, which, in combination, boosted protein expression by 5.5~99-fold from 8~72 hours compared to a conventional linear mRNA containing only a m7G-rG cap and m¹Ψ (FIG. 9I).

### Oligonucleotide Conjugation Using CuAAC

Azide/alkyne-labeled oligonucleotides at a final concentration of ~200 μM were mixed with modified 1.5× click chemistry buffer ([Lumiprobe, 61150] containing additive 5% SUPERase Inhibitor, 5% DMSO, and 5% 10 mM dNTP mix [ThermoFisher Scientific, 18427089]) that was briefly degassed by argon purging for 20 mins prior to the reaction. For a typical 100 μL reaction, 33 μL of oligonucleotide solution was mixed with 66 μL of click chemistry buffer and 4 μL of 100 mM freshly prepared ascorbic acid solution [Sigma Aldrich, A5960] was added immediately prior to the reaction. The mixture was incubated at 37° C. for 1 hr and quenched by addition of 1 μL of 500 mM EDTA (pH 8.0). The reaction was first purified using Monarch RNA Cleanup Kit [NEB, T2040] and then subjected to RNase-free HPLC purification using method 2.

### Example 4: 5′ Modifications Enhance mRNA Translation Via Enhanced Binding to Initiation Factors and Resistance to Decapping Enzymes

To unbiasedly screen for protein binders recognizing synthetic 5′ modifications, Applicant performed pull-down analysis using SILAC (stable isotope labeling by amino acids in cell culture). HEK293T cytoplasmic lysates were incubated with magnetic streptavidin beads coated by 20 nt 5′-capped/3′-biotinylated synthetic oligonucleotides to pull down RNA binding proteins (RBPs) that recognize the cap-proximal modifications. The synthetic oligo substrates contained either a canonical mono-m7G-rG cap, or the optimized combination of 5′ chemical modifications, where the modification-specific protein enrichment was measured by isotope ratio on quantitative mass spectrometry (qMS) (FIG. 14A). Satisfactorily, compared to the Cap-0 oligo, optimized 5′ modifications led to dramatic enrichment of major proteins involved in the eIF4-eIF3 dependent translation initiation pathway including eIF4E (15.2-fold enrichment), eIF4G1 (42.1-fold enrichment), eIF4G3 (27.9-fold enrichment), eIF3A (5.8-fold enrichment), and eIF4A1 (2.5-fold enrichment), except for eIF2B (5.1-fold repulsion), potentially due to the oligo length and eIF2B's relative downstream position during translation initiation (FIG. 14B). Interestingly, Applicant also observe significant enrichment of proteins that interact peripherally with the eIF4 complex such as the poly(A)-binding proteins (PABPC1, 5.5-fold enrichment; PABPC4, 5.5-fold enrichment) and ribosomal proteins (up to 2.9-fold enrichment), indicating that the cap/5′ UTR modifications not only enhanced interactions of direct cap binders but also stabilized the overall eIF4-mRNA complex to interact with the tail and recruit ribosomal subunits. As predicted by Ingenuity Pathway Analysis (IPA) of the enrichment protein list in the optimized 5′ modification group, the stabilized binding of eIF complexes via 5′ RNA modifications leads to upregulation of translation initiation (FIG. 14C), supporting Applicant's experimental observation of enhanced translation efficiency for modified RNA.

Accordingly, Applicant further investigated the effects of a selected pool of cap/5′ UTR modifications on eIF4E binding. Using fluorophore-labeled capped oligos, Applicant quantified their affinity to eIF4E through electrophoretic mobility shift assay (EMSA) (FIGS. 14D and 9A-9F). Consistent with prior expression tests, the incorporation of LNA on both the m⁷G and the first base significantly increased eIF4E binding affinity, lowering the dissociation constant (K_{d}) from 2,019±96 nM to 1,425±126 nM (FIGS. 9B and 9D). However, the 6×2′OMe 5′ UTR modification reduced eIF4E affinity to 2,446±140 nM despite significantly enhancing protein expression in cell culture (FIG. 9C), and its K_{d} was rescued by the addition of the LNAm⁷G cap (1,570±116 nM) (FIG. 9E).

In addition to enrichment of initiation factors during pull-down analysis, Applicant also observed increased levels of proteins involved in nonsense mediated decay such as XRN2 (10.2-fold enrichment), PARN (8.0-fold enrichment), and CASC3 (5.4-fold enrichment) in agreement of the general understanding that activated translation is concurrent with upregulated cotranslational decay. To clarify whether the cap/5′ UTR modifications increased resistance to degradation machinery, as opposed to simply affinity, Applicant evaluated the effects of cap/5′ UTR modifications on resistance against human decapping enzyme 2 (hDcp2), the primary decapping enzyme responsible for the first irreversible step in 5′-to-3′ mRNA decay prior to exoribonuclease (XRN) digestion. Capped 23-nt oligos with various modification patterns were subjected to hDcp2 decapping and analyzed at different time intervals (FIGS. 14E and 9I). LNA modifications on both the cap and +1 nucleotide dramatically increased stability against hDcp2 decapping, with 71%~73% of capped transcripts remaining intact after 60 mins, compared to complete decapping of the unmodified transcript within just 15 mins. Conversely, the introduction of 2′OMe at the +1 position slightly increased susceptibility towards decapping. However, additional 2′OMe incorporation at first 6 bases or the entire oligo increased decapping resistance to a similar level as LNA modification, which may be relevant to a site-dependent effect of cap-1/cap-2 methylation in marking “aged” mRNA for turnover. Collectively, both increased affinity for eIF4E and resistance against hDcp2 decapping contributed to the superior performance of LNA-modified oligos in cell-based expression tests. Other significantly enriched proteins for the modified oligos include proteins involved in ribosomal quality control process (PRRC2A and PRRC2C, USP10, ATXN2), or other secondary binders of eIFs and ribosomal proteins (MKRN2). Among the repelled proteins, the nuclear cap-binding protein responsible for capped mRNA nuclear export NCBP1 was repelled by 32.8-fold, indicating that the cap/5′-UTR modifications selectively enhanced binding by eIFs but not other cap-binding proteins unrelated to translation initiation. Another dramatically repelled protein was DHX15 (15.1-fold repulsion), a reported coreceptor involved in RIG-I-like receptor (RLR) sensing for antiviral response, indicating the the optimized 5′-modifications are not immunogenic. Indeed, cellular transfection of the modified mRNA and subsequent qPCR analysis of Mx1 and ISG15 revealed that both LNA and 2′OMe modifications led to negligible immunogenicity when introduced onto m1-modified mRNA compared to the poly(I: C) positive control, suggesting their suitability for therapeutic applications (FIG. 9J).

### Example 5: Multi-Capping Enhances Translation Efficiency Through eIF4E

To gain insights into the mechanism of how the topologically branched internal cap initiates mRNA translation, Applicant used fluorophore-labeled mono- or dual-capped oligonucleotides and measured their affinity to eIF4E through EMSA. A dual-capped oligo effectively enhanced eIF4E affinity, reducing K_{d} value from 2,214±139 nM to 1,414±135 nM (FIGS. 14D, 9F, and 9G), and such enhanced binding affinity was further confirmed by competitive EMSA experiment with equal stoichiometry of one-capped/dual-capped oligos against varying amounts of eIF4E (FIG. 9H). Incorporation of both caps as LNAm7G further lowered the K_{d} to 1,276±43 nM (FIG. 14D).

Applicant then performed in situ RNA profiling experiments to confirm whether dual-capping indeed enhanced translation efficiency on mRNA. Utilizing paired STARmap and RIBOmap, which detected total mRNA and ribosome-bound mRNA at single-cell level, respectively, Applicant approximated the translation efficiency (TE) using the ratio of the two measurements. Both values were normalized against the internal transfection control of Renilla luciferase (RLuc) to estimate TE values (FIG. 14F-G). Such in situ profiling techniques also allowed filtration of large copies of non-translating mRNA trapped in lipofectamine transfection vesicles within endosomes to more accurately capture the status of translating cytosolic mRNAs. As expected, while total mRNA level was similar for both constructs, ribosome-bound mRNA levels were significantly higher for the dual-capped transcripts over the entire cell population, yielding ~1.8-fold higher TE (FIG. 14H-J).

Mechanistically, both eIF4E and eIF3D have been reported as cap-binding proteins in translation initiation. eIF4E is responsible for canonical cap-dependent translation initiation, whereas eIF3D initiates translation through a non-canonical pathway via directly recruiting eIF3. To rule out the possibility of eIF3D engagement in dual-capped mRNA translation, Applicant tested one/two-capped synthetic mRNA in Hela cells after eIF3D knockdown (KD). The translation of both dual-capped Fluc mRNA and co-transfected mono-capped Rluc mRNA was significantly reduced, but the extent of reduction was similar, indicating a substantial but comparable role of eIF3D in both cases as an essential core of the eIF3 complex downstream of eIF4 binding (FIG. 9K-9M). Furthermore, Applicant tested the interaction of dual-capped mRNA with eIFs using the rabbit reticulocyte lysate (RRL) in vitro translation system with the presence of 4EGI-1, an inhibitor of translation initiation by blocking eIF4E and eIF4G interaction. Translation of both the dual-capped and mono-capped mRNA was significantly reduced, supporting the necessity of eIF4E/4G interactions in translation of dual-capped mRNA (FIG. 9N-9P). Together, Applicant concluded that dual-capped mRNA exhibited increased translatability mainly through enhanced canonical cap-binding activities of eIF4E without specific engagement of eIF3D cap-binding.

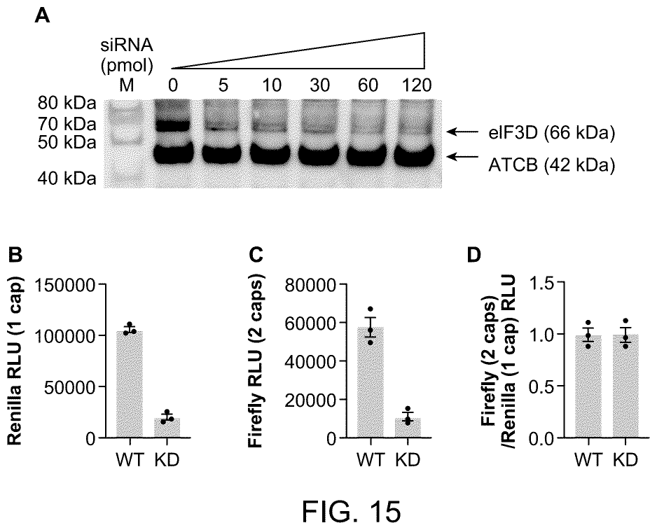

To verify the engagement of eIF3D, its expression was knocked down in HeLa cells using an siRNA cocktail (FIG. 15A). Translation of both dual-capped Fluc mRNA and co-transfected Rluc mRNA was significantly reduced, but the extent of reduction was similar, indicating a substantial but comparable level of eIF3D in both cases (FIGS. 15B-15C and 14K. Lastly, dual-capped mRNA was tested in a rabbit reticulocyte lysate (RRL) in vitro translation system, along with the addition of 4EGI-1, an inhibitor of translation initiation through blocking the interaction between eIF4E and eIF4G. The introduction of 600 μM 4EGI-1 coupled with a reduction of RRL % to 30% almost completely halted translation of both mono- and dual-capped mRNA (FIG. 14L). Noticeably, while reducing RRL % to 30% alone ablated translation of mono-capped mRNA, dual-capped mRNA was still observably translated, indicating its ability to translate even at lower eIF concentration. Taken together, it was concluded that dual-capped mRNA exhibited increased translatability mainly through enhanced engagement of eIF4E.

mRNA Profiling in Transfected Cell Culture with STARmap/RIBOmap

Modified Fluc mRNA and unmodified Rluc mRNA (transfection control) were cotransfected to HeLa cells seeded in 24-well plates. After a 6-hr incubation, the transfection mixture was removed, and cells were rinsed with 1×DPBS and trypsinized to reseed into two glass-bottom 96-well plates ([MatTek, PBK96G-1.5-5-F] poly-D-lysine [Sigma-Aldrich, A-003-M] coated). Each well of transfected cells was reseeded equally into two 96-well plates as technical replicates with one measured by STARmap (Firefly)+STARmap (Renilla) and the other measured by RIBOmap (Firefly)+STARmap (Renilla) using previously published procedures (Wang et al. (2018); Zeng et al. (2022)). Briefly, 24 hours post-transfection, culture media was removed and cells were washed with 150 uL PBS. Cells were then fixed with 1.6% PFA [Electron Microscope Sciences, 15710-S]/PBS [Gibco, 10010-023] at room temperature for 15 min and permeabilized with pre-chilled methanol at −20° C. for one hour. Methanol was then removed, and the cells were rehydrated with PBSTR/Glycine/YtRNA (PBS with 0.1% Tween-20 [TEKNOVA INC, 100216-360], 0.5% SUPERaseIn [Invitrogen, AM2696], 100 mM glycine, 1% yeast tRNA) at RT for 5 min followed by one PBSTR wash. Samples were then hybridized with SNAIL probes targeting Firefly and Renilla luciferase mRNA sequences in the hybridization buffer (2×SSC [Sigma-Aldrich, S6639], 10% formamide [Calbiochem, 655206], 1% Tween-20, 20 mM RVC [ribonucleoside vanadyl complex, NEB, S1402S], 0.5% SUPERaseIn, and 1% yeast tRNA, 100 nM each probe) in a 40° C. humidified oven with shaking and parafilm wrapping overnight. For RIBOmap quantification, SNAIL probes targeting Firefly luciferase were replaced with RIBOmap primer/padlock probes and splint probes targeting 18S rRNA, with the rest of the protocols remaining the same. After hybridization, the cells were then washed with PBSTR twice at 37° C. (20 min each wash) and high salt wash buffer (PBSTR with 4×SSC) once at 37° C. before rinsing once with PBSTR at room temperature. A ligation reaction was performed for 2 hours at room temperature to circularize padlock probes that were adjacent to the primer in ligation buffer (1×T4 DNA Ligase buffer, T4 DNA ligase, 0.1 Weiss U/μL [ThermoFisher Scientific, EL0012], 0.5 mg/mL Ultrapure BSA [ThermoFisher Scientific, AM2618], 0.5% SUPERaseIn). Cells were washed twice with PBSTR, and rolling circle amplification (RCA) was carried out in RCA buffer (1×Phi29 buffer, 0.2 Weiss U/μL Phi29 DNA polymerase [ThermoFisher Scientific, EP0094], 250 μM dNTP mix [ThermoFisher Scientific, 18427089], 0.5 mg/mL BSA, 0.5% SUPERaseIn) at 30° C. for 2 hours, followed by two washes with PBST. Samples were then stained with fluorescent detection oligo in the wash and imaging buffer (2×SSC, 10% formamide, 100 nM per detection probe, 1×DAPI [Molecular Probes, D1306]) at room temperature for 1 h. Confocal imaging stacks were taken with a Leica Stellaris 8 with a 60× oil objective at a pixel size of 283×283 nm. A 14-μm stack is imaged with 2 μm/step×7 steps. Three independent transfections were conducted for each condition, of which at least four representative FOVs were taken. The same imaging setting was used for all the samples to be compared. Excitation/detection wavelengths are as follows: DAPI, Diode 405 nm/~ [420-489] nm; Alexa546, WLL 557 nm/~ [569-612] nm; Alexa647, WLL 653 nm/~ [668-738] nm. MATLAB 2021a and CellProfiler 4.0.7 were used for the amplicon count-based fluorescence image analysis. First, the centroids of amplicons in each fluorescent channel (Firefly, Renilla) were identified by finding extended maxima on images. Then a 3*3*3 voxel volume centering the centroid of each fluorescent dot was defined. Within each voxel volume, the integrated intensities in the Firefly and Renilla channels were calculated, and the ratio between Firefly intensity and Renilla intensity was used for amplicon classification. For STARmap quantification, all the measurements were pooled together and the distribution of log (Firefly/Renilla) values were plotted. The corresponding ratio values at the nadirs (local minimum) on the distribution plot were identified as cutoff values. The first cutoff value smaller than 0 was noted as cutoff1, and the first cutoff value greater than 0 was noted as cutoff2. For RIBOmap quantification, cutoff1 was assigned as −0.3 and cutoff2 was assigned as 0.3. Any amplicon with a log (Firefly/Renilla) value smaller than cutoff1 was identified as a Renilla amplicon. Any amplicon with a log (Firefly/Renilla) value larger than cutoff2 was identified as a Firefly amplicon. Any amplicon with a log (Firefly/Renilla) value between cutoff1 and cutoff2 was identified as a granule. Amplicon classification information, as well as the location of every amplicon, was saved in a file. In each figure, the ratio between the number of Firefly amplicons and the number of Renilla amplicons were calculated and used to reflect the amount of Firefly luciferase mRNAs. Cell segmentation was performed by first identifying DAPI stained nuclei as primary objects and all channels were merged and subsequently converted to a grayscale image as secondary objects, and the segmentation masks were saved as uint16 images. Applicant then assigned amplicons to cells according to where they were located on the masks. The ratio between the number of Firefly amplicons and the number of Renilla amplicons in each cell was calculated and used to reflect the amount of Firefly luciferase mRNAs in a single cell.

### In Vitro Translation Assay

Firefly luciferase reporter mRNA with one cap and Nanoluc mRNA with two caps were synthesized according to protocol mentioned above. The in vitro translation was conducted using Rabbit Reticulocyte Lysate (Nuclease-treated) [Promega, L4960] according to manufacturer's protocol with some modifications. 30% or 40% of rabbit reticulocyte lysate, 4% of SUPERaseIn RNase Inhibitor, amino acid mixture and indicated mRNA were added to the reaction with 4EGI-1 inhibitor (600 μM) or DMSO as control in 50 μL reaction. The reactions were incubated at 30° C. for an hour. Then, 5 L of the reaction mixture was diluted by 10-fold with PBS. The fluorescence of firefly luciferase was measured with Dual-Glo Luciferase Assay System [Promega, E2920] and the fluorescence of Nanoluc was measured with Nano-Glo Luciferase Assay System [Promega, N1110] according to manufacturer's protocol.

eIF3d Knock-Down

4 sets of siRNA targeting human eIF3d were designed and ordered from Qiagen and suspended as 10 μM stock and mixed. siRNAs were transfected into HeLa cells in a 6-well plate using RNAiMAX lipofectamine kit [Invitrogen, 13778100]. 2 days after transfection, half of the cells were reseeded to 96-well plates for dual-luciferase activity assay and the other half were subjected to western blot for eIF3d KD quantification. Briefly, the cells were washed with ice-cold PBS, then 800 μL/well lysis buffer (RIPA buffer [Sigma Aldrich, R0278-50ML]+protease inhibitor cocktail [Sigma Aldrich, P8340-1ML]). The mixture was transferred to a tube, and agitated at 4° C. for 30 min. The mixture was then centrifuged at 4° C. for 20 mins at 12000 xg and protein concentrations in the supernatant were quantified with Qubit BR protein kit [Invitrogen, A50668]. 10 μg of total protein was loaded for each lane on NuPage 4-12% gel and run at 100 V for 2 hrs, after which was transferred to membrane (10V, 1.0A, 30 mins) [Trans-Blot Turbo, 1704159] and blocked at room temperature for 1 hr. The membrane was then incubated with anti-ACTB-HRP [Biolegend, 664803] and anti-eIF3D-Rabbit IgG [ProteinTech, 10219-1-Ab] in the blocking buffer at 4° C. overnight. The membrane was washed 3 times with PBST, and incubated with anti-rabbit IgG-HRP [Cell signaling, 7074P2] in the blocking buffer for 1 hr at room temperature. The membrane was washed 3 times with TBS and signals were developed with SuperSignal West Dura [Thermo Scientific, XC340766].

### Example 6: Branched Capping Drives Robust Translation of Circular RNA

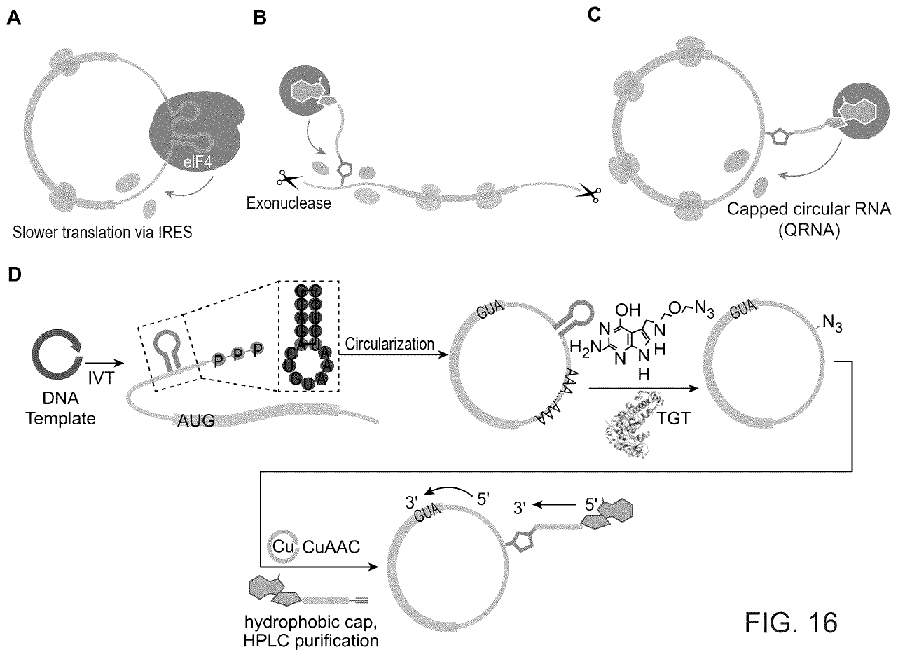

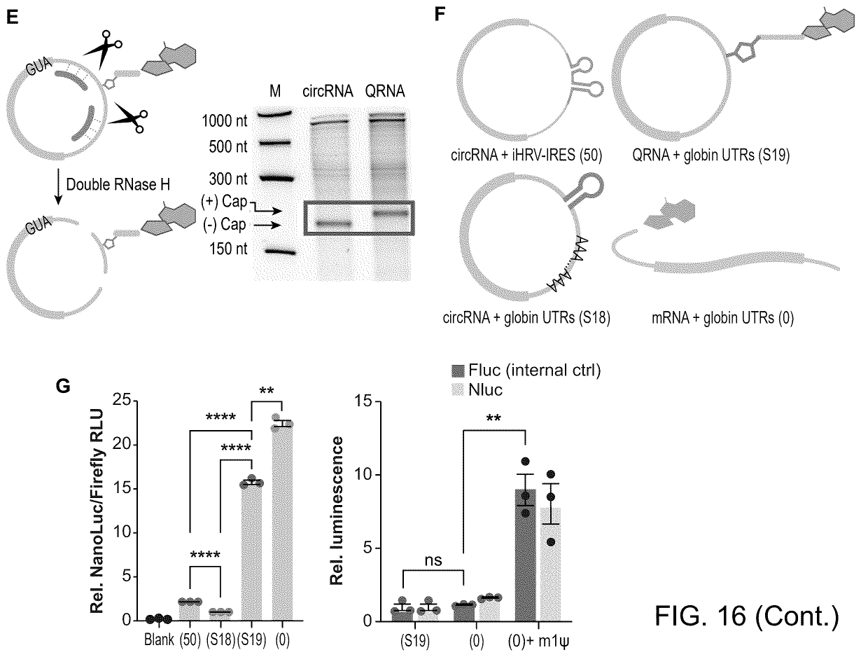

Because branched cap structures enhanced translation on linear transcripts, this approach was implemented to drive translation of circular RNA molecules (circRNAs). Conventionally, circRNAs lack a cap and poly-A tail and require an internal ribosome entry site (IRES) for translation initiation (FIG. 16A). However, the initiation rate via IRES is known to be slower than the canonical cap-dependent mechanism (Koch et al. “IRES and cap translation with single-molecule resolution in live cells.” Nat. Struct. Mol. Biol. 27, 1095-1104 (2020)). While the branched cap could not inhibit the exonuclease degradation of the uncapped mRNA “stem” (FIG. 10D), it has been suggested that circRNAs may possess enhanced exonuclease resistance and stability in vivo (Wesselhoeft et al. “Engineering circular RNA for potent and stable translation in eukaryotic cells.” Nat. Commun. 9, 2629 (2018); Chen et al. “Engineering circular RNA for enhanced protein production.” Nat. Biotechnol. (2022), doi: 10.1038/s41587-022-01393-0). Consequently, it was tested whether a branched capping strategy could simultaneously maintain high stability of circRNA while harnessing the cap-dependent translation initiation mechanism to enhance its translatability (FIGS. 16B and 16C). The capped-circular mRNA was named “QRNA” due to its resemblance to the letter “Q”.

In terms of QRNA synthesis, achieving simultaneous RNA circularization and incorporation of a site-specific click chemistry handle posed a major challenge. As proof-of-concept, a minimal RNA encoding HiBiT was synthesized, and its sequence engineered to include only a single cytosine in its 5′ UTR. This allowed for the introduction of an azide handle by substituting CTP in the 5′ UTR with azide-labeled CTP (5-Azido-PEG4-CTP) during IVT. This minimal mRNA was then circularized with T4 RNA ligase using homology regions in the 5′ and 3′ UTRs, yielding an azide-labeled circRNA. The product was then purified through HPLC and its circular shape confirmed by RNase R resistance. A m7G-capped, OU-labeled oligonucleotide was then conjugated to azide-circRNA to generate a minimal QRNA (FIGS. 11D and 11E, construct S17). Surprisingly, QRNA exhibited significantly enhanced translation compared to its circRNA precursor (FIG. 11F).

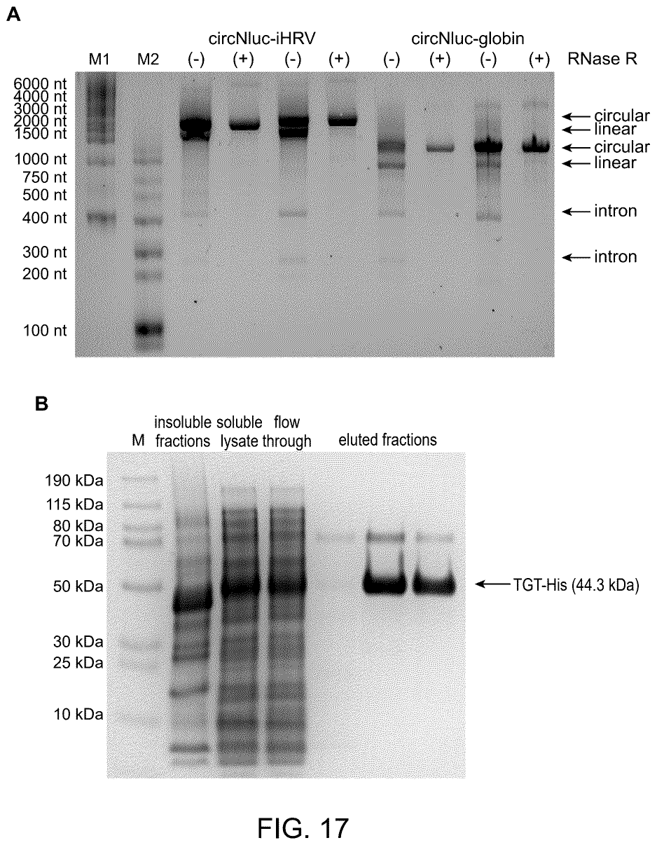

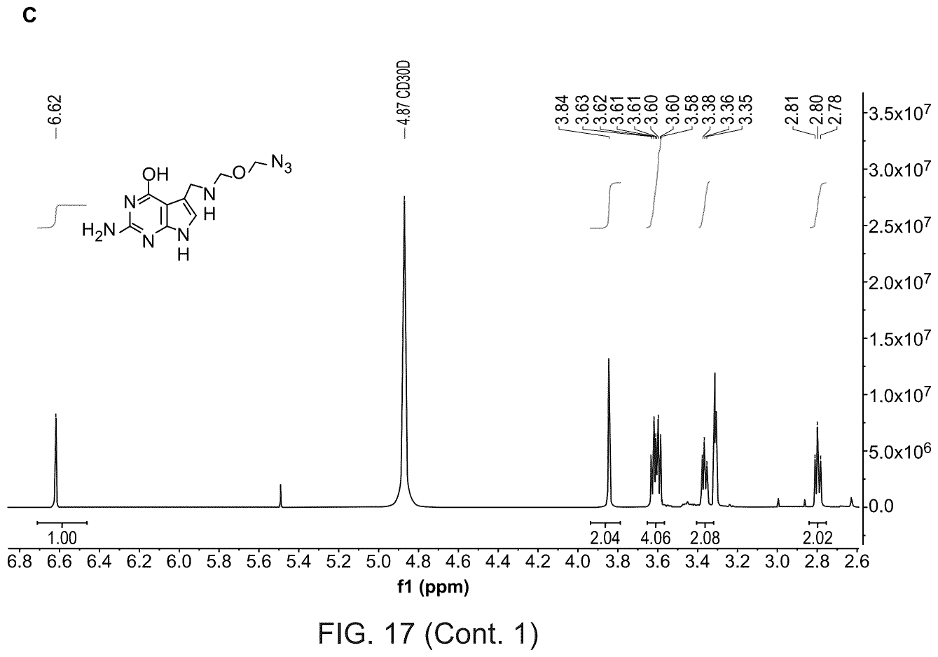

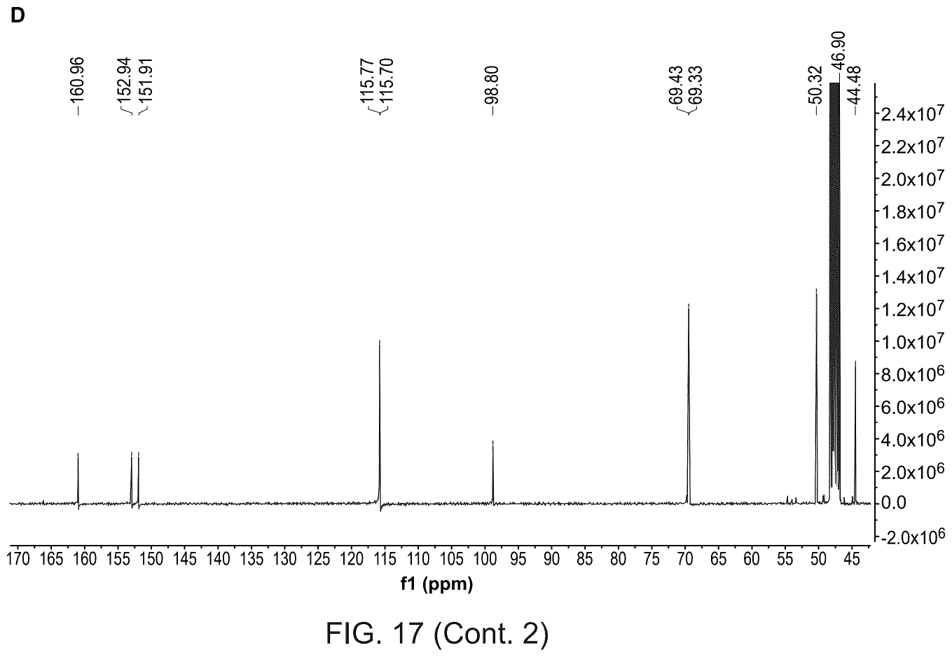

In order to make QRNA synthesis applicable to longer transcripts that contain more than one cytosine, an alternative workflow was developed. Specifically, mRNA encoding Nano luciferase (Nluc) was synthesized and circularized through intron back-splicing (FIG. 17A). For the incorporation of click-chemistry handles, a minimal hairpin sequence was introduced upstream of the coding sequence (CDS) capable of site-specific recognition and labeling by tRNA guanine transglycosylase (TGT) using pre-queuosine 1 (preQ1) (FIG. 17B). Utilizing TGT and synthetic preQ1-azide, a single azide handle was introduced onto the circRNA and clicked with capped-OU oligonucleotide (FIG. 16D). To purify QRNA from unclicked circRNA precursors, a hydrophobic Bn7G cap analog was used to increased QRNA retention on RP-HPLC, and the purified product was characterized by a double RNase H assay (FIG. 1E). QRNA translated much more efficiently than its circRNA counterpart (with and without IRES), though less efficiently than m7G-capped linear mRNA (FIG. 16F). Furthermore, as synthesis of the circRNA precursor relies on ribozyme splicing, which is incompatible with m1Y, QRNA was only compatible with uridine, while the expression of linear mRNA was significantly boosted after 100% m1Y′ replacement. Such discrepancy was not due to the inherent translatability of the QRNA construct, but rather the toxicity of pure uridine-containing RNAs, which was evidenced by the significant reduction in the expression of control Fluc mRNA when co-transfected with uridine-containing linear or QRNA Nanoluc reporter (FIG. 16G). Nonetheless, QRNA holds promise as a therapeutic platform based on its high translatability and stability.

Circular mRNA Synthesis and Characterization

DNA templates were cloned as described above, PCR amplified, and gel purified to be used as IVT templates. CircRNAs were synthesized using the HiScribe T7 High Yield RNA Synthesis Kit [NEB, E2040S]. Post IVT, DNA templates were digested with Turbo DNase [ThermoFisher, AM2238]. The reaction mixture was heated to 70° C. for 5 min and then immediately cooled on ice for 3 min, after which GTP was added to a final concentration of 2 mM, and the reaction mixture was incubated at 55° C. for 15 min. CircRNA was enriched by treatment with RNase R [Lucigen Corporation, RNR07250] for 1.5 hr, and the products were column purified. CircRNA products were characterized by gel electrophoresis.

Synthesis of preQ1-Azide

100 mg of preQ1-HCl [Princeton Bio, PBMR232165-0.5 g] was suspended in 0.5 mL anhydrous DMF, to which 179 μL DBU was added dropwise at 0° C. which allowed the salt to dissolve, and 85 mg of bromo-PEG1-azide [Broadpharm, BP-24132] was then added. The reaction was allowed to stir overnight under argon protection. Solvents were removed in vacuo, and preQ1-azide was isolated by flash chromatography as a light brown solid (1:1:0.1 DCM/MeOH/NH₃). LC-MS (ESI−): calculated 291.30, found 291.26. 1H NMR (400 MHz, CD3OD) δ 6.62 (s, 1H), 3.84 (s, 2H), 3.61 (m, 2H), 3.36 (m, 2H), 2.80 (m, 2H). 13C NMR (126 MHz, CD3OD) δ 160.91, 152.94, 151.91, 115.77, 115.70, 98.80, 69.43, 69.33, 50.32, 45.90, 44.48.

### QRNA Synthesis and Characterization

CircRNA bearing a TGT hairpin was synthesized as described above. TGT enzyme was expressed in E. Coli as previously described in Alexander et al. “Site-specific covalent labeling of RNA by enzymatic transglycosylation.” J. Am. Chem. Soc. 137, 12756-12759 (2015). To label the circRNA with preQ1-azide, 1 μM of circRNA, 100 UM of preQ1-azide, 10 μM of TGT, 10 μL of SUPERase-In RNase inhibitor were incubated in 1× TGT reaction buffer (100 mM HEPES, pH 7.3, 5 mM DTT, and 20 mM MgCl₂) in a total of 100 μL reaction at 37° C. for 2 hours. The labeled circRNA was purified and subjected to click reaction with Bn7G-capped alkyne labeled oligo using the general condition for click reaction for 30 mins. The circRNA was then subjected to RP-HPLC purification to remove the linearized portions (method 3), pooled and desalted, and subjected to another round of RP-HPLC purification to isolate the QRNA product (method 4). QRNA product was characterized by RNase H assay with 2 primers upstream/downstream the TGT site.

### Example 7: Modified Multi-Capped mRNA Exhibited Enhanced Expression In Vivo

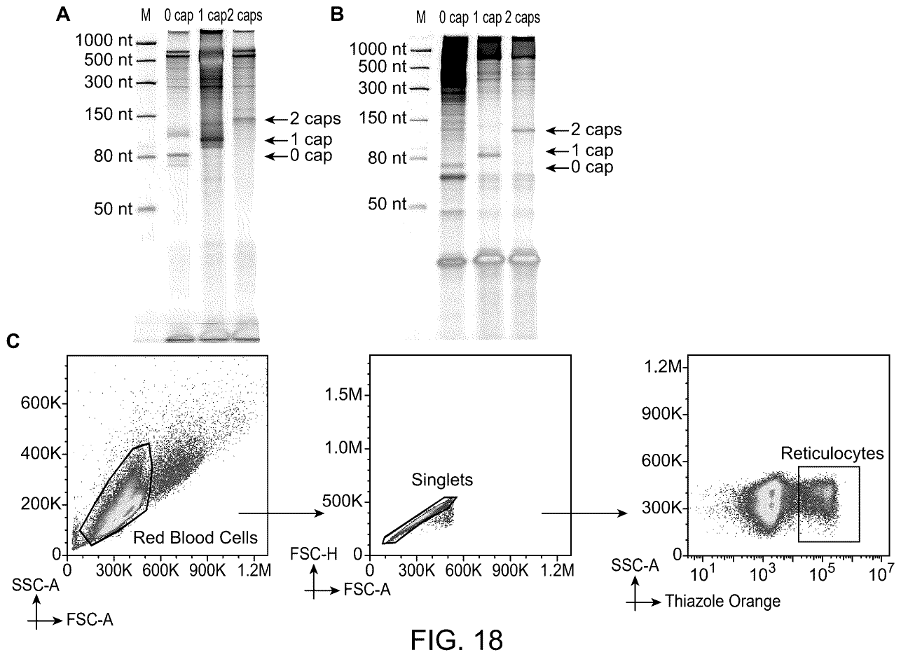

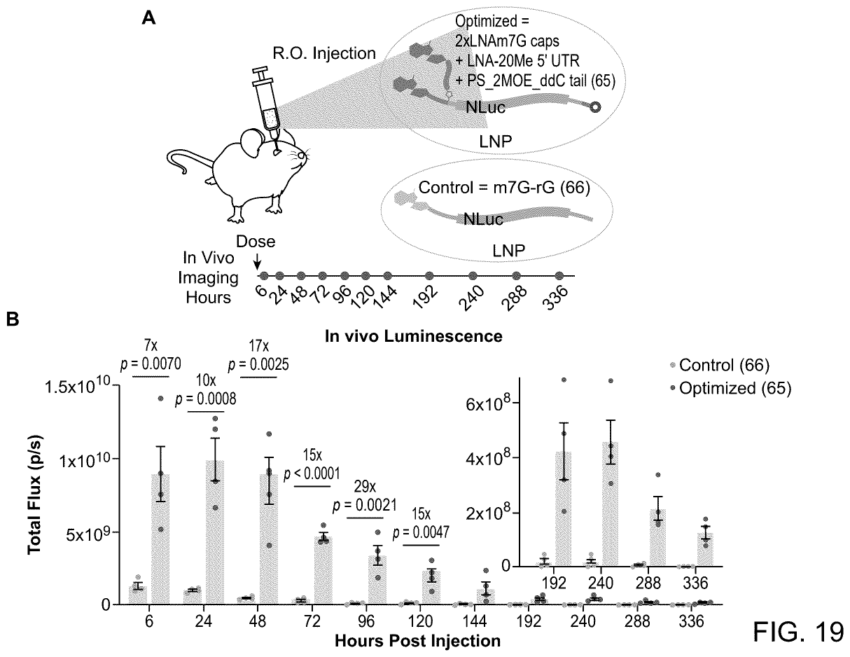

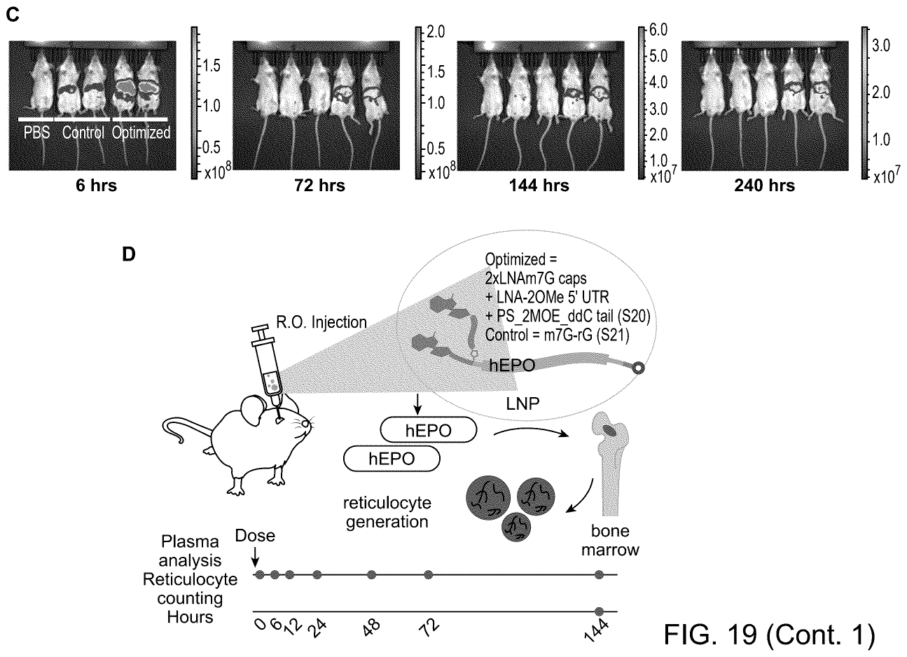

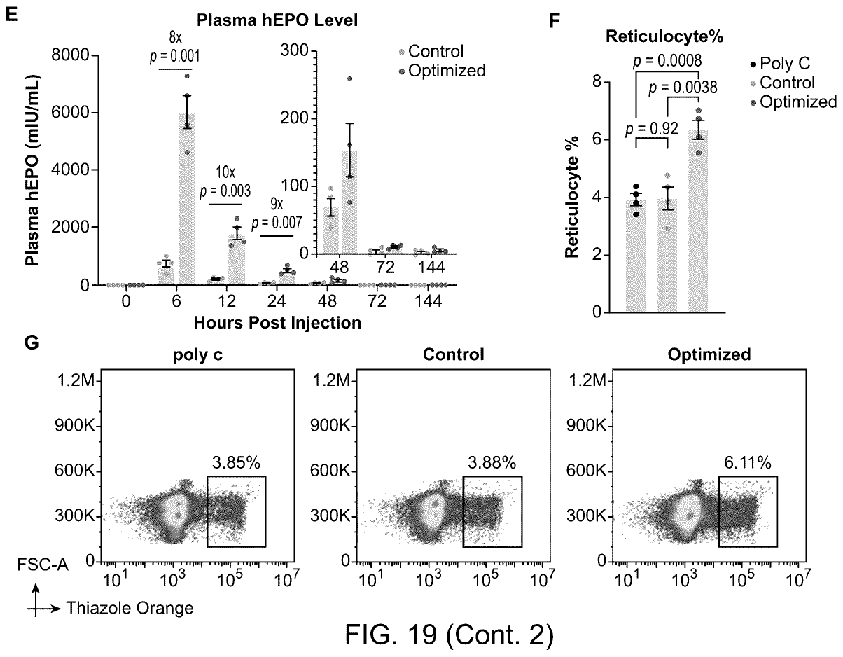

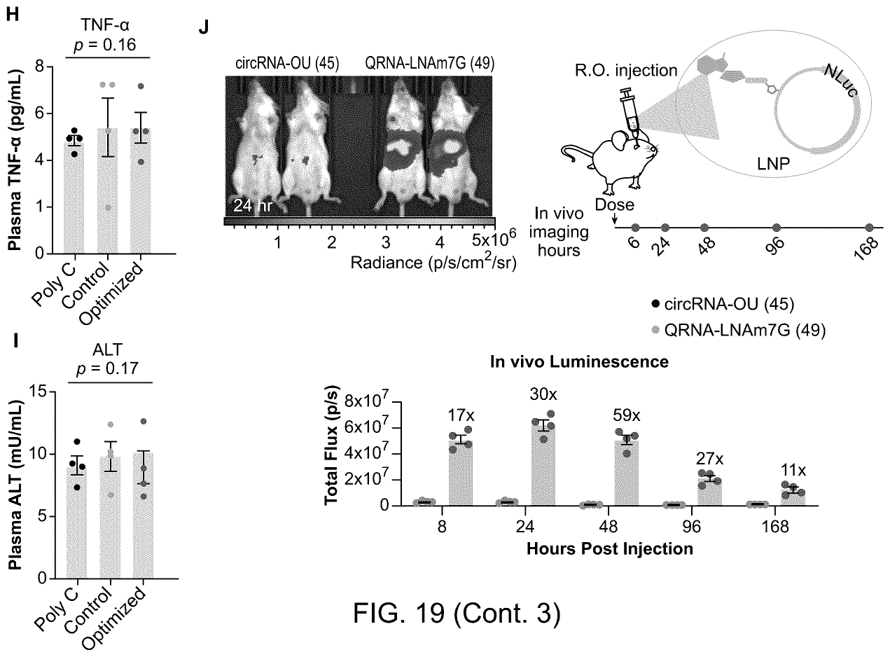

The modified mRNAs produced by the methods described herein were evaluated in vivo. Using LEGO, dual-capped mRNA encoding Nluc was synthesized that combined the most effective aforementioned translation-enhancing motifs: two LNAm7G caps, LNA-2OMe-modified 5′ UTR region, and PS-2MOE_ddC-modified poly-A tail. The translation of this mRNA was compared with mono-capped mRNA with an m7G-rG cap (FIG. 18A). Both constructs contained 100% replacement of uridine with m1ψ and were synthesized and purified using the same ligation-based workflow to ensure complete chemical capping. The mRNAs were then encapsulated in lipid nanoparticles (LNPs) using SM-102 based formulation and delivered to the liver of mice via retro-orbital (RO) injection. Mice were continuously imaged at the indicated time points following LNP administration (FIG. 19A). The bioluminescence from the mono-capped construct peaked at 6 hours (Flux=1.3±0.2×10⁹ p/s), but quickly declined to baseline level at 96 hours. Signals from the dual-capped mRNA peaked at 24 hours (Flux=1.0±0.1×10¹⁰ p/s), remained highly expressed until 144 hours (Flux=1.2±0.4×10⁹ p/s), and remained detectable for as long as 14 days post-injection, suggesting its enhanced and prolonged expression (FIGS. 19B and 13C).

To demonstrate the therapeutic potential of dual-capped mRNA, mono- and dual-capped mRNA molecules encoding human erythropoietin (hEPO) were synthesized. hEPO is a secreted peptide hormone used to treat anemia by stimulating red blood cell (RBC) production and hemoglobin biosynthesis (FIGS. 18B and 19D). hEPO functions similarly in mice and humans, although the therapeutic threshold is much higher in humans than in mice (Kariko et al., “Increased erythropoiesis in mice injected with submicrogram quantities of pseudouridine-containing mRNA encoding erythropoietin.” Mol. Ther. 20, 948-953 (2012)). Following RO administration of mRNA-LNP, peak plasma hEPO expression was observed at 6 hours in the dual-capped group (6009±578 mlU/mL) significantly outperforming the mono-capped group (733±123 mlU/mL), and the expression remained detectable until day 6 (FIG. 19E). On day 6 post-administration, plasma reticulocyte counts were evaluated to assess therapeutic outcomes. The dual-capped mRNA significantly increased reticulocyte counts by 64% compared to the poly(C)-LNP-treated group (3.9±0.2% to 6.4±0.2% of total red blood cells), while the effects of mono-capped mRNA were non-significant (4±0.3%) (FIGS. 19F, 19G, and 18C). Finally, the immunogenicity of the dual-capped mRNA was tested by measuring plasma tumor necrosis factor alpha (TNF-α) and toxicity measured by monitoring plasma alanine transaminase (ALT), as intravenous delivery readily targeted the liver. No significant changes were observed among all groups for either biomarker, indicating the suitability of dual-capped modified mRNAs for therapeutic applications (FIGS. 19H and 191).

Applicant then evaluated the QRNA design in vivo using the same NLuc-based assay. Applicant observed substantially higher expression (17×, 30×, 59× more translation at 8 hrs, 24 hrs, and 48 hrs respectively) of NLuc from m¹ψ substituted, LNAm7G capped QRNA at all time points, in comparison to the uncapped m¹ψ-circRNA precursor (circRNA-OU) (FIG. 19J). Applicant were able to recapitulate the extended half-life translation kinetics of circRNA in the QRNA construct, with translation peaking at 24 hours compared to a conventional m¹Ψ modified linear mRNA, which peaks at 6 hours in vivo (FIG. 19A). It is still worth noting that although the current iteration of QRNA underperformed the optimized dual-capped mRNA with 5′/3′-modifications, it still holds promise as a lead structure for further engineering in other dimensions such as sequence context and other UTR modifications. Applicant thus proceeded with the optimized dual-capped mRNA for subsequent therapeutic demonstrations beyond luciferase reporters.

### Mice

All animal procedures followed animal care guidelines approved by the Institutional Animal Care and Use Committee (IACUC) of the Broad Institute of MIT and Harvard under animal protocol #0255-08-19. Animal experiments were conducted in compliance with IACUC policies and NIH guidelines. The BALB/cj (male, 6~8 weeks old) mice used for in vivo NanoLuc assay in this study were purchased from The Jackson Laboratory (JAX). The C57BL/6 mice (female, 7 weeks old), used for hEPO assay in this study were purchased from The Jackson Laboratory (JAX).

In Vivo Delivery of mRNA

mRNA diluted in 50 mM citrate buffer (pH 4) and lipid mix (37.5 mM in ethanol) containing 50 mol % SM-102, 10 mol % DSPC, 38.5 mol % Cholesterol, and 1.5 mol % DMG-PEG2000 were assembled into LNP using a NanoAssemblr Spark instrument [Precision Nanosystems]. Upon formulation, mRNA-LNPs were diluted in 12 mL 1×PBS solution and the buffer was exchanged by concentrating with a 30 kDa spin filter [MilliporeSigma, UFC901008] to remove residue ethanol. Concentrations and encapsulation efficiency of mRNA were determined using Quant-it RiboGreen RNA Assay Kit [ThermoFisher, R11490]. 1 μg NanoLuc or hEPO mRNA-LNP in a total volume of 100 μL was injected through retro orbital injection into each mouse. Four mice were used for each condition of each experiment (NanoLuc imaging, hEPO ELISA, and reticulocyte counting). For NanoLuc imaging, PBS injection was used as negative control. For hEPO/reticulocyte experiments, poly(C)-LNP complex was used as negative control.

### In Vivo Imaging of Nanoluc

In vivo activity of NanoLuc was measured using a Competent IVIS-Perkin Elmer IVIS Spectrum CT System [Perkin Elmer]. At each time point, mice were anesthetized with isoflurane. The fluorofurimazine substrate with poloxamer-407 [Promega, CS320501] was freshly reconstituted in PBS to a final concentration of ~5.1 mM and injected intraperitoneally (i.p.) into the mice at a dose of ~22 nmol per gram body weight. Mice were imaged 5 mins post injection using auto exposure settings. The luminescent activity was quantified using Aura 4.0 imaging software. The background was determined by including mice injected with poly C-LNP negative control and subtracted from each measurement.

Quantification of Plasma hEPO

At the indicated time points, approximately 100 μL of blood was collected with EDTA-coated capillary tubes from each mouse and then transferred to an EDTA-coated tube. The collected blood samples were centrifuged at 2000 g for 10 min, followed by transferring the resulting plasma into another tube. The hEPO levels in the plasma samples were measured by Human Erythropoietin (EPO) ELISA kit [LEGEND MAX, 442907]. Final concentrations for hEPO were adjusted based on the volume and dilution fold.

### Plasma Reticulocyte Counting

10 μL plasma sample was added into 1 mL of the Reticulocyte Reagent [BD Bioscience] at the indicated time points and the mixture was incubated at room temperature for 30 minutes with protection from light. The samples were analyzed with Beckman CytoFLEX LX Flow Cytometer with 100,000-event records for each sample. Thiazole orange positive cells are defined as reticulocytes.

### In Vivo Toxicity Assay

To evaluate the in vivo toxicity and immune response, plasma collected on day 7 post mRNA-LNP administration for the hEPO group. ALT and TNF-alpha were measured using ELISA kits for ALT [G-Biosciences, IT5508] and TNF-alpha [R&D Systems, MTA00B] per manufacturer's protocols.

### Example 8: LEGO Enabled Site-Specific Chemical and Topological Modification of Circular RNA

The present Example illustrates a workflow for the synthesis of circular RNA with site-specific chemical and topological modifications.

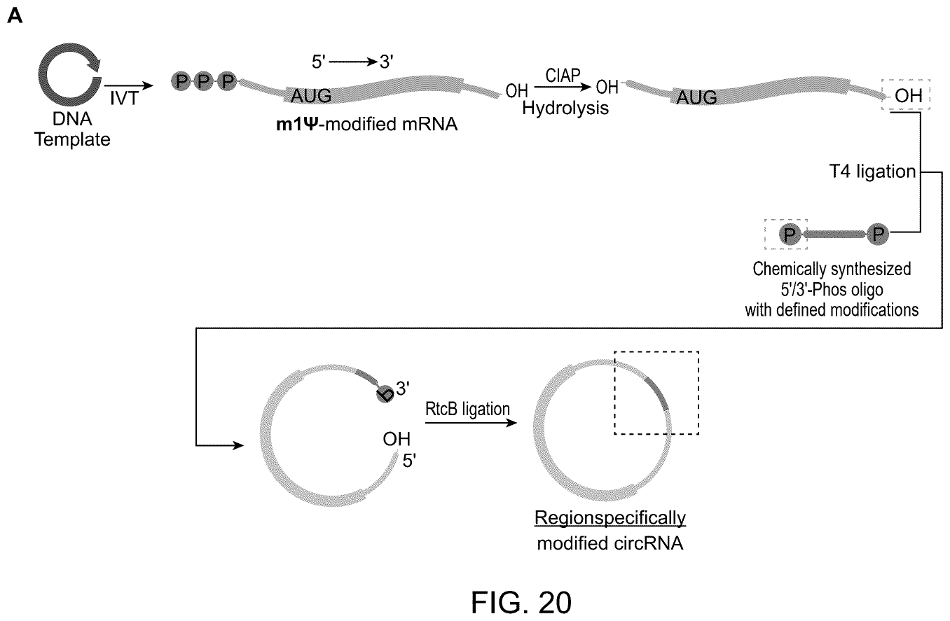

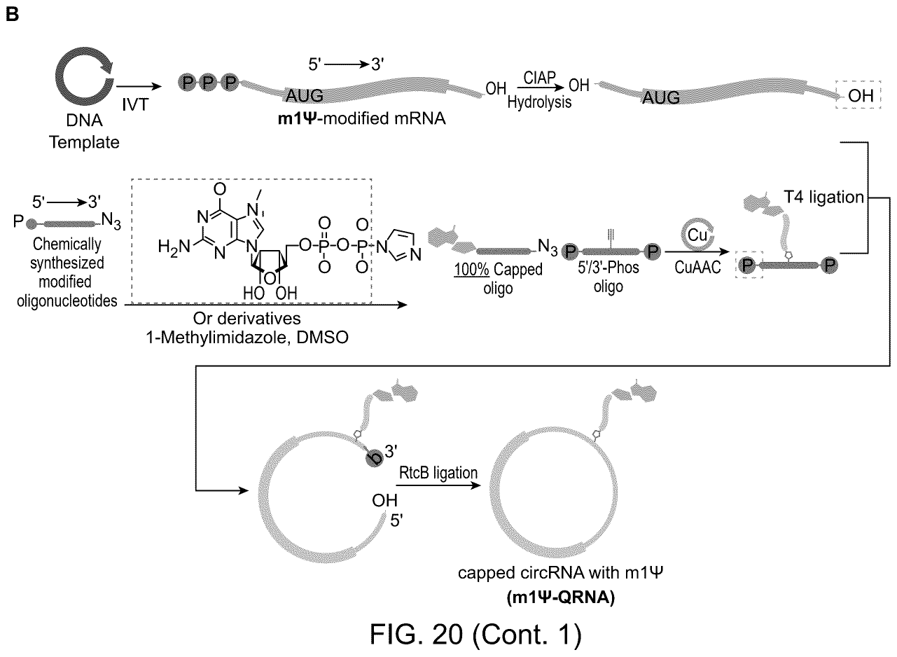

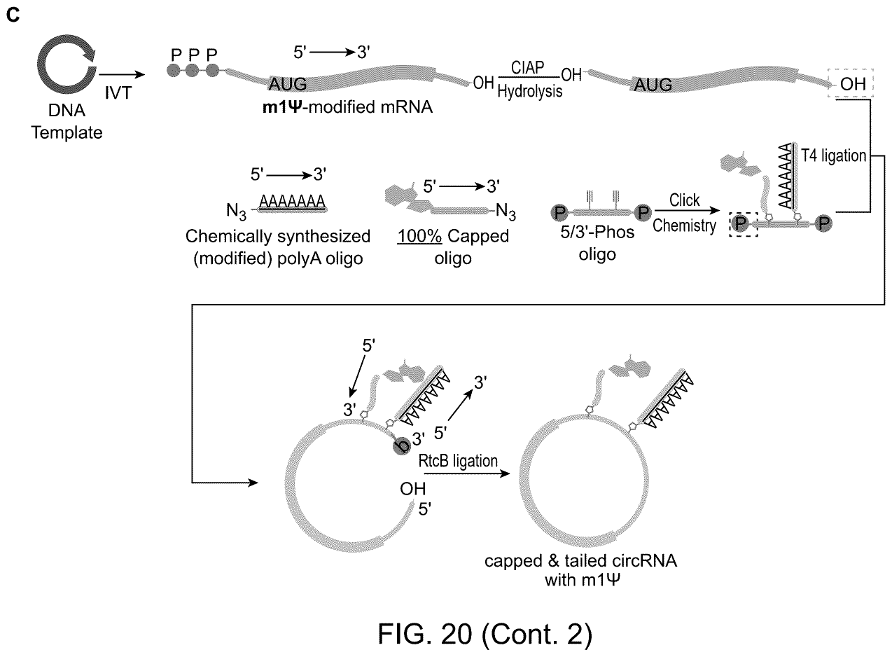

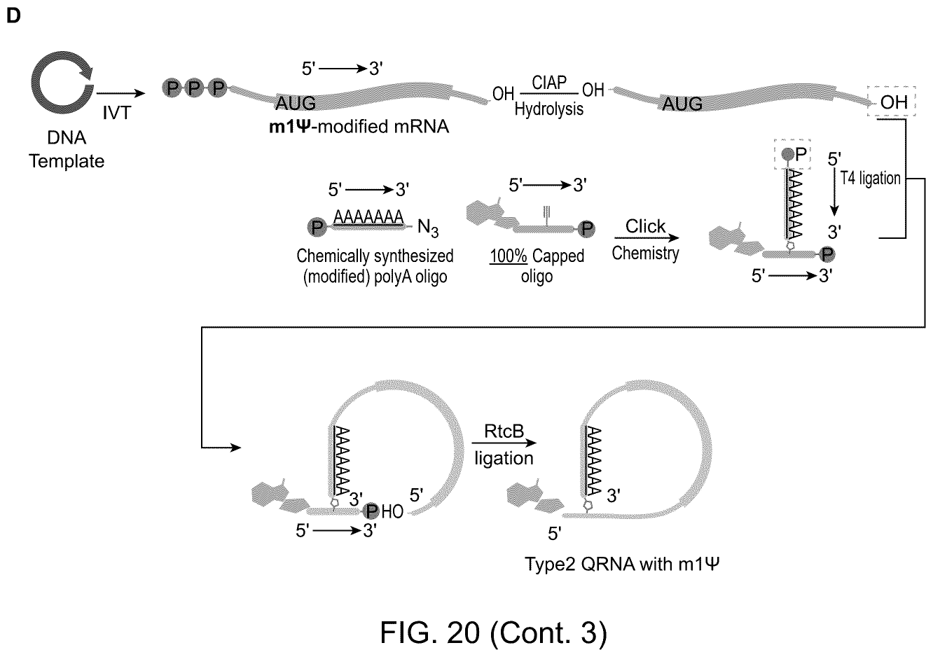

As shown in FIG. 20A, synthetic linear mRNA was prepared from a DNA template and IVT, allowing incorporation of either unmodified NTP or modified NTP such as m1Y′. The triphosphorylated mRNA after IVT was then treated with pyrophosphatases such as Calf Intestinal Alkaline Phosphatase (CIAP) to yield 5′/3′-hydroxyl mRNA. A chemically synthesized oligonucleotide with defined modification patterns and 5′/3′-phosphates was then ligated to the 5′/3′-hydroxyl mRNA by T4 RNA ligase, which ligates the 5′-phosphate on the oligo and 3′-hydroxyl of the mRNA. The ligation product was then further treated with RtcB ligase, which ligates the 5′-hydroxyl and 3′-phosphate ends to enable intramolecular circularization. As shown in FIG. 20B, the chemically synthesized oligo described in FIG. 20A could be further replaced by a branched cap oligo conjugated through click chemistry. The branched cap oligo was chemically synthesized and capped, and contained combinatorial patterns of chemical modifications including cap modifications, base modifications, phosphodiester linkage modifications, and sugar backbone modifications. Sequential T4 RNA ligase ligation and RtcB ligation yield modified capped circRNA (QRNA) compatible with modifications such as m1ψ.

As shown in FIG. 20C the branched capped oligo in FIG. 20B could be further replaced by a multi-branched oligo with both branched cap and branched poly(A) tails, where both the branched capped oligo and the branched poly(A) oligo are chemically synthesized and modified/unmodified. The branched poly(A) oligo holds the click chemistry handle on its 5′ end, and the branched capped oligo holds the click chemistry handle on its 3′ end to allow optimal topology. Sequential T4 RNA ligase ligation and RtcB ligation yield modified circRNA with both branched caps and branched poly(A) tails. The number of branched caps and tails could be more than 1.

As shown in FIG. 20D, the branched capped oligo in FIG. 20B could be further replaced by a modified branched oligo, which consists of a capped oligo with an internal click chemistry handle and 3′phosphate conjugated to a poly(A) oligo with 5′phosphate and 3′click chemistry handle. The oligo was ligated to the 5′/3′-hydroxyl mRNA by T4 RNA ligase followed by ligation of RtcB ligase, yielding chemically modified Type 2 QRNA. For all the RtcB ligation steps in FIG. 20A-D, a complementary sequence proximal to the 5′/3′-ends is required to allow efficient circularization.

### Example 9: Chemically Modified QRNA Workflow Validation

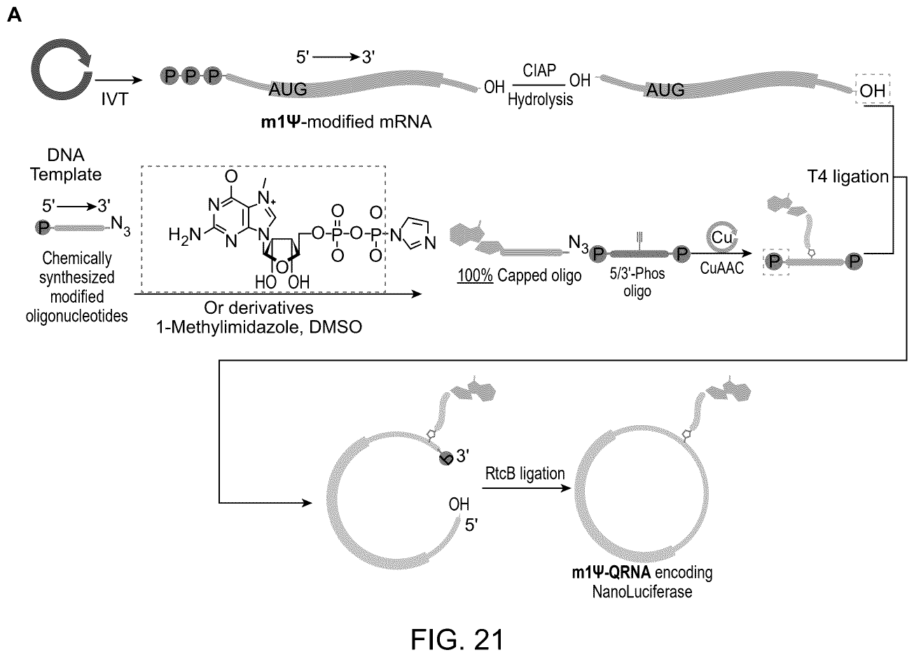

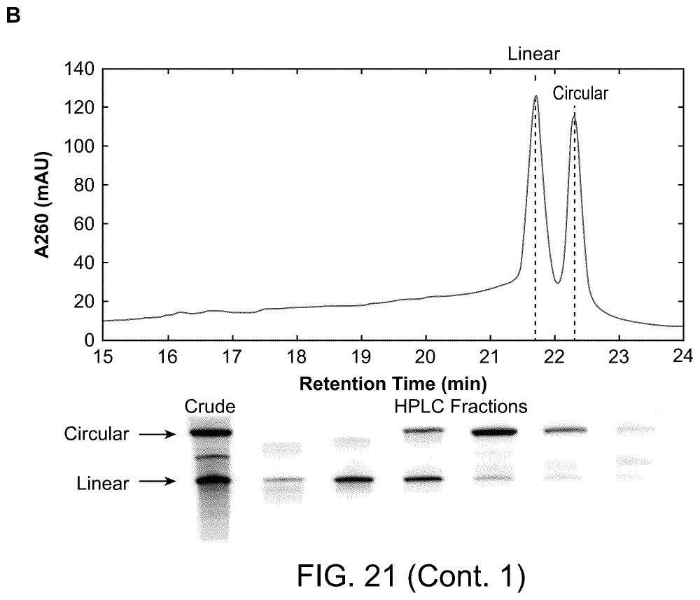

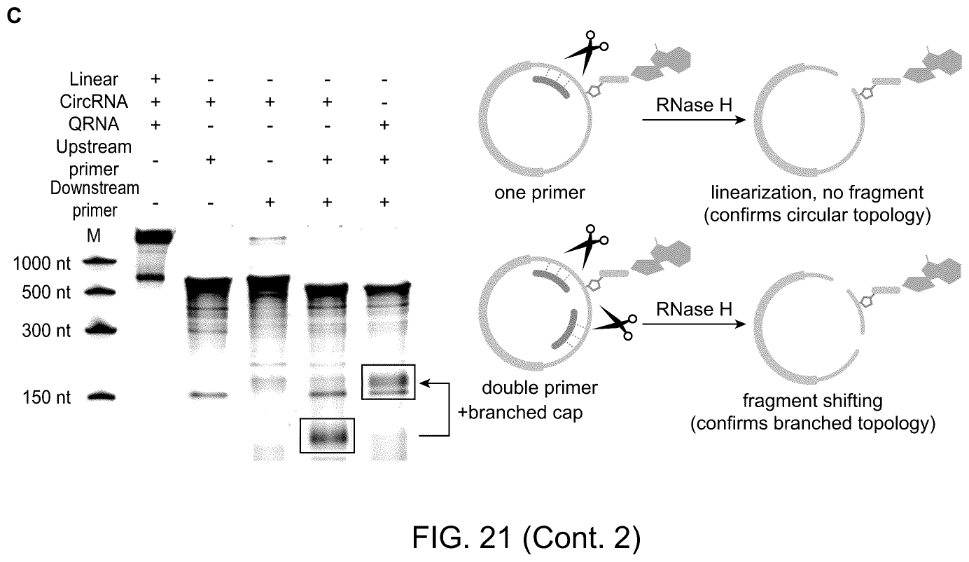

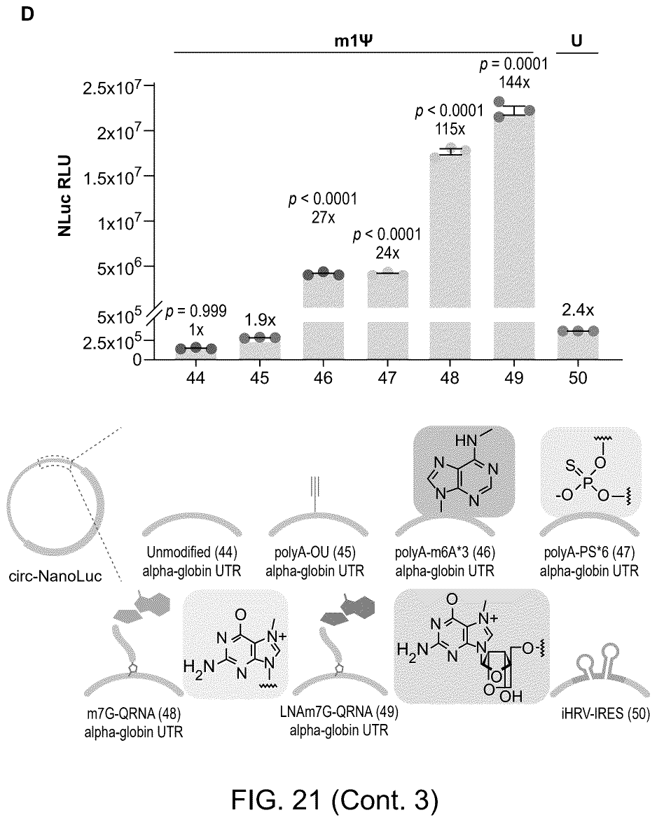

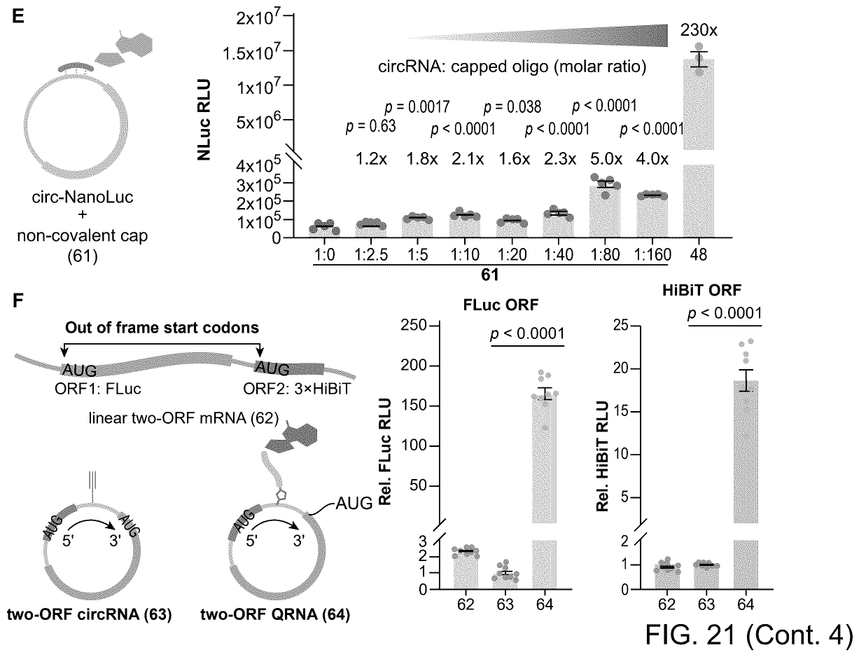

To validate the feasibility of modified QRNA synthesis workflow presented in FIG. 20, synthetic linear mRNA encoding NanoLuciferase (NLuc) was prepared from DNA template by IVT with 100% m1 substitution. The triphosphorylated mRNA after IVT was then treated with Calf Intestinal Alkaline Phosphatase (CIAP) [Promega, M1821] to yield 5′/3′-hydroxyl mRNA. A chemically synthesized oligonucleotide with a branched m7G cap connected by copper catalyzed azide-alkyne cycloaddition (CuAAC) bearing 5′/3′-phosphates was then ligated to the 5′/3′-hydroxyl mRNA by T4 RNA ligase [Promega, M1051], then by RtcB ligase [NEB, M0458S] to afford the NLuc QRNA with 100% m1Ψ modifications (FIG. 21A). The circularization products were isolated by HPLC purification using previously described conditions and characterized by gel electrophoresis where successful circularization was confirmed by slower mobility on gel and longer retention time on HPLC (FIG. 21B). To further confirm capping on the circular transcript, a double RNase H assay was performed where two primers were designed to flank the branched region of the QRNA (FIG. 21C). When only one primer was used, no fragmentation occurred but the transcript was linearized to the same length as the linear precursor, confirming successful circularization. When both primers were used, the fragments flanking the branched region were produced, where successful capping on the QRNA was indicated by the further gel shift of its fragment.

Using the workflow described in FIG. 21A, site-specific modifications were introduced to NLuc circRNA including no modification (construct 44), 5-octadiynyl deoxyuridine (OU) handle (construct 45), m6A cluster (construct 46), phosphorothioate cluster (construct 47), m7G branched cap (construct 48), and LNAm7G branched cap (construct 49). These sets of modified circRNA were transfected to HeLa cells along with an unmodified circRNA bearing iHRV-IRES prepared by traditional back-splicing workflow (construct 50). Both chemical capping and modification incorporation significantly increased the protein production with the LNAm7G QRNA yielded up to 144-fold more protein at 8 hrs (FIG. 21D).

To further investigate if the branched cap for the QRNA could be incorporated in a non-covalent manner, a short capped RNA (20 bp in length) was synthesized and designed such that it is complementary to the circRNA sequence (construct 61). Observable enhancement of protein production (up to 5 fold) occurred at a high ratio of capped oligo: circRNA (up to 80:1 molar ratio) while the level of increase was significantly less than covalent incorporation of the cap (FIG. 21E). Nonetheless, Applicant envisions site-specifically incorporation of homology-enhancing modifications such as LNA on the circRNA transcript may hold potential for higher response to translation enhancement by non-covalent caps.

Finally, Applicant evaluated if the branched cap on the QRNA has the ability to initiate translation of open reading frames (ORFs) upstream of it. Previous literature has reported the possibility of ribosome to back-scan in 3′ to 5′ direction rather than the typical 5′ to 3′ direction. Applicant wondered if such a mechanism would allow the branched cap on QRNA to initiate translation of multiple ORFs located on opposite sides, which is not possible for either traditional 5′ cap (as there is no upstream from the 5′ end) or IRES-based translation due to its defined directionality. To this end, mRNA containing two luciferase ORFs that were out of frame from each other was synthesized by IVT (construct 62) and circularized by workflow described in FIG. 21A to yield OU-only circRNA (construct 63) and QRNA (construct 64) such that the branched cap was in between the HiBiT ORF and FLuc ORF. mRNAs were transfected into HeLa cells. FLuc and HiBiT bioluminometry were evaluated 8 hrs post transfection. Interestingly, translation of both the HiBiT and FLuc ORFs were enhanced by QRNA, although the downstream FLuc ORF was still translated more efficiently due to preferred 5′ to 3′ directionality (FIG. 21F). Applicant thus showed that QRNA has the ability to synthesize multiple proteins on a single RNA transcript.

### Example 10: Branched Capping Drives Translation Via a “Cap-Proximal” Mechanism

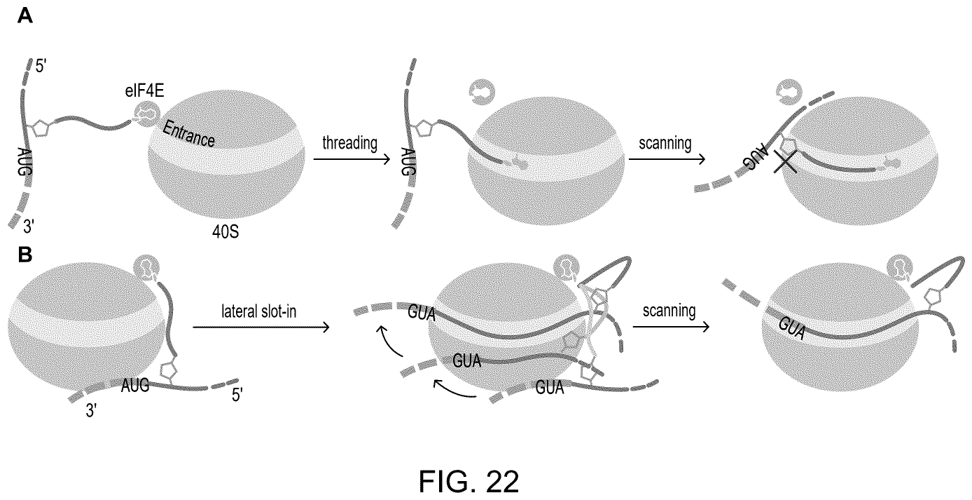

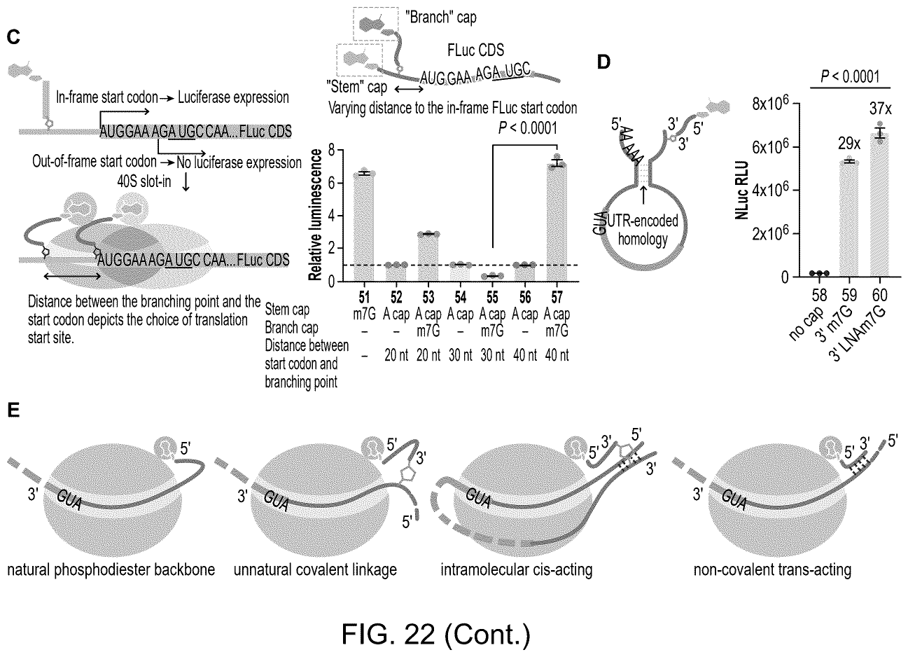

This study revealed that an internal cap, when covalently attached via branched topology, is capable of initiating translation on either linear or circular mRNAs through eIF4E-dependent mechanisms. Such unexpected findings provided additional insights to current understandings of translation initiation models. In the “threading” model of translation initiation, the eIF4-40S scanning requires continuous phosphodiester backbones and are accommodated by dissociation of eIF4E at the entry site, thus requiring eIF4E dissociation/reassociation cycles for each round of translation initiation. However, the unnatural triazole linkage between the branched cap and the mRNA stem may be prohibitive in the “threading” action due to disruption of continuing phosphodiester bonds (FIG. 22A). In the “slot-in” model, eIF4E-bound mRNA enters into the 40S mRNA-binding cleft through lateral slot-in as in the case of IRES-dependent translation initiation. The branched topology of the internal cap may maintain continuous eIF4E-cap engagement while directly slotting the mRNA stem into the 40S binding cleft (FIG. 22B). Such mechanism is structurally supported by location of eIF4F adjacent to eIF3E, -K, and -L on the mRNA exit side of 43S pre-initiation complex and more recent discovery of additional eIF4F-eIF3C and ribosomal protein eS7 interactions, which are universally conserved in eukaryotes.

To experimentally probe the potential ribosome slot-in mechanism induced by the branched cap, Applicant specifically designed a reporter FLuc with an out-of-frame start codon 8-nt downstream the in-frame start codon to probe the position of ribosome landing upon branched-cap induced slot-in (FIG. 22C). Specifically, Applicant introduced a series of synthetic 30-nt 5′ UTRs with various distances from the branching position to the FLuc start codon (F-AUG). The mRNA stem was capped by a non-functional adenosine cap (A cap) to slow down exonuclease degradation, and the branch was capped by the canonical m⁷G cap for translation initiation. When placing the branched cap 40-nt upstream the F-AUG, it exhibited similar FLuc translation as a canonical m7G cap in linear topology, in agreement with “the first AUG” rule. However, placing the branch cap 30-nt upstream the F-AUG resulted in no or even lower translation than the A cap control, indicating the 40S might have landed beyond the first F-AUG and opted for the out-of-frame start codon 8 nt downstream F-AUG (38 nt from the branching point). Interestingly, placing the branch cap 20-nt upstream the F-AUG partially rescued FLuc translation but less efficient than the 40-nt upstream branched cap, potentially through Brownian-ratchet bi-directional ribosomal scanning process. In total, these results suggest that the ribosome lands at 30~38 nt downstream the branching site of the branched cap.

Next, intrigued by the significant translation driven by branched cap attached in 3′ UTR of the minimal HiBIT QRNA (FIG. 11E), Applicant examined if Applicant could topologically invert the natural 5′-cap/3′-poly(A) feature of mRNA to a synthetically 3′-cap/5′-poly(A) mRNA and induce translation through UTR-encoded homology (FIG. 22D) for longer transcripts. Satisfactorily, the 3′-cap/5′-poly(A) design of NLuc mRNA led to 29-fold higher expression than the uncapped control, albeit the expression was low due to lack of end protection against 5′ XRNs. Modifying the 3′-cap from m7G-rG to LNAm7G-LNA+5×OMe further increased the expression to 37-fold, indicating that it likely still proceeded through an eIF4E-dependent mechanism.

Furthermore, given that 3′-cap can efficiently initiate translation via the proximity between the 5′/3′-UTRs by homology base-pairing, Applicant hypothesized that a “trans-acting intermolecular cap” that hybridizes to the circRNA 5′-UTR without covalent linkage might also drive translation. Applicant thus inserted a 20-nt sequence in m¹ψ-NLuc-circRNA (without IRES) for the hybridization with a complementary capped oligonucleotide at varying molar ratios (FIG. 21E). Interestingly, the non-covalent trans-acting caps also enhanced protein production by up to 5-fold at 1:80 mRNA to trans-acting cap ratio, while still significantly lower than the QRNA, potentially due to RNA helicase activity of eIFs or ribosome scanning to unwind the hybridized RNA duplex.

Lastly, to test whether the branched cap could drive the translation for upstream AUG via either proximity looping or bidirectional ribosome scanning to opt for upstream open reading frames (ORFs), Applicant generated a two-ORF reporter mRNA that contained a 5′ full-length FLuc ORF and a 3′ 3×HiBIT ORF with an out-of-frame start codon from the FLuc ORF (construct 62). The two-ORF mRNA was circularized (construct 63) and contained a branched cap (construct 64) where the LNAm7G cap was located 77 nt upstream of the FLuc ORF and 240 nt downstream of the 3×HiBiT (34 amino acids) ORF (FIG. 21F). Compared to the circRNA (uncapped QRNA control), QRNA exhibited higher expression of both the FLuc ORF by 166-fold and the HiBiT ORF by 18.7-fold. These results indicated the ability of the internal branched cap to induce translation of an upstream ORF through either direct 3D proximity of the branched cap with upstream UTR and start codon or through 1D ribosome 3′ to 5′ back scanning, providing potential avenues for replacing IRES for encoding two proteins of interest onto a single mRNA transcript.

Collectively, these observations suggest a general “cap-proximal” mechanism, permitting cap and 5′ UTR sequences connected to mRNA via natural phosphodiester backbone, unnatural covalent linkages, or noncovalent hybridization, to effectively initiate translation (FIG. 22E). Such a model is first supported by dual-capped mRNA and QRNA, and further supported by successful translation initiation driven by proximal but noncovalent cap structures mediated by either intramolecular proximity (a distal cis-acting “3′-cap” base pairing with the 5′ end of mRNA, FIG. 22D) or intermolecular proximity (a trans-acting, non-covalent 5′-cap, FIG. 21E). Such a mechanism may provide inspiration for future therapeutic mRNA architectures.

### Example 11: Modified mRNA for In Vivo Applications in Mouse Disease Models

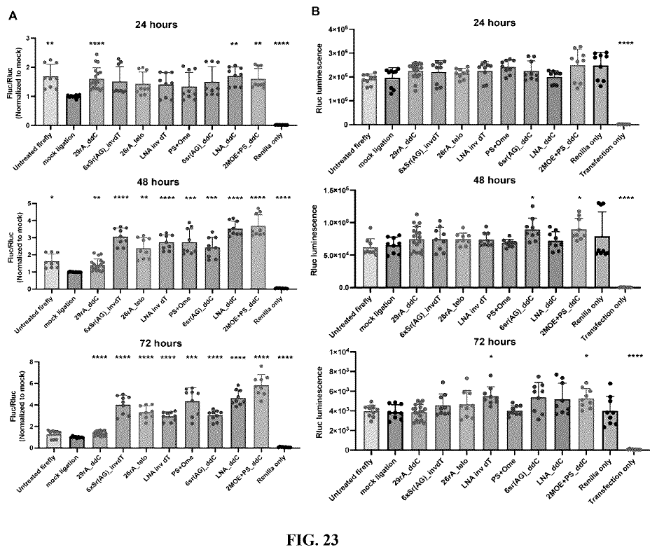

Applicant expanded a chemical screen for mocRNA by testing additional modifications commonly used in RNA stabilization, such as 2′ O-methyl(2′OMe), locked nucleic acids (LNA), 2′ methoxyethyl(2′MOE). Applicant ligated a panel of modified oligos to firefly luciferase mRNAs to generate messenger oligonucleotide-conjugated RNAs (mocRNAs) containing various modifications. Applicant co-transfected the Firefly luciferase mocRNAs with an unmodified Renilla luciferase internal control and measured expression at 24, 48, and 72 hours. Applicant observed that phosphorothioate modifications combined with 2′ methoxyethyl (2MOE+PS_ddC) mocRNAs resulted in higher expression at 72 hours than the other modifications tested (FIGS. 23A and 23B). Applicant proceeded with these modifications for the following mocRNA in vivo tests.

RNA vaccination studies in animal models, particularly mice, have become more popular following the clinical emergence of mRNA vaccines. Applicant established workflows to deliver mRNA to mice for the in vivo demonstration of mRNA activity.

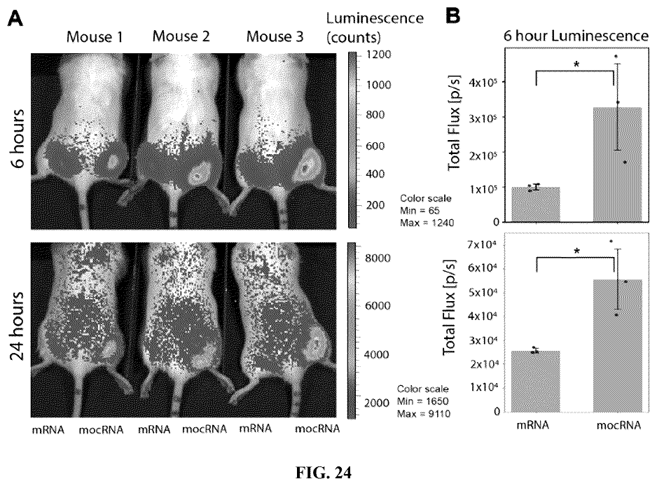

Applicant began in vivo mouse studies by measuring the expression of mRNA and mocRNAs encoding a Firefly luciferase reporter. Firefly luciferase-encoding mRNA or 2MOE+PS_ddC mocRNA was incubated with commercially available pre-formed lipid nanoparticles (In vivo-jetRNA) and injected pairwise intramuscularly into opposite flanks of mice. At 6 hours, Applicant observed a ~3-fold increase in luciferase expression from mocRNA compared to unmodified mRNA, indicating that mocRNA had increased mRNA stability in vivo. However, by 24 hours, the luciferase signal of unligated mRNA in some mice approached background levels, making quantification more difficult and indicating a lack of delivery efficiency (FIGS. 24A and 24B).

Premade lipid nanoparticles (LNPs) resulted in some variability in expression between replicates (data not shown) and between experiments. To improve reproducibility and delivery efficiency across experiments, Applicant pursued an mRNA/LNP encapsulation strategy that more closely matched the industry standard, in which mRNAs are mixed into a lipid solution rapidly and encapsulated into LNPs. Applicant used commercial instruments for the microfluidic preparation of mRNA LNP formulations. The instrument produces controlled mixing of mRNA (aqueous phase) with a miscible organic phase (ethanol), in which the ionic lipids are dissolved. This reproducibly generates mRNA: LNP nanoparticles of controlled size, which can be efficiently delivered for in vivo applications.

Applicant used an SM-102 based lipid mixture, based on work by Hassett et al., Mol. Ther. Nucleic Acids 15, 1-11 (2019). Applicant validated a microfluidic LNP workflow by testing the expression of unligated, cotranscriptionally capped (CleanCap AG) in vitro transcribed Firefly luciferase mRNA via various routes of delivery in mice. Mice were injected intratumorally, subcutaneously, intradermally, or intramuscularly and imaged at varying timepoints for luciferase expression.

Intratumoral injections were performed on syngeneic mouse melanoma tumors (D4M.3A.3, “parental” in C57BL/6 mice) grown to 50 mm³ after subcutaneous seeding into the mouse flank.

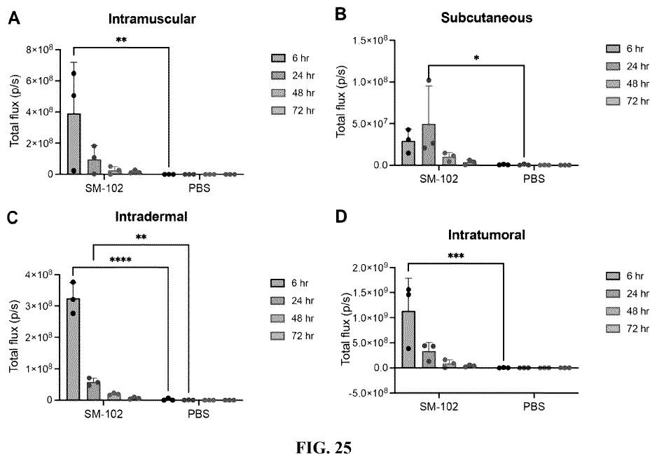

Applicant observed improved reproducibility between mice and significant signal above background at 24 hours for some conditions (FIG. 25), compared to the pre-formulated LNP solution (In vivo-jetRNA) used previously.

### Example 12: 3′-Capped Multi-Cap RNA

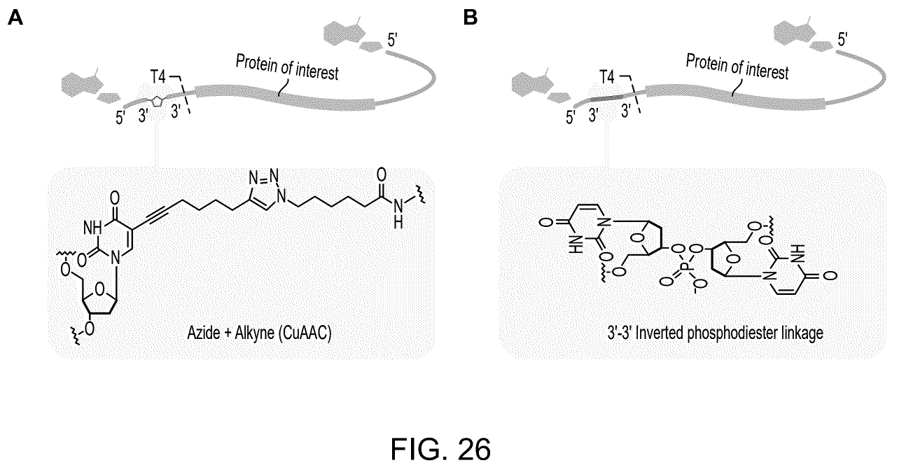

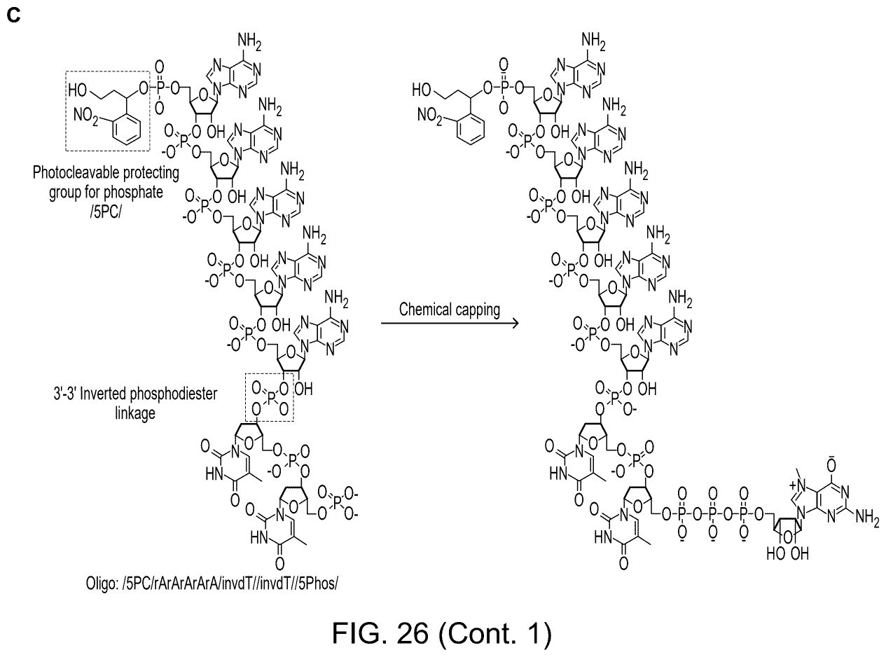

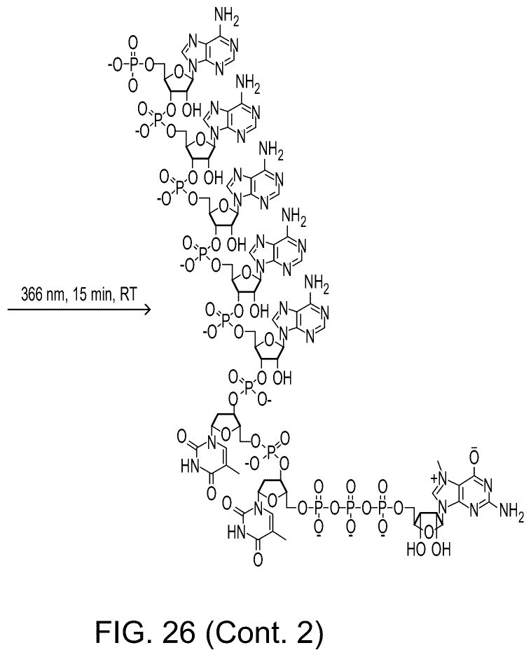

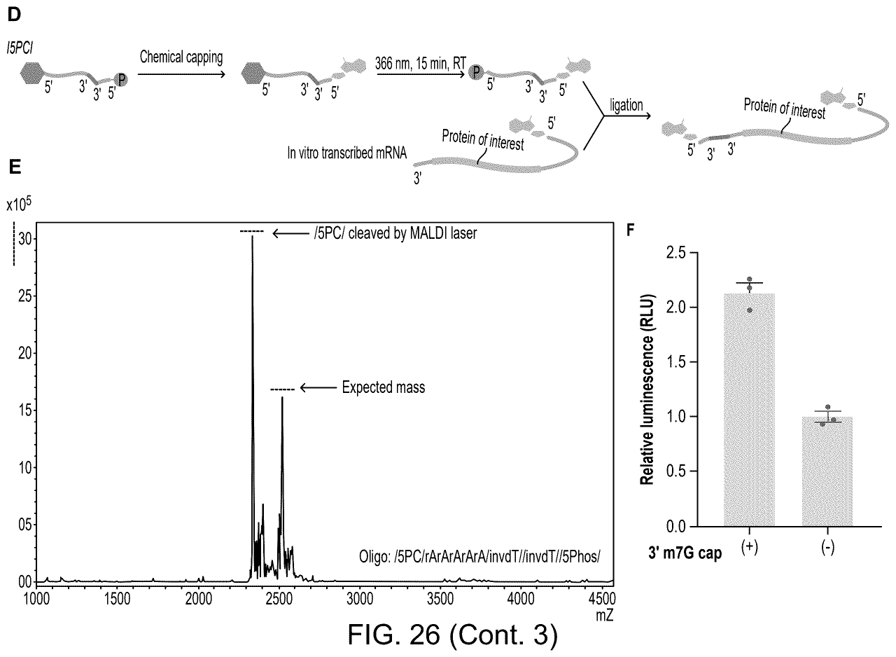

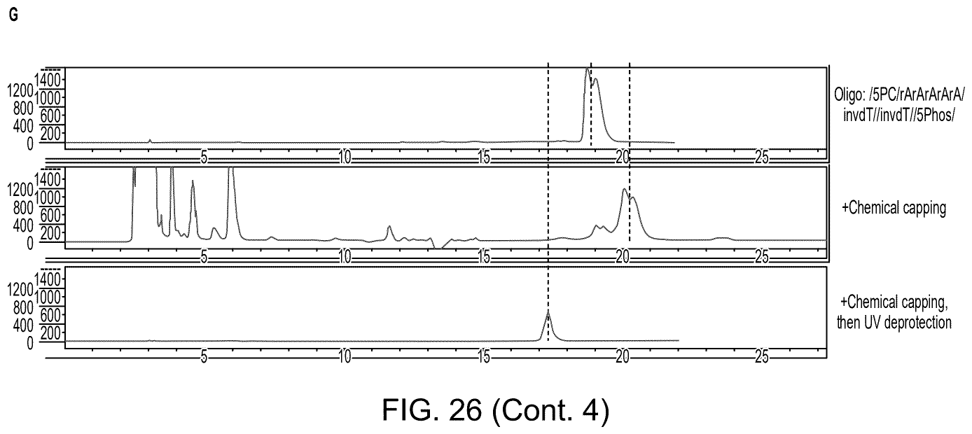

Following Applicant's demonstration of cap proximal translation initiation in which a 3′ cap could initiate translation by homology induced proximity to the start codon (see Example 10), Applicant envisioned that simultaneous introduction of an m7G cap on both ends of an mRNA could be beneficial. Such a 3′ cap could have both a role in translation initiation and a role in protecting the RNA against degradation by exonucleases. As the m7G cap contains a 5′ to 5′ triphosphate linkage between two 5′ hydroxyl groups of the sugar ring, chemical inversion of the 3′ end of the mRNA is required. Such inversion could be achieved through click chemistry (FIG. 26A), or achieved through a 3′ to 3′ phosphodiester linkage that could be introduced by solid phase chemical synthesis of the synthetic oligonucleotide (FIG. 26B).

As a proof of concept for 3′ capping, Applicant synthesized a minimal synthetic oligonucleotide (sequence=/5PC/rArArArArA/invdT//invdT//5Phos/, see FIG. 26C-26), where/5PC/stands for a photocleavable nitro-benzene protecting group for phosphate incorporated through its phosphoramidate precursor (glenresearch.com/photo-regulation-cleavage/10-4920.html), and/invdT/stands for a deoxy thymine base synthesized from the inverted dT phosphoramidate (glenresearch.com/reverse-synthesis-phosphoramidites/10-0301.html). A protecting group, such as the/5PC/protecting group, is required as the chemical capping reagent will react with all phosphates on the oligo, while an intact 5′ phosphate is required for subsequent ligation to the mRNA. Other photocleavable protecting groups can be used, such as one or more of this described in Tavakoli & Min (2022), Photochemical modifications for DNA/RNA oligonucleotides, RSC Advances, (12) 6484-6507.

The overall workflow for generating a 5′/3′ capped mRNA therefore involves chemical capping of one of the phosphate on the synthetic oligo, followed by photo-deprotection of the other phosphate by irradiation with 365 nm light to reveal the other 5′ phosphate moiety, and finally ligating the deprotected oligo to an mRNA using T4 RNA ligase (FIG. 26D). The identity of the synthetic oligonucleotide (/5PC/rArArArArA/invdT//invdT//5Phos/) was confirmed by MALDI-TOF (FIG. 26E), and successful chemical capping is confirmed by the longer retention time on HPLC due to addition of the m7G base (FIG. 26F, second chromatograph), and successful photo-deprotection is confirmed by the significantly shorter retention time on HPLC due to loss of the hydrophobic nitro-benzene ring (FIG. 26F, third chromatograph). Lastly, we ligated this oligo onto a Firefly luciferase mRNA capped with a non-functional adenosine cap (A-cap), and addition of the 3′-capped oligo significantly increased the protein expression (FIG. 26G).

### Example 13: Topological expansion by photochemical crosslink and ligation

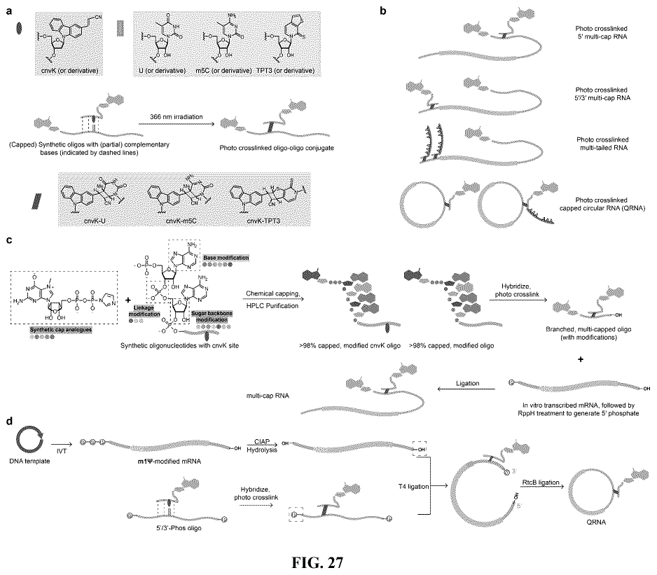

The click chemistry used for oligo topology expansion can be replaced by using a photochemical crosslinking reaction, facilitated by DNA/RNA hybridization. One of the synthetic oligos can contain a photoactive alkene such as the cyanovinyl moiety in cnvK and cnvD (see cambio.co.uk/library/images/html_images/Glen_structures/GR30-2.pdf), or the alkene in furan or pyrone in coumarin or psoralen (See pubs.rsc.org/en/content/articlepdf/2022/ra/d1ra05951c FIGS. 11, 12, 13, and 14). The other synthetic oligonucleotides can be partially complementary to the photocrosslinker-containing oligo, where the photocrosslinking group is within the complementary region. The photocrosslinking group is then able to undergo [2+2] photocycloaddition with the acceptor base at −1/0/+1 position from the photocrosslinking moiety, and the acceptor base must contain an activated alkene such as U/C/m5C or an unnatural base as TPT3 upon UV-A (~366 nm) irradiation for 1~60 seconds (FIG. 27A). The topologically modified RNAs containing the photo crosslinking linkages can then be generated by the ligation workflow described herein (FIG. 27B-27D).

### Example 14: Effect of TMO-Modification on mRNA Translation Efficiency

In order to examine the effect of thiomorpholino oligo (TMO) ribose modification on translation efficiency, Applicant synthesized mRNA Fluc reporter construct containing the sequence of SEQ ID NO: 159, and also an m7G 5′ cap, and a 5′UTR having a TMO ribose modification on the first nucleotide (m7G+TMO:/5Phos//TMO-A/rGrArGrArArUrArArArArA (SEQ ID NO: 160)). The synthesized mRNA was expressed in HeLa cells, and reporter luminescence was measured as a proxy for translation efficiency. A synthesized mRNA construct containing an unmodified 5′UTR (m7G+UnmodA:/5Phos/rArGrArGrArArUrArArArArA (SEQ ID NO: 161)) was assessed as a control.

(SEQ ID NO: 159)
AGAAAAAGGAATAAACTAGTATTCTTCTGGTCCCCACAGACTCAGA
GAGAACCCGCCACCATGGAAGATGCCAAAAACATTAAGAAGGGCC
CAGCGCCATTCTACCCACTCGAAGACGGGACCGCCGGCGAGCAGC
TGCACAAAGCCATGAAGCGCTACGCCCTGGTGCCCGGCACCATCG
CCTTTACCGACGCACATATCGAGGTGGACATTACCTACGCCGAGT
ACTTCGAGATGAGCGTTCGGCTGGCAGAAGCTATGAAGCGCTATG
GGCTGAATACAAACCATCGGATCGTGGTGTGCAGCGAGAATAGCT
TGCAGTTCTTCATGCCCGTGTTGGGTGCCCTGTTCATCGGTGTGG
CTGTGGCCCCAGCTAACGACATCTACAACGAGCGCGAGCTGCTGA
ACAGCATGGGCATCAGCCAGCCCACCGTCGTATTCGTGAGCAAGA
AAGGGCTGCAAAAGATCCTCAACGTGCAAAAGAAGCTACCGATCA
TACAAAAGATCATCATCATGGATAGCAAGACCGACTACCAGGGCT
TCCAAAGCATGTACACCTTCGTGACTTCCCATTTGCCACCCGGCT
TCAACGAGTACGACTTCGTGCCCGAGAGCTTCGACCGGGACAAAA
CCATCGCCCTGATCATGAACAGTAGTGGCAGTACCGGATTGCCCA
AGGGCGTAGCCCTACCGCACCGCACCGCTTGTGTCCGATTCAGTC
ATGCCCGCGACCCCATCTTCGGCAACCAGATCATCCCCGACACCG
CTATCCTCAGCGTGGTGCCATTTCACCACGGCTTCGGCATGTTCA
CCACGCTGGGCTACTTGATCTGCGGCTTTCGGGTCGTGCTCATGT
ACCGCTTCGAGGAGGAGCTATTCTTGCGCAGCTTGCAAGACTATA
AGATTCAATCTGCCCTGCTGGTGCCCACACTATTTAGCTTCTTCG
CTAAGAGCACTCTCATCGACAAGTACGACCTAAGCAACTTGCACG
AGATCGCCAGCGGCGGGGCGCCGCTCAGCAAGGAGGTAGGTGAGG
CCGTGGCCAAACGCTTCCACCTACCAGGCATCCGCCAGGGCTACG
GCCTGACAGAAACAACCAGCGCCATTCTGATCACCCCCGAAGGGG
ACGACAAGCCTGGCGCAGTAGGCAAGGTGGTGCCCTTCTTCGAGG
CTAAGGTGGTGGACTTGGACACCGGTAAGACACTGGGTGTGAACC
AGCGCGGCGAGCTGTGCGTCCGTGGCCCCATGATCATGAGCGGCT
ACGTTAACAACCCCGAGGCTACAAACGCTCTCATCGACAAGGACG
GCTGGCTGCACAGCGGCGACATCGCCTACTGGGACGAGGACGAGC
ACTTCTTCATCGTGGACCGGCTGAAGAGCCTGATCAAATACAAGG
GCTACCAGGTAGCCCCAGCCGAACTGGAGAGCATCCTGCTGCAAC
ACCCCAACATCTTCGACGCCGGGGTCGCCGGCCTGCCCGACGACG
ATGCCGGCGAGCTGCCCGCCGCAGTCGTCGTGCTGGAACACGGTA
AAACCATGACCGAGAAGGAGATCGTGGACTATGTGGCCAGCCAGG
TTACAACCGCCAAGAAGCTGCGCGGTGGTGTTGTGTTCGTGGACG
AGGTGCCTAAAGGACTGACCGGCAAGTTGGACGCCCGCAAGATCC
GCGAGATTCTCATTAAGGCCAAGAAGGGCGGCAAGATCGCCGTGT
GATAATAGCTCGAGGCTGGAGCCTCGGTGGCCATGCTTCTTGCCC
CTTGGGCCTCCCCCCAGCCCCTCCTCCCCTTCCTGCACCCGTACC
CCCGTGGTCTTTGAATAAAGTCTGAGTGGGCGGCACAATTGAAAA
AAAAAAAAAAAAAAAAAAAAAAAAAGCATATGACTAAAAAAAAAA
AAAAAAAAAAAAAAAAAAAAAAAAAAAAAAAAAAAAAAAAA

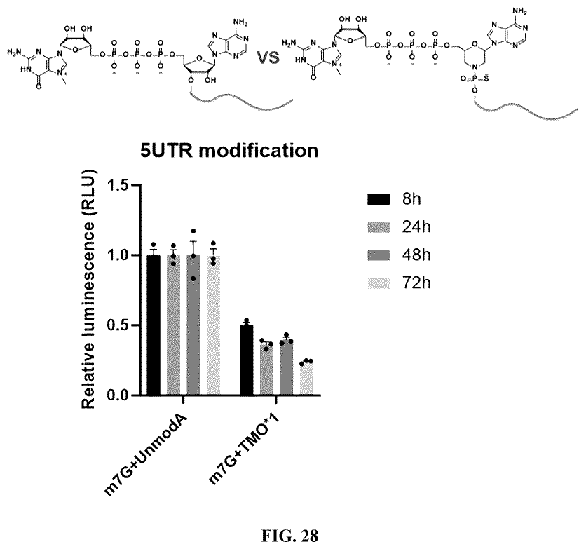

As shown in FIG. 28, a first-nucleotide 5′-UTR TMO modification altered the translation efficiency of the reporter construct relative to that observed of the control reporter construct that did not contain the first-nucleotide 5′-UTR TMO modification.

### Example 15. Evaluation of the Effect of Internal Branched Cap Positioning

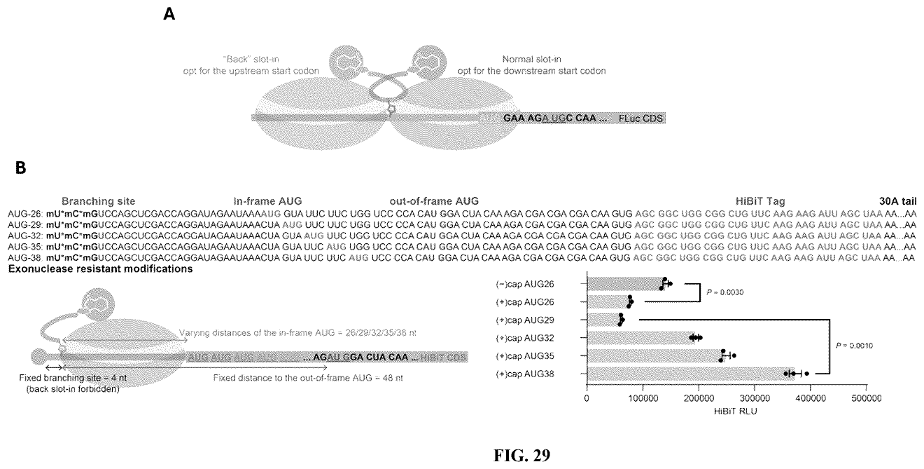

To further probe the exact ribosome landing site as affected by the position of the branch oligo, Applicant designed several chemically synthesized mRNA constructs in which, rather than moving the branching site, the in-frame start codon was placed at different positions (see FIG. 29A-29B). All constructs contained 5′-exonuclease resistant modifications, a fixed branching site at the +4 base (which does not permit back slot-in), a fixed out-of-frame start codon 46 nt downstream the branching site, a 30A polyA tail (SEQ ID NO: 199), and an in-frame start codon encoding the HiBiT tag 26/29/32/35/38 nt downstream the branching site. Translation activity of these mRNA constructs was evaluated in the rabbit reticulocyte lysate (RRL) in vitro translation system for 1 hour. In agreement with the results presented in FIG. 22C, the branched cap did not efficiently induce translation when the distance between AUG to the branching site was shorter than 30 nt. These results indicate that (1) the optimal distance between the branching point and the desired downstream AUG is >35 nt, as the landing of the ribosome recruited by the branched cap was 32~35 nt downstream of the branching site for normal slot-in; and (2) the branching site of the branched cap should optimally be 5~25 nt from the 5′-terminus to avoid back slot-in, if not desired.

### Sequences

The following tables contain sequences of oligonucleotides used in the experiments shown in each referenced figure. Nucleotide modifications are denoted as follows:
r=ribose sugar
m=2OMe-modified sugar
*=phosphorothioate (PS) linkage
+=locked nucleic acid (LNA)
i2FA=internal 2′-deoxy fluoro
iN6Me=internal N6-methyladenosine
ibetaL=internal L-adenosine
i5OCTdU=internal
3 AzideN=3′ 6-diazynyl-N-heptylhexanamide

TABLE 1
Sequences used in FIG. 8 and FIG. 9

| | | | | SEQ ID |
|---|---|---|---|---|
| FIG # | Identifier | 5′ Cap | Sequence (5′ to 3′) | NO: |
| 8B | G (0) | m7G | /5Phos/rGrGrArGrArArUrArArArArA | 1 |
| 8B/9A/ | A (1) | m7G | /5Phos/rArGrArGrArArUrArArArArA | 2 |
| 91/9J | | | | |
| 8B | U (2) | m7G | /5Phos/rUrGrArGrArArUrArArArArA | 3 |
| 8B | C (3) | m7G | /5Phos/rCrGrArGrArArUrArArArArA | 4 |
| 8B | I (4) | m7G | /5Phos/rIrGrArGrArArUrArArArArA | 5 |
| 8B | m6A (5) | m7G | /5Phos//iN6Me-rA/rGrArGrArArUrArArArArA | 6 |
| 8C | PS (6) | m7G | /5Phos/rA*rGrArGrArArUrArArArArA | 7 |
| 8C | PSx6 (7) | m7G | /5Phos/rA*rG*rA*G*rA*rA*rUrArArArArA | 8 |
| 8D | 2FA (8) | m7G | /5Phos//i2FA/rGrArGrArArUrArArArArA | 9 |
| 8D | LA (9) | m7G | /5Phos//ibetaL-rA/rGrArGrArArUrArArArArA | 10 |
| 8D | dA (10) | m7G | /5Phos/ArGrArGrArArUrArArArArA | 11 |
| 8D/9I/ | 2OMe | m7G | /5Phos/mArGrArGrArArUrArArArArA | 12 |
| 9J | (11) | | | |
| 8D | 2MOE | m7G | /5Phos//i2MOErA/rGrArGrArArUrArArArArA | 13 |
| | (12) | | | |
| 8D/9B/ | LNA | m7G | /5Phos/+ArGrArGrArArUrArArArArA | 14 |
| 91/9J | (13) | | | |
| 8D | dAx6 | m7G | /5Phos/AGAGAArUrArArArArA | 15 |
| | (14) | | | |
| 8D/9C/ | 2OMex6 | m7G | /5Phos/mAmGmAmGmAmArUrArArArArA | 16 |
| 9I | (15) | | | |
| 8D | 2MOEx6 | m7G | /5Phos//i2MOErA//i2MOErG//i2MOErA//i2MOErG// | 17 |
| | (16) | | i2MOErA//i2MOErA/rUrArArArArA | |
| 8D | LNAx6 | m7G | /5Phos/+A+G+A+G+A+ArUrArArArArA | 18 |
| | (17) | | | |
| 8E | Bn7G | Bn7G | /5Phos/rArGrArGrArArUrArArArArA | 19 |
| | (18) | | | |
| 8E | ClBn7G | CIBn7G | /5Phos/rArGrArGrArArUrArArArArA | 20 |
| | (19) | | | |
| 8E | ClBnOEt | ClBnO | /5Phos/rArGrArGrArArUrArArArArA | 21 |
| | 7G (20) | Et7G | | |
| 8E/9I | LNAm7 | LNAm7 | /5Phos/rArGrArGrArArUrArArArArA | 22 |
| | G (21) | G | | |
| 8F/9D/ | LNAm7 | LNAm7 | /5Phos/+ArGrArGrArArUrArArArArA | 23 |
| 91/9J | G-LNA | G | | |
| | (22) | | | |
| 8F | LNAm7 | LNAm7 | /5Phos/+A+G+A+G+A+ArUrArArArArA | 24 |
| | G- | G | | |
| | LNAx6 | | | |
| | (23) | | | |
| 8F/9E/ | LNAm7 | LNAm7 | /5Phos/mAmGmAmGmAmArUrArArArArA | 25 |
| 9I | G- | G | | |
| | 2OMex6 | | | |
| | (24) | | | |
| 91/9J | m7G- | m7G | /5Phos/mAmGmAmGmAmAmUmAmAmAmAmAmAmGmGmGm | 26 |
| | 23xOMe | | AmAmUmAmAmAmC | |
| | (43) | | | |

TABLE 2
Primer sequences used in FIG. 9

| | | SEQ ID |
|---|---|---|
| Sequence Name | Sequence (5′ to 3′) | NO: |
| GAPDH Forward | AAGGTCGGAGTCAACGGATT | 27 |
| GAPDH Reverse | TGAGGTCAATGAAGGGGTCA | 28 |
| Mx11 Forward | GGCTGTTTACCAGACTCCGACA | 29 |
| Mx1 Reverse | CACAAAGCCTGGCAGCTCTCTA | 30 |
| ISG15 Forward | CTCTGAGCATCCTGGTGAGGAA | 31 |
| ISG15 Reverse | AAGGTCAGCCAGAACAGGTCGT | 32 |

TABLE 3
Sequences used in FIG. 10

| FIG | | | Sequence (5′ to 3′) - Azide | Sequence (5′ to 3′) - Alkyne | SEQ ID |
|---|---|---|---|---|---|
| # | Identifier | 5′ Cap | Oligo | Oligo | NO: |
| 10E | (25) | — | — | — | - |
| 10E, F | (26) | m7G | /5Phos/rArGrArGrArUrArA/3Azide | rArArA/i5OCTdU/rArArArArArA | 33 |
| | | | N/ | | |
| 10E, F | (27) | m7G | | /5Phos/rArGrArUrArArA/i5OCTdU/ | 34 |
| | | | | rArArArArArA | |
| 10G | No OU | m7G | | /5Phos/rArGrArUrArArArArArArAr | 35 |
| | (34) | | | ArA | |
| 10G | 2 nt (S8) | m7G | | /5Phos/rA/i5OCTdU/rGrArUrArArA | 36 |
| | | | | rArArArArArA | |
| 10G | 5 nt (S9) | m7G | | /5Phos/rArGrArU/i5OCTdU/rArArA | 37 |
| | | | | rArArArArArA | |
| 10G | 8 nt (28) | m7G | | /5Phos/rArGrArUrArArA/i5OCTdU/ | 38 |
| | | | | rArArArArArA | |
| 10G | 11 nt | m7G | | /5Phos/rArGrArUrArArArArArA/i5 | 39 |
| | (S10) | | | OCTdU/rArArArArA | |
| 10H | No OU | m7G | | /5Phos/rArGrArUrArArArArArArAr | 35 |
| | (34) | | | ArA | |
| 10H | 2 nt (35) | m7G | /5Phos/rArGrArGrArUrArA/3Azide | /5Phos/rA/i5OCTdU/rGrArUrArArA | 40 |
| | | | N/ | rArArArArArA | |
| 10H | 5 nt (36) | m7G | /5Phos/rArGrArGrArUrArA/3Azide | /5Phos/rArGrArU/i5OCTdU/rArArA | 41 |
| | | | N/ | rArArArArArA | |
| 10F | 8 nt (31) | m7G | /5Phos/rArGrArGrArUrArA/3Azide | /5Phos/rArGrArUrArArA/i5OCTdU/ | 42 |
| | | | N/ | rArArArArArA | |
| 10H | 11 nt (37) | m7G | /5Phos/rArGrArGrArUrArA/3Azide | /5Phos/rArGrArUrArArArArArA/i5 | 43 |
| | | | N/ | OCTdU/rArArArArA | |
| 10I | No OU | m7G | | /5Phos/rArGrArUrArArA/i5OCTdU/ | 38 |
| | (28) | | | rArArArArArA | |
| 10I | 2 nt (29) | m7G | /5Phos/rA-azide | /5Phos/rArGrArUrArArA/i5OCTdU/ | 44 |
| | | | | rArArArArArA | |
| 10I | 5 nt (30) | m7G | /5Phos/rArGrArGrA/3AzideN/ | /5Phos/rArGrArUrArArA/i5OCTdU/ | 45 |
| | | | | rArArArArArA | |
| | 8 nt (31) | m7G | /5Phos/rArGrArGrArUrArA/3Azide | /5Phos/rArGrArUrArArA/i5OCTdU/ | 42 |
| | | | N/ | rArArArArArA | |
| 10I | 11 nt (32) | m7G | /5Phos/rArGrArGrArUrArArArCrU | /5Phos/rArGrArUrArArA/i5OCTdU/ | 46 |
| | | | /3AzideN/ (SEQ ID NO: 170) | rArArArArArA | |
| 10I | 15 nt (33) | m7G | /5Phos/rArGrArGrArUrArArArCrU | /5Phos/rArGrArUrArArA/i5OCTdU/ | 47 |
| | | | rArGrUrA/3AzideN/ (SEQ ID | rArArArArArA | |
| | | | NO: 171) | | |
| 10J | (−PS) (31) | m7G | /5Phos/rArGrArGrArUrArA/3Azide | /5Phos/rArGrArUrArArA/i5OCTdU/ | 42 |
| | | | N/ | rArArArArArA | |
| 10J | (+) PS | m7G | /5Phos/rA*rG*rA*rG*rA*rU*rA*r | /5Phos/rArGrArUrArArA/i5OCTdU/ | 48 |
| | (S11) | | A/3AzideN/ | rArArArArArA | |
| 10K | Linear | m7G | | /5Phos/rGrGrArUrArArA/i5OCTdU/ | 49 |
| | | (38) | | rArArArArArA | |
| | | | | - | |
| 10K | Branched | LNAm7 | /5Phos/+AmGmAmGmAmUrArA/3 | /5Phos/+AmGmAmUmAmArA/i50 | 50 |
| | | G (39) | AzideN/ | CTdU/rArArArArArA | |

TABLE 4
Sequences used in FIG. 11

| FIG | | 5′ | | SEQ ID | Sequence (5′ to 3′)- |
|---|---|---|---|---|---|
| # | Identifier | Cap | Sequence (5′ to 3′) | NO: | Branching Oligo |
| 11B | HibiT 1OU | | /5Phos/rArGrArUrArArArCrUrArG/i5 | 51 | |
| | (chemically | | OctdU/rArUrUrCrUrUrCrUrGrGr | | |
| | synthesized | | UrCrCrCrCrArCrArGrArCrUrCrA | | |
| | RNA) (S4) | | rGrArGrArGrArArCrCrCrGrCrCr | | |
| | | | ArCrCrArUrGrGrUrGrArGrCrGr | | |
| | | | GrCrUrGrGrCrGrGrCrUrGrUrUrC | | |
| | | | rArArGrArArGrArUrUrArGrCrUr | | |
| | | | GrArArArArArArArArArArArAr | | |
| | | | ArArArArArArArA | | |
| 11B | Capped | m7G | Phos/rArGrArUrArArArCrUrArG/i5Octd | 52 | |
| | HibiT_1OU | | U/rArUrUrCrUrUrCrUrGrGrUrCrCrCr | | |
| | (chemically | | CrArCrArGrArCrUrCrArGrArGrArGr | | |
| | synthesized | | ArArCrCrCrGrCrCrArCrCrArUrGrGr | | |
| | RNA) (S5) | | UrGrArGrCrGrGrCrUrGrGrCrGrGrCr | | |
| | | | UrGrUrUrCrArArGrArArGrArUrUrAr | | |
| | | | GrCrUrGrArArArArArArArArArArAr | | |
| | | | ArArArArArArArArA | | |
| 11B | Capped | m7G | hos/rArGrArUrArArArCrUrArG/i5Octd | 53 | /5Phos/rArGrArGrArUrArAr |
| | HibiT_1OU | | U/rArUrUrCrUrUrCrUrGrGrUrCrCrCr | | ArCrU/3AzideN/ (SEQ |
| | (chemically | | CrArCrArGrArCrUrCrArGrArGrArGr | | ID NO: 170) |
| | synthesized | | ArArCrCrCrGrCrCrArCrCrArUrGrGr | | |
| | RNA) + | | UrGrArGrCrGrGrCrUrGrGrCrGrGrCr | | |
| | branch (S6) | | UrGrUrUrCrArArGrArArGrArUrUrAr | | |
| | | | GrCrUrGrArArArArArArArArArArAr | | |
| | | | ArArArArArArArArA | | |
| 11B | Capped | m7G | Phos/rArGrArUrArArArCrUrArG/i5Octd | 54 | m7G+/5Phos/rArGrArGrArU |
| | HibiT_1OU | | U/rArUrUrCrUrUrCrUrGrGrUrCrCrCr | | rArArArCrU/3AzideN/ |
| | (chemically | | CrArCrArGrArCrUrCrArGrArGrArGr | | (SEQ ID NO: 172) |
| | (S7) | | ArArCrCrCrGrCrCrArCrCrArUrGrGr | | |
| | synthesized | | UrGrArGrCrGrGrCrUrGrGrCrGrGrCr | | |
| | RNA) + | | UrGrUrUrCrArArGrArArGrArUrUrAr | | |
| | branched | | GrCrUrGrArArArArArArArArArArAr | | |
| | cap | | ArArArArArArArArA | | |
| 11E | HibiT_azide | | AAAAAAAAAAGAAAAAAAAGAA | 55 | |
| | (IVT | | TAAATTTGTATTATTAATATAATT | | |
| | template) | | AAATTAAAATTAAGAGAAGGGA | | |
| | (S15) | | AGATGGTGAGTGGATGGAGATTA | | |
| | | | TTTAAGAAGATTAGTTAGTAATTT | | |
| | | | TAATTTAATTATATTAATAATACA | | |
| | | | AATTTAAAAAAAAAAAAGAAAA | | |
| | | | AAAAAAAAAAAAAAAAAAAAA | | |
| 11E | HibiT_azide | | AAAAAAAAAAGAAAAAAAAGAA | 56 | |
| | (IVT | | TAAATTTGTATTATTAATATAATT | | |
| | template)_ | | AAATTAAAATTAAGAGAAGGGA | | |
| | circular | | AGATGGTGAGTGGATGGAGATTA | | |
| | (S15) | | TTTAAGAAGATTAGTTAGTAATTT | | |
| | | | TAATTTAATTATATTAATAATACA | | |
| | | | AATTTAAAAAAAAAAAAGAAAA | | |
| | | | AAAAAAAAAAAAAAAAAAAAA | | |
| 11E | HibiT_azide | | AAAAAAAAAAGAAAAAAAAGAA | 57 | m7G+/5Phos/rArGrArGrArA |
| | (IVT | | TAAATTTGTATTATTAATATAATT | | rA/35OCTdU/ |
| | template)_ | | AAATTAAAATTAAGAGAAGGGA | | |
| | branched | | AGATGGTGAGTGGATGGAGATTA | | |
| | (S16) | | TTTAAGAAGATTAGTTAGTAATTT | | |
| | | | TAATTTAATTATATTAATAATACA | | |
| | | | AATTTAAAAAAAAAAAAGAAAA | | |
| | | | AAAAAAAAAAAAAAAAAAAAA | | |
| 11E | HibiT_azide | | GGAAAAAAAAAAGAAAAAAAAG | 58 | m7G+/5Phos/rArGrArGrArA |
| | (IVT | | AATAAATTTGTATTATTAATA | | rA/35OCTdU/ |
| | template)_ | | TAATTAAATTAAAATTAAGA | | |
| | QRNA | | GAAGGGAAGATGGTGAGTGG | | |
| | (S17) | | ATGGAGATTATTTAAGAAGA | | |
| | | | TTAGTTAGTAATTTTAATTTA | | |
| | | | ATTATATTAATAATACAAATT | | |
| | | | TAAAAAAAAAAAAGAAAAA | | |
| | | | AAAAAAAAAAAAAAAAAAA | | |
| | | | A | | |

TABLE 5
Sequences used in FIG. 12

| FIG | | | SEQ ID |
|---|---|---|---|
| # | Identifier | Sequence (5′ to 3′) | NO: |
| 12B | Fluc-globin | AGGGAATAAACTAGTATTCTTCTGGTCCCCACAGACTCAGAGAGAACCCGC | 59 |
| | | CACCATGGAAGATGCCAAAAACATTAAGAAGGGCCCAGCGCCATTCTA | |
| | | CCCACTCGAAGACGGGACCGCCGGCGAGCAGCTGCACAAAGCCATGAA | |
| | | GCGCTACGCCCTGGTGCCCGGCACCATCGCCTTTACCGACGCACATATC | |
| | | GAGGTGGACATTACCTACGCCGAGTACTTCGAGATGAGCGTTCGGCTGG | |
| | | CAGAAGCTATGAAGCGCTATGGGCTGAATACAAACCATCGGATCGTGG | |
| | | TGTGCAGCGAGAATAGCTTGCAGTTCTTCATGCCCGTGTTGGGTGCCCT | |
| | | GTTCATCGGTGTGGCTGTGGCCCCAGCTAACGACATCTACAACGAGCGC | |
| | | GAGCTGCTGAACAGCATGGGCATCAGCCAGCCCACCGTCGTATTCGTGA | |
| | | GCAAGAAAGGGCTGCAAAAGATCCTCAACGTGCAAAAGAAGCTACCGA | |
| | | TCATACAAAAGATCATCATCATGGATAGCAAGACCGACTACCAGGGCT | |
| | | TCCAAAGCATGTACACCTTCGTGACTTCCCATTTGCCACCCGGCTTCAA | |
| | | CGAGTACGACTTCGTGCCCGAGAGCTTCGACCGGGACAAAACCATCGC | |
| | | CCTGATCATGAACAGTAGTGGCAGTACCGGATTGCCCAAGGGCGTAGC | |
| | | CCTACCGCACCGCACCGCTTGTGTCCGATTCAGTCATGCCCGCGACCCC | |
| | | ATCTTCGGCAACCAGATCATCCCCGACACCGCTATCCTCAGCGTGGTGC | |
| | | CATTTCACCACGGCTTCGGCATGTTCACCACGCTGGGCTACTTGATCTG | |
| | | CGGCTTTCGGGTCGTGCTCATGTACCGCTTCGAGGAGGAGCTATTCTTG | |
| | | CGCAGCTTGCAAGACTATAAGATTCAATCTGCCCTGCTGGTGCCCACAC | |
| | | TATTTAGCTTCTTCGCTAAGAGCACTCTCATCGACAAGTACGACCTAAG | |
| | | CAACTTGCACGAGATCGCCAGCGGCGGGGCGCCGCTCAGCAAGGAGGT | |
| | | AGGTGAGGCCGTGGCCAAACGCTTCCACCTACCAGGCATCCGCCAGGG | |
| | | CTACGGCCTGACAGAAACAACCAGCGCCATTCTGATCACCCCCGAAGG | |
| | | GGACGACAAGCCTGGCGCAGTAGGCAAGGTGGTGCCCTTCTTCGAGGC | |
| | | TAAGGTGGTGGACTTGGACACCGGTAAGACACTGGGTGTGAACCAGCG | |
| | | CGGCGAGCTGTGCGTCCGTGGCCCCATGATCATGAGCGGCTACGTTAAC | |
| | | AACCCCGAGGCTACAAACGCTCTCATCGACAAGGACGGCTGGCTGCAC | |
| | | AGCGGCGACATCGCCTACTGGGACGAGGACGAGCACTTCTTCATCGTG | |
| | | GACCGGCTGAAGAGCCTGATCAAATACAAGGGCTACCAGGTAGCCCCA | |
| | | GCCGAACTGGAGAGCATCCTGCTGCAACACCCCAACATCTTCGACGCCG | |
| | | GGGTCGCCGGCCTGCCCGACGACGATGCCGGCGAGCTGCCCGCCGCAG | |
| | | TCGTCGTGCTGGAACACGGTAAAACCATGACCGAGAAGGAGATCGTGG | |
| | | ACTATGTGGCCAGCCAGGTTACAACCGCCAAGAAGCTGCGCGGTGGTG | |
| | | TTGTGTTCGTGGACGAGGTGCCTAAAGGACTGACCGGCAAGTTGGACG | |
| | | CCCGCAAGATCCGCGAGATTCTCATTAAGGCCAAGAAGGGCGGCAAGA | |
| | | TCGCCGTGTGATAATAGCTCGAGGCTGGAGCCTCGGTGGCCATGCTTCT | |
| | | TGCCCCTTGGGCCTCCCCCCAGCCCCTCCTCCCCTTCCTGCACCCGTACC | |
| | | CCCGTGGTCTTTGAATAAAGTCTGAGTGGGCGGCACAATTGAAAAAAA | |
| | | AAAAAAAAAAAAAAAAAAAAAAGCATATGACTAAAAAAAAAAAAAAA | |
| | | AAAAAAAAAAAAAAAAAAAAAAAAAAAAAAAAAAAA | |
| 12D, | Fluc- | AGAAAAAAAAAAAAAAAGGAATAAACTAGTATTCTTCTGGTCCCCACAGAC | 60 |
| 12E | globin + 15A | TCAGAGAGAACCCGCCACCATGGAAGATGCCAAAAACATTAAGAAGGG | |
| | | CCCAGCGCCATTCTACCCACTCGAAGACGGGACCGCCGGCGAGCAGCT | |
| | | GCACAAAGCCATGAAGCGCTACGCCCTGGTGCCCGGCACCATCGCCTTT | |
| | | ACCGACGCACATATCGAGGTGGACATTACCTACGCCGAGTACTTCGAG | |
| | | ATGAGCGTTCGGCTGGCAGAAGCTATGAAGCGCTATGGGCTGAATACA | |
| | | AACCATCGGATCGTGGTGTGCAGCGAGAATAGCTTGCAGTTCTTCATGC | |
| | | CCGTGTTGGGTGCCCTGTTCATCGGTGTGGCTGTGGCCCCAGCTAACGA | |
| | | CATCTACAACGAGCGCGAGCTGCTGAACAGCATGGGCATCAGCCAGCC | |
| | | CACCGTCGTATTCGTGAGCAAGAAAGGGCTGCAAAAGATCCTCAACGT | |
| | | GCAAAAGAAGCTACCGATCATACAAAAGATCATCATCATGGATAGCAA | |
| | | GACCGACTACCAGGGCTTCCAAAGCATGTACACCTTCGTGACTTCCCAT | |
| | | TTGCCACCCGGCTTCAACGAGTACGACTTCGTGCCCGAGAGCTTCGACC | |
| | | GGGACAAAACCATCGCCCTGATCATGAACAGTAGTGGCAGTACCGGAT | |
| | | TGCCCAAGGGCGTAGCCCTACCGCACCGCACCGCTTGTGTCCGATTCAG | |
| | | TCATGCCCGCGACCCCATCTTCGGCAACCAGATCATCCCCGACACCGCT | |
| | | ATCCTCAGCGTGGTGCCATTTCACCACGGCTTCGGCATGTTCACCACGC | |
| | | TGGGCTACTTGATCTGCGGCTTTCGGGTCGTGCTCATGTACCGCTTCGA | |
| | | GGAGGAGCTATTCTTGCGCAGCTTGCAAGACTATAAGATTCAATCTGCC | |
| | | CTGCTGGTGCCCACACTATTTAGCTTCTTCGCTAAGAGCACTCTCATCGA | |
| | | CAAGTACGACCTAAGCAACTTGCACGAGATCGCCAGCGGGGGGCGCC | |
| | | GCTCAGCAAGGAGGTAGGTGAGGCCGTGGCCAAACGCTTCCACCTACC | |
| | | AGGCATCCGCCAGGGCTACGGCCTGACAGAAACAACCAGCGCCATTCT | |
| | | GATCACCCCCGAAGGGGACGACAAGCCTGGCGCAGTAGGCAAGGTGGT | |
| | | GCCCTTCTTCGAGGCTAAGGTGGTGGACTTGGACACCGGTAAGACACTG | |
| | | GGTGTGAACCAGCGCGGCGAGCTGTGCGTCCGTGGCCCCATGATCATG | |
| | | AGCGGCTACGTTAACAACCCCGAGGCTACAAACGCTCTCATCGACAAG | |
| | | GACGGCTGGCTGCACAGCGGCGACATCGCCTACTGGGACGAGGACGAG | |
| | | CACTTCTTCATCGTGGACCGGCTGAAGAGCCTGATCAAATACAAGGGCT | |
| | | ACCAGGTAGCCCCAGCCGAACTGGAGAGCATCCTGCTGCAACACCCCA | |
| | | ACATCTTCGACGCCGGGGTCGCCGGCCTGCCCGACGACGATGCCGGCG | |
| | | AGCTGCCCGCCGCAGTCGTCGTGCTGGAACACGGTAAAACCATGACCG | |
| | | AGAAGGAGATCGTGGACTATGTGGCCAGCCAGGTTACAACCGCCAAGA | |
| | | AGCTGCGCGGTGGTGTTGTGTTCGTGGACGAGGTGCCTAAAGGACTGAC | |
| | | CGGCAAGTTGGACGCCCGCAAGATCCGCGAGATTCTCATTAAGGCCAA | |
| | | GAAGGGCGGCAAGATCGCCGTGTGATAATAGCTCGAGGCTGGAGCCTC | |
| | | GGTGGCCATGCTTCTTGCCCCTTGGGCCTCCCCCCAGCCCCTCCTCCCCT | |
| | | TCCTGCACCCGTACCCCCGTGGTCTTTGAATAAAGTCTGAGTGGGCGGC | |
| | | ACAATTGAAAAAAAAAAAAAAAAAAAAAAAAAAAAAGCATATGACTA | |
| | | AAAAAAAAAAAAAAAAAAAAAAAAAAAAAAAAAAAAAAAAAAAAAA | |
| | | AAAA | |
| 12 | 5′ RNase H | GAATGGCGCTGGGCCCTTCTTAATGT | 61 |
| | primer_Fluc | | |

TABLE 6
Sequences used in FIG. 13

| FIG | Identi- | | SEQ ID |
|---|---|---|---|
| # | fier | Sequence (5′ to 3′) | NO: |
| 13E | Un- | — | — |
| | modified | | |
| | (S12) | | |
| 13E | PS_ddC | /5Phos/rArArArArArArArArArArArArArArArArArArArArArArArA*rA*rA*rA* | 62 |
| | (S13) | rA*rA*/3ddC/ | |
| 13E | PS_2MOE_ | /5Phos/rArArArArArArArArArArArArArArArArArArArArArArA/i2MOErA/*/ | 63 |
| | ddC | i2MOErA/*/i2MOErA/*/i2MOErA/*/i2MOErA/*/i2MOErA/*/3ddC/ | |
| | (S14) | | |

TABLE 7
Sequences used in FIG. 14

| FIG | | | SEQ ID |
|---|---|---|---|
| # | Identifier | Sequence | NO: |
| 14 | mfirefly_luc_ | /5Phos/ACTTTACGGGGTTGTTAACGTAGCCGCTAATTATTACTGAAGTCGT | 64 |
| | STARmap_546_ | AGACTAAGATA | |
| | padlock_1 | | |
| 14 | mfirefly_luc_ | /5Phos/ACTTTAGAGTGCTCTTAGCGAAGAAGCTAATTATTACTGAAGTCGT | 65 |
| | STARmap_546_ | AGACTAAGATA | |
| | padlock_2 | | |
| 14 | mfirefly_luc_ | /5Phos/ACTTTAGGGCAATCCGGTACTGCCACTAATTATTACTGAAGTCGTA | 66 |
| | STARmap_546_ | GACTAAGATA | |
| | padlock_3 | | |
| 14 | mfirefly_luc_ | /5Phos/ACTTTATTGAGGATCTTTTGCAGCCCTAATTATTACTGAAGTCGTA | 67 |
| | STARmap_546_ | GACTAAGATA | |
| | padlock_4 | | |
| 14 | mfirefly_luc_ | /5Phos/ACTTTATTGTATTCAGCCCATAGCGCTTAATTATTACTGAAGTCGTA | 68 |
| | STARmap_546_ | GACTAAGATA | |
| | padlock_5 | | |
| 14 | renilla_ | /5Phos/ACACTAATTCCGATCAGATCAGGGATAATTATTACTGAAACATACA | 69 |
| | STARmap_647_ | CTAAAGATA | |
| | padlock_1 | | |
| 14 | renilla_ | /5Phos/ACACTAGCTCGATGTGAGGCACGACGTAATTATTACTGAAACATAC | 70 |
| | STARmap_647_ | ACTAAAGATA | |
| | padlock_2 | | |
| 14 | renilla_ | /5Phos/ACACTATTAGCTCCCTCGACAATAGCGTAATTATTACTGAAACATA | 71 |
| | STARmap_647_ | CACTAAAGATA | |
| | padlock_3 | | |
| 14 | renilla_ | /5Phos/ACACTAAGCCATTCCCGCTCTTGCCGAATTATTACTGAAACATACA | 72 |
| | STARmap_647_ | CTAAAGATA | |
| | padlock_4 | | |
| 14 | mfirefly_luc_ | /5Phos/AAGATAACATCAGCATCCTACGGGGTTGTTAACGTAGCCGCTATTA | 73 |
| | RIBO_padlock_ | CATCAGCATACACTA | |
| | 1 | | |
| 14 | mfirefly_luc_ | /5Phos/AAGATAACATCAGCATCCTAGAGTGCTCTTAGCGAAGAAGCTATT | 74 |
| | RIBO_padlock_ | ACATCAGCATACACTA | |
| | 2 | | |
| 14 | mfirefly_luc_ | /5Phos/AAGATAACATCAGCATCCTAGGGCAATCCGGTACTGCCACTATTAC | 75 |
| | RIBO_padlock_ | ATCAGCATACACTA | |
| | 3 | | |
| 14 | mfirefly_luc_ | /5Phos/AAGATAACATCAGCATCCTATTGAGGATCTTTTGCAGCCCTATTAC | 76 |
| | RIBO_padlock_ | ATCAGCATACACTA | |
| | 4 | | |
| 14 | mfirefly_luc_ | /5Phos/AAGATAACATCAGCATCCTATTGTATTCAGCCCATAGCGCTTATTA | 77 |
| | RIBO_padlock_ | CATCAGCATACACTA | |
| | 5 | | |
| 14 | mfirefly_luc_ | TGTCGATGAGAGCGTTTGTAGCGTGGATGCTGAT | 78 |
| | RIBO_primer_ | | |
| | 1 | | |
| 14 | mfirefly_luc_ | TGCTTAGGTCGTACTTGTCGATGTGGATGCTGAT | 79 |
| | RIBO_primer_ | | |
| | 2 | | |
| 14 | mfirefly_luc_ | CGGTGCGGTAGGGCTACGCCCTGTGGATGCTGAT | 80 |
| | RIBO_primer_ | | |
| | 3 | | |
| 14 | mfirefly_luc_ | TGATCGGTAGCTTCTTTTGCACGTGGATGCTGAT | 81 |
| | RIBO_primer_ | | |
| | 4 | | |
| 14 | mfirefly_luc_ | CGCTGCACACCACGATCCGATGGTGGATGCTGAT | 82 |
| | RIBO_primer_ | | |
| | 5 | | |
| 14 | RIBO_ | ACAAAATAGAACCGCGGTCCTATTCAAAAAAAAAAAAAAAAAAAAAAAA | 83 |
| | splintprobe_1 | AAAAAAAAAAAAAAAAAAAAAAAAAATATCTTTAGT*G*T*/3InvdT/ | |
| 14 | RIBO_ | CATCGTTTATGGTCGGAACTACGACAAAAAAAAAAAAAAAAAAAAAAAA | 84 |
| | splintprobe_2 | AAAAAAAAAAAAAAAAAAAAAAAAAATATCTTTAGT*G*T*/3InvdT/ | |
| 14 | RIBO_ | AGGTTTCCCGTGTTGAGTCAAATTAAAAAAAAAAAAAAAAAAAAAAAAA | 85 |
| | splintprobe_3 | AAAAAAAAAAAAAAAAAAAAAAAAATATCTTTAGT*G*T*/3InvdT/ | |
| 14 | RIBO_ | TGTTATTGCTCAATCTCGGGTGGCTAAAAAAAAAAAAAAAAAAAAAAAA | 86 |
| | splintprobe_4 | AAAAAAAAAAAAAAAAAAAAAAAAAATATCTTTAGT*G*T*/3InvdT/ | |
| 14 | RIBO_ | AGATAGTCAAGTTCGACCGTCTTCTAAAAAAAAAAAAAAAAAAAAAAAA | 87 |
| | splintprobe_5 | AAAAAAAAAAAAAAAAAAAAAAAAAATATCTTTAGT*G*T*/3InvdT/ | |
| 14 | AF546 | TCGTAGACTAAGATAACAT | 88 |
| 14 | AF647 | CATACACTAAAGATAACAT | 89 |

TABLE 8
Constructs used in FIG. 14

| | | | | | SEQ |
|---|---|---|---|---|---|
| | Identi- | | Sequence (5′ to 3′)-Azide | Sequence (5′ to 3′)-Alkyne | ID |
| FIG # | fier | 5′ Cap | Oligo | Oligo | NO: |
| 14D | 2xLNAm7G | LNAm7G | /5Phos/rArGrArGrArUrArA/ | /5Phos/rArGrArUrArArA/i5OCTdU/ | 120 |
| | (42) | | 3AzideN/ | rArArArArArA | |
| 14D, | 2xm7G | m7G | /5Phos/rArGrArGrArUrArA/ | /5Phos/rArGrArUrArArA/i5OCTdU/ | 121 |
| G-J | (31) | | 3AzideN/ | rArArArArArA | |
| 14D, E | LNAm7G- | LNAm7G | — | /5Phos/+ArGrArGrArArUrArArArA | 122 |
| | LNA | | | rA | |
| | (22) | | | | |
| 14D, E | LNAm7G- | LNAm7G | — | /5Phos/mAmGmAmGmAmArUrAr | 123 |
| | 2OMex6 | | | ArArArA | |
| | (24) | | | | |
| 14D, E | m7G-LNA | m7G | — | /5Phos/+ArGrArGrArArUrArArArA | 124 |
| | (13) | | | rA | |
| 14D, E | m7G-rA | m7G | — | /5Phos/rArGrArGrArArUrArArArAr | 125 |
| | (1) | | | A | |
| 14D, | 1xm7G-OU | m7G | | /5Phos/rArGrArUrArArA/i5OCTdU/ | 126 |
| G-J | (28) | | | rArArArArArA | |
| 14D, E | mm7G- | m7G | — | /5Phos/mAmGmAmGmAmArUrAr | 127 |
| | 6x2OMe | | | ArArArA | |
| | (15) | | | | |
| 14E | m7G- | m7G | — | /5Phos/mAmGmAmGmAmAmUm | 128 |
| | 23x2OMe | | | AmAmAmAmAmAmGmGmG | |
| | (43) | | | mAmAmUmAmAmAmC | |
| 14E | LNAm7G- | LNAm7G | — | /5Phos/rArGrArGrArArUrArArArAr | 129 |
| | rA | | | A | |
| | (21) | | | | |
| 14E | m7G- | m7G | — | /5Phos/mArGrArGrArArUrArArAr | 130 |
| | 2OMe | | | ArA | |
| | (11) | | | | |

TABLE 9
Sequences used in FIG. 15

| | | | SEQ ID |
|---|---|---|---|
| FIG # | Identifier | Sequence (5′ to 3′) | NO: |
| 15 | eIF3D_siRNA 1 | TACCAGCGGAATCGAATGAAGA | 90 |
| 15 | eIF3D_siRNA 2 | TCAGTTGATGAAGATGCGCTA | 91 |
| 15 | eIF3D_siRNA 3 | TACGAGGGCCTTTGATGCTTA | 92 |
| 15 | eIF3D_siRNA 4 | CGCCTCTGTTGCGTACCGTTA | 93 |

TABLE 10
Sequences used in FIG. 16

| FIG | | | SEQ ID |
|---|---|---|---|
| # | Identifier | Sequence (5′ to 3′) | NO: |
| 16D | Alkyne oligo | /5Phos/+ArGrArGrArArA/35OCTdU/ | |
| 16E | RNase H | GCTAGCTCCAGGGTGTGGC | 95 |
| | primer | | |
| | upstream | | |
| 16E | RNase H | GGGACCAGAAGAATACTAGTTTATTCGAT | 96 |
| | primer | | |
| | downstream | | |
| 16F | circRNA | GGGCGAATTGGAGCTCGGGAATTCTAGAGAAAATTTCGTCTGGATTAGTT | 97 |
| | (iHRV) | ACTTATCGTGTAAAATCTGATAAATGGAATTGGTTCTACATAAATGCCTA | |
| | | ACGACTATCCCTTTGGGGAGTAGGGTCAAGTGACTCGAAACGATAGACA | |
| | | ACTTGCTTTAACAAGTTGGAGATATAGTCTGCTCTGCATGGTGACATGCA | |
| | | GCTGGATATAATTCCGGGGTAAGATTAACGACCTTATCTGAACATAACGC | |
| | | TACCGTTTAATATTGCGTCATATAAAAAAAAAAAACCAAAAAAAAAAAC | |
| | | AAAAAAAAAAAATAATTGACTAATATCTTAAAACAGCGGATGGGTACCC | |
| | | CACCATCCGACCCACTGGGTGTAGTACTCTGGTACTTCGTACCTTTGTAC | |
| | | GCCTGTTCTTCCCATTGTACCCTTCCTGAACTTCCAACCCAAGTAACGTTA | |
| | | GAAGCTCAACATTTAGTACAACAGGAAGCACCACATCCAGTGGTGTTTA | |
| | | GTACAAGCACTTCTGTTTCCCCGGAGCGAGGTATAGGCTGTACCCACTGC | |
| | | CAAAAACCTTTAACCGTTATCCGCCAACCAACTACGTAAAAGCTAGTAGT | |
| | | ATTATGTTTTTAACTAGGCGTTCGATCAGGTGGATTTCCCCTCCACTAGTT | |
| | | TGGTCGATGAGGCTAGGAATTCCCCACGGGTGACCGTGTCCTAGCCTGCG | |
| | | TGGCGGCCAACCCAGCCCACTCACTATTTGTTTTCGCGCCCAGTTGCAAA | |
| | | AAGTGTCGGGGCTGGGACGCCTTTTTATAGACATGGTGTGAAGACTCGC | |
| | | ATGTGCTTGGTTGTGATTCCTCCGGCCCCTGAATGCGGCTAACCTTAACC | |
| | | CTGGAGCCTTGTGTCACAAACCAGTGATGATAAGGTCGTAATGAGCAAT | |
| | | TCCGGGACGGGACCGACTACTTTGGGTGTCCGTGTTTCTTATTTTTCTTAT | |
| | | TATTGTCTTATGGTCACAGCATATATATAACATATACTGTGATCATGGAT | |
| | | GGTCTTCACACTCGAAGATTTCGTTGGGGACTGGCGACAGACAGCCGGC | |
| | | TACAACCTGGACCAAGTCCTTGAACAGGGAGGTGTGTCCAGTTTGTTTCA | |
| | | GAATCTCGGGGTGTCCGTAACTCCGATCCAAAGGATTGTCCTGAGCGGTG | |
| | | AAAATGGGCTGAAGATCGACATCCATGTCATCATCCCGTATGAAGGTCT | |
| | | GAGCGGCGACCAAATGGGCCAGATCGAAAAAATTTTTAAGGTGGTGTAC | |
| | | CCTGTGGATGATCATCACTTTAAGGTGATCCTGCACTATGGCACACTGGT | |
| | | AATCGACGGGGTTACGCCGAACATGATCGACTATTTCGGACGGCCGTAT | |
| | | GAAGGCATCGCCGTGTTCGACGGCAAAAAGATCACTGTAACAGGGACCC | |
| | | TGTGGAACGGCAACAAAATTATCGACGAGCGCCTGATCAACCCCGACGG | |
| | | CTCCCTGCTGTTCCGAGTAACCATCAACGGAGTGACCGGCTGGCGGCTGT | |
| | | GCGAACGCATTCTGGCGTAGTAGTCTCGAGCTGGTACTGCATGCACGCA | |
| | | ATGCTAGCTGCCCCTTTCCCGTCCTGGGTACCCCGAGTCTCCCCCGACCT | |
| | | CGGGTCCCAGGTATGCTCCCACCTCCACCTGCCCCACTCACCACCTCTGC | |
| | | TAGTTCCAGACACCTCCCAAGCACGCAGCAATGCAGCTCAAAACGCTTA | |
| | | GCCTAGCCACACCCCCACGGGAAACAGCAGTGATTAACCTTTAGCAATA | |
| | | AACGAAAGTTTAACTAAGCTATACTAACCCCAGGGTTGGTCAATTTCGTG | |
| | | CCAGCCACACCCTGGAGCTAGCCTCAGTAGATGTTTTCTTGGGTTAATTG | |
| | | AGGCCTGAGTATAAGGTGACTTATACTTGTAATCTATCTAAACGGGGAAC | |
| | | CTCTCTAGTAGACAATCCCGTGCTAAATTGTAGGACTGCCCTTTAATAAA | |
| | | TACTTCTATATTTAAAGAGGTATTTATGAAAAGCGGAATTTATCAGATTA | |
| | | AAAATACTTTCTCTAGAGTCGACCTGCAGG | |
| 16F | circRNA | ACTTGCTTTAACAAGTTGGAGATATAGTCTGCTCTGCATGGTGACATGCA | 98 |
| | (globin) | GCTGGATATAATTCCGGGGTAAGATTAACGACCTTATCTGAACATAACGC | |
| | | TACCGTTTAATATTGCGTCATATAAAAAAAAAAACCAAAAAAAAAAACA | |
| | | AAAAAAAAAATAATTGACTAATTAAGCAGACTGTAAATCTGCTAAAATC | |
| | | GAATAAACTAGTATTCTTCTGGTCCCCACAGACTCAGAGAGAACCCGCC | |
| | | ACCATGGTCTTCACACTCGAAGATTTCGTTGGGGACTGGCGACAGACAG | |
| | | CCGGCTACAACCTGGACCAAGTCCTTGAACAGGGAGGTGTGTCCAGTTT | |
| | | GTTTCAGAATCTCGGGGTGTCCGTAACTCCGATCCAAAGGATTGTCCTGA | |
| | | GCGGTGAAAATGGGCTGAAGATCGACATCCATGTCATCATCCCGTATGA | |
| | | AGGTCTGAGCGGCGACCAAATGGGCCAGATCGAAAAAATTTTTAAGGTG | |
| | | GTGTACCCTGTGGATGATCATCACTTTAAGGTGATCCTGCACTATGGCAC | |
| | | ACTGGTAATCGACGGGGTTACGCCGAACATGATCGACTATTTCGGACGG | |
| | | CCGTATGAAGGCATCGCCGTGTTCGACGGCAAAAAGATCACTGTAACAG | |
| | | GGACCCTGTGGAACGGCAACAAAATTATCGACGAGCGCCTGATCAACCC | |
| | | CGACGGCTCCCTGCTGTTCCGAGTAACCATCAACGGAGTGACCGGCTGG | |
| | | CGGCTGTGCGAACGCATTCTGGCGTAGTAGTCTCGAGCTGGTACTGCATG | |
| | | CACGCAATGCTAGCTGCCCCTTTCCCGTCCTGGGTACCCCGAGTCTCCCC | |
| | | CGACCTCGGGTCCCAGGTATGCTCCCACCTCCACCTGCCCCACTCACCAC | |
| | | CTCTGCTAGTTCCAGACACCTCCCAAGCACGCAGCAATGCAGCTCAAAA | |
| | | CGCTTAGCCTAGCCACACCCCCACGGGAAACAGCAGTGATTAACCTTTA | |
| | | GCAATAAACGAAAGTTTAACTAAGCTATACTAACCCCAGGGTTGGTCAA | |
| | | TTTCGTGCCAGCCACACCCTGGAGCTAGCAAAAAAAAAAAAAAAAAAAA | |
| | | AAAAAAAAAAAAAAAAAAACTCAGTAGATGTTTTCTTGGGTTAATTGAG | |
| | | GCCTGAGTATAAGGTGACTTATACTTGTAATCTATCTAAACGGGGAACCT | |
| | | CTCTAGTAGACAATCCCGTGCTAAATTGTAGGACTGCCCTTTAATAAATA | |
| | | CTTCTATATTTAAAGAGGTATTTATGAAAAGCGGAATTTATCAGATTAAA | |
| | | AATACTTTCTCTAGAGTCGACCTGCAG | |
| 16F | QRNA | ACTTGCTTTAACAAGTTGGAGATATAGTCTGCTCTGCATGGTGACATGCA | 99 |
| | (globin) | GCTGGATATAATTCCGGGGTAAGATTAACGACCTTATCTGAACATAACGC | |
| | | TACCGTTTAATATTGCGTCATATAAAAAAAAAAACCAAAAAAAAAAACA | |
| | | AAAAAAAAAATAATTGACTAATTAAGCAGACTGTAAATCTGCTAAAATC | |
| | | GAATAAACTAGTATTCTTCTGGTCCCCACAGACTCAGAGAGAACCCGCC | |
| | | ACCATGGTCTTCACACTCGAAGATTTCGTTGGGGACTGGCGACAGACAG | |
| | | CCGGCTACAACCTGGACCAAGTCCTTGAACAGGGAGGTGTGTCCAGTTT | |
| | | GTTTCAGAATCTCGGGGTGTCCGTAACTCCGATCCAAAGGATTGTCCTGA | |
| | | GCGGTGAAAATGGGCTGAAGATCGACATCCATGTCATCATCCCGTATGA | |
| | | AGGTCTGAGCGGCGACCAAATGGGCCAGATCGAAAAAATTTTTAAGGTG | |
| | | GTGTACCCTGTGGATGATCATCACTTTAAGGTGATCCTGCACTATGGCAC | |
| | | ACTGGTAATCGACGGGGTTACGCCGAACATGATCGACTATTTCGGACGG | |
| | | CCGTATGAAGGCATCGCCGTGTTCGACGGCAAAAAGATCACTGTAACAG | |
| | | GGACCCTGTGGAACGGCAACAAAATTATCGACGAGCGCCTGATCAACCC | |
| | | CGACGGCTCCCTGCTGTTCCGAGTAACCATCAACGGAGTGACCGGCTGG | |
| | | CGGCTGTGCGAACGCATTCTGGCGTAGTAGTCTCGAGCTGGTACTGCATG | |
| | | CACGCAATGCTAGCTGCCCCTTTCCCGTCCTGGGTACCCCGAGTCTCCCC | |
| | | CGACCTCGGGTCCCAGGTATGCTCCCACCTCCACCTGCCCCACTCACCAC | |
| | | CTCTGCTAGTTCCAGACACCTCCCAAGCACGCAGCAATGCAGCTCAAAA | |
| | | CGCTTAGCCTAGCCACACCCCCACGGGAAACAGCAGTGATTAACCTTTA | |
| | | GCAATAAACGAAAGTTTAACTAAGCTATACTAACCCCAGGGTTGGTCAA | |
| | | TTTCGTGCCAGCCACACCCTGGAGCTAGCAAAAAAAAAAAAAAAAAAAA | |
| | | AAAAAAAAAAAAAAAAAAACTCAGTAGATGTTTTCTTGGGTTAATTGAG | |
| | | GCCTGAGTATAAGGTGACTTATACTTGTAATCTATCTAAACGGGGAACCT | |
| | | CTCTAGTAGACAATCCCGTGCTAAATTGTAGGACTGCCCTTTAATAAATA | |
| | | CTTCTATATTTAAAGAGGTATTTATGAAAAGCGGAATTTATCAGATTAAA | |
| | | AATACTTTCTCTAGAGTCGACCTGCAG | |
| 16G | mRNA | AGGAATAAACTAGTATTCTTCTGGTCCCCACAGACTCAGAGAGAACCCG | 100 |
| | (globin, U) | CCACCATGGGATCCGTCTTCACACTCGAAGATTTCGTTGGGGACTGGCGA | |
| | | CAGACAGCCGGCTACAACCTGGACCAAGTCCTTGAACAGGGAGGTGTGT | |
| | | CCAGTTTGTTTCAGAATCTCGGGGTGTCCGTAACTCCGATCCAAAGGATT | |
| | | GTCCTGAGCGGTGAAAATGGGCTGAAGATCGACATCCATGTCATCATCC | |
| | | CGTATGAAGGTCTGAGCGGCGACCAAATGGGCCAGATCGAAAAAATTTT | |
| | | TAAGGTGGTGTACCCTGTGGATGATCATCACTTTAAGGTGATCCTGCACT | |
| | | ATGGCACACTGGTAATCGACGGGGTTACGCCGAACATGATCGACTATTTC | |
| | | GGACGGCCGTATGAAGGCATCGCCGTGTTCGACGGCAAAAAGATCACTG | |
| | | TAACAGGGACCCTGTGGAACGGCAACAAAATTATCGACGAGCGCCTGAT | |
| | | CAACCCCGACGGCTCCCTGCTGTTCCGAGTAACCATCAACGGAGTGACC | |
| | | GGCTGGCGGCTGTGCGAACGCATTCTGGCGTAGTGATAATAGCTCGAGG | |
| | | CTGGAGCCTCGGTGGCCATGCTTCTTGCCCCTTGGGCCTCCCCCCAGCCC | |
| | | CTCCTCCCCTTCCTGCACCCGTACCCCCGTGGTCTTTGAATAAAGTCTGA | |
| | | GTGGGCGGCACAATTGAAAAAAAAAAAAAAAAAAAAAAAAAAAAAAA | |
| | | AAGCATATGACTAAAAAAAAAAAAAAAAAAAAAAAAAAAAAAAAAAA | |
| | | AAAAAAAAAAAAAAAAAAAAAAAAA | |

TABLE 11
FIG. 16 Constructs

| | | 5′ | 5′UTR | 3′UTR | SEQ ID |
|---|---|---|---|---|---|
| FIG # | Identifier | Cap | | | NO: |
| 16F, G | circRNA + | - | rUrUrArArArArCrArGrCrGrGr | rCrUrCrGrArGrCrUrGrGrUrArCr | 131 |
| | IHRV-IRES | | ArUrGrGrGrUrArCrCrCrCrArC | UrGrCrArUrGrCrArCrGrCrArAr | |
| | (50) | | rCrArUrCrCrGrArCrCrCrArCrU | UrGrCrUrArGrCrUrGrCrCrCrCrU | |
| | | | rGrGrGrUrGrUrArGrUrArCrUr | rUrUrCrCrCrGrUrCrCrUrGrGrGr | |
| | | | CrUrGrGrUrArCrUrUrCrGrUrA | UrArCrCrCrCrGrArGrUrCrUrCrC | |
| | | | rCrCrUrUrUrGrUrArCrGrCrCr | rCrCrCrGrArCrCrUrCrGrGrGrUr | |
| | | | UrGrUrUrCrUrUrCrCrCrArUrU | CrCrCrArGrGrUrArUrGrCrUrCrC | |
| | | | rGrUrArCrCrCrUrUrCrCrUrGr | rCrArCrCrUrCrCrArCrCrUrGrCr | |
| | | | ArArCrUrUrCrCrArArCrCrCrAr | CrCrCrArCrUrCrArCrCrArCrCrU | |
| | | | ArGrUrArArCrGrUrUrArGrArA | rCrUrGrCrUrArGrUrUrCrCrArGr | |
| | | | rGrCrUrCrArArCrArUrUrUrAr | ArCrArCrCrUrCrCrCrArArGrCrA | |
| | | | GrUrArCrArArCrArGrGrArArG | rCrGrCrArGrCrArArUrGrCrArGr | |
| | | | rCrArCrCrArCrArUrCrCrArGrU | CrUrCrArArArArCrGrCrUrUrAr | |
| | | | rGrGrUrGrUrUrUrArGrUrArCr | GrCrCrUrArGrCrCrArCrArCrCrC | |
| | | | ArArGrCrArCrUrUrCrUrGrUrU | rCrCrArCrGrGrGrArArArCrArGr | |
| | | | rUrCrCrCrCrGrGrArGrCrGrAr | CrArGrUrGrArUrUrArArCrCrUr | |
| | | | GrGrUrArUrArGrGrCrUrGrUrA | UrUrArGrCrArArUrArArArCrGr | |
| | | | rCrCrCrArCrUrGrCrCrArArArA | ArArArGrUrUrUrArArCrUrArAr | |
| | | | rArCrCrUrUrUrArArCrCrGrUr | GrCrUrArUrArCrUrArArCrCrCrC | |
| | | | UrArUrCrCrGrCrCrArArCrCrAr | rArGrGrGrUrUrGrGrUrCrArArUr | |
| | | | ArCrUrArCrGrUrArArArArGrC | UrUrCrGrUrGrCrCrArGrCrCrArC | |
| | | | rUrArGrUrArGrUrArUrUrArUr | rArCrCrCrUrGrGrArGrCrUrArGr | |
| | | | GrUrUrUrUrUrArArCrUrArGrG | C | |
| | | | rCrGrUrUrCrGrArUrCrArGrGr | | |
| | | | UrGrGrArUrUrUrCrCrCrCrUrC | | |
| | | | rCrArCrUrArGrUrUrUrGrGrUr | | |
| | | | CrGrArUrGrArGrGrCrUrArGrG | | |
| | | | rArArUrUrCrCrCrCrArCrGrGr | | |
| | | | GrUrGrArCrCrGrUrGrUrCrCrU | | |
| | | | rArGrCrCrUrGrCrGrUrGrGrCr | | |
| | | | GrGrCrCrArArCrCrCrArGrCrCr | | |
| | | | CrArCrUrCrArCrUrArUrUrUrG | | |
| | | | rUrUrUrUrCrGrCrGrCrCrCrAr | | |
| | | | GrUrUrGrCrArArArArArGrUrG | | |
| | | | rUrCrGrGrGrGrCrUrGrGrGrAr | | |
| | | | CrGrCrCrUrUrUrUrUrArUrArG | | |
| | | | rArCrArUrGrGrUrGrUrGrArAr | | |
| | | | GrArCrUrCrGrCrArUrGrUrGrC | | |
| | | | rUrUrGrGrUrUrGrUrGrArUrUr | | |
| | | | CrCrUrCrCrGrGrCrCrCrCrUrGr | | |
| | | | ArArUrGrCrGrGrCrUrArArCrC | | |
| | | | rUrUrArArCrCrCrUrGrGrArGr | | |
| | | | CrCrUrUrGrUrGrUrCrArCrArA | | |
| | | | rArCrCrArGrUrGrArUrGrArUr | | |
| | | | ArArGrGrUrCrGrUrArArUrGrA | | |
| | | | rGrCrArArUrUrCrCrGrGrGrAr | | |
| | | | CrGrGrGrArCrCrGrArCrUrArC | | |
| | | | rUrUrUrGrGrGrUrGrUrCrCrGr | | |
| | | | UrGrUrUrUrCrUrUrArUrUrUrU | | |
| | | | rUrCrUrUrArUrUrArUrUrGrUr | | |
| | | | CrUrUrArUrGrGrUrCrArCrArG | | |
| | | | rCrArUrArUrArUrArUrArArCr | | |
| | | | ArUrArUrArCrUrGrUrGrArUrC | | |
| | | | (SEQ ID NO: 173) | | |
| 16F, G | circRNA + | | rArCrUrUrGrCrUrUrUrArArCr | rUrArGrUrCrUrCrGrArGrCrUrGr | 132 |
| | globin UTRs | | ArArGrUrUrGrGrArGrArUrArU | GrUrArCrUrGrCrArUrGrCrArCr | |
| | (S18) | | rArGrUrCrUrGrCrUrCrUrGrCr | GrCrArArUrGrCrUrArGrCrUrGr | |
| | | | ArUrGrGrUrGrArCrArUrGrCrA | CrCrCrCrUrUrUrCrCrCrGrUrCrC | |
| | | | rGrCrUrGrGrArUrArUrArArUr | rUrGrGrGrUrArCrCrCrCrGrArGr | |
| | | | UrCrCrGrGrGrGrUrArArGrArU | UrCrUrCrCrCrCrCrGrArCrCrUrC | |
| | | | rUrArArCrGrArCrCrUrUrArUr | rGrGrGrUrCrCrCrArGrGrUrArUr | |
| | | | CrUrGrArArCrArUrArArCrGrC | GrCrUrCrCrCrArCrCrUrCrCrArC | |
| | | | rUrArCrCrGrUrUrUrArArUrAr | rCrUrGrCrCrCrCrArCrUrCrArCr | |
| | | | UrUrGrCrGrUrCrArUrArUrArA | CrArCrCrUrCrUrGrCrUrArGrUrU | |
| | | | rArArArArArArArArArCrCrAr | rCrCrArGrArCrArCrCrUrCrCrCr | |
| | | | ArArArArArArArArArArCrArA | ArArGrCrArCrGrCrArGrCrArAr | |
| | | | rArArArArArArArArArUrArAr | UrGrCrArGrCrUrCrArArArArCr | |
| | | | UrUrGrArCrUrArArUrUrArArG | GrCrUrUrArGrCrCrUrArGrCrCrA | |
| | | | rCrArGrArCrUrGrUrArArArUr | rCrArCrCrCrCrCrArCrGrGrGrAr | |
| | | | CrUrGrCrUrArArArArUrCrGrA | ArArCrArGrCrArGrUrGrArUrUr | |
| | | | rArUrArArArCrUrArGrUrArUr | ArArCrCrUrUrUrArGrCrArArUr | |
| | | | UrCrUrUrCrUrGrGrUrCrCrCrCr | ArArArCrGrArArArGrUrUrUrAr | |
| | | | ArCrArGrArCrUrCrArGrArGrA | ArCrUrArArGrCrUrArUrArCrUr | |
| | | | rGrArArCrCrCrGrCrCrArCrC | ArArCrCrCrCrArGrGrGrUrUrGr | |
| | | | (SEQ ID NO: 174) | GrUrCrArArUrUrUrCrGrUrGrCr | |
| | | | | CrArGrCrCrArCrArCrCrCrUrGrG | |
| | | | | rArGrCrUrArGrCrArArArArArAr | |
| | | | | ArArArArArArArArArArArArAr | |
| | | | | ArArArArArArArArArArArArAr | |
| | | | | ArArArArArArArCrUrCrArGrUr | |
| | | | | ArGrArUrGrUrUrUrUrCrUrUrGr | |
| | | | | GrGrUrUrArArUrUrGrArGrGrCr | |
| | | | | CrUrGrArGrUrArUrArArGrGrUr | |
| | | | | GrArCrUrUrArUrArCrUrUrGrUr | |
| | | | | ArArUrCrUrArUrCrUrArArArCr | |
| | | | | GrGrGrGrArArCrCrUrCrUrCrUr | |
| | | | | ArGrUrArGrArCrArArUrCrCrCr | |
| | | | | GrUrGrCrUrArArArUrUrGrUrAr | |
| | | | | GrGrArCrUrGrCrCrCrUrUrUrAr | |
| | | | | ArUrArArArUrArCrUrUrCrUrAr | |
| | | | | UrArUrUrUrArArArGrArGrGrUr | |
| | | | | ArUrUrUrArUrGrArArArArGrCr | |
| | | | | GrGrArArUrUrUrArUrCrArGrAr | |
| | | | | UrUrArArArArArUrArCrUrUrUr | |
| | | | | CrUrCrUrArGrArGrUrCrGrArCrC | |
| | | | | rUrGrCrArG | |
| 16F, G | QRNA + | Bn7G | rArCrUrUrGrCrUrUrUrArArCr | rUrArGrUrCrUrCrGrArGrCrUrGr | 133 |
| | globin UTRs | | ArArGrUrUrGrGrArGrArUrArU | GrUrArCrUrGrCrArUrGrCrArCr | |
| | (S19) | | rArGrUrCrUrGrCrUrCrUrGrCr | GrCrArArUrGrCrUrArGrCrUrGr | |
| | | | ArUrGrGrUrGrArCrArUrGrCrA | CrCrCrCrUrUrUrCrCrCrGrUrCrC | |
| | | | rGrCrUrGrGrArUrArUrArArUr | rUrGrGrGrUrArCrCrCrCrGrArGr | |
| | | | UrCrCrGrGrGrGrUrArArGrArU | UrCrUrCrCrCrCrCrGrArCrCrUrC | |
| | | | rUrArArCrGrArCrCrUrUrArUr | rGrGrGrUrCrCrCrArGrGrUrArUr | |
| | | | CrUrGrArArCrArUrArArCrGrC | GrCrUrCrCrCrArCrCrUrCrCrArC | |
| | | | rUrArCrCrGrUrUrUrArArUrAr | rCrUrGrCrCrCrCrArCrUrCrArCr | |
| | | | UrUrGrCrGrUrCrArUrArUrArA | CrArCrCrUrCrUrGrCrUrArGrUrU | |
| | | | rArArArArArArArArArCrCrAr | rCrCrArGrArCrArCrCrUrCrCrCr | |
| | | | ArArArArArArArArArArCrArA | ArArGrCrArCrGrCrArGrCrArAr | |
| | | | rArArArArArArArArArUrArAr | UrGrCrArGrCrUrCrArArArArCr | |
| | | | UrUrGrArCrUrArArUrUrArArG | GrCrUrUrArGrCrCrUrArGrCrCrA | |
| | | | rCrArGrArCrUrGrUrArArArUr | rCrArCrCrCrCrCrArCrGrGrGrAr | |
| | | | CrUrGrCrUrArArArArUrCrGrA | ArArCrArGrCrArGrUrGrArUrUr | |
| | | | rArUrArArArCrUrArGrUrArUr | ArArCrCrUrUrUrArGrCrArArUr | |
| | | | UrCrUrUrCrUrGrGrUrCrCrCrCr | ArArArCrGrArArArGrUrUrUrAr | |
| | | | ArCrArGrArCrUrCrArGrArGrA | ArCrUrArArGrCrUrArUrArCrUr | |
| | | | rGrArArCrCrCrGrCrCrArCrC | ArArCrCrCrCrArGrGrGrUrUrGr | |
| | | | (SEQ ID NO: 174) | GrUrCrArArUrUrUrCrGrUrGrCr | |
| | | | | CrArGrCrCrArCrArCrCrCrUrGrG | |
| | | | | rArGrCrUrArGrCrArArArArArAr | |
| | | | | ArArArArArArArArArArArArAr | |
| | | | | ArArArArArArArArArArArArAr | |
| | | | | ArArArArArArArCrUrCrArGrUr | |
| | | | | ArGrArUrGrUrUrUrUrCrUrUrGr | |
| | | | | GrGrUrUrArArUrUrGrArGrGrCr | |
| | | | | CrUrGrArGrUrArUrArArGrGrUr | |
| | | | | GrArCrUrUrArUrArCrUrUrGrUr | |
| | | | | ArArUrCrUrArUrCrUrArArArCr | |
| | | | | GrGrGrGrArArCrCrUrCrUrCrUr | |
| | | | | ArGrUrArGrArCrArArUrCrCrCr | |
| | | | | GrUrGrCrUrArArArUrUrGrUrAr | |
| | | | | GrGrArCrUrGrCrCrCrUrUrUrAr | |
| | | | | ArUrArArArUrArCrUrUrCrUrAr | |
| | | | | UrArUrUrUrArArArGrArGrGrUr | |
| | | | | ArUrUrUrArUrGrArArArArGrCr | |
| | | | | GrGrArArUrUrUrArUrCrArGrAr | |
| | | | | UrUrArArArArArUrArCrUrUrUr | |
| | | | | CrUrCrUrArGrArGrUrCrGrArCrC | |
| | | | | rUrGrCrArG | |
| 16F, G | (0) | m7G | rArGrArArUrArArArCrUrAr | rGrArUrArArUrArGrCrUrCrGrA | 134 |
| | | | GrUrArUrUrCrUrUrCrUrGr | rGrGrCrUrGrGrArGrCrCrUr | |
| | | | GrUrCrCrCrCrArCrArGrArC | CrGrGrUrGrGrCrCrArUrGr | |
| | | | rUrCrArGrArGrArGrArArCr | CrUrUrCrUrUrGrCrCrCrCr | |
| | | | CrCrGrCrCrArCrC (SEQ ID | UrUrGrGrGrCrCrUrCrCrCr | |
| | | | NO: 175) | CrCrCrArGrCrCrCrCrUrCrC | |
| | | | | rUrCrCrCrCrUrUrCrCrUrGr | |
| | | | | CrArCrCrCrGrUrArCrCrCrC | |
| | | | | rCrGrUrGrGrUrCrUrUrUrGr | |
| | | | | ArArUrArArArGrUrCrUrGr | |
| | | | | ArGrUrGrGrGrCrGrGrCrAr | |
| | | | | CrArArUrUrGrArArArArAr | |
| | | | | ArArArArArArArArArArAr | |
| | | | | ArArArArArArArArArArAr | |
| | | | | ArArGrCrArUrArUrGrArCr | |
| | | | | UrArArArArArArArArArAr | |
| | | | | ArArArArArArArArArArAr | |
| | | | | ArArArArArArArArArArAr | |
| | | | | ArArArArArArArArArArAr | |
| | | | | ArArArArArArArArArArAr | |
| | | | | A | |

TABLE 12
Sequences used in FIG. 18

| FIG | | | SEQ ID |
|---|---|---|---|
| # | Identifier | Sequence (5′ to 3′) | NO: |
| 18 | 5′ Rnase H | GCCAGTCCCCAACGAAATC | 103 |
| | primer_Nluc | | |
| 18 | 5′ Rnase H | GCAACTGGCCCGAAAGCACTGGTACTGAAGGAG | 104 |
| | primer_hEPO | | |

TABLE 13
Sequences used in FIG. 19

| FIG | | | SEQ ID |
|---|---|---|---|
| # | Identifier | Sequence (5′ to 3′) | NO: |
| 19 | NanoLuc | AGAAAAAAAAAAAAAATAAACTAGTATTCTTCTGGTCCCCACAGACTCA | 105 |
| | | GAGAGAACCCGCCACCATGGGATCCATGGGATCCGTCTTCACACTCGAA | |
| | | GATTTCGTTGGGGACTGGCGACAGACAGCCGGCTACAACCTGGACCAA | |
| | | GTCCTTGAACAGGGAGGTGTGTCCAGTTTGTTTCAGAATCTCGGGGTGT | |
| | | CCGTAACTCCGATCCAAAGGATTGTCCTGAGCGGTGAAAATGGGCTGAA | |
| | | GATCGACATCCATGTCATCATCCCGTATGAAGGTCTGAGCGGCGACCAA | |
| | | ATGGGCCAGATCGAAAAAATTTTTAAGGTGGTGTACCCTGTGGATGATC | |
| | | ATCACTTTAAGGTGATCCTGCACTATGGCACACTGGTAATCGACGGGGT | |
| | | TACGCCGAACATGATCGACTATTTCGGACGGCCGTATGAAGGCATCGCC | |
| | | GTGTTCGACGGCAAAAAGATCACTGTAACAGGGACCCTGTGGAACGGC | |
| | | AACAAAATTATCGACGAGCGCCTGATCAACCCCGACGGCTCCCTGCTGT | |
| | | TCCGAGTAACCATCAACGGAGTGACCGGCTGGCGGCTGTGCGAACGCA | |
| | | TTCTGGCGTAGTGATAATAGCTCGAGGCTGGAGCCTCGGTGGCCATGCT | |
| | | TCTTGCCCCTTGGGCCTCCCCCCAGCCCCTCCTCCCCTTCCTGCACCCGT | |
| | | ACCCCCGTGGTCTTTGAATAAAGTCTGAGTGGGCGGCACAATTGAAAAA | |
| | | AAAAAAAAAAAAAAAAAAAAAAAAAAAAGCATATGACTAAAAAAAAA | |
| | | AAAAAAAAAAAAAAAAAAAAAAAAAAAAAAAAAAAAAAAAAAAAAA | |
| | | AAAAA | |
| 19 | hEPO | AGAAAAAAAAAAAAAATAAACTAGTATTCTTCTGGTCCCCACAGACTCA | 106 |
| | | GAGAGAACCCGCCACCATGAACTCCTTCAGTACCAGTGCTTTCGGGCCA | |
| | | GTTGCTTTTAGTCTCGGACTGCTGCTGGTGCTGCCAGCCGCCTTCCCAGC | |
| | | CCCCGGGGTCCATGAATGTCCAGCATGGCTCTGGTTGTTGTTGAGCTTGT | |
| | | TGAGCCTCCCCCTTGGCCTGCCCGTTTTGGGTGCCCCTCCTAGACTGATT | |
| | | TGCGATTCTAGGGTGCTGGAACGCTATTTGCTTGAGGCTAAGGAAGCCG | |
| | | AGAATATCACCACAGGATGCGCTGAACATTGTAGTTTGAATGAGAACAT | |
| | | AACAGTGCCCGATACCAAAGTGAATTTTTACGCTTGGAAGAGGATGGA | |
| | | GGTGGGCCAGCAAGCCGTAGAGGTTTGGCAGGGCCTGGCCCTGCTGAG | |
| | | CGAAGCCGTGCTGAGAGGACAAGCCCTGTTGGTTAACAGTTCCCAGCCC | |
| | | TGGGAGCCCCTCCAGCTCCATGTCGACAAGGCTGTATCTGGTCTCCGCA | |
| | | GTCTCACTACCCTCCTGAGGGCTCTGGGAGCACAAAAGGAAGCAATAA | |
| | | GCCCCCCCGATGCTGCAAGCGCCGCTCCCCTGCGAACTATCACTGCCGA | |
| | | CACCTTTCGGAAACTGTTCCGCGTGTACTCCAATTTCCTGAGAGGAAAG | |
| | | TTGAAACTTTACACAGGAGAGGCCTGTCGCACTGGTGACCGCGTGATAA | |
| | | TAGCTCGAGGCTGGAGCCTCGGTGGCCATGCTTCTTGCCCCTTGGGCCT | |
| | | CCCCCCAGCCCCTCCTCCCCTTCCTGCACCCGTACCCCCGTGGTCTTTGA | |
| | | ATAAAGTCTGAGTGGGCGGCACAATTGAAAAAAAAAAAAAAAAAAAA | |
| | | AAAAAAAAAAAAAGCATATGACTAAAAAAAAAAAAAAAAAAAAAAAA | |
| | | AAAAAAAAAAAAAAAAAAAAAAAAAAAAAAAAAAAA | |

TABLE 14
FIG. 19 Constructs

| | Identi- | | Sequence (5′ to 3′)- | Sequence (5′ to 3′)- | SEQ ID | Tail |
|---|---|---|---|---|---|---|
| FIG # | fier | 5′ Cap | Azide Oligo | Alkyne Oligo | NO: | Modification |
| 19A | (65) | LNAm7G | /5Phos/+AmGmAmG | /5Phos/+AmGmAmUm | 135 | /5Phos/rArArAr |
| | | | mAmUmAmA/3AzideN/ | AmArA/i5OCTdU/rArA | | ArA/i2MOErA/* |
| | | | | rArArArA | | /i2MOErA/*/ |
| | | | | | | i2MOErA/*/ |
| | | | | | | i2MOErA/*/ |
| | | | | | | i2MOErA/*/ |
| | | | | | | i2MOErA/*/3ddC/ |
| | | | | | | (SEQ ID |
| | | | | | | NO: 176) |
| 19A | (66) | m7G | — | /5Phos/rGrGrArGrArAr | 136 | |
| | | | | UrArArArArA | | |
| 19D | (S20) | LNAm7G | /5Phos/+AmGmAmG | /5Phos/+AmGmAmUm | 137 | /5Phos/rArArAr |
| | | | mAmUmAmA/ | AmArA/i5OCTdU/rArA | | ArA/i2MOErA/* |
| | | | 3AzideN/ | rArArArA | | /i2MOErA/*/ |
| | | | | | | i2MOErA/*/ |
| | | | | | | i2MOErA/*/ |
| | | | | | | i2MOErA/*/ |
| | | | | | | i2MOErA/*/ |
| | | | | | | 3ddC/ (SEQ ID |
| | | | | | | NO: 176) |
| 19D | (S21) | m7G | — | /5Phos/rGrGrArGrArAr | 138 | — |
| | | | | UrArArArArA | | |
| 19 | (45) | — | — | /5Phos/rArArArArA/ | 139 | — |
| | | | | CTdU/rArArArArA/ | | |
| | | | | i5O3Phos/ | | |
| 19J | (49) | LNAm7G | /5Phos/+AmGmAmG | /5Phos/rArArArArA/ | 140 | — |
| | | | mAmUmAmA/ | i5OCTdU/rArArArArA/ | | |
| | | | 3AzideN/ | 3Phos/ | | |

TABLE 15
mRNA Sequences used in FIG. 21

| | | | SEQ ID |
|---|---|---|---|
| FIG # | Identifier | Sequence (5′ to 3′) | NO: |
| 21D- | NanoLuc | AGAAAAAAAAAAAAAATAAACTAGTATTCTTCTGGTCCCCACAGACTCA | 107 |
| E | mRNA | GAGAGAACCCGCCACCATGGGATCCATGGGATCCGTCTTCACACTCGAA | |
| | sequence used | GATTTCGTTGGGGACTGGCGACAGACAGCCGGCTACAACCTGGACCAAG | |
| | for all construct | TCCTTGAACAGGGAGGTGTGTCCAGTTTGTTTCAGAATCTCGGGGTGTCC | |
| | in these panels | GTAACTCCGATCCAAAGGATTGTCCTGAGCGGTGAAAATGGGCTGAAGA | |
| | | TCGACATCCATGTCATCATCCCGTATGAAGGTCTGAGCGGCGACCAAAT | |
| | | GGGCCAGATCGAAAAAATTTTTAAGGTGGTGTACCCTGTGGATGATCAT | |
| | | CACTTTAAGGTGATCCTGCACTATGGCACACTGGTAATCGACGGGGTTAC | |
| | | GCCGAACATGATCGACTATTTCGGACGGCCGTATGAAGGCATCGCCGTG | |
| | | TTCGACGGCAAAAAGATCACTGTAACAGGGACCCTGTGGAACGGCAACA | |
| | | AAATTATCGACGAGCGCCTGATCAACCCCGACGGCTCCCTGCTGTTCCGA | |
| | | GTAACCATCAACGGAGTGACCGGCTGGCGGCTGTGCGAACGCATTCTGG | |
| | | CGTAGTGATAATAGCTCGAGGCTGGAGCCTCGGTGGCCATGCTTCTTGCC | |
| | | CCTTGGGCCTCCCCCCAGCCCCTCCTCCCCTTCCTGCACCCGTACCCCCGT | |
| | | GGTCTTTGAATAAAGTCTGAGTGGGCGGCACAATTGAAAAA | |
| 21D | 7 | Same as Table 16, circRNA (iHRV) | 108 |
| 21F | FLuc-3xHiBiT | AGAAAAAAAAAAAAAAAGAATAAACTAGTATTCTTCTGGTCCCCACAGA | 109 |
| | mRNA | CTCAGAGAGAACCCGCCACCATGGAAGATGCCAAAAACATTAAGAAGG | |
| | sequence used | GCCCAGCGCCATTCTACCCACTCGAAGACGGGACCGCCGGCGAGCAGCT | |
| | for all construct | GCACAAAGCCATGAAGCGCTACGCCCTGGTGCCCGGCACCATCGCCTTT | |
| | in these panels | ACCGACGCACATATCGAGGTGGACATTACCTACGCCGAGTACTTCGAGA | |
| | | TGAGCGTTCGGCTGGCAGAAGCTATGAAGCGCTATGGGCTGAATACAAA | |
| | | CCATCGGATCGTGGTGTGCAGCGAGAATAGCTTGCAGTTCTTCATGCCCG | |
| | | TGTTGGGTGCCCTGTTCATCGGTGTGGCTGTGGCCCCAGCTAACGACATC | |
| | | TACAACGAGCGCGAGCTGCTGAACAGCATGGGCATCAGCCAGCCCACCG | |
| | | TCGTATTCGTGAGCAAGAAAGGGCTGCAAAAGATCCTCAACGTGCAAAA | |
| | | GAAGCTACCGATCATACAAAAGATCATCATCATGGATAGCAAGACCGAC | |
| | | TACCAGGGCTTCCAAAGCATGTACACCTTCGTGACTTCCCATTTGCCACC | |
| | | CGGCTTCAACGAGTACGACTTCGTGCCCGAGAGCTTCGACCGGGACAAA | |
| | | ACCATCGCCCTGATCATGAACAGTAGTGGCAGTACCGGATTGCCCAAGG | |
| | | GCGTAGCCCTACCGCACCGCACCGCTTGTGTCCGATTCAGTCATGCCCGC | |
| | | GACCCCATCTTCGGCAACCAGATCATCCCCGACACCGCTATCCTCAGCGT | |
| | | GGTGCCATTTCACCACGGCTTCGGCATGTTCACCACGCTGGGCTACTTGA | |
| | | TCTGCGGCTTTCGGGTCGTGCTCATGTACCGCTTCGAGGAGGAGCTATTC | |
| | | TTGCGCAGCTTGCAAGACTATAAGATTCAATCTGCCCTGCTGGTGCCCAC | |
| | | ACTATTTAGCTTCTTCGCTAAGAGCACTCTCATCGACAAGTACGACCTAA | |
| | | GCAACTTGCACGAGATCGCCAGCGGCGGGGCGCCGCTCAGCAAGGAGGT | |
| | | AGGTGAGGCCGTGGCCAAACGCTTCCACCTACCAGGCATCCGCCAGGGC | |
| | | TACGGCCTGACAGAAACAACCAGCGCCATTCTGATCACCCCCGAAGGGG | |
| | | ACGACAAGCCTGGCGCAGTAGGCAAGGTGGTGCCCTTCTTCGAGGCTAA | |
| | | GGTGGTGGACTTGGACACCGGTAAGACACTGGGTGTGAACCAGCGCGGC | |
| | | GAGCTGTGCGTCCGTGGCCCCATGATCATGAGCGGCTACGTTAACAACC | |
| | | CCGAGGCTACAAACGCTCTCATCGACAAGGACGGCTGGCTGCACAGCGG | |
| | | CGACATCGCCTACTGGGACGAGGACGAGCACTTCTTCATCGTGGACCGG | |
| | | CTGAAGAGCCTGATCAAATACAAGGGCTACCAGGTAGCCCCAGCCGAAC | |
| | | TGGAGAGCATCCTGCTGCAACACCCCAACATCTTCGACGCCGGGGTCGC | |
| | | CGGCCTGCCCGACGACGATGCCGGCGAGCTGCCCGCCGCAGTCGTCGTG | |
| | | CTGGAACACGGTAAAACCATGACCGAGAAGGAGATCGTGGACTATGTGG | |
| | | CCAGCCAGGTTACAACCGCCAAGAAGCTGCGCGGTGGTGTTGTGTTCGT | |
| | | GGACGAGGTGCCTAAAGGACTGACCGGCAAGTTGGACGCCCGCAAGATC | |
| | | CGCGAGATTCTCATTAAGGCCAAGAAGGGCGGCAAGATCGCCGTGTGAT | |
| | | AATAGAGAAAGAGAACAACAAGAAGAACAACAAAGCCACCATGGTTTC | |
| | | TGGATGGCGCCTCTTCAAGAAAATTTCAGTAAGTGGTTGGCGGTTGTTTA | |
| | | AAAAGATTTCTGTTAGCGGATGGAGGCTTTTCAAAAAAATCAGCTAGTA | |
| | | ATGACTCGAGGCTGGAGCCTCGGTGGCCATGCTTCTTGCCCCTTGGGCCT | |
| | | CCCCCCAGCCCCTCCTCCCCTTCCTGCACCCGTACCCCCGTGGTCTTTGA | |
| | | ATAAAGTCTGAGTGGGCGGCACAATTGAAAAAAA | |

TABLE 16
oligo Sequences used in FIG. 21 to ligate in between 5′ & 3′ of the mRNA sequence
listed in Table 15 to circularize the RNA

| FIG | | | | | Sequence (5′ to 3′)- |
|---|---|---|---|---|---|
| # | Identi- | | | SEQ ID | Branching oligo, if |
| | fier | 5′ Cap | Sequence (5′ to 3′) | NO: | any |
| 21D | 44 | | /5Phos/rArArArArArArArArArArAr | 110 | |
| | | | ArArA/3Phos/ | | |
| 21D | 45 | | /5Phos/rArArArArArArA/i5OCTdU/ | 111 | |
| | | | rArArArArArA/3Phos/ | | |
| 21D | 46 | | /5Phos/rArArArArA/iN6Me- | 112 | |
| | | | rA//iN6Me-rA//iN6Me- | | |
| | | | rA/rArArArArA/3Phos/ | | |
| 21D | 47 | | /5Phos/rArArArArArA*rA*rA*rA*r | 113 | |
| | | | A*rA*rArArA/3Phos/ | | |
| 21D | 48 | m7G | /5Phos/rArArArArArArA/i5OCTdU/ | 114 | m7G+/5Phos/+AmGmAmG |
| | | | rArArArArArA/3Phos/ | | mAmUrArA/3AzideN/ |
| 21D | 49 | LNAm7G | /5Phos/rArArArArArArA/i5OCTdU/ | 115 | LNAm7G+/5Phos/+AmGmA |
| | | | rArArArArArA/3Phos/ | | mGmAmUrArA/3AzideN/ |
| 21D | 50 | — | — | | — |
| 21E | 61 | | /5Phos/rArArArArArCrUrUrArCrCr | 116 | m7G+/5Phos/rUrCrUrGrUrU |
| | | | ArArGrCrGrArArGrArArCrArGrAr | | rCrUrUrCrGrCrUrUrGr |
| | | | ArArArA/3Phos/ | | GrUrArArG (SEQ ID |
| | | | | | NO: 177) non covalently |
| | | | | | annealed, rather than |
| | | | | | branching |
| 21F | 62 | | | 117 | |
| 21F | 63 | | /5Phos/rArArArArArArA/i5OCTdU/ | 118 | |
| | | | rArArArArArA/3Phos/ | | |
| 21F | 64 | LNAm7G | /5Phos/rArArArArArArA/i5OCTdU/ | 119 | LNAm7G+/5Phos/+AmGmA |
| | | | rArArArArArA/3Phos/ | | mGmAmUrArA/3AzideN/ |

TABLE 17
FIG. 22 Constructs

| FIG | Identi- | 5′ Cap | Sequence (5′ to 3′)- | SEQ ID | Sequence (5′ to 3′)- |
|---|---|---|---|---|---|
| # | fier | (stem/branch) | Stem Oligo | NO: | Branching Oligo |
| 22C | 51 | m7G/- | /5Phos/rArGrArArUrArArA | 141 | — |
| | | | rCrUrArGrUrArUrUrCrUrU | | |
| | | | rCrUrGrG/i5OCTdU/rCrCr | | |
| | | | CrCrArA | | |
| 22C | 52 | A/- | /5Phos/rArGrArArUrArArA | 142 | /5Phos/rArGrArGrArUrArA/ |
| | | | rCrUrArGrUrArUrUrCrUrU | | 3AzideN/ |
| | | | rCrUrGrG/i5OCTdU/rCrCr | | |
| | | | CrCrArA | | |
| 22C | 53 | A/m7G | /5Phos/rArGrArArUrArArA | 143 | /5Phos/rArGrArGrArUrArA/ |
| | | | rCrUrArGrUrArUrUrCrUrU | | 3AzideN/ |
| | | | rCrUrGrG/i5OCTdU/rCrCr | | |
| | | | CrCrArA | | |
| 22C | 54 | A/- | /5Phos/rArGrArArUrArArA | 144 | /5Phos/rArGrArGrArUrArA/ |
| | | | rCrUrArGrA/i5OCTdU/rUr | | 3AzideN/ |
| | | | UrCrUrUrCrUrGrGrUrCrCr | | |
| | | | CrCrArA | | |
| 22C | 55 | A/m7G | /5Phos/rArGrArArUrArArA | 145 | /5Phos/rArGrArGrArUrArA/ |
| | | | rCrUrArGrA/i5OCTdU/rUr | | 3AzideN/ |
| | | | UrCrUrUrCrUrGrGrUrCrCr | | |
| | | | CrCrArA | | |
| 22C | 56 | A/- | /5Phos/rArGrA/i5OCTdU/ | 146 | /5Phos/rArGrArGrArUrArA/ |
| | | | rUrArArArCrUrArGrUrArU | | 3AzideN/ |
| | | | rUrCrUrUrCrUrGrGrUrCrC | | |
| | | | rCrCrArA | | |
| 22C | 57 | A/m7G | /5Phos/rArGrA/i5OCTdU/ | 147 | /5Phos/rArGrArGrArUrArA/ |
| | | | rUrArArArCrUrArGrUrArU | | 3AzideN/ |
| | | | rUrCrUrUrCrUrGrGrUrCrC | | |
| | | | rCrCrArA | | |
| 22D | 58 | — | — | | — |
| 22D | 59 | -/m7G | /5Phos/rArArArArA/ | 148 | /5Phos/rArGrArGrArUrArA/ |
| | | | i5OCTdU/rArArArArA/ | | 3AzideN/ |
| | | | 3Phos/ | | |
| 22D | 60 | -/LNAm7G | /5Phos/rArArArArA/ | 149 | /5Phos/rArGrArGrArUrArA/ |
| | | | i5OCTdU/rArArArArA/ | | 3AzideN/ |
| | | | 3Phos/ | | |

TABLE 18
FIG. 23 Constructs

| FIG # | Identifier | Oligo Modification Sequence | SEQ ID NO: |
|---|---|---|---|
| 23 | 29rA_ddC | /5Phos/rArArA rArArA rArArA rArArA rArArA rArArA | 150 |
| | | rArArA rArArA rArArA rArA/3ddC/ | |
| 23 | 6xSr(AG)_invdT | /5Phos/rArArA rArArA rArArA rArArA rArArA rArArA | 151 |
| | | rArArA rArArA* rA*rA*rG* rG*rG*/3InvdT/ | |
| 23 | 26rA_telo | /5Phos/rArArA rArArA rArArA rArArA rArArA rArArA | 152 |
| | | rArArA rArArA rArAT AGG GTT AGG GTT AGG | |
| | | GT*T* A*G*G* G*/3InvdT/ | |
| 23 | LNA inv dT | /5Phos/rArArA rArArA rArArA rArArA rArArA rArArA | 153 |
| | | rArArA rArA+A+A+A+G +G+G/3InvdT/ | |
| 23 | PS + Ome | /5Phos/rArArA rArArA rArArA rArArA rArArA rArAmA | 154 |
| | | mAmAmA mAmAmA*mA*mA*mG* | |
| | | mG*mG*/3InvdT/ | |
| 23 | 6sr(AG)_ddC | /5Phos/rArArA rArArA rArArA rArArA rArArA rArArA | 155 |
| | | rArArA rArArA*rA*rA*rG* rG*rG*/3ddC/ | |
| 23 | LNA_ddC | /5Phos/rArArA rArArA rArArA rArArA rArArA rArArA | 156 |
| | | rArArA rArA+A+A+A+G +G+G/3ddC/ | |
| 23 | 2MOE + PS_ddC | /5Phos/rArArA rArArA rArArA rArArA rArArA rArArA | 157 |
| | | rArArArArA/i2MOErA/*/i2MOErA/*/i2MOErA/*/ | |
| | | i2MOErG//i2MOErG/*/i2MOErG/*/3ddC/ | |

TABLE 19
FIG. 29 Constructs

| | | Stem oligo | | Branch oligo | | SEQ ID |
|---|---|---|---|---|---|---|
| FIG # | Construct | modification | Cap | modification | CDS | NO: |
| 29 | (-)cap AUG- | mU*mC*mG/ | — | /5Phos/rArGr | rArGrArArUrArArArArUrG | 162 |
| | 26 | iOCTdU/rCrCr | | ArGrAr | rGrUrA rUrUrC rUrUrC | |
| | | ArGrCrUrCrGr | | UrArA/ | rUrGrG rUrCrC rCrCrA | |
| | | ArCrCrArGr | | 3AzideN/ | rCrArU rGrGrA rCrUrA | |
| | | GrArU (SEQ | | | rCrArA rArGrA rCrGrA | |
| | | ID NO: 178) | | | rCrGrA rCrGrA rCrArA | |
| | | | | | rGrUrG rArGrC rGrGrC | |
| | | | | | rUrGrG rCrGrG rCrUrG | |
| | | | | | rUrUrC rArArG rArArG | |
| | | | | | rArUrU rArGrC rUrArA | |
| | | | | | rArArArArArArArArArArArA | |
| | | | | | rArArArArArArArArArArArA | |
| | | | | | rArArArArArA | |
| 29 | (+)cap AUG- | mU*mC*mG/iOC | m7G | /5Phos/rArGr | rArGrArArUrArArArArUrG | 163 |
| | 26 | TdU/rCrCrArGrC | | ArGrAr | rGrUrA rUrUrC rUrUrC | |
| | | rUrCrGrArCrCrA | | UrArA/ | rUrGrG rUrCrC rCrCrA | |
| | | rGrGrArU (SEQ | | 3AzideN/ | rCrArU rGrGrA rCrUrA | |
| | | ID NO: 178) | | | rCrArA rArGrA rCrGrA | |
| | | | | | rCrGrA rCrGrA rCrArA | |
| | | | | | rGrUrG rArGrC rGrGrC | |
| | | | | | rUrGrG rCrGrG rCrUrG | |
| | | | | | rUrUrC rArArG rArArG | |
| | | | | | rArUrU rArGrC rUrArA | |
| | | | | | rArArArArArArArArArArArA | |
| | | | | | rArArArArArArArArArArArA | |
| | | | | | rArArArArArA | |
| 29 | (+)cap AUG- | mU*mC*mG/iOC | m7G | /5Phos/rArGr | rArGrArArUrArArArCrUrA | 164 |
| | 29 | TdU/rCrCrArGrC | | ArGrAr | rArUrG rUrUrC rUrUrC | |
| | | rUrCrGrArCrCrA | | UrArA/3 | rUrGrG rUrCrC rCrCrA | |
| | | rGrGrArU (SEQ | | AzideN/ | rCrArU rGrGrA rCrUrA | |
| | | ID NO: 178) | | | rCrArA rArGrA rCrGrA | |
| | | | | | rCrGrA rCrGrA rCrArA | |
| | | | | | rGrUrG rArGrC rGrGrC | |
| | | | | | rUrGrG rCrGrG rCrUrG | |
| | | | | | rUrUrC rArArG rArArG | |
| | | | | | rArUrU rArGrC rUrArA | |
| | | | | | rArArArArArArArArArArArA | |
| | | | | | rArArArArArArArArArArArA | |
| | | | | | rArArArArArA | |
| 29 | (+)cap AUG- | mU*mC*mG/ | m7G | /5Phos/rArGr | rArGrArArUrArArArCrUrA | 165 |
| | 32 | iOCTdU/rCrCrAr | | ArGrAr | rGrUrA rArUrG rUrUrC | |
| | | GrCrUrCrGrArCr | | UrArA/ | rUrGrG rUrCrC rCrCrA | |
| | | CrArGrGrArU (SEQ | | 3AzideN/ | rCrArU rGrGrA rCrUrA | |
| | | ID NO: 178) | | | rCrArA rArGrA rCrGrA | |
| | | | | | rCrGrA rCrGrA rCrArA | |
| | | | | | rGrUrG rArGrC rGrGrC | |
| | | | | | rUrGrG rCrGrG rCrUrG | |
| | | | | | rUrUrC rArArG rArArG | |
| | | | | | rArUrU rArGrC rUrArA | |
| | | | | | rArArArArArArArArArArArA | |
| | | | | | rArArArArArArArArArArArA | |
| | | | | | rArArArArArA | |
| 29 | (+)cap AUG- | mU*mC*mG/iOC | m7G | /5Phos/rArGr | rArGrArArUrArArArCrUrA | 166 |
| | 35 | TdU/rCrCrAr | | ArGrAr | rGrUrA rUrUrC rArUrG | |
| | | GrCrUrCrGr | | UrArA/ | rUrGrG rUrCrC rCrCrA | |
| | | ArCrCrArGr | | 3AzideN/ | rCrArU rGrGrA rCrUrA | |
| | | GrArU (SEQ | | | rCrArA rArGrA rCrGrA | |
| | | ID NO: 178) | | | rCrGrA rCrGrA rCrArA | |
| | | | | | rGrUrG rArGrC rGrGrC | |
| | | | | | rUrGrG rCrGrG rCrUrG | |
| | | | | | rUrUrC rArArG rArArG | |
| | | | | | rArUrU rArGrC rUrArA | |
| | | | | | rArArArArArArArArArArArA | |
| | | | | | rArArArArArArArArArArArA | |
| | | | | | rArArArArArA | |
| 29 | (+)cap AUG- | mU*mC*mG/iOC | m7G | /5Phos/rArGr | rArGrArArUrArArArCrUrA | 167 |
| | 38 | TdU/rCrCrArGrC | | ArGrAr | rGrUrA rUrUrC rUrUrC | |
| | | rUrCrGrArCrCrA | | UrArA/ | rArUrG rUrCrC rCrCrA | |
| | | rGrGrArU (SEQ | | 3AzideN/ | rCrArU rGrGrA rCrUrA | |
| | | ID NO: 178) | | | rCrArA rArGrA rCrGrA | |
| | | | | | rCrGrA rCrGrA rCrArA | |
| | | | | | rGrUrG rArGrC rGrGrC | |
| | | | | | rUrGrG rCrGrG rCrUrG | |
| | | | | | rUrUrC rArArG rArArG | |
| | | | | | rArUrU rArGrC rUrArA | |
| | | | | | rArArArArArArArArArArArA | |
| | | | | | rArArArArArArArArArArArA | |
| | | | | | rArArArArArA | |

1. A method of producing a composition of capped RNA transcripts, comprising:
(a) synthesizing a 5′-phosphorylated RNA oligonucleotide in a solvent system comprising a nonpolar counterion;
(b) capping the 5′-phosphorylated RNA oligonucleotide, thereby producing a capped RNA oligonucleotide;
(c) purifying the capped RNA oligonucleotide, thereby producing a composition comprising a purified capped RNA oligonucleotide, wherein at least 95% of the total RNA oligonucleotides in the composition are capped RNA oligonucleotides; and
(d) ligating the purified capped RNA oligonucleotide to an RNA precursor, thereby producing a capped RNA transcript.

2.-5. (canceled)

6. The method of claim 1, wherein the capping is performed through an anhydrous reaction between the 5′-phosphorylated RNA oligonucleotide and a capping nucleotide conjugated to imidazole in the presence of 1-methylimidazole.

7. The method of claim 1, wherein the RNA precursor comprises one or more of a 5′ untranslated region (5′ UTR) comprising an unstructured region, a 3′ exonuclease-resistant modification, a poly-A tail, an open reading frame (ORF), and a therapeutic nucleic acid.

8.-11. (canceled)

12. The method of claim 1, wherein at least 95% of the capped RNA transcripts produced in step (d) comprise a 5′ cap.

13.-15. (canceled)

16. The method of claim 1, further comprising circularizing the capped RNA transcript.

17. (canceled)

18. The method of claim 1, further comprising:
(i) introducing one or more azide handles onto the 5′-phosphorylated RNA oligonucleotide, and
(ii) conjugating one or more 5′ cap regions to the 5′-phosphorylated RNA oligonucleotide via click chemistry with the one or more azide handles.

19.-24. (canceled)

25. The method of claim 1, wherein at least one of the 5′-phosphorylated RNA oligonucleotide and the RNA precursor comprises one or more modified nucleotides, wherein the one or more modified nucleotides comprise one or more modified sugars, one or more modified phosphates, one or more modified nucleobases, or any combination thereof.

26.-46. (canceled)

47. The method of claim 1, wherein the capped RNA oligonucleotide comprises a 5′ cap selected from the group consisting of 7-methyguanosine (m7G), N7,3′-O-dimethyl-guanosine-5′-triphosphate-5′-guanosine (m7G-3′m-ppp-G), N7,2′-O-dimethyl-guanosine-5′-triphosphate-5′-guanosine (m7Gm-ppp-G), 7-benzylguanosine (Bn7G), chlorobenzylguanosine (ClBn7G), m7G bearing an LNA sugar (m7G-LNA), chlorobenzyl-O-ethoxyguanosine (ClBnOEt7G), 7-(4-chlorophenoxyethyl)-guanosine, 7-ethyl guanosine (e7G), 7-propyl guanosine (p7G), 7-isopropyl guanosine (ip7G), 7-butyl guanosine (b7G), 7-isobutyl guanosine (ib7G), 7-cyclopentyl guanosine (cp7G), 7-(carboxymethyl) guanosine (cm7G), 7-(2-phenylethyl) guanosine [7-(2-PhEt)G], 7-(1-phenylethyl) guanosine [7-(1-PhEt)G], m⁷Gppp_{BH3}G (D1 and D2 stereoisomers), m⁷Gpp_{BH3}G (D1 and D2 stereoisomers), m⁷Gp_{BH3}G (D1 and D2 stereoisomers), m⁷Gpp_{BH3}pm⁷G, m₂^{7,2′-O}GpppBH3G (D1 and D2 stereoisomers), m₂7^{,2′-O}Gpp_{BH3}PG (D1 and D2 diastereomers), m₂^{7,2′-O}GppspG (D1 and D2 diastereomers), N-Arylmethyl analogs, glyceryl, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2′-inverted nucleotide moiety, 3′-2 ‘-inverted abasic moiety, 1,4-butanediol phosphate, 3’-phosphoramidate, hexylphosphate, aminohexyl phosphate, 3′-phosphate, 3′-phosphorothioate, phosphorodithioate, cap1, cap2, cap3, cap4, ARCA, modified ARCA, inosine, N1-methylguanosine, LNA-guanosine, 2-azido-guanosine, and a bridging or non-bridging methylphosphonate moiety.

48.-66. (canceled)

67. A capped RNA transcript produced by the method of claim 1.

68.-71. (canceled)

72. A composition comprising the capped RNA transcript of claim 67.

73.-78. (canceled)

79. A method of preventing or treating a disease in a subject, comprising introducing an effective amount of the capped RNA transcript of claim 67 to the subject.

80.-84. (canceled)

85. A method of screening for altered translation efficiency or RNA stability conferred by one or more nucleotide modifications in an RNA transcript, comprising:
(a) synthesizing a 5′-phosphorylated RNA oligonucleotide comprising one or more modified nucleotides in a solvent system comprising a nonpolar counterion;
(b) chemically capping the 5′-phosphorylated RNA oligonucleotide, thereby producing a capped RNA oligonucleotide;
(c) purifying the capped RNA oligonucleotide;
(d) ligating the capped RNA oligonucleotide to an RNA precursor comprising an open reading frame (ORF) encoding a protein, thereby producing a capped RNA transcript;
(e) expressing the capped RNA transcript in a cell; and
(f) measuring expression of the protein in the cell.

86.-166. (canceled)

167. An RNA molecule comprising (i) one or more modified nucleotides at position +3 or higher with reference to a 5′ terminus of the RNA molecule, and (ii) at least one 5′ cap.

168. The RNA molecule of claim 167 comprising two or more 5′ caps, wherein the two or more 5′ caps are conjugated to the RNA molecule via click chemistry or a photochemical crosslinking reaction.

169.-200. (canceled)

201. The RNA molecule of claim 167, further comprising one or more of an open reading frame (ORF), one or more poly-A tails, and a therapeutic nucleic acid.

202.-208. (canceled)

209. A vector comprising the RNA molecule of claim 167.

210. A delivery agent comprising the RNA molecule of claim 167, wherein the delivery agent comprises a lipid, a peptide, a protein, an antibody, a carbohydrate, a nanoparticle, or a microparticle.

211.-213. (canceled)

214. A composition comprising the RNA molecule of claim 167.

215.-220. (canceled)

221. A method of preventing or treating a disease in a subject, comprising introducing an effective amount of the RNA molecule of claim 167 to the subject.

222.-224. (canceled)

225. A kit comprising the composition of claim 220, a device for administering the composition to a subject, and/or instructions for administering the composition to the subject.


SEQUENCE LISTING (199 sequences)

SEQ ID NO: 1 - RNA, 12 nt, synthetic construct
  source 1..12: mol_type = other RNA
  modified_base 1: mod_base = OTHER; note = 5'-phosphate modified nucleotide
ggagaataaaaa

SEQ ID NO: 2 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 3 - RNA, 12 nt, synthetic construct
  source 1..12: mol_type = other RNA
  modified_base 1: mod_base = OTHER; note = 5'-phosphate modified nucleotide
tgagaataaaaa

SEQ ID NO: 4 - RNA, 12 nt, synthetic construct
  source 1..12: mol_type = other RNA
  modified_base 1: mod_base = OTHER; note = 5'-phosphate modified nucleotide
cgagaataaaaa

SEQ ID NO: 5 - RNA, 12 nt, synthetic construct
  source 1..12: mol_type = other RNA
  modified_base 1: mod_base = OTHER; note = 5'-phosphate modified Inosine
ngagaataaaaa

SEQ ID NO: 6 - RNA, 12 nt, synthetic construct
  source 1..12: mol_type = other RNA
  modified_base 1: mod_base = OTHER; note = 5'-phosphate modified N6-methyladenosine
agagaataaaaa

SEQ ID NO: 7 - RNA, 12 nt, synthetic construct
  source 1..12: mol_type = other RNA
  modified_base 1: mod_base = OTHER; note = 5'-phosphate modified nucleotide with phosphorothioate linkage
agagaataaaaa

SEQ ID NO: 8 - RNA, 12 nt, synthetic construct
  source 1..12: mol_type = other RNA
  modified_base 1: mod_base = OTHER; note = 5'-phosphate modified nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
agagaataaaaa

SEQ ID NO: 9 - RNA, 12 nt, synthetic construct
  source 1..12: mol_type = other RNA
  modified_base 1: mod_base = OTHER; note = 5'-phosphate-2'-fluoro modified nucleotide
agagaataaaaa

SEQ ID NO: 10 - RNA, 12 nt, synthetic construct
  source 1..12: mol_type = other RNA
  modified_base 1: mod_base = OTHER; note = 5'-phosphate modified L-adenosine
agagaataaaaa

SEQ ID NO: 11 - RNA, 12 nt, synthetic construct
  source 1..12: mol_type = other RNA
  modified_base 1: mod_base = OTHER; note = 5'-phosphate modified nucleotide
  misc_feature 1: note = DNA
  misc_feature 2..12: note = RNA
agagaataaaaa

SEQ ID NO: 12 - RNA, 12 nt, synthetic construct
  source 1..12: mol_type = other RNA
  modified_base 1: mod_base = OTHER; note = 5'-phosphate-2'-O-methyl modified nucleotide
agagaataaaaa

SEQ ID NO: 13 - RNA, 12 nt, synthetic construct
  source 1..12: mol_type = other RNA
  modified_base 1: mod_base = OTHER; note = 5'-phosphate-2'-O-methoxyethyl modified nucleotide
agagaataaaaa

SEQ ID NO: 14 - RNA, 12 nt, synthetic construct
  source 1..12: mol_type = other RNA
  modified_base 1: mod_base = OTHER; note = 5'-phosphate modified Locked nucleic acid
agagaataaaaa

SEQ ID NO: 15 - RNA, 12 nt, synthetic construct
  source 1..12: mol_type = other RNA
  modified_base 1: mod_base = OTHER; note = 5'-phosphate modified nucleotide
  misc_feature 1..6: note = DNA
  misc_feature 7..12: note = RNA
agagaataaaaa

SEQ ID NO: 16 - RNA, 12 nt, synthetic construct
  source 1..12: mol_type = other RNA
  modified_base 1: mod_base = OTHER; note = 5'-phosphate-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
agagaataaaaa

SEQ ID NO: 17 - RNA, 12 nt, synthetic construct
  source 1..12: mol_type = other RNA
  modified_base 1: mod_base = OTHER; note = 5'-phosphate-2'-O-methoxyethyl modified nucleotide
  modified_base 2: mod_base = OTHER; note = 2'-O-methoxyethyl modified nucleotide
  modified_base 3: mod_base = OTHER; note = 2'-O-methoxyethyl modified nucleotide
  modified_base 4: mod_base = OTHER; note = 2'-O-methoxyethyl modified nucleotide
  modified_base 5: mod_base = OTHER; note = 2'-O-methoxyethyl modified nucleotide
  modified_base 6: mod_base = OTHER; note = 2'-O-methoxyethyl modified nucleotide
agagaataaaaa

SEQ ID NO: 18 - RNA, 12 nt, synthetic construct
  source 1..12: mol_type = other RNA
  modified_base 1: mod_base = OTHER; note = 5'-phosphate modified Locked nucleic acid
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
agagaataaaaa

SEQ ID NO: 19 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 20 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 21 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 22 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 23 - RNA, 12 nt, synthetic construct
  source 1..12: mol_type = other RNA
  modified_base 1: mod_base = OTHER; note = 5'-phosphate modified Locked nucleic acid
agagaataaaaa

SEQ ID NO: 24 - RNA, 12 nt, synthetic construct
  source 1..12: mol_type = other RNA
  modified_base 1: mod_base = OTHER; note = 5'-phosphate modified Locked nucleic acid
  modified_base 2: mod_base = OTHER; note = Locked nucleic acid
  modified_base 3: mod_base = OTHER; note = Locked nucleic acid
  modified_base 4: mod_base = OTHER; note = Locked nucleic acid
  modified_base 5: mod_base = OTHER; note = Locked nucleic acid
  modified_base 6: mod_base = OTHER; note = Locked nucleic acid
agagaataaaaa

SEQ ID NO: 25 - RNA, 12 nt, synthetic construct
  source 1..12: mol_type = other RNA
  modified_base 1: mod_base = OTHER; note = 5'-phosphate-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
agagaataaaaa

SEQ ID NO: 26 - RNA, 23 nt, synthetic construct
  source 1..23: mol_type = other RNA
  modified_base 1: mod_base = OTHER; note = 5'-phosphate-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
agagaataaaaaagggaataaac

SEQ ID NO: 27 - DNA, 20 bp, synthetic construct
  source 1..20: mol_type = other DNA
aaggtcggagtcaacggatt

SEQ ID NO: 28 - DNA, 20 bp, synthetic construct
  source 1..20: mol_type = other DNA
tgaggtcaatgaaggggtca

SEQ ID NO: 29 - DNA, 22 bp, synthetic construct
  source 1..22: mol_type = other DNA
ggctgtttaccagactccgaca

SEQ ID NO: 30 - DNA, 22 bp, synthetic construct
  source 1..22: mol_type = other DNA
cacaaagcctggcagctctcta

SEQ ID NO: 31 - DNA, 22 bp, synthetic construct
  source 1..22: mol_type = other DNA
ctctgagcatcctggtgaggaa

SEQ ID NO: 32 - DNA, 22 bp, synthetic construct
  source 1..22: mol_type = other DNA
aaggtcagccagaacaggtcgt

SEQ ID NO: 33 - DNA, 10 bp, synthetic construct
  source 1..10: mol_type = other DNA
  modified_base 4: mod_base = OTHER; note = 5-Octadiynyl modified uracil
  misc_feature 1..3: note = RNA
  misc_feature 4: note = DNA
  misc_feature 5..10: note = RNA
aaataaaaaa

SEQ ID NO: 34 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 35 - RNA, 13 nt, synthetic construct
  source 1..13: mol_type = other RNA
  modified_base 1: mod_base = OTHER; note = 5'-phosphate modified nucleotide
agataaaaaaaaa

SEQ ID NO: 36 - DNA, 14 bp, synthetic construct
  source 1..14: mol_type = other DNA
  modified_base 1: mod_base = OTHER; note = 5'-phosphate modified nucleotide
  modified_base 2: mod_base = OTHER; note = 5-Octadiynyl modified uracil
  misc_feature 1: note = RNA
  misc_feature 2: note = DNA
  misc_feature 3..14: note = RNA
atgataaaaaaaaa

SEQ ID NO: 37 - DNA, 14 bp, synthetic construct
  source 1..14: mol_type = other DNA
  modified_base 1: mod_base = OTHER; note = 5'-phosphate modified nucleotide
  modified_base 5: mod_base = OTHER; note = 5-Octadiynyl modified uracil
  misc_feature 1..4: note = RNA
  misc_feature 5: note = DNA
  misc_feature 6..14: note = RNA
agattaaaaaaaaa

SEQ ID NO: 38 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 39 - DNA, 16 bp, synthetic construct
  source 1..16: mol_type = other DNA
  modified_base 1: mod_base = OTHER; note = 5'-phosphate modified nucleotide
  modified_base 11: mod_base = OTHER; note = 5-Octadiynyl modified uracil
  misc_feature 1..10: note = RNA
  misc_feature 11: note = DNA
  misc_feature 12..16: note = RNA
agataaaaaataaaaa

SEQ ID NO: 40 - DNA, 14 bp, synthetic construct
  source 1..14: mol_type = other DNA
  modified_base 1: mod_base = OTHER; note = 5'-phosphate modified nucleotide
  modified_base 2: mod_base = OTHER; note = 5-Octadiynyl modified uracil
  misc_feature 1: note = RNA
  misc_feature 2: note = DNA
  misc_feature 3..14: note = RNA
atgataaaaaaaaa

SEQ ID NO: 41 - DNA, 14 bp, synthetic construct
  source 1..14: mol_type = other DNA
  modified_base 1: mod_base = OTHER; note = 5'-phosphate modified nucleotide
  modified_base 5: mod_base = OTHER; note = 5-Octadiynyl modified uracil
  misc_feature 1..4: note = RNA
  misc_feature 5: note = DNA
  misc_feature 6..14: note = RNA
agattaaaaaaaaa

SEQ ID NO: 42 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 43 - DNA, 16 bp, synthetic construct
  source 1..16: mol_type = other DNA
  modified_base 1: mod_base = OTHER; note = 5'-phosphate modified nucleotide
  modified_base 11: mod_base = OTHER; note = 5-Octadiynyl modified uracil
  misc_feature 1..10: note = RNA
  misc_feature 11: note = DNA
  misc_feature 12..16: note = RNA
agataaaaaataaaaa

SEQ ID NO: 44 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 45 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 46 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 47 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 48 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 49 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
ggataaataaaaaa

SEQ ID NO: 50 - DNA, 14 bp, synthetic construct
  source 1..14: mol_type = other DNA
  modified_base 1: mod_base = OTHER; note = 5'-phosphate modified Locked nucleic acid
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 51 - DNA, 110 bp, synthetic construct
  source 1..110: mol_type = other DNA
  modified_base 1: mod_base = OTHER; note = 5'-phosphate modified nucleotide
  modified_base 12: mod_base = OTHER; note = 5-Octadiynyl modified uracil
  misc_feature 1..11: note = RNA
  misc_feature 12: note = DNA
  misc_feature 13..110: note = RNA
agataaactagtattcttctggtccccacagactcagagagaacccgccaccatggtgagcggctggcggctgttcaagaagattagctgaaaaaaaaaaaaaaaaaaaa

SEQ ID NO: 52 - DNA, 110 bp, synthetic construct; identical to SEQ ID NO: 51
  source 1..110: mol_type = other DNA
  modified_base 1: mod_base = OTHER; note = 5'-phosphate modified nucleotide
  modified_base 12: mod_base = OTHER; note = 5-Octadiynyl modified uracil
  misc_feature 1..11: note = RNA
  misc_feature 12: note = DNA
  misc_feature 13..110: note = RNA

SEQ ID NO: 53 - DNA, 110 bp, synthetic construct; identical to SEQ ID NO: 51
  source 1..110: mol_type = other DNA
  modified_base 1: mod_base = OTHER; note = 5'-phosphate modified nucleotide
  modified_base 12: mod_base = OTHER; note = 5-Octadiynyl modified uracil
  misc_feature 1..11: note = RNA
  misc_feature 12: note = DNA
  misc_feature 13..110: note = RNA

SEQ ID NO: 54 - DNA, 110 bp, synthetic construct; identical to SEQ ID NO: 51
  source 1..110: mol_type = other DNA
  modified_base 1: mod_base = OTHER; note = 5'-phosphate modified nucleotide
  modified_base 12: mod_base = OTHER; note = 5-Octadiynyl modified uracil
  misc_feature 1..11: note = RNA
  misc_feature 12: note = DNA
  misc_feature 13..110: note = RNA

SEQ ID NO: 55 - DNA, 184 bp, synthetic construct
  source 1..184: mol_type = other DNA
ggaaaaaaaaaagaaaaaaaagaataaatttgtattattaatataattaaattaaaattaagagaagggaagatggtgagtggatggagattatttaagaagattagttagtaattttaatttaattatattaataatacaaatttaaaaaaaaaaaagaaaaaaaaaaaaaaaaaaaaaaaaa

SEQ ID NO: 56 - DNA, 184 bp, synthetic construct; identical to SEQ ID NO: 55
  source 1..184: mol_type = other DNA

SEQ ID NO: 57 - DNA, 184 bp, synthetic construct; identical to SEQ ID NO: 55
  source 1..184: mol_type = other DNA

SEQ ID NO: 58 - DNA, 184 bp, synthetic construct; identical to SEQ ID NO: 55
  source 1..184: mol_type = other DNA

SEQ ID NO: 59 - DNA, 1927 bp, synthetic construct
  source 1..1927: mol_type = other DNA
agggaataaactagtattcttctggtccccacagactcagagagaacccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtgataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaagcatatgactaaaaaaaaaaaaaaaaaaaaaaaaaaaaaaaaaaaaaaaaaaaaaaaaaaa

SEQ ID NO: 60 - DNA, 1942 bp, synthetic construct
  source 1..1942: mol_type = other DNA
agaaaaaaaaaaaaaaaggaataaactagtattcttctggtccccacagactcagagagaacccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtgataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaagcatatgactaaaaaaaaaaaaaaaaaaaaaaaaaaaaaaaaaaaaaaaaaaaaaaaaaaa

SEQ ID NO: 61 - DNA, 26 bp, synthetic construct
  source 1..26: mol_type = other DNA
gaatggcgctgggcccttcttaatgt

SEQ ID NO: 62 - DNA, 30 bp, synthetic construct
  source 1..30: mol_type = other DNA
  modified_base 1: mod_base = OTHER; note = 5'-phosphate modified nucleotide
  modified_base 24: mod_base = OTHER; note = Nucleotide with phosphorothioate linkage
  modified_base 25: mod_base = OTHER; note = Nucleotide with phosphorothioate linkage
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
  modified_base 29: mod_base = OTHER; note = Nucleotide with phosphorothioate linkage
  modified_base 30: mod_base = OTHER; note = 3'-Dideoxy-cytidine
  misc_feature 1..29: note = RNA
  misc_feature 30: note = DNA
aaaaaaaaaaaaaaaaaaaaaaaaaaaaac

SEQ ID NO: 63 - DNA, 30 bp, synthetic construct; identical to SEQ ID NO: 62
  source 1..30: mol_type = other DNA
  modified_base 1: mod_base = OTHER; note = 5'-phosphate modified nucleotide
  modified_base 24: mod_base = OTHER; note = 2'-O-methoxyethyl modified nucleotide with phosphorothioate linkage
  modified_base 25: mod_base = OTHER; note = 2'-O-methoxyethyl modified nucleotide with phosphorothioate linkage
  modified_base 26: mod_base = OTHER; note = 2'-O-methoxyethyl modified nucleotide with phosphorothioate linkage
  modified_base 27: mod_base = OTHER; note = 2'-O-methoxyethyl modified nucleotide with phosphorothioate linkage
  modified_base 28: mod_base = OTHER; note = 2'-O-methoxyethyl modified nucleotide with phosphorothioate linkage
  modified_base 29: mod_base = OTHER; note = 2'-O-methoxyethyl modified nucleotide with phosphorothioate linkage
  modified_base 30: mod_base = OTHER; note = 3'-Dideoxy-cytidine
  misc_feature 1..29: note = RNA
  misc_feature 30: note = DNA

SEQ ID NO: 64 - DNA, 57 bp, synthetic construct
  source 1..57: mol_type = other DNA
  modified_base 1: mod_base = OTHER; note = 5'-phosphate modified nucleotide
actttacggggttgttaacgtagccgctaattattactgaagtcgtagactaagata

SEQ ID NO: 65 - DNA, 57 bp, synthetic construct
  source 1..57: mol_type = other DNA
  modified_base 1: mod_base = OTHER; note = 5'-phosphate modified nucleotide
actttagagtgctcttagcgaagaagctaattattactgaagtcgtagactaagata

SEQ ID NO: 66 - DNA, 56 bp, synthetic construct
  source 1..56: mol_type = other DNA
  modified_base 1: mod_base = OTHER; note = 5'-phosphate modified nucleotide
actttagggcaatccggtactgccactaattattactgaagtcgtagactaagata

SEQ ID NO: 67 - DNA, 56 bp, synthetic construct
  source 1..56: mol_type = other DNA
  modified_base 1: mod_base = OTHER; note = 5'-phosphate modified nucleotide
actttattgaggatcttttgcagccctaattattactgaagtcgtagactaagata

SEQ ID NO: 68 - DNA, 57 bp, synthetic construct
  source 1..57: mol_type = other DNA
  modified_base 1: mod_base = OTHER; note = 5'-phosphate modified nucleotide
actttattgtattcagcccatagcgcttaattattactgaagtcgtagactaagata

SEQ ID NO: 69 - DNA, 55 bp, synthetic construct
  source 1..55: mol_type = other DNA
  modified_base 1: mod_base = OTHER; note = 5'-phosphate modified nucleotide
acactaattccgatcagatcagggataattattactgaaacatacactaaagata

SEQ ID NO: 70 - DNA, 56 bp, synthetic construct
  source 1..56: mol_type = other DNA
  modified_base 1: mod_base = OTHER; note = 5'-phosphate modified nucleotide
acactagctcgatgtgaggcacgacgtaattattactgaaacatacactaaagata

SEQ ID NO: 71 - DNA, 57 bp, synthetic construct
  source 1..57: mol_type = other DNA
  modified_base 1: mod_base = OTHER; note = 5'-phosphate modified nucleotide
acactattagctccctcgacaatagcgtaattattactgaaacatacactaaagata

SEQ ID NO: 72 - DNA, 55 bp, synthetic construct
  source 1..55: mol_type = other DNA
  modified_base 1: mod_base = OTHER; note = 5'-phosphate modified nucleotide
acactaagccattcccgctcttgccgaattattactgaaacatacactaaagata

SEQ ID NO: 73 - DNA, 61 bp, synthetic construct
  source 1..61: mol_type = other DNA
  modified_base 1: mod_base = OTHER; note = 5'-phosphate modified nucleotide
aagataacatcagcatcctacggggttgttaacgtagccgctattacatcagcatacacta

SEQ ID NO: 74 - DNA, 61 bp, synthetic construct
  source 1..61: mol_type = other DNA
  modified_base 1: mod_base = OTHER; note = 5'-phosphate modified nucleotide
aagataacatcagcatcctagagtgctcttagcgaagaagctattacatcagcatacacta

SEQ ID NO: 75 - DNA, 60 bp, synthetic construct
  source 1..60: mol_type = other DNA
  modified_base 1: mod_base = OTHER; note = 5'-phosphate modified nucleotide
aagataacatcagcatcctagggcaatccggtactgccactattacatcagcatacacta

SEQ ID NO: 76 - DNA, 60 bp, synthetic construct
  source 1..60: mol_type = other DNA
  modified_base 1: mod_base = OTHER; note = 5'-phosphate modified nucleotide
aagataacatcagcatcctattgaggatcttttgcagccctattacatcagcatacacta

SEQ ID NO: 77 - DNA, 61 bp, synthetic construct
  source 1..61: mol_type = other DNA
  modified_base 1: mod_base = OTHER; note = 5'-phosphate modified nucleotide
aagataacatcagcatcctattgtattcagcccatagcgcttattacatcagcatacacta

SEQ ID NO: 78 - DNA, 34 bp, synthetic construct
  source 1..34: mol_type = other DNA
tgtcgatgagagcgtttgtagcgtggatgctgat

SEQ ID NO: 79 - DNA, 34 bp, synthetic construct
  source 1..34: mol_type = other DNA
tgcttaggtcgtacttgtcgatgtggatgctgat

SEQ ID NO: 80 - DNA, 34 bp, synthetic construct
  source 1..34: mol_type = other DNA
cggtgcggtagggctacgccctgtggatgctgat

SEQ ID NO: 81 - DNA, 34 bp, synthetic construct
  source 1..34: mol_type = other DNA
tgatcggtagcttcttttgcacgtggatgctgat

SEQ ID NO: 82 - DNA, 34 bp, synthetic construct
  source 1..34: mol_type = other DNA
cgctgcacaccacgatccgatggtggatgctgat

SEQ ID NO: 83 - DNA, 87 bp, synthetic construct
  source 1..87: mol_type = other DNA
  modified_base 85: mod_base = OTHER; note = Nucleotide with phosphorothioate linkage
  modified_base 86: mod_base = OTHER; note = Nucleotide with phosphorothioate linkage
acaaaatagaaccgcggtcctattcaaaaaaaaaaaaaaaaaaaaaaaaaaaaaaaaaaaaaaaaaaaaaaaaaatatctttagtgt

SEQ ID NO: 84 - DNA, 87 bp, synthetic construct
  source 1..87: mol_type = other DNA
  modified_base 85: mod_base = OTHER; note = Nucleotide with phosphorothioate linkage
  modified_base 86: mod_base = OTHER; note = Nucleotide with phosphorothioate linkage
catcgtttatggtcggaactacgacaaaaaaaaaaaaaaaaaaaaaaaaaaaaaaaaaaaaaaaaaaaaaaaaaatatctttagtgt

SEQ ID NO: 85 - DNA, 86 bp, synthetic construct
  source 1..86: mol_type = other DNA
  modified_base 84: mod_base = OTHER; note = Nucleotide with phosphorothioate linkage
  modified_base 85: mod_base = OTHER; note = Nucleotide with phosphorothioate linkage
aggtttcccgtgttgagtcaaattaaaaaaaaaaaaaaaaaaaaaaaaaaaaaaaaaaaaaaaaaaaaaaaaaatatctttagtgt

SEQ ID NO: 86 - DNA, 87 bp, synthetic construct
  source 1..87: mol_type = other DNA
  modified_base 85: mod_base = OTHER; note = Nucleotide with phosphorothioate linkage
  modified_base 86: mod_base = OTHER; note = Nucleotide with phosphorothioate linkage
tgttattgctcaatctcgggtggctaaaaaaaaaaaaaaaaaaaaaaaaaaaaaaaaaaaaaaaaaaaaaaaaaatatctttagtgt

SEQ ID NO: 87 - DNA, 87 bp, synthetic construct
  source 1..87: mol_type = other DNA
  modified_base 85: mod_base = OTHER; note = Nucleotide with phosphorothioate linkage
  modified_base 86: mod_base = OTHER; note = Nucleotide with phosphorothioate linkage
agatagtcaagttcgaccgtcttctaaaaaaaaaaaaaaaaaaaaaaaaaaaaaaaaaaaaaaaaaaaaaaaaaatatctttagtgt

SEQ ID NO: 88 - DNA, 19 bp, synthetic construct
  source 1..19: mol_type = other DNA
tcgtagactaagataacat

SEQ ID NO: 89 - DNA, 19 bp, synthetic construct
  source 1..19: mol_type = other DNA
catacactaaagataacat

SEQ ID NO: 90 - RNA, 22 nt, synthetic construct
  source 1..22: mol_type = other RNA
taccagcggaatcgaatgaaga

SEQ ID NO: 91 - RNA, 21 nt, synthetic construct
  source 1..21: mol_type = other RNA
tcagttgatgaagatgcgcta

SEQ ID NO: 92 - RNA, 21 nt, synthetic construct
  source 1..21: mol_type = other RNA
tacgagggcctttgatgctta

SEQ ID NO: 93 - RNA, 21 nt, synthetic construct
  source 1..21: mol_type = other RNA
cgcctctgttgcgtaccgtta

SEQ ID NO: 94 - skipped

SEQ ID NO: 95 - DNA, 19 bp, synthetic construct
  source 1..19: mol_type = other DNA
gctagctccagggtgtggc

SEQ ID NO: 96 - DNA, 29 bp, synthetic construct
  source 1..29: mol_type = other DNA
gggaccagaagaatactagtttattcgat

SEQ ID NO: 97 - RNA, 2018 nt, synthetic construct
  source 1..2018: mol_type = other RNA
gggcgaattggagctcgggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaacaaaaaaaaaaaataattgactaatatcttaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcccactcactatttgttttcgcgcccagttgcaaaaagtgtcggggctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtagtctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcctcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcagg

SEQ ID NO: 98 - RNA, 1311 nt, synthetic construct
  source 1..1311: mol_type = other RNA
acttgctttaacaagttggagatatagtctgctctgcatggtgacatgcagctggatataattccggggtaagattaacgaccttatctgaacataacgctaccgtttaatattgcgtcatataaaaaaaaaaaccaaaaaaaaaaacaaaaaaaaaaataattgactaattaagcagactgtaaatctgctaaaatcgaataaactagtattcttctggtccccacagactcagagagaacccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtagtctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 99 - RNA, 1311 nt, synthetic construct; identical to SEQ ID NO: 98
  source 1..1311: mol_type = other RNA

SEQ ID NO: 100 - RNA, 811 nt, synthetic construct
  source 1..811: mol_type = other RNA
aggaataaactagtattcttctggtccccacagactcagagagaacccgccaccatgggatccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtgataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaa

SEQ ID NO: 101-102 - skipped (2 entries)

SEQ ID NO: 103 - DNA, 19 bp, synthetic construct
  source 1..19: mol_type = other DNA
gccagtccccaacgaaatc

SEQ ID NO: 104 - DNA, 33 bp, synthetic construct
  source 1..33: mol_type = other DNA
gcaactggcccgaaagcactggtactgaaggag

SEQ ID NO: 105 - DNA, 831 bp, synthetic construct
  source 1..831: mol_type = other DNA
agaaaaaaaaaaaaaataaactagtattcttctggtccccacagactcagagagaacccgccaccatgggatccatgggatccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtgataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaa

SEQ ID NO: 106 - DNA, 964 bp, synthetic construct
  source 1..964: mol_type = other DNA
agaaaaaaaaaaaaaataaactagtattcttctggtccccacagactcagagagaacccgccaccatgaactccttcagtaccagtgctttcgggccagttgcttttagtctcggactgctgctggtgctgccagccgccttcccagcccccggggtccatgaatgtccagcatggctctggttgttgttgagcttgttgagcctcccccttggcctgcccgttttgggtgcccctcctagactgatttgcgattctagggtgctggaacgctatttgcttgaggctaaggaagccgagaatatcaccacaggatgcgctgaacattgtagtttgaatgagaacataacagtgcccgataccaaagtgaatttttacgcttggaagaggatggaggtgggccagcaagccgtagaggtttggcagggcctggccctgctgagcgaagccgtgctgagaggacaagccctgttggttaacagttcccagccctgggagcccctccagctccatgtcgacaaggctgtatctggtctccgcagtctcactaccctcctgagggctctgggagcacaaaaggaagcaataagcccccccgatgctgcaagcgccgctcccctgcgaactatcactgccgacacctttcggaaactgttccgcgtgtactccaatttcctgagaggaaagttgaaactttacacaggagaggcctgtcgcactggtgaccgcgtgataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaa

SEQ ID NO: 107 - RNA, 733 nt, synthetic construct
  source 1..733: mol_type = other RNA
agaaaaaaaaaaaaaataaactagtattcttctggtccccacagactcagagagaacccgccaccatgggatccatgggatccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtgataatagctcgaggctggagcctcggtggccatgcttcttgccccttgggcctccccccagcccctcctccccttcctgcacccgtacccccgtggtctttgaataaagtctgagtgggcggcacaattgaaaaa

SEQ ID NO: 108 - skipped

SEQ ID NO: 109 - RNA, 2004 nt, synthetic construct
  source 1..2004: mol_type = other RNA
agaaaaaaaaaaaaaaagaataaactagtattcttctggtccccacagactcagagagaacccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtgataatagagaaagagaacaacaagaagaacaacaaagccaccatggtttctggatggcgcctcttcaagaaaatttcagtaagtggttggcggttgtttaaaaagatttctgttagcggatggaggcttttcaaaaaaatcagctagtaatgactcgaggctggagcctcggtggccatgcttcttgccccttgggcctccccccagcccctcctccccttcctgcacccgtacccccgtggtctttgaataaagtctgagtgggcggcacaattgaaaaaaa

SEQ ID NO: 110 - RNA, 14 nt, synthetic construct
  source 1..14: mol_type = other RNA
  modified_base 1: mod_base = OTHER; note = 5'-phosphate modified nucleotide
  modified_base 14: mod_base = OTHER; note = 3'-phosphate modified nucleotide
aaaaaaaaaaaaaa

SEQ ID NO: 111 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  modified_base 14: mod_base = OTHER; note = 3'-phosphate modified nucleotide
  misc_feature 1..7: note = RNA
  misc_feature 8: note = DNA
  misc_feature 9..14: note = RNA
aaaaaaataaaaaa

SEQ ID NO: 112 - RNA, 13 nt, synthetic construct
  source 1..13: mol_type = other RNA
  modified_base 1: mod_base = OTHER; note = 5'-phosphate modified nucleotide
  modified_base 6: mod_base = OTHER; note = N6-methyladenosine
  modified_base 7: mod_base = OTHER; note = N6-methyladenosine
  modified_base 8: mod_base = OTHER; note = N6-methyladenosine
  modified_base 13: mod_base = OTHER; note = 3'-phosphate modified nucleotide
aaaaaaaaaaaaa

SEQ ID NO: 113 - RNA, 14 nt, synthetic construct
  source 1..14: mol_type = other RNA
  modified_base 1: mod_base = OTHER; note = 5'-phosphate modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 14: mod_base = OTHER; note = 3'-phosphate modified nucleotide
aaaaaaaaaaaaaa

SEQ ID NO: 114 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  modified_base 14: mod_base = OTHER; note = 3'-phosphate modified nucleotide
  misc_feature 1..7: note = RNA
  misc_feature 8: note = DNA
  misc_feature 9..14: note = RNA
aaaaaaataaaaaa

SEQ ID NO: 115 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  modified_base 14: mod_base = OTHER; note = 3'-phosphate modified nucleotide
  misc_feature 1..7: note = RNA
  misc_feature 8: note = DNA
  misc_feature 9..14: note = RNA
aaaaaaataaaaaa

SEQ ID NO: 116 - RNA, 29 nt, synthetic construct
  source 1..29: mol_type = other RNA
  modified_base 1: mod_base = OTHER; note = 5'-phosphate modified nucleotide
  modified_base 29: mod_base = OTHER; note = 3'-phosphate modified nucleotide
aaaaacttaccaagcgaagaacagaaaaa

SEQ ID NO: 117 - skipped

SEQ ID NO: 118 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  modified_base 14: mod_base = OTHER; note = 3'-phosphate modified nucleotide
  misc_feature 1..7: note = RNA
  misc_feature 8: note = DNA
  misc_feature 9..14: note = RNA
aaaaaaataaaaaa

SEQ ID NO: 119 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  modified_base 14: mod_base = OTHER; note = 3'-phosphate modified nucleotide
  misc_feature 1..7: note = RNA
  misc_feature 8: note = DNA
  misc_feature 9..14: note = RNA
aaaaaaataaaaaa

SEQ ID NO: 120 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 121 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 122 - RNA, 12 nt, synthetic construct
  source 1..12: mol_type = other RNA
  modified_base 1: mod_base = OTHER; note = 5'-phosphate modified Locked nucleic acid
agagaataaaaa

SEQ ID NO: 123 - RNA, 12 nt, synthetic construct
  source 1..12: mol_type = other RNA
  modified_base 1: mod_base = OTHER; note = 5'-phosphate-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
agagaataaaaa

SEQ ID NO: 124 - RNA, 12 nt, synthetic construct
  source 1..12: mol_type = other RNA
  modified_base 1: mod_base = OTHER; note = 5'-phosphate modified Locked nucleic acid
agagaataaaaa

SEQ ID NO: 125 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 126 - DNA, 14 bp, synthetic construct
  source 1..14: mol_type = other DNA
  modified_base 1: mod_base = OTHER; note = 5'-phosphate modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 127 - RNA, 12 nt, synthetic construct
  source 1..12: mol_type = other RNA
  modified_base 1: mod_base = OTHER; note = 5'-phosphate-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
agagaataaaaa

SEQ ID NO: 128 - RNA, 23 nt, synthetic construct
  source 1..23: mol_type = other RNA
  modified_base 1: mod_base = OTHER; note = 5'-phosphate-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
agagaataaaaaagggaataaac

SEQ ID NO: 129 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 130 - RNA, 12 nt, synthetic construct
  source 1..12: mol_type = other RNA
  modified_base 1: mod_base = OTHER; note = 5'-phosphate-2'-O-methyl modified nucleotide
agagaataaaaa

SEQ ID NO: 131 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 132 - RNA, 545 nt, synthetic construct
  source 1..545: mol_type = other RNA
tagtctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 133 - RNA, 545 nt, synthetic construct; identical to SEQ ID NO: 132
  source 1..545: mol_type = other RNA

SEQ ID NO: 134 - RNA, 225 nt, synthetic construct
  source 1..225: mol_type = other RNA
gataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaagcatatgactaaaaaaaaaaaaaaaaaaaaaaaaaaaaaaaaaaaaaaaaaaaaaaaaaaaaaaa

SEQ ID NO: 135 - DNA, 14 bp, synthetic construct
  source 1..14: mol_type = other DNA
  modified_base 1: mod_base = OTHER; note = 5'-phosphate modified Locked nucleic acid
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 136 - RNA, 12 nt, synthetic construct
  source 1..12: mol_type = other RNA
  modified_base 1: mod_base = OTHER; note = 5'-phosphate modified nucleotide
ggagaataaaaa

SEQ ID NO: 137 - DNA, 14 bp, synthetic construct
  source 1..14: mol_type = other DNA
  modified_base 1: mod_base = OTHER; note = 5'-phosphate modified Locked nucleic acid
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 8: mod_base = OTHER; note = 5-Octadiynyl modified uracil
  misc_feature 1..7: note = RNA
  misc_feature 8: note = DNA
  misc_feature 9..14: note = RNA
agataaataaaaaa

SEQ ID NO: 138 - RNA, 12 nt, synthetic construct
  source 1..12: mol_type = other RNA
  modified_base 1: mod_base = OTHER; note = 5'-phosphate modified nucleotide
ggagaataaaaa

SEQ ID NO: 139 - DNA, 11 bp, synthetic construct
  source 1..11: mol_type = other DNA
  modified_base 1: mod_base = OTHER; note = 5'-phosphate modified nucleotide
  modified_base 6: mod_base = OTHER; note = 5-Octadiynyl modified uracil
  modified_base 11: mod_base = OTHER; note = 3'-phosphate modified nucleotide
  misc_feature 1..5: note = RNA
  misc_feature 6: note = DNA
  misc_feature 7..11: note = RNA
aaaaataaaaa

SEQ ID NO: 140 - DNA, 11 bp, synthetic construct
  source 1..11: mol_type = other DNA
  modified_base 1: mod_base = OTHER; note = 5'-phosphate modified nucleotide
  modified_base 6: mod_base = OTHER; note = 5-Octadiynyl modified uracil
  modified_base 11: mod_base = OTHER; note = 3'-phosphate modified nucleotide
  misc_feature 1..5: note = RNA
  misc_feature 6: note = DNA
  misc_feature 7..11: note = RNA
aaaaataaaaa

SEQ ID NO: 141 - DNA, 30 bp, synthetic construct
  source 1..30: mol_type = other DNA
  modified_base 1: mod_base = OTHER; note = 5'-phosphate modified nucleotide
  modified_base 24: mod_base = OTHER; note = 5-Octadiynyl modified uracil
  misc_feature 1..23: note = RNA
  misc_feature 24: note = DNA
  misc_feature 25..30: note = RNA
agaataaactagtattcttctggtccccaa

SEQ ID NO: 142 - DNA, 30 bp, synthetic construct; identical to SEQ ID NO: 141
  source 1..30: mol_type = other DNA
  modified_base 1: mod_base = OTHER; note = 5'-phosphate modified nucleotide
  modified_base 24: mod_base = OTHER; note = 5-Octadiynyl modified uracil
  misc_feature 1..23: note = RNA
  misc_feature 24: note = DNA
  misc_feature 25..30: note = RNA

SEQ ID NO: 143 - DNA, 30 bp, synthetic construct; identical to SEQ ID NO: 141
  source 1..30: mol_type = other DNA
  modified_base 1: mod_base = OTHER; note = 5'-phosphate modified nucleotide
  modified_base 24: mod_base = OTHER; note = 5-Octadiynyl modified uracil
  misc_feature 1..23: note = RNA
  misc_feature 24: note = DNA
  misc_feature 25..30: note = RNA

SEQ ID NO: 144 - DNA, 30 bp, synthetic construct
  source 1..30: mol_type = other DNA
  modified_base 1: mod_base = OTHER; note = 5'-phosphate modified nucleotide
  modified_base 14: mod_base = OTHER; note = 5-Octadiynyl modified uracil
  misc_feature 1..13: note = RNA
  misc_feature 14: note = DNA
  misc_feature 15..30: note = RNA
agaataaactagatttcttctggtccccaa

SEQ ID NO: 145 - DNA, 30 bp, synthetic construct; identical to SEQ ID NO: 144
  source 1..30: mol_type = other DNA
  modified_base 1: mod_base = OTHER; note = 5'-phosphate modified nucleotide
  modified_base 14: mod_base = OTHER; note = 5-Octadiynyl modified uracil
  misc_feature 1..13: note = RNA
  misc_feature 14: note = DNA
  misc_feature 15..30: note = RNA

SEQ ID NO: 146 - DNA, 30 bp, synthetic construct
  source 1..30: mol_type = other DNA
  modified_base 1: mod_base = OTHER; note = 5'-phosphate modified nucleotide
  modified_base 4: mod_base = OTHER; note = 5-Octadiynyl modified uracil
  misc_feature 1..3: note = RNA
  misc_feature 4: note = DNA
  misc_feature 5..30: note = RNA
agattaaactagtattcttctggtccccaa

SEQ ID NO: 147 - DNA, 30 bp, synthetic construct; identical to SEQ ID NO: 146
  source 1..30: mol_type = other DNA
  modified_base 1: mod_base = OTHER; note = 5'-phosphate modified nucleotide
  modified_base 4: mod_base = OTHER; note = 5-Octadiynyl modified uracil
  misc_feature 1..3: note = RNA
  misc_feature 4: note = DNA
  misc_feature 5..30: note = RNA

SEQ ID NO: 148 - DNA, 11 bp, synthetic construct
  source 1..11: mol_type = other DNA
  modified_base 1: mod_base = OTHER; note = 5'-phosphate modified nucleotide
  modified_base 6: mod_base = OTHER; note = 5-Octadiynyl modified uracil
  modified_base 11: mod_base = OTHER; note = 3'-phosphate modified nucleotide
  misc_feature 1..5: note = RNA
  misc_feature 6: note = DNA
  misc_feature 7..11: note = RNA
aaaaataaaaa

SEQ ID NO: 149 - DNA, 11 bp, synthetic construct
  source 1..11: mol_type = other DNA
  modified_base 1: mod_base = OTHER; note = 5'-phosphate modified nucleotide
  modified_base 6: mod_base = OTHER; note = 5-Octadiynyl modified uracil
  modified_base 11: mod_base = OTHER; note = 3'-phosphate modified nucleotide
  misc_feature 1..5: note = RNA
  misc_feature 6: note = DNA
  misc_feature 7..11: note = RNA
aaaaataaaaa

SEQ ID NO: 150 - DNA, 30 bp, synthetic construct; identical to SEQ ID NO: 62
  source 1..30: mol_type = other DNA
  modified_base 1: mod_base = OTHER; note = 5'-phosphate modified nucleotide
  modified_base 30: mod_base = OTHER; note = 3'-Dideoxy-cytidine
  misc_feature 1..29: note = RNA
  misc_feature 30: note = DNA

SEQ ID NO: 151 - RNA, 29 nt, synthetic construct
  source 1..29: mol_type = other RNA
  modified_base 1: mod_base = OTHER; note = 5'-phosphate modified nucleotide
  modified_base 24: mod_base = OTHER; note = Nucleotide with phosphorothioate linkage
  modified_base 25: mod_base = OTHER; note = Nucleotide with phosphorothioate linkage
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
aaaaaaaaaaaaaaaaaaaaaaaaaaggg

SEQ ID NO: 152 - DNA, 49 bp, synthetic construct
  source 1..49: mol_type = other DNA
  modified_base 1: mod_base = OTHER; note = 5'-phosphate modified nucleotide
  modified_base 44: mod_base = OTHER; note = Nucleotide with phosphorothioate linkage
  modified_base 45: mod_base = OTHER; note = Nucleotide with phosphorothioate linkage
  modified_base 46: mod_base = OTHER; note = Nucleotide with phosphorothioate linkage
  modified_base 47: mod_base = OTHER; note = Nucleotide with phosphorothioate linkage
  modified_base 48: mod_base = OTHER; note = Nucleotide with phosphorothioate linkage
  misc_feature 1..26: note = RNA
  misc_feature 27..49: note = DNA
aaaaaaaaaaaaaaaaaaaaaaaaaatagggttagggttagggttaggg

SEQ ID NO: 153 - RNA, 29 nt, synthetic construct
  source 1..29: mol_type = other RNA
  modified_base 1: mod_base = OTHER; note = 5'-phosphate modified nucleotide
  modified_base 24: mod_base = OTHER; note = Locked nucleic acid
  modified_base 25: mod_base = OTHER; note = Locked nucleic acid
  modified_base 26: mod_base = OTHER; note = Locked nucleic acid
  modified_base 27: mod_base = OTHER; note = Locked nucleic acid
  modified_base 28: mod_base = OTHER; note = Locked nucleic acid
  modified_base 29: mod_base = OTHER; note = Locked nucleic acid
aaaaaaaaaaaaaaaaaaaaaaaaaaggg

SEQ ID NO: 154 - RNA, 29 nt, synthetic construct
  source 1..29: mol_type = other RNA
  modified_base 1: mod_base = OTHER; note = 5'-phosphate modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide with phosphorothioate linkage
  modified_base 25: mod_base = OTHER; note = 2'-O-methyl modified nucleotide with phosphorothioate linkage
  modified_base 26: mod_base = OTHER; note = 2'-O-methyl modified nucleotide with phosphorothioate linkage
  modified_base 27: mod_base = OTHER; note = 2'-O-methyl modified nucleotide with phosphorothioate linkage
  modified_base 28: mod_base = OTHER; note = 2'-O-methyl modified nucleotide with phosphorothioate linkage
  modified_base 29: mod_base = OTHER; note = 2'-O-methyl modified nucleotide
aaaaaaaaaaaaaaaaaaaaaaaaaaggg

SEQ ID NO: 155 - DNA, 30 bp, synthetic construct
  source 1..30: mol_type = other DNA
  modified_base 1: mod_base = OTHER; note = 5'-phosphate modified nucleotide
  modified_base 24: mod_base = OTHER; note = Nucleotide with phosphorothioate linkage
  modified_base 25: mod_base = OTHER; note = Nucleotide with phosphorothioate linkage
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
  modified_base 29: mod_base = OTHER; note = Nucleotide with phosphorothioate linkage
  modified_base 30: mod_base = OTHER; note = 3'-Dideoxy-cytidine
  misc_feature 1..29: note = RNA
  misc_feature 30: note = DNA
aaaaaaaaaaaaaaaaaaaaaaaaaagggc

SEQ ID NO: 156 - DNA, 30 bp, synthetic construct; identical to SEQ ID NO: 155
  source 1..30: mol_type = other DNA
  modified_base 1: mod_base = OTHER; note = 5'-phosphate modified nucleotide
  modified_base 24: mod_base = OTHER; note = Locked nucleic acid
  modified_base 25: mod_base = OTHER; note = Locked nucleic acid
  modified_base 26: mod_base = OTHER; note = Locked nucleic acid
  modified_base 27: mod_base = OTHER; note = Locked nucleic acid
  modified_base 28: mod_base = OTHER; note = Locked nucleic acid
  modified_base 29: mod_base = OTHER; note = Locked nucleic acid
  modified_base 30: mod_base = OTHER; note = 3'-Dideoxy-cytidine
  misc_feature 1..29: note = RNA
  misc_feature 30: note = DNA

SEQ ID NO: 157 - DNA, 30 bp, synthetic construct; identical to SEQ ID NO: 155
  source 1..30: mol_type = other DNA
  modified_base 1: mod_base = OTHER; note = 5'-phosphate modified nucleotide
  modified_base 24: mod_base = OTHER; note = 2'-O-methoxyethyl modified nucleotide with phosphorothioate linkage
  modified_base 25: mod_base = OTHER; note = 2'-O-methoxyethyl modified nucleotide with phosphorothioate linkage
  modified_base 26: mod_base = OTHER; note = 2'-O-methoxyethyl modified nucleotide with phosphorothioate linkage
  modified_base 27: mod_base = OTHER; note = 2'-O-methoxyethyl modified nucleotide with phosphorothioate linkage
  modified_base 28: mod_base = OTHER; note = 2'-O-methoxyethyl modified nucleotide with phosphorothioate linkage
  modified_base 29: mod_base = OTHER; note = 2'-O-methoxyethyl modified nucleotide with phosphorothioate linkage
  modified_base 30: mod_base = OTHER; note = 3'-Dideoxy-cytidine
  misc_feature 1..29: note = RNA
  misc_feature 30: note = DNA

SEQ ID NO: 158 - skipped

SEQ ID NO: 159 - RNA, 1932 nt, synthetic construct
  source 1..1932: mol_type = other RNA
agaaaaaggaataaactagtattcttctggtccccacagactcagagagaacccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtgataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaagcatatgactaaaaaaaaaaaaaaaaaaaaaaaaaaaaaaaaaaaaaaaaaaaaaaaaaaa

SEQ ID NO: 160 - RNA, 12 nt, synthetic construct
  source 1..12: mol_type = other RNA
  modified_base 1: mod_base = OTHER; note = 5'-phosphate thiomorpholino oligo modified nucleotide
agagaataaaaa

SEQ ID NO: 161 - RNA, 12 nt, synthetic construct
  source 1..12: mol_type = other RNA
  modified_base 1: mod_base = OTHER; note = 5'-phosphate modified nucleotide
agagaataaaaa

SEQ ID NO: 162 - RNA, 122 nt, synthetic construct
  source 1..122: mol_type = other RNA
agaataaaatggtattcttctggtccccacatggactacaaagacgacgacgacaagtgagcggctggcggctgttcaagaagattagctaaaaaaaaaaaaaaaaaaaaaaaaaaaaaaaa

SEQ ID NO: 163 - RNA, 122 nt, synthetic construct; identical to SEQ ID NO: 162
  source 1..122: mol_type = other RNA

SEQ ID NO: 164 - RNA, 122 nt, synthetic construct
  source 1..122: mol_type = other RNA
agaataaactaatgttcttctggtccccacatggactacaaagacgacgacgacaagtgagcggctggcggctgttcaagaagattagctaaaaaaaaaaaaaaaaaaaaaaaaaaaaaaaa

SEQ ID NO: 165 - RNA, 122 nt, synthetic construct
  source 1..122: mol_type = other RNA
agaataaactagtaatgttctggtccccacatggactacaaagacgacgacgacaagtgagcggctggcggctgttcaagaagattagctaaaaaaaaaaaaaaaaaaaaaaaaaaaaaaaa

SEQ ID NO: 166 - RNA, 122 nt, synthetic construct
  source 1..122: mol_type = other RNA
agaataaactagtattcatgtggtccccacatggactacaaagacgacgacgacaagtgagcggctggcggctgttcaagaagattagctaaaaaaaaaaaaaaaaaaaaaaaaaaaaaaaa

SEQ ID NO: 167 - RNA, 122 nt, synthetic construct
  source 1..122: mol_type = other RNA
agaataaactagtattcttcatgtccccacatggactacaaagacgacgacgacaagtgagcggctggcggctgttcaagaagattagctaaaaaaaaaaaaaaaaaaaaaaaaaaaaaaaa

SEQ ID NO: 168 - RNA, 500 nt, synthetic construct
  source 1..500: mol_type = other RNA
  misc_difference 1..500: note = This sequence may encompass 25-500 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 169 - RNA, 10 nt, synthetic construct
  source 1..10: mol_type = other RNA
aaaaaaaaaa

SEQ ID NO: 170 - RNA, 11 nt, synthetic construct
  source 1..11: mol_type = other RNA
  modified_base 1: mod_base = OTHER; note = 5'-phosphate modified nucleotide
  modified_base 11: mod_base = OTHER; note = 3' 6-diazynyl-N-heptylhexanamide modified nucleotide
agagataaact

SEQ ID NO: 171 - RNA, 15 nt, synthetic construct
  source 1..15: mol_type = other RNA
  modified_base 1: mod_base = OTHER; note = 5'-phosphate modified nucleotide
  modified_base 15: mod_base = OTHER; note = 3' 6-diazynyl-N-heptylhexanamide modified nucleotide
agagataaactagta

SEQ ID NO: 172 - RNA, 12 nt, synthetic construct
  source 1..12: mol_type = other RNA
  modified_base 1: mod_base = OTHER; note = 5'-phosphate modified 7-methylguanosine
  modified_base 12: mod_base = OTHER; note = 3' 6-diazynyl-N-heptylhexanamide modified nucleotide
gagagataaact

SEQ ID NO: 173 - RNA, 666 nt, synthetic construct
  source 1..666: mol_type = other RNA
ttaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcccactcactatttgttttcgcgcccagttgcaaaaagtgtcggggctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatc

SEQ ID NO: 174 - RNA, 250 nt, synthetic construct
  source 1..250: mol_type = other RNA
acttgctttaacaagttggagatatagtctgctctgcatggtgacatgcagctggatataattccggggtaagattaacgaccttatctgaacataacgctaccgtttaatattgcgtcatataaaaaaaaaaaccaaaaaaaaaaacaaaaaaaaaaataattgactaattaagcagactgtaaatctgctaaaatcgaataaactagtattcttctggtccccacagactcagagagaacccgccacc

SEQ ID NO: 175 - RNA, 53 nt, synthetic construct
  source 1..53: mol_type = other RNA
agaataaactagtattcttctggtccccacagactcagagagaacccgccacc

SEQ ID NO: 176 - DNA, 12 bp, synthetic construct
  source 1..12: mol_type = other DNA
  modified_base 1: mod_base = OTHER; note = 5'-phosphate modified nucleotide
  modified_base 6: mod_base = OTHER; note = 2'-O-methoxyethyl modified nucleotide with phosphorothioate linkage
  modified_base 7: mod_base = OTHER; note = 2'-O-methoxyethyl modified nucleotide with phosphorothioate linkage
  modified_base 8: mod_base = OTHER; note = 2'-O-methoxyethyl modified nucleotide with phosphorothioate linkage
  modified_base 9: mod_base = OTHER; note = 2'-O-methoxyethyl modified nucleotide with phosphorothioate linkage
  modified_base 10: mod_base = OTHER; note = 2'-O-methoxyethyl modified nucleotide with phosphorothioate linkage
  modified_base 11: mod_base = OTHER; note = 2'-O-methoxyethyl modified nucleotide with phosphorothioate linkage
  modified_base 12: mod_base = OTHER; note = 3'-Dideoxy-cytidine
  misc_feature 1..11: note = RNA
  misc_feature 12: note = DNA
aaaaaaaaaaac

SEQ ID NO: 177 - RNA, 21 nt, synthetic construct
  source 1..21: mol_type = other RNA
  modified_base 1: mod_base = OTHER; note = 5'-phosphate modified 7-methylguanosine
gtctgttcttcgcttggtaag

SEQ ID NO: 178 - DNA, 20 bp, synthetic construct
  source 1..20: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 5-Octadiynyl modified uracil
  misc_feature 1..3: note = RNA
  misc_feature 4: note = DNA
  misc_feature 5..20: note = RNA
tcgtccagctcgaccaggat

SEQ ID NO: 179 - RNA, 13 nt, synthetic construct
  source 1..13: mol_type = other RNA
aaaaaaaaaaaaa

SEQ ID NO: 180 - RNA, 17 nt, synthetic construct
  source 1..17: mol_type = other RNA
gcagactgtaaatctgc

SEQ ID NO: 181 - RNA, 35 nt, synthetic construct
  source 1..35: mol_type = other RNA
gagaataaactagtattcttctggtccccacagac

SEQ ID NO: 182 - RNA, 47 nt, synthetic construct
  source 1..47: mol_type = other RNA
aagtctgagtgggcggccaattgaaaaaaaaaaaaaaaaaaaaaaaa

SEQ ID NO: 183 - RNA, 16 nt, synthetic construct
  source 1..16: mol_type = other RNA
gggcaagttggacgcc

SEQ ID NO: 184 - RNA, 24 nt, synthetic construct
  source 1..24: mol_type = other RNA
cccttgggcctccccccagcccct

SEQ ID NO: 185 - RNA, 26 nt, synthetic construct
  source 1..26: mol_type = other RNA
aaaaaaaaaaaaaaaaaaaaaaaaaa

SEQ ID NO: 186 - RNA, 50 nt, synthetic construct
  source 1..50: mol_type = other RNA
agaaaaaaaaaaaaaaaggaataaactagtattcttctggtccccacaga

SEQ ID NO: 187 - RNA, 30 nt, synthetic construct
  source 1..30: mol_type = other RNA
tctggtgggcggccaattgaaaaaaaaaaa

SEQ ID NO: 188 - RNA, 11 nt, synthetic construct
  source 1..11: mol_type = other RNA
ggcaagttgga

SEQ ID NO: 189 - RNA, 15 nt, synthetic construct
  source 1..15: mol_type = other RNA
cctccccccagcccc

SEQ ID NO: 190 - RNA, 15 nt, synthetic construct
  source 1..15: mol_type = other RNA
atggaaagatgccaa

SEQ ID NO: 191 - RNA, 12 nt, synthetic construct
  source 1..12: mol_type = other RNA
atggaaagatgc

SEQ ID NO: 192 - RNA, 15 nt, synthetic construct
  source 1..15: mol_type = other RNA
atggaaagatgccaa

SEQ ID NO: 193 - RNA, 142 nt, synthetic construct
  source 1..142: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
tcgtccagctcgaccaggatagaataaaatggtattcttctggtccccacatggactacaaagacgacgacgacaagtgagcggctggcggctgttcaagaagattagctaaaaaaaaaaaaaaaaaaaaaaaaaaaaaaaa

SEQ ID NO: 194 - RNA, 142 nt, synthetic construct
  source 1..142: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
tcgtccagctcgaccaggatagaataaactaatgttcttctggtccccacatggactacaaagacgacgacgacaagtgagcggctggcggctgttcaagaagattagctaaaaaaaaaaaaaaaaaaaaaaaaaaaaaaaa

SEQ ID NO: 195 - RNA, 142 nt, synthetic construct
  source 1..142: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
tcgtccagctcgaccaggatagaataaactagtaatgttctggtccccacatggactacaaagacgacgacgacaagtgagcggctggcggctgttcaagaagattagctaaaaaaaaaaaaaaaaaaaaaaaaaaaaaaaa

SEQ ID NO: 196 - RNA, 142 nt, synthetic construct
  source 1..142: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
tcgtccagctcgaccaggatagaataaactagtattcatgtggtccccacatggactacaaagacgacgacgacaagtgagcggctggcggctgttcaagaagattagctaaaaaaaaaaaaaaaaaaaaaaaaaaaaaaaa

SEQ ID NO: 197 - RNA, 142 nt, synthetic construct
  source 1..142: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
tcgtccagctcgaccaggatagaataaactagtattcttcatgtccccacatggactacaaagacgacgacgacaagtgagcggctggcggctgttcaagaagattagctaaaaaaaaaaaaaaaaaaaaaaaaaaaaaaaa

SEQ ID NO: 198 - RNA, 13 nt, synthetic construct
  source 1..13: mol_type = other RNA
agatggactacaa

SEQ ID NO: 199 - RNA, 30 nt, synthetic construct
  source 1..30: mol_type = other RNA
aaaaaaaaaaaaaaaaaaaaaaaaaaaaaa